SEQUENCE LISTING (417 sequences)

SEQ ID NO: 1 - DNA, 23 bp, Arabidopsis thaliana
  source 1..23: mol_type = unassigned DNA
ctatccatcctgagtttcattgg

SEQ ID NO: 2 - DNA, 23 bp, Arabidopsis thaliana
  source 1..23: mol_type = unassigned DNA
aagaatctgtaaagctcaggagg

SEQ ID NO: 3 - DNA, 23 bp, Arabidopsis thaliana
  source 1..23: mol_type = unassigned DNA
cttgcagagagaaatcacagtgg

SEQ ID NO: 4 - DNA, 23 bp, Arabidopsis thaliana
  source 1..23: mol_type = unassigned DNA
gcttacacagagaatcacagagg

SEQ ID NO: 5 - DNA, 400 bp, synthetic construct
  misc_feature 1..400: note = Oligo-1: GEiGS_mir173_si-GFP_1
  source 1..400: mol_type = other DNA
gcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtacttaagtcgtgctgcttcatgtggagtggtcaaaaaagttgtagttttcttaaagtctctttcctctccacataagcaggacgagttaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatgg

SEQ ID NO: 6 - DNA, 400 bp, synthetic construct
  misc_feature 1..400: note = Oligo-2: GEiGS_mir173_si-GFP_2
  source 1..400: mol_type = other DNA
gcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtacttagttgtactccagcttgtgccagtggtcaaaaaagttgtagttttcttaaagtctctttcctctggcaaagctgcagtacaactaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatgg

SEQ ID NO: 7 - DNA, 400 bp, synthetic construct
  misc_feature 1..400: note = Oligo-3: GEiGS_mir173_si-PDS_1
  source 1..400: mol_type = other DNA
gcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttatccacacaaactacctgcaagtggtcaaaaaagttgtagttttcttaaagtctctttcctcttgcagtagttagtgtggataaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatgg

SEQ ID NO: 8 - DNA, 400 bp, synthetic construct
  misc_feature 1..400: note = Oligo-4: GEiGS_mir173_si-PDS_2
  source 1..400: mol_type = other DNA
gcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttgacaatccagccaatccagcagtggtcaaaaaagttgtagttttcttaaagtctctttcctctgctgattggcaggattgtcaaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatgg

SEQ ID NO: 9 - DNA, 400 bp, synthetic construct
  misc_feature 1..400: note = Oligo-5: GEiGS_mir390a_si-GFP_1
  source 1..400: mol_type = other DNA
agaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaaagtcgtgctgcttcatgtggatgatgatcacattcgttatctattttttccacatgaagaagcacgacttgattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaa

SEQ ID NO: 10 - DNA, 400 bp, synthetic construct
  misc_feature 1..400: note = Oligo-6: GEiGS_mir390a_si-GFP_2
  source 1..400: mol_type = other DNA
agaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaagttgtactccagcttgtgccatgatgatcacattcgttatctattttttggcacaagcttgagtacaactgattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaa

SEQ ID NO: 11 - DNA, 400 bp, synthetic construct
  misc_feature 1..400: note = Oligo-7: GEiGS_mir390a_si-PDS_1
  source 1..400: mol_type = other DNA
agaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatatccacacaaactacctgcaatgatgatcacattcgttatctatttttttgcaggtagtgtgtgtggatagattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaa

SEQ ID NO: 12 - DNA, 400 bp, synthetic construct
  misc_feature 1..400: note = Oligo-8: GEiGS_mir390a_si-PDS_2
  source 1..400: mol_type = other DNA
agaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatgacaatccagccaatccagcatgatgatcacattcgttatctattttttgctggattggatggattgtcagattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaa

SEQ ID NO: 13 - DNA, 1005 bp, synthetic construct
  misc_feature 1..1005: note = CaMV-35S-promoter nucleic acid sequence
  source 1..1005: mol_type = other DNA
tttggagaggacaggcttcttgagatccttcaacaattaccaacaacaacaaacaacaaacaacattacaattactatttacaattacagtcgactctagaggatccatggtgagcaagggcgaggagctgttcaccggggtggtgcccatcctggtcgagctggacggcgacgtaaacggccacaagttcagcgtgagaggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctctttcaaggacgacggcacttacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcactgccgacaagcagaagaacggcatcaaggccaacttcaagatccgccacaacgttgaggacggcagcgtgcagctcgccgaccactaccagcagaacacccccatcggcgacggccccgtgctgctgcccgacaaccactacctgagcacccagtccgttctgagcaaagaccccaacgagaagcgcgatcacatggtcctgctggagttcgtgaccgccgccgggatcactctcggcatggacgagctgtacaagtaaagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcc

SEQ ID NO: 14 - DNA, 855 bp, synthetic construct
  misc_feature 1..855: note = NOS terminator nucleic acid sequence
  source 1..855: mol_type = other DNA
gctcgtccatgccgagagtgatcccggcggcggtcacgaactccagcaggaccatgtgatcgcgcttctcgttggggtctttgctcagaacggactgggtgctcaggtagtggttgtcgggcagcagcacggggccgtcgccgatgggggtgttctgctggtagtggtcggcgagctgcacgctgccgtcctcaacgttgtggcggatcttgaagttggccttgatgccgttcttctgcttgtcggcagtgatatagacgttgtggctgttgaagttgtactccagcttgtgccccaggatgttgccgtcctccttgaagtcgatgcccttcagctcgatgcggttcaccagggtgtcgccctcgaacttcacctcggcgcgggtcttgtaagtgccgtcgtccttgaaagagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgttggtggcatcgccctcgccctcgcctctcacgctgaacttgtggccgtttacgtcgccgtccagctcgaccaggatgggcaccaccccggtgaacagctcctcgcccttgctcaccatggatcctctagagtcgactgtaattgtaaatagtaattgtaatgttgtttgttgtttgttgttgttggtaattgttgaaggatctcaagaagcctgtcctctccaaatgaaatgaacttccttatatagaggaagggtcttgcgaa

SEQ ID NO: 15 - DNA, 215 bp, synthetic construct
  misc_feature 1..215: note = CaMV-35S terminator nucleic acid sequence
  source 1..215: mol_type = other DNA
cgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggc

SEQ ID NO: 16 - DNA, 216 bp, synthetic construct
  misc_feature 1..216: note = G7-ter nucleic acid sequence
  source 1..216: mol_type = other DNA
gatcccccgtcgacagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagat

SEQ ID NO: 17 - DNA, 516 bp, synthetic construct
  misc_feature 1..516: note = CsVMV promoter nucleic acid sequence
  source 1..516: mol_type = other DNA
ccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgatgacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgt

SEQ ID NO: 18 - DNA, 636 bp, Arabidopsis thaliana
  source 1..636: mol_type = unassigned DNA
gtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtagattaatctgagtttttctgattaacag

SEQ ID NO: 19 - DNA, 4140 bp, synthetic construct
  misc_feature 1..4140: note = pco_Cas9_NLS nucleic acid sequence
  source 1..4140: mol_type = other DNA
atggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttga

SEQ ID NO: 20 - DNA, 263 bp, synthetic construct
  misc_feature 1..263: note = AtuNos ter nucleic acid sequence
  source 1..263: mol_type = other DNA
gtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcga

SEQ ID NO: 21 - DNA, 1147 bp, synthetic construct
  misc_feature 1..1147: note = Synthetic construct clone eGFP-OsP5SM_E/R eGFP (eGFP) gene
  source 1..1147: mol_type = other DNA
atgtctagagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggaggtagatttatgcatcctcttgtcatgagaagtcgaattgttcccattctgtgtgttgcagctacagatggagatacatagagatactcgtggattttgcttagtgttgagttttgttctggttgtgaactaaaagtttatacatttgcaggaaataaatagccttttgtttaaatcaaaaggtcttacctatgttagtgtgaagcattggatcccaaagaactccaaaatgcgatgaggcatatttaatcttgtctggactagtaacaggttgggatgaccacctgtgaagctccaacaggattgcctcctcacgcaatgtttgaggtctgatgttcaatagcttgttttgtttcactttgctttggactttcttttcgccaatgagctatgtttctgatggttttcactcttttggtgtgtagagaaccatcttcttcaaggacgacggcaactacaagacccgcgccgaggtgaagttcgagggcgacaccctggtgaaccgcatcgagctgaagggcatcgacttcaaggaggacggcaacacctggggcacaagctggagtacaactacaacagccacaacgtctatatcatggccgacaagcagaagaaggcatcaaggtgaacttcaagatccgccacaacatcgaggacggcagcgtgcagctcgccgaccactacagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 22 - DNA, 2074 bp, Arabidopsis thaliana
  source 1..2074: mol_type = unassigned DNA
caattatgtgttaaagatacaaacttttgtctgatttgcttccaccggtttcacctaagatactcaattttcttactttttgtgtgttttgtaattctaattcttttatagcttcaatttttagattcattgaagcagttgtgagttaagttggagaaaatggttgtgtttgggaatgtttctgcggcgaatttgccttatcaaaacgggtttttggaggcactttcatctggaggttgtgaactaatgggacatagctttagggttcccacttctcaagcgcttaagacaagaacaaggaggaggagtactgctggtcctttgcaggtagtttgtgtggatattccaaggccagagctagagaacactgtcaatttcttggaagctgctagtttatctgcatccttccgtagtgctcctcgtcctgctaagcctttgaaagttgtaattgctggtgctggattggctggattgtcaactgcaaagtacctggctgatgcaggccacaaacctctgttgcttgaagcaagagatgttcttggtggaaagatagctgcatggaaggatgaagatggggactggtatgagactggtttacatattttcttcggtgcttatccgaatgtgcagaatttatttggagaacttgggatcaatgatcggttgcagtggaaggaacactccatgatttttgctatgccaagtaaacctggagaatttagtagatttgacttcccagatgtcctaccagcacccttaaatggtatttgggctattttgcggaacaacgagatgctgacatggccagagaaaataaagtttgctattggacttttgccagccatggtcggcggtcaggcttatgttgaggcccaagatggtttatcagtcaaagaatggatggaaaagcagggagtacctgagcgcgtgaccgacgaggtgtttattgccatgtcaaaggcgctaaactttataaaccctgatgaactgtcaatgcaatgcattttgatagctttgaaccggtttcttcaggaaaaacatggttccaagatggcattcttggatggtaatcctccggaaaggctttgtatgccagtagtggatcatattcgatcactaggtggggaagtgcaacttaattctaggataaagaaaattgagctcaatgacgatggcacggttaagagtttcttactcactaatggaagcactgtcgaaggagacgcttatgtgtttgccgctccagtcgatatcctgaagctccttttaccagatccctggaaagaaataccgtacttcaagaaattggataaattagttggagtaccagttattaatgttcatatatggtttgatcgaaaactgaagaacacatatgatcacctactctttagcagaagtaaccttctgagcgtgtatgccgacatgtccttaacttgtaaggaatattacgatcctaaccggtcaatgctggagctagtatttgcaccagcagaggaatggatatcacggactgattctgacatcatagatgcaacaatgaaagaacttgagaaactcttccctgatgaaatctcagctgaccaaagcaaagctaaaattctgaagtaccatgtcgttaagactccaagatctgtgtacaagaccatcccaaactgtgaaccatgtcgtcctctacaaagatcacctattgaaggattctacttagctggagattacacaaaacagaagtacttagcttccatggaaggcgctgtcctctctggcaaattctgctctcagtctattgttcaggattacgagctactggctgcgtctggaccaagaaagttgtcggaggcaacagtatcatcatcatgagaagaggacaaaacttaaagatgatttgcttgtaagcattattatttgtgtataaatctcattgcaatccaaacttaaccttactctcttcagtaaatgaatctcacagatttgacatctcacgtttctgtcaattttataatttttaaaaagtaattactgtcgaccttttgtaatcatagtgatttatcattatgtctctctttttaaaacc

SEQ ID NO: 23 - DNA, 1419 bp, Arabidopsis thaliana
  source 1..1419: mol_type = unassigned DNA
ggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtgagacggatatcaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacatactagagaaagaggagaaatactagatggcttcctccgaggacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaggacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaggacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaggactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatacgtctcagttttagagctagaaatagcaagttaaaataaggctagtccgttatcaacttgaaaaagtggcaccgagtcggtgctttttttctagacccagctttcttgtacaaagttggcattacgct

SEQ ID NO: 24 - DNA, 1286 bp, synthetic construct
  misc_feature 1..1286: note = U6III-synthetic pol 3 promoter for sgRNA expression
  source 1..1286: mol_type = other DNA
ggagtatgatcaaaagtcccacatcgatcaggtgatatatagcagcttagtttatataatgatagagtcgacatagcgattgggagacgcaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacatactagagaaagaggagaaatactagatggcttcctccgaggacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaggacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaggacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaggactacaccatcgttgaacagtacgaacgtgctgaaggtcgtcactccaccggtgcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatacgtctccgttttagagctagaaatagcaagttaaaataaggctagtccgttatcaacttgaaaaagtggcaccgagtcggtgctttttttctagacccagctttcttgtacaaagttggcattacgct

SEQ ID NO: 25 - DNA, 2074 bp, Arabidopsis thaliana; identical to SEQ ID NO: 22
  source 1..2074: mol_type = unassigned DNA

SEQ ID NO: 26 - DNA, 2290 bp, Arabidopsis thaliana
  source 1..2290: mol_type = unassigned DNA
ctttggtgggcaaaaacatattagctgagaggtcaatttctttttcccctaaaccaaattacgttgagatgcatggtctctctctactcaattaaccaaataaggaaaagaatcatatggtcatcaattcgtaaatcaaaattttaatttgtgtggtatttaatccatctacatgtttcgtaagcaacaaaagagcttggtctgaaaaccaaacaagaccatatgggcactcgaatactccattttgttatcggctacttccactagcctcctccttcgctgcgtctcctgtttctctacttcacgattactcgctagattcattgaagcagttgtgagttaagttggagaaaatggttgtgtttgggaatgtttctgcggcgaatttgccttatcaaaacgggtttttggaggcactttcatctggaggttgtgaactaatgggacatagctttagggttcccacttctcaagcgcttaagacaagaacaaggaggaggagtactgctggtcctttgcaggtagtttgtgtggatattccaaggccagagctagagaacactgtcaatttcttggaagctgctagtttatctgcatccttccgtagtgctcctcgtcctgctaagcctttgaaagttgtaattgctggtgctggattggctggattgtcaactgcaaagtacctggctgatgcaggccacaaacctctgttgcttgaagcaagagatgttcttggtggaaagatagctgcatggaaggatgaagatggggactggtatgagactggtttacatattttcttcggtgcttatccgaatgtgcagaatttatttggagaacttgggatcaatgatcggttgcagtggaaggaacactccatgatttttgctatgccaagtaaacctggagaatttagtagatttgacttcccagatgtcctaccagcacccttaaatggtatttgggctattttgcggaacaacgagatgctgacatggccagagaaaataaagtttgctattggacttttgccagccatggtcggcggtcaggcttatgttgaggcccaagatggtttatcagtcaaagaatggatggaaaagcagggagtacctgagcgcgtgaccgacgaggtgtttattgccatgtcaaaggcgctaaactttataaaccctgatgaactgtcaatgcaatgcattttgatagctttgaaccggtttcttcaggaaaaacatggttccaagatggcattcttggatggtaatcctccggaaaggctttgtatgccagtagtggatcatattcgatcactaggtggggaagtgcaacttaattctaggataaagaaaattgagctcaatgacgatggcacggttaagagtttcttactcactaatggaagcactgtcgaaggagacgcttatgtgtttgccgctccagtcgatatcctgaagctccttttaccagatccctggaaagaaataccgtacttcaagaaattggataaattagttggagtaccagttattaatgttcatatatggtttgatcgaaaactgaagaacacatatgatcacctactctttagcagaagtaaccttctgagcgtgtatgccgacatgtccttaacttgtaaggaatattacgatcctaaccggtcaatgctggagctagtatttgcaccagcagaggaatggatatcacggactgattctgacatcatagatgcaacaatgaaagaacttgagaaactcttccctgatgaaatctcagctgaccaaagcaaagctaaaattctgaagtaccatgtcgttaagactccaagatctgtgtacaagaccatcccaaactgtgaaccatgtcgtcctctacaaagatcacctattgaaggattctacttagctggagattacacaaaacagaagtacttagcttccatggaaggcgctgtcctctctggcaaattctgctctcagtctattgttcaggattacgagctactggctgcgtctggaccaagaaagttgtcggaggcaacagtatcatcatcatgagaagaggacaaaacttaaagatgatttgcttgtaagcattattatttgtgtataaatctcattgcaatccaaacttaaccttactctcttcagtaaatgaatctcacagatttgacatctcacgtttctgtcaattttataatttttaaaaagtaattactgtcgaccttttgtaatcatagtgatttatcattatgtctctctttttaaaaccttttctggtacaaattataaaa

SEQ ID NO: 27 - protein, 241 aa, synthetic construct
  REGION 1..241: note = eGFP [synthetic construct] amino acid sequence
MSRVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 28 - RNA, 107 nt, Arabidopsis thaliana
  source 1..107: mol_type = unassigned RNA
gtagagaagaatctgtaaagctcaggagggatagcgccatgatgatcacattcgttatctattttttggcgctatccatcctgagtttcattggctcttcttactac

SEQ ID NO: 29 - RNA, 102 nt, Arabidopsis thaliana
  source 1..102: mol_type = unassigned RNA
taagtactttcgcttgcagagagaaatcacagtggtcaaaaaagttgtagttttcttaaagtctctttcctctgtgattctctgtgtaagcgaaagagcttg

SEQ ID NO: 30 - DNA, 711 bp, synthetic construct
  misc_feature 1..711: note = nucleic acid sequence encoding mCherry
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggcccagtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 31 - DNA, 4100 bp, synthetic construct
  misc_feature 1..4100: note = Plasmid-1: GEiGS_ mir173_si-GFP_1
  source 1..4100: mol_type = other DNA
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtacttaagtcgtgctgcttcatgtggagtggtcaaaaaagttgtagttttcttaaagtctctttcctctccacataagcaggacgagttaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 32 - DNA, 4100 bp, synthetic construct
  misc_feature 1..4100: note = Plasmid-2: GEiGS_ mir173_si-GFP_2
  source 1..4100: mol_type = other DNA
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtacttagttgtactccagcttgtgccagtggtcaaaaaagttgtagttttcttaaagtctctttcctctggcaaagctgcagtacaactaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 33 - DNA, 4100 bp, synthetic construct
  misc_feature 1..4100: note = Plasmid-3: GEiGS_ mir173_si-PDS_1
  source 1..4100: mol_type = other DNA
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttatccacacaaactacctgcaagtggtcaaaaaagttgtagttttcttaaagtctctttcctcttgcagtagttagtgtggataaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 34 - DNA, 4100 bp, synthetic construct
  misc_feature 1..4100: note = Plasmid-4: GEiGS_ mir173_si-PDS_2
  source 1..4100: mol_type = other DNA
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttgacaatccagccaatccagcagtggtcaaaaaagttgtagttttcttaaagtctctttcctctgctgattggcaggattgtcaaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 35 - DNA, 4000 bp, synthetic construct
  misc_feature 1..4000: note = Plasmid-5: GEiGS_ mir390a _si-GFP_1
  source 1..4000: mol_type = other DNA
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaaagtcgtgctgcttcatgtggatgatgatcacattcgttatctattttttccacatgaagaagcacgacttgattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 36 - DNA, 4000 bp, synthetic construct
  misc_feature 1..4000: note = Plasmid-6: GEiGS_ mir390a _si-GFP_2
  source 1..4000: mol_type = other DNA
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaagttgtactccagcttgtgccatgatgatcacattcgttatctattttttggcacaagcttgagtacaactgattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 37 - DNA, 4000 bp, synthetic construct
  misc_feature 1..4000: note = Plasmid-7: GEiGS_ mir390a _si-PDS_1
  source 1..4000: mol_type = other DNA
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatatccacacaaactacctgcaatgatgatcacattcgttatctatttttttgcaggtagtgtgtgtggatagattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 38 - DNA, 4000 bp, synthetic construct
  misc_feature 1..4000: note = Plasmid-8: GEiGS_ mir390a _si-PDS_2
  source 1..4000: mol_type = other DNA
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatgacaatccagccaatccagcatgatgatcacattcgttatctattttttgctggattggatggattgtcagattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 39 - DNA, 22 bp, synthetic construct
  misc_feature 1..22: note = Single strand oligonucleotide
  source 1..22: mol_type = other DNA
ttcgcttgcagagagaaatcac

SEQ ID NO: 40 - DNA, 21 bp, synthetic construct
  misc_feature 1..21: note = Single strand oligonucleotide
  source 1..21: mol_type = other DNA
aagctcaggagggatagcgcc

SEQ ID NO: 41 - DNA, 23 bp, synthetic construct
  misc_feature 1..23: note = Single strand oligonucleotide
  source 1..23: mol_type = other DNA
cttgcagagagaaatcacagtgg

SEQ ID NO: 42 - DNA, 23 bp, synthetic construct
  misc_feature 1..23: note = Single strand oligonucleotide
  source 1..23: mol_type = other DNA
gcttacacagagaatcacagagg

SEQ ID NO: 43 - DNA, 23 bp, synthetic construct
  misc_feature 1..23: note = Single strand oligonucleotide
  source 1..23: mol_type = other DNA
aagaatctgtaaagctcaggagg

SEQ ID NO: 44 - DNA, 23 bp, synthetic construct
  misc_feature 1..23: note = Single strand oligonucleotide
  source 1..23: mol_type = other DNA
ctatccatcctgagtttcattgg

SEQ ID NO: 45 - DNA, 21 bp, synthetic construct
  misc_feature 1..21: note = Single strand oligonucleotide
  source 1..21: mol_type = other DNA
aagtcgtgctgcttcatgtgg

SEQ ID NO: 46 - DNA, 22 bp, synthetic construct
  misc_feature 1..22: note = Single strand oligonucleotide
  source 1..22: mol_type = other DNA
ccacataagcaggacgagttaa

SEQ ID NO: 47 - DNA, 21 bp, synthetic construct
  misc_feature 1..21: note = Single strand oligonucleotide
  source 1..21: mol_type = other DNA
agttgtactccagcttgtgcc

SEQ ID NO: 48 - DNA, 21 bp, synthetic construct
  misc_feature 1..21: note = Single strand oligonucleotide
  source 1..21: mol_type = other DNA
gcaaagctgcagtacaactaa

SEQ ID NO: 49 - DNA, 21 bp, synthetic construct
  misc_feature 1..21: note = Single strand oligonucleotide
  source 1..21: mol_type = other DNA
tatccacacaaactacctgca

SEQ ID NO: 50 - DNA, 21 bp, synthetic construct
  misc_feature 1..21: note = Single strand oligonucleotide
  source 1..21: mol_type = other DNA
gcagtagttagtgtggataaa

SEQ ID NO: 51 - DNA, 21 bp, synthetic construct
  misc_feature 1..21: note = Single strand oligonucleotide
  source 1..21: mol_type = other DNA
tgacaatccagccaatccagc

SEQ ID NO: 52 - DNA, 21 bp, synthetic construct
  misc_feature 1..21: note = Single strand oligonucleotide
  source 1..21: mol_type = other DNA
ctgattggcaggattgtcaaa

SEQ ID NO: 53 - DNA, 21 bp, synthetic construct
  misc_feature 1..21: note = Single strand oligonucleotide
  source 1..21: mol_type = other DNA
taaagatcggcaacacatgat

SEQ ID NO: 54 - DNA, 19 bp, synthetic construct
  misc_feature 1..19: note = Single strand oligonucleotide
  source 1..19: mol_type = other DNA
tcagtgttggcgatcttta

SEQ ID NO: 55 - DNA, 21 bp, synthetic construct
  misc_feature 1..21: note = Single strand oligonucleotide
  source 1..21: mol_type = other DNA
tgacctttcttgggtttagcc

SEQ ID NO: 56 - DNA, 19 bp, synthetic construct
  misc_feature 1..19: note = Single strand oligonucleotide
  source 1..19: mol_type = other DNA
gctaacccatgaaaggtca

SEQ ID NO: 57 - DNA, 102 bp, synthetic construct
  misc_feature 1..102: note = Single strand oligonucleotide
  source 1..102: mol_type = other DNA
taagtactttcgcttgcagagagaaatcacagtggtcaaaaaagttgtagttttcttaaagtctctttcctctgtgattctctgtgtaagcgaaagagcttg

SEQ ID NO: 58 - DNA, 102 bp, synthetic construct
  misc_feature 1..102: note = Single strand oligonucleotide
  source 1..102: mol_type = other DNA
taagtacttaagtcgtgctgcttcatgtggagtggtcaaaaaagttgtagttttcttaaagtctctttcctctccacataagcaggacgagttaagagcttg

SEQ ID NO: 59 - DNA, 102 bp, synthetic construct
  misc_feature 1..102: note = Single strand oligonucleotide
  source 1..102: mol_type = other DNA
taagtacttagttgtactccagcttgtgccagtggtcaaaaaagttgtagttttcttaaagtctctttcctctggcaaagctgcagtacaactaagagcttg

SEQ ID NO: 60 - DNA, 102 bp, synthetic construct
  misc_feature 1..102: note = Single strand oligonucleotide
  source 1..102: mol_type = other DNA
taagtactttatccacacaaactacctgcaagtggtcaaaaaagttgtagttttcttaaagtctctttcctcttgcagtagttagtgtggataaagagcttg

SEQ ID NO: 61 - DNA, 102 bp, synthetic construct
  misc_feature 1..102: note = Single strand oligonucleotide
  source 1..102: mol_type = other DNA
taagtactttgacaatccagccaatccagcagtggtcaaaaaagttgtagttttcttaaagtctctttcctctgctgattggcaggattgtcaaagagcttg

SEQ ID NO: 62 - DNA, 102 bp, synthetic construct
  misc_feature 1..102: note = Single strand oligonucleotide
  source 1..102: mol_type = other DNA
taagtactttaaagatcggcaacacatgatagtggtcaaaaaagttgtagttttcttaaagtctctttcctctgatcagtgttggcgatctttaagagcttg

SEQ ID NO: 63 - DNA, 102 bp, synthetic construct
  misc_feature 1..102: note = Single strand oligonucleotide
  source 1..102: mol_type = other DNA
taagtactttgacctttcttgggtttagccagtggtcaaaaaagttgtagttttcttaaagtctctttcctctgggctaacccatgaaaggtcaagagcttg

SEQ ID NO: 64 - DNA, 107 bp, synthetic construct
  misc_feature 1..107: note = Single strand oligonucleotide
  source 1..107: mol_type = other DNA
gtagagaagaatctgtaaagctcaggagggatagcgccatgatgatcacattcgttatctattttttggcgctatccatcctgagtttcattggctcttcttactac

SEQ ID NO: 65 - DNA, 107 bp, synthetic construct
  misc_feature 1..107: note = Single strand oligonucleotide
  source 1..107: mol_type = other DNA
gtagagaagaatctgtaaagtcgtgctgcttcatgtggatgatgatcacattcgttatctattttttccacatgaagaagcacgacttgattggctcttcttactac

SEQ ID NO: 66 - DNA, 107 bp, synthetic construct
  misc_feature 1..107: note = Single strand oligonucleotide
  source 1..107: mol_type = other DNA
gtagagaagaatctgtaagttgtactccagcttgtgccatgatgatcacattcgttatctattttttggcacaagcttgagtacaactgattggctcttcttactac

SEQ ID NO: 67 - DNA, 107 bp, synthetic construct
  misc_feature 1..107: note = Single strand oligonucleotide
  source 1..107: mol_type = other DNA
gtagagaagaatctgtatatccacacaaactacctgcaatgatgatcacattcgttatctatttttttgcaggtagtgtgtgtggatagattggctcttcttactac

SEQ ID NO: 68 - DNA, 107 bp, synthetic construct
  misc_feature 1..107: note = Single strand oligonucleotide
  source 1..107: mol_type = other DNA
gtagagaagaatctgtatgacaatccagccaatccagcatgatgatcacattcgttatctattttttgctggattggatggattgtcagattggctcttcttactac

SEQ ID NO: 69 - DNA, 107 bp, synthetic construct
  misc_feature 1..107: note = Single strand oligonucleotide
  source 1..107: mol_type = other DNA
gtagagaagaatctgtataaagatcggcaacacatgatatgatgatcacattcgttatctattttttatcatgtgttaccgatctttacattggctcttcttactac

SEQ ID NO: 70 - DNA, 107 bp, synthetic construct
  misc_feature 1..107: note = Single strand oligonucleotide
  source 1..107: mol_type = other DNA
gtagagaagaatctgtatgacctttcttgggtttagccatgatgatcacattcgttatctattttttggctaaaccccagaaaggtcacattggctcttcttactac

SEQ ID NO: 71 - DNA, 4100 bp, synthetic construct
  misc_feature 1..4100: note = Single strand oligonucleotide
  source 1..4100: mol_type = other DNA
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttcgcttgcagagagaaatcacagtggtcaaaaaagttgtagttttcttaaagtctctttcctctgtgattctctgtgtaagcgaaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 72 - DNA, 4100 bp, synthetic construct; identical to SEQ ID NO: 31
  misc_feature 1..4100: note = Single strand oligonucleotide
  source 1..4100: mol_type = other DNA

SEQ ID NO: 73 - DNA, 4100 bp, synthetic construct; identical to SEQ ID NO: 32
  misc_feature 1..4100: note = Single strand oligonucleotide
  source 1..4100: mol_type = other DNA

SEQ ID NO: 74 - DNA, 4100 bp, synthetic construct; identical to SEQ ID NO: 33
  misc_feature 1..4100: note = Single strand oligonucleotide
  source 1..4100: mol_type = other DNA

SEQ ID NO: 75 - DNA, 4100 bp, synthetic construct; identical to SEQ ID NO: 34
  misc_feature 1..4100: note = Single strand oligonucleotide
  source 1..4100: mol_type = other DNA

SEQ ID NO: 76 - DNA, 4100 bp, synthetic construct
  misc_feature 1..4100: note = Single strand oligonucleotide
  source 1..4100: mol_type = other DNA
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttaaagatcggcaacacatgatagtggtcaaaaaagttgtagttttcttaaagtctctttcctctgatcagtgttggcgatctttaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 77 - DNA, 4100 bp, synthetic construct
  misc_feature 1..4100: note = Single strand oligonucleotide
  source 1..4100: mol_type = other DNA
cacttatcatttagacagtagattttaaatttgtatttacaatttcaaaactgaaattcatttgtaatcaaagaaaaacaaaacaagaaaagggaggaggagtgggatttgggttgcttaacagtattatatatacacgtcgttagttaatcaataaattatgagcaggtgtagtagctatgaacatggttttaccaaattggtaaattcatgtcattgttgtagcactttagcgaggccagcaaaatggcgttcttggaaagggcatggcggcccgtggccgccagggatatgtgtgcagctagaaggattagaaggagtaacttcacctttttgtagaaactgtaaattgccaagacgctcgtttggtaaataccttaacgcttcgtttgctggaatgtgtaccaccattagtagaagaaagtataataagactattaatgcaagagattttttcatattccttttctaagagtagaatggaatgaataaatgaatgaatgaaatagggtttttcttgtttagatcctgtcgcacccgagaataatagaagctgaattaattggtgaagagttttatggtggcgatgttgtatttatagagggaaatgatttcaagtttacaatgggatttttaatttgttttgttgactattatcttgcggagagatcttgactgttgacatgtatgtagtgtttgttattaaaataaatgcatttttgtagacccgattcttaaatatgttaggcatggtcaaattgttagactgtaaaagcttgagtagagacatcggtcaactttgtttacaagaattctctataaaatattatacaaattgatcggtagaaattagatggaacattttgaattaatgtttgacaatgtataaattggtttggtcaatatctaggaatgaaaacaagagctgcttcaaagttgttccattcttaagtatacatagaggtggcttgatgggaagtttcatggaagtgtagttttatatctacttccaaattcgttgatctgtttcttatctcaagctaaacatgtttagaaaagatgtgttaaaaacatgtgacaaaaaaagagtcaaatgtttggcaaacaaggtacttagatttttggcatctattcaaatataatagaatcccaaaattatgatatttgtcaatcaaatttgaaaaaaaaaaaaaaaaaaaaaaagattctacaaaagatcaaacgtattgccgtagatttatcataattttaattctttcacactaccactagtccactaccatatagtgatgagataatccataatcataaataagatataactttcgaattcttgtttttgttgcctcaaagttgttggatcattattttttacgtaaatgtggcttaatgagaatttatgtttgtgggaattgtagtttgcttccaactttttttttttttttttgaacacgtagtttgcttccaacttagtttatctttttcttatttcaagttaaacatgtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttgacctttcttgggtttagccagtggtcaaaaaagttgtagttttcttaaagtctctttcctctgggctaacccatgaaaggtcaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagaccccggtgtagtaaattgacctcacaattcactattgtattgtatacgtacatttcaagcgtaattaaaccctcatatttttatacgctttaaatataattggcctttaattagctcaaataaactagatgtcgtacgtgatcacggtggatgaaatcaatggtattatgaaaagactgtacatgatttcaaatattttaatgtggtcgtaaaaattgttgtttatagtggaaattgaagacaacaacgttactgaaacacatacagattgaaatttcgatcatttacttgcaaagtgttaccgtggaggcgtggcgtggacgtaaggtaccataatggtttgtgttacagtcacgccactacactcgaattcaagctaccatattataatacgttgttgattagaaaaaatagtcgccaattttctttaaaacaaatttcagtttttatttgtcagcaaaaaaaacttactaacacaacggaagaacacaaaaattagggagttgctcacagagcaaaagtaatagaaatgggaaaagctaatatacgtccgagtaggaaactaatcttgcaaaaactgatgaaagcaatcagaagccttgacgtttgtctggagagaggaattgtgttttggatcactttgttcagtttgttgtggatcgtcttccgttacgttctcaccaaaaatatttcataatgaaacaaaaaaattaattaataatggtagcttagaatgccaagttgagaacagatttgcagtttgtcccggaatgatcaagtagagcattcatagtgtcttgccaatatggtgtgatcaacgaagtttgacaaaaccgtgaagatataggaacatgtaatcatgcggctctccatacaatacatcttgttgacaaagttcccaagacctcattctacaaaccaatgtttcttttttctttttctttttggtgatagtttttgcaatcaaatgttgtaaaactatgattggaaatactactgtattttaccgaaaactttattatatatagtcattaacactcattaccaatcacataatcaatagtctattttgattatacaacttttaaacaataaagacatgtttatacagatttggtttaaattagtactccctatattttagaaaattttattttgtttcatattatagaatgtcttggaaagttctatgtaaatttaaatgtatttagtaacttgtgacgattttatattatgtagtcttttttaggatttgttgattttttaaaataaattttttaaagaaaaaaaacaaattattttaataaacatgcctttaccttatacagtttatattttgaaagagagatagtatgttttaggatatatttaaagaaaaaaaaataactctttaattta

SEQ ID NO: 78 - DNA, 4000 bp, synthetic construct
  misc_feature 1..4000: note = Single strand oligonucleotide
  source 1..4000: mol_type = other DNA
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtaaagctcaggagggatagcgccatgatgatcacattcgttatctattttttggcgctatccatcctgagtttcattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 79 - DNA, 4000 bp, synthetic construct; identical to SEQ ID NO: 35
  misc_feature 1..4000: note = Single strand oligonucleotide
  source 1..4000: mol_type = other DNA

SEQ ID NO: 80 - DNA, 4000 bp, synthetic construct; identical to SEQ ID NO: 36
  misc_feature 1..4000: note = Single strand oligonucleotide
  source 1..4000: mol_type = other DNA

SEQ ID NO: 81 - DNA, 4000 bp, synthetic construct; identical to SEQ ID NO: 37
  misc_feature 1..4000: note = Single strand oligonucleotide
  source 1..4000: mol_type = other DNA

SEQ ID NO: 82 - DNA, 4000 bp, synthetic construct; identical to SEQ ID NO: 38
  misc_feature 1..4000: note = Single strand oligonucleotide
  source 1..4000: mol_type = other DNA

SEQ ID NO: 83 - DNA, 4000 bp, synthetic construct
  misc_feature 1..4000: note = Single strand oligonucleotide
  source 1..4000: mol_type = other DNA
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtataaagatcggcaacacatgatatgatgatcacattcgttatctattttttatcatgtgttaccgatctttacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 84 - DNA, 4000 bp, synthetic construct
  misc_feature 1..4000: note = Single strand oligonucleotide
  source 1..4000: mol_type = other DNA
accctcattgattttggtggccaaactcttgatcgtattcttttaggaacagtaattaattttaaatttgagaaaaatgtagtgatgaaagtgagatatatgggacgcactattgaaagcattgtccacaccgcatgatgtgagattgatgtgaatatggtaactaataacacccagactcacttttatgagaaaatgtatcttaaaaaatgttattcatcaaagcttagatgcgctataggggaacataatcattatttgaaaagaaccaattcaaatatttttttttttaagagttaaaatcctatataacataaccatccaaactttgggcatgaacacaacaatattttttttcgtttttgataaattctttcaattgcaatcaaaacttaccagctatacaaatgtttgctgcatccaatctaatacaccgttaaacaataaaatttgttatctctcaaatctgatcatctattccaacctgactgttttattctagtattttaaggccaggtataacgaaaacaaagaaaaaagttcagcgaccaagacattttgacatcaggctcaaacggtcaaacccacgaaaacttttacctcatttcttaaccacgcagtgttatttgaatagccttaataggctaatacaaaacaacaagccatcgattacagttttaactataatattacaaaatcttaaaccaaaacaagaaaagatatatattccgtaaaattaaaataatatttttaatatagtcattatgtaagttagctctttcgacaaaatcattataaattaggccattttgtaagttagttcttttatcgacaagccattataacttaggtaattttgtaagttagctggactataaccaatttttttgtttcacatctagagtataaaacacatatatattgaccgtacaactttagtcaaattagaaactctgttttatctgcagaaaagaaaaaaaaagaggatgaattatgtaaatacttcaggattagaaataatgtcatcgtatatctcttgatatgaatacatattttacttgactagtaccgtagtcggcaggaaaatgacacaaacaaccatctaaaaagataaagtaagaactaaaaagtcttgacattcattagtttaatcattttctgttaacatatatggaaaaaacaaacttcaccgttatttacctgaatttacctatttgggtaagaattgtacctctggacctctagtattttatatacacctaaaaatataattttggtcgggaaaatataactctgtttaattaattaaattttcagtattgtgtaagtgtaattatagaaatcaattttatccgcgtaacaaaataatataaaattatactttcaaatccacgaatatatattgtgaagtctcatagtttgcaaataagcattggtcttcggccgacaaaaaaaaaagcattggtcttcgaatattttgaatatcggaccaatggtatataattaataatatgtggtatataaatacatatttatttaaatcacaataggatatgcaatgtgtgtatatatacatatacgtaattaaagtccgggttaaactgatagcatatattataatagatgcatctataattgttcgtcaacaaaagcattatcatgtattttgaattaagcttccttcatttctgtgaatcaaaaggcctcagaagaagaaactaaagtcaaacaatcaacgggatccaataaatcacatctggactatatagtatcaatactttccacactaaaaaagctaagaaatttaataaatacattattataggggggaaaaaaaggtagtcatcagatatatattttggtaagaaaatatagaaatgaataatttcacgtttaacgaagaggagatgacgtgtgttccttcgaacccgagttttgttcgtctataaatagcaccttctcttctccttcttcctcacttccatctttttagcttcactatctctctataatcggttttatctttctctaagtcacaacccaaaaaaacaaagtagagaagaatctgtatgacctttcttgggtttagccatgatgatcacattcgttatctattttttggctaaaccccagaaaggtcacattggctcttcttactacaatgaaaaaggccgaggcaaaacgcctaaaatcacttgagaatcaattctttttactgtccatttaagctatcttttataaacgtgtcttattttctatctcttttgtttaaactaagaaactatagtattttgtctaaaacaaaacatgaaagaacagattagatctcatctttagtctctttctccggtgcagtcagccaccgtcgggtaagtttcatctgtattttattaattaattacaattattagtgttcttattaccgttttggtaaaattagttaattaatgtcggtccaaacattctaaaccaattattctgaaaagggtgaacgccaatcagttatatacaatattcttacattaaagtagaatcggagatgttacatactaaccaaaagttacatatactagtatcattttctttaagatttgtttaggatttactcactttatagtgctcgcggttggggtcagggtaagtggggaacagatatgctctgcatgaaccacgtggaccaacatgcatatacacagttacatttattttttctagtaggttcatttgcaaattttccagtatcttgtatgttatcttcatggtgagttttttggtcgcatattcttggtgattcttcttctacgttttcactttcttcttcagagaccttattaatatgttaattgcatgaatttatgtaacattcaagaaaaagagatcgaatacaagaaaccggaaatttaatttctaatttgaattctatgcatgtttgtttctttgccaatttagtacgaatatatttatggtccggagttctaaccaaagatagttgctctgtatatagaagttatacatctcgcttgtttaaactaaataactatttagtttgtcttggtagtccagtgactttctgcaaacattgggatttataatatagtcaaaacgttgggatatgtagtattcattttcgttgtaaaaatcattatattttgcaaaggttgcaacgtaaaacgttgtattgaatttaggagaaagtagccatttacaatgaattttagtgacaaactgttgtatacagcctgtgccactaagtatgtctatatctaaatttggaatggatgataaccagtattggtcaataatggataatttggtatttagaattactaatttggtattttagtgacaaactgttgtatggaatttaggagaacgcgttcccaagctatatattatatgtatttcattctttcatatgttatattatgaattgttataaaaaagaccaatgagcaacggtttaaggtttaatcagtaatcgatccgaccaatcatatacaagttcctaacacatcttctaactactgagtaaaatagtaatgctattacaaaggttttttcttttcaaaatacagcctaatgctactacaaaattctaatgttataaaataatcataggagaagtaaaccccggtatttaattaataccttacaaacttacattattgataaagttgaatgaagagttatatggtccatttagtattatttacttaatatgatactagtgttagacgtgttactgtatttcaacgtgcataatcaggttgttcaaaaagagagaggataggtcttcttcttctgtaatttgctgggaccgcaagtcatgtgaggctgctctgctgatgctgcacggcgtcgcaactctctcaataaattcttttaactaacgctccaattttcgatgtaaattggctgacttcaattctatgccttactcttgtactctatgtttctttatctattaaaatccaactctgcttttgaacccaaaaaacaa

SEQ ID NO: 85 - DNA, 19 bp, synthetic construct
  misc_feature 1..19: note = Single strand oligonucleotide
  source 1..19: mol_type = other DNA
acaccctgggaattggttt

SEQ ID NO: 86 - DNA, 20 bp, synthetic construct
  misc_feature 1..20: note = Single strand oligonucleotide
  source 1..20: mol_type = other DNA
gtatgcgccaataagaccac

SEQ ID NO: 87 - DNA, 20 bp, synthetic construct
  misc_feature 1..20: note = Single strand oligonucleotide
  source 1..20: mol_type = other DNA
gtactgctggtcctttgcag

SEQ ID NO: 88 - DNA, 20 bp, synthetic construct
  misc_feature 1..20: note = Single strand oligonucleotide
  source 1..20: mol_type = other DNA
aggagcactacggaaggatg

SEQ ID NO: 89 - DNA, 22 bp, synthetic construct
  misc_feature 1..22: note = Single strand oligonucleotide
  source 1..22: mol_type = other DNA
gttgagagtgttggagaaggag

SEQ ID NO: 90 - DNA, 21 bp, synthetic construct
  misc_feature 1..21: note = Single strand oligonucleotide
  source 1..21: mol_type = other DNA
ctcggtgttgatcctgagaag

SEQ ID NO: 91 - DNA, 23 bp, synthetic construct
  misc_feature 1..23: note = Single strand oligonucleotide
  source 1..23: mol_type = other DNA
agcttccttcatttctgtgaatc

SEQ ID NO: 92 - DNA, 21 bp, synthetic construct
  misc_feature 1..21: note = Single strand oligonucleotide
  source 1..21: mol_type = other DNA
ctgttccccacttaccctgac

SEQ ID NO: 93 - DNA, 21 bp, synthetic construct
  misc_feature 1..21: note = Single strand oligonucleotide
  source 1..21: mol_type = other DNA
ctcctcatctgattccttctc

SEQ ID NO: 94 - DNA, 21 bp, synthetic construct
  misc_feature 1..21: note = Single strand oligonucleotide
  source 1..21: mol_type = other DNA
ctgttccccacttaccctgac

SEQ ID NO: 95 - DNA, 23 bp, synthetic construct
  misc_feature 1..23: note = Single strand oligonucleotide
  source 1..23: mol_type = other DNA
gtgtgtggaaagtttatcaacac

SEQ ID NO: 96 - DNA, 27 bp, synthetic construct
  misc_feature 1..27: note = Single strand oligonucleotide
  source 1..27: mol_type = other DNA
gttagtatgtaacatctccgattctac

SEQ ID NO: 97 - DNA, 20 bp, synthetic construct
  misc_feature 1..20: note = Single strand oligonucleotide
  source 1..20: mol_type = other DNA
agagtggtcgtgtgagttgc

SEQ ID NO: 98 - DNA, 25 bp, synthetic construct
  misc_feature 1..25: note = Single strand oligonucleotide
  source 1..25: mol_type = other DNA
ccagtcaagatttcatggatctgtt

SEQ ID NO: 99 - DNA, 28 bp, synthetic construct
  misc_feature 1..28: note = Single strand oligonucleotide
  source 1..28: mol_type = other DNA
ctaacataatcgagaacagatggaagac

SEQ ID NO: 100 - DNA, 32 bp, synthetic construct
  misc_feature 1..32: note = Single strand oligonucleotide
  source 1..32: mol_type = other DNA
ttctcatgtccttgatttatcaatttaacaac

SEQ ID NO: 101 - DNA, 20 bp, synthetic construct
  misc_feature 1..20: note = Single strand oligonucleotide
  source 1..20: mol_type = other DNA
agagtggtcgtgtgagttgc

SEQ ID NO: 102 - DNA, 26 bp, synthetic construct
  misc_feature 1..26: note = Single strand oligonucleotide
  source 1..26: mol_type = other DNA
cgcatcagatctatcaaacacataga

SEQ ID NO: 103 - RNA, 277 nt, synthetic construct
  misc_feature 1..277: note = Single strand oligonucleotide
  source 1..277: mol_type = other RNA
gctactctggtgaaccatcgttggccatgcttgccttattctcccatggcaatcttactacggccgtaggaaacttctccggacaagcgaataggcttcacagactctacgaaaacccctaaaacaaacactaacaaagggactgaagagcctaccaccacccaatgcgtcgattactgacgcacgtacgtggtcagcgaaggcgctgagctcgtggtattggcagcggtgaacagaataatcaaacaagccaacggtggtatcatccagaacatct

SEQ ID NO: 104 - RNA, 110 nt, synthetic construct
  misc_feature 1..110: note = Single strand oligonucleotide
  source 1..110: mol_type = other RNA
cgcaaagaaagaggaggaggatgatgagtaaacaaatgcactctgcatgttcgatgcacatgcatctcttgcttgctcattgatccatctttcttgctccggcaccgagt

SEQ ID NO: 105 - RNA, 277 nt, synthetic construct
  misc_feature 1..277: note = Single strand oligonucleotide
  source 1..277: mol_type = other RNA
gaatctgttctcatcttttccttaggggtgtttacctgttctgccacgttatctcttctttccttgattccgacggaaccaaataatagtggccaggacagacagacaacgaaaacccaaaaaacaaacacgaacaatgttagtcaattctggcgtccaacataacgcatgcaacacgtatgcgcggacgttgggagtggaagcattcgccggaaggaaacagcaaaggtgaagagaatagagagtgcgttgaggaggaggatgatgagtaatgacg

SEQ ID NO: 106 - RNA, 277 nt, synthetic construct
  misc_feature 1..277: note = Single strand oligonucleotide
  source 1..277: mol_type = other RNA
gaccaggttatgaccccgtgggttcaagacatgccactagcagccgagctaagcctgccgtcactaaacaacctttgtctagagaatacgctatgctacaaataagcgacgaaaaccctacaaacaatctccaacaacgctcgttaatagtatactgccgcgaattgcgctaaacactagcgcatgcaagcggcgagcttacgagctccgatgacggtaacgccaaggtcgaagtgctggttcatattgggattcgtggggttcactaatccgccct

SEQ ID NO: 107 - RNA, 277 nt, synthetic construct
  misc_feature 1..277: note = Single strand oligonucleotide
  source 1..277: mol_type = other RNA
ggcccaggtacgatggagtccattcaggacttgctagcaacgcacgcgaggggcgattggtgtgtcattgaggcttagcgacagataatatataacataagacgtcctacgaaaaacctacaaacaaacactaacaaaggaagtcaagtgttatcaacctcatctagcttctagaacagaagctcccacgaggtgagtgtaggcattcgcaatattgataggtaactggcgaatcgttgctccaaatcaggatggattctattcgatatttagagaa

SEQ ID NO: 108 - RNA, 110 nt, synthetic construct
  misc_feature 1..110: note = Single strand oligonucleotide
  source 1..110: mol_type = other RNA
cgcaaagaaactcaaagaaaattttcgataaacaaatgcacttcgcatgaacgatgcgtttgcatcccttgcttgttgaagtatttatctttgaggctccggcaccgatt

SEQ ID NO: 109 - RNA, 208 nt, synthetic construct
  misc_feature 1..208: note = Single strand oligonucleotide
  source 1..208: mol_type = other RNA
ttggtgggagggctaggctggcggtacgttattctgaaaaaatattacggacgtgatgcaagcacatatggatgttgcatgacacggccggtgcatgtgaacaaaaacaaaaccaaggcaaaagagcgagagagagagcatgcattgcaaatctgtcgtgtttttttatgggtggcagccaccctggatgcggcatccttcagaccga

SEQ ID NO: 110 - RNA, 277 nt, synthetic construct
  misc_feature 1..277: note = Single strand oligonucleotide
  source 1..277: mol_type = other RNA
gctacggttatggccctacgggtacgagacgagctgatgtcacccgagttactcctacatacgctgtatccaacggatccagagacaataggggtcaagattacgagatagaaaacccagcacacaatattgaacatttttttagagttgatccacaccacatcgtgcagttaacacaattgcaagcacgtggtgagcgcagacgttcgactgtatgtaaagacaaagtcgaaatgatgtttctcatcggtattcgtggggttcactaatcctatcc

SEQ ID NO: 111 - RNA, 277 nt, synthetic construct
  misc_feature 1..277: note = Single strand oligonucleotide
  source 1..277: mol_type = other RNA
gcatctttgcgtccgtcttccgtgctcgacttgccggtacccgccatgacaaacctccgagctatgtgtagtgcctacccatataacacccatctctaaacgtattctaagaaaaaccacaaaacaatcgcgaacaagggaggcgactagagatccacctcatcgagctttgactactaaagctgctacggggtcgggtaagggcccgaggagtttggaaagtaagtggtgaaggggtgctacagatttgtatggaagatgagactgcaaaagtacc

SEQ ID NO: 112 - RNA, 105 nt, synthetic construct
  misc_feature 1..105: note = Single strand oligonucleotide
  source 1..105: mol_type = other RNA
gaagagaagaatctgtaaagtttagaaaggataaacaaaccacgatcacacacatcgatatacttttgtttgtccattctgagcttcactggctcttcttacttc

SEQ ID NO: 113 - RNA, 208 nt, synthetic construct
  misc_feature 1..208: note = Single strand oligonucleotide
  source 1..208: mol_type = other RNA
tcggtcgggtagttacgctggcatcgattgatggatagtaataataaagtcagtcttgcgagcacagatggatgtatcatcacaccacctgtggatgactacaaaaagaaaaccaaggcaaaagagcgagagagagagcccgcagggcaacggtttcgttgttactataccgttgcagccaccgtaaaacctccctctgccgaaccga

SEQ ID NO: 114 - RNA, 277 nt, synthetic construct
  misc_feature 1..277: note = Single strand oligonucleotide
  source 1..277: mol_type = other RNA
gagtcggttatggccctgtgaattcaggtcatgcgtccctctgcctagtgaagccacccctcgctggggatgtcgtccacagagaacagaacggtctgcagacgcactacgaaaaccattgaaactagcccaaacaaagtgggtcaccagattgccagctcgcggtgctcacagaacgtgagcaggaaagggttgaggccatggtctcgtccgaggggtaagccacagtggaaaagagggaacataatgggattcgtggggttcactaatccttgca

SEQ ID NO: 115 - RNA, 341 nt, synthetic construct
  misc_feature 1..341: note = Single strand oligonucleotide
  source 1..341: mol_type = other RNA
aacgagatgccaacttctcaagattgcaaaagcaaccagctagtacgttggtacacaccaatgggataaccggtcagcaattagatcctgttaccatcaacaggatccgagggctcatccccgatgaggaatcggatcggccatcagatctcgagcaagaaccaagaggtctgccggtatatctccttagaatccaaacacgaccagggttggacccgttccttcctcgcccccaaaaagtaagggcggaggaatgagttcctggttgtttcagggagagaatcagcatcatcggcgttgattggtaccggctttgcagctggaggggttggcaactcgcc

SEQ ID NO: 116 - RNA, 103 nt, synthetic construct
  misc_feature 1..103: note = Single strand oligonucleotide
  source 1..103: mol_type = other RNA
agagagagctacaggccggcgctggagagcatacgcaggtaattttattagaaaataactttgcaggtgcgtgctctctaagcgtcggcctgcgccatcttgg

SEQ ID NO: 117 - RNA, 341 nt, synthetic construct
  misc_feature 1..341: note = Single strand oligonucleotide
  source 1..341: mol_type = other RNA
tgggggagggtccaggcatcagggatgaaaagcaagcagctaccctgttgagacagttcgacaatactaccctaaagcagtgtgattgaattactgagaatttgatccttggactcgttgcaagcgagcaaaagttggggatgtaacttcgcgaggaagcaggaagcggagtcatgggttatcgccagataaaccaagcacttcgtcagccccagggtgacctatgtggcccccgaaaattaagggcctaggtcacgttgtgacggagtttactggtgggtttgatgagctcagtatgtcaagggacccatggttcgtgatagtgtctggacctaccccgt

SEQ ID NO: 118 - RNA, 341 nt, synthetic construct
  misc_feature 1..341: note = Single strand oligonucleotide
  source 1..341: mol_type = other RNA
agagtgatttattcgtggggttcactaatccgtaagcaacgctgatgccatcataggatggtaatgaagccggatagatacaagttgctatttccaataatagcaaccatggaggctgcgcagcagctcaaatggttcggggtcagatcatcgggcaatgacgaagatgatcataggggtagcgcgtgagaagccaaccacgctctgcgatcgagccttaactcgagcgcccacaaaaaggaagggtggagttaagccttgtagagcgtttccgcgcgtggtagtacagccgcgcagtattacgactcctgtggttagtaagtccgcgagtaagacactgg

SEQ ID NO: 119 - RNA, 239 nt, synthetic construct
  misc_feature 1..239: note = Single strand oligonucleotide
  source 1..239: mol_type = other RNA
gcggaggtcaagttctcctcattataaacatgaaggtgtctctcgtcctaacacacagcgatcaacaaaaagtaactaacccacacatatggtagaaaccaaaaccataacgtggatacccttattctgcgtataagtttgacaaaattcgggggaaggaaccatgtttgtattgggcggagcaagcgcgtggctcctagagcacgtcggccacaaagcacgttggggaccaagaacgg

SEQ ID NO: 120 - RNA, 94 nt, synthetic construct
  misc_feature 1..94: note = Single strand oligonucleotide
  source 1..94: mol_type = other RNA
gggaagtcttggattgtggagaatttaaaaagaaataactttcgtcacgaacgaaagcaattattttttaacaccaccacagcctaagattttc

SEQ ID NO: 121 - RNA, 225 nt, synthetic construct
  misc_feature 1..225: note = Single strand oligonucleotide
  source 1..225: mol_type = other RNA
caacagaagagacacccccgaggagagaacctgctcatctttaatgttttttttgaaaacctgaccagcgccgctcctgcctgcaacaaccatccatcagaacaaaaagttcgtatggcgaaaggccattgatggtgggcaggtctccccttggggaagtctagcgtcaacgagctggggtctgatggtttcacaattgctaaaacttgtgcttcaggtgacaaa

SEQ ID NO: 122 - RNA, 239 nt, synthetic construct
  misc_feature 1..239: note = Single strand oligonucleotide
  source 1..239: mol_type = other RNA
ggggagagcatggattcgtggttcctgtggatagccccgcacataatcgcacttcgtcccataaacaaaaacaaaagaaaccacacaacctgtagaaaccaaaacaggtggcaccgatcacgacccggtgccgccgctcagacatgagttatgtgtacacagattcgtggggttcactaatcctaaccacccagctcgtagcgggtcgggcgtatcacttgggatcggctcaagatcgg

SEQ ID NO: 123 - RNA, 341 nt, synthetic construct
  misc_feature 1..341: note = Single strand oligonucleotide
  source 1..341: mol_type = other RNA
accgtgagatttttgaggtgagcgagggtagtcaaacagacgatggtcagtaatattattggcgctacgccctttatgtaccatggtgcgggccgggattcgtatcacctggaatcatttcgaatgatgaaaggggagggattagaacgtacgaccaactagcaagtgtgttgagggatcatctcctttagctccaatcactagcacaggatgtccttgctctattctgcccccaaaaataaagggcatagggtaaagactgtgttagtgctagggagacattcttaagcgctgcataggaatcggtccttctaccctcttgatctcaaagatcacacgta

SEQ ID NO: 124 - RNA, 96 nt, synthetic construct
  misc_feature 1..96: note = Single strand oligonucleotide
  source 1..96: mol_type = other RNA
gacaactggttattggtgcggggcgttaagaaaacggtgctcaatactttttgagctccgctttctgaacgttctgtattgataacgagtgccacg

SEQ ID NO: 125 - RNA, 341 nt, synthetic construct
  misc_feature 1..341: note = Single strand oligonucleotide
  source 1..341: mol_type = other RNA
cgtgggagataaaattatttttgatacactagagagcatctacagttaaaataaagattttgagtagcaccccacaggtttcagtacttttggctaaataaaagtaccaggttttctttcctaaagaataattggtgggggactaagatctcgcgcaattatgaagaggtctgtgggatcagctccctcgaagccaatcacgcttaagggcccaactagatcgtctcggccccctaaaacgaagggttacggtttacttgcttgagcgtttcagggggtgatgcgttaccaacggagacgtatgggtcccgcagtgtgggatggtggttttattacccaag

SEQ ID NO: 126 - RNA, 341 nt, synthetic construct
  misc_feature 1..341: note = Single strand oligonucleotide
  source 1..341: mol_type = other RNA
gctgggaggggttagtggagttacacggatgggaaccaccctaccttgatataaacatattaaccatagacagagagggagaaggtctctggtcatgctcagagacccaaggggtctattctatagacgaattgctttgtaggtgttggctcgcgaaactaaaaagagttgagtcgggaaatcgccagattgaccaatctcacacattggggtaacgggaccatcctcgcccacaaaaaggaagggcggtggtcctattaggtgtgtggcgactggcgagggttcgcagctacgcagtggatcgttcccgacattcgtggggttcactaatcctacccaca

SEQ ID NO: 127 - RNA, 193 nt, synthetic construct
  misc_feature 1..193: note = Single strand oligonucleotide
  source 1..193: mol_type = other RNA
gggttcttccagaagggcggacaaccaacggcgaccgccccctagggaccccaggcggacgttaagttaggatttcccatttacattcggatgggatgtgggcgagccgccgtcacgaaaacggcggcagcgccaagagcgctggacgttcgcagaagacagaacctggttgctcgctcttctggaagaacct

SEQ ID NO: 128 - RNA, 193 nt, synthetic construct
  misc_feature 1..193: note = Single strand oligonucleotide
  source 1..193: mol_type = other RNA
gggttttttcagaagggtggacatccatcgcccatctccgcttgtgccccccaggagaagggtacgccaggatttcggatagagtgacctattcgaggagtttccgccgctgcctcgaaagcagcggctgctccaactgagcagaagaagctcttaaaacagaacctggatgctcgctcttctggaagaacct

SEQ ID NO: 129 - RNA, 193 nt, synthetic construct
  misc_feature 1..193: note = Single strand oligonucleotide
  source 1..193: mol_type = other RNA
gggttctttcaggagggtgaacatcccacgccgacctctcattattgcccccaggaggatggtacaactggtcgtctatttgaagtgctaagtggaggtgccccggctgctgacccgaaatcagcagcagctccaaaagagctgggggggcgctgaatccagaaccgggatgctcgctcttctggaagaacct

SEQ ID NO: 130 - RNA, 193 nt, synthetic construct
  misc_feature 1..193: note = Single strand oligonucleotide
  source 1..193: mol_type = other RNA
gcggtggtattgctttttcatcagcctgcgcccatcacaccagggggcagacatgtgaagggcaatacaggactactagcttaaagacgggctggttgggtagcagctgcaggcgcgaaatctgcagccgccccaaaagggcgggggctgcccacaaggcagaacaaggctgttggaggggtaatgccattgc

SEQ ID NO: 131 - RNA, 216 nt, synthetic construct
  misc_feature 1..216: note = Single strand oligonucleotide
  source 1..216: mol_type = other RNA
gggaatcagacatggcgagatttcatcgggatctggggcgagggcgagggcgtgacagtggggagcgcagtcgatacatgcaccaggcacacccagtatcgccttggaaagcaaggcgcactgggtccgccggatgcatgtgacgagtgcaacgcgcccgctgcaagcatcgcgactcgttccagattccgatgattctccgtcgcactgatgccc

SEQ ID NO: 132 - RNA, 89 nt, synthetic construct
  misc_feature 1..89: note = Single strand oligonucleotide
  source 1..89: mol_type = other RNA
catgctatcggtgcttcgtacggtatttgcagagagcacatcgaacaccgatggcctccgtggatgtcgtatgtggtgccgttagtatg

SEQ ID NO: 133 - RNA, 142 nt, synthetic construct
  misc_feature 1..142: note = Single strand oligonucleotide
  source 1..142: mol_type = other RNA
gcggcctgaaacaccgtttgattggtgtgctggggtcaacgtgtccaatgccagaagacgtcagcaagggaagaacaggtgttcctgggcgatgaacgcgttggtgcttctacatcaaccgaaggtaaactttcacgcaacg

SEQ ID NO: 134 - RNA, 186 nt, synthetic construct
  misc_feature 1..186: note = Single strand oligonucleotide
  source 1..186: mol_type = other RNA
gtggcgtgacctgagcaagtattcgtggggttcactaatcctgaccgacccttgcgcaggccaggcgtccatgattggtaagtcaccccacaaagtgcaagaccagaatggacagccaggttgccttaaccagcagagggtcggcgagcgttggtgaggaccgccgatgcatgaccaccacgccac

SEQ ID NO: 135 - RNA, 216 nt, synthetic construct
  misc_feature 1..216: note = Single strand oligonucleotide
  source 1..216: mol_type = other RNA
tggatacacgcgtgggaagatctggtcttcttgaccggtcttggttaggtcatgacgggggtccgcgcagtccactcaggcgctcggcacccccgctaccttcacggaaagtgtgaagcggcggggcagccagtcgcctgagaggagtgcaacgcagcccccgcaagaataacagaggactggtcaagaagactattctgtttcggagtgtagcca

SEQ ID NO: 136 - RNA, 83 nt, synthetic construct
  misc_feature 1..83: note = Single strand oligonucleotide
  source 1..83: mol_type = other RNA
tacgtatattgtgtagttaatttactcaggtgtgtgtggggttacggtgcacctccaatagtgaagactatcagcatcacgta

SEQ ID NO: 137 - RNA, 140 nt, synthetic construct
  misc_feature 1..140: note = Single strand oligonucleotide
  source 1..140: mol_type = other RNA
cccggagtttgtgggcaagtccgctcgtacgacttcaaggaacagcctccgaaaacaggagtacatgtgagatagaataattcctgggccggctccctgttgtcttgttgtgcgaacaagacatgccgggagctcccggg

SEQ ID NO: 138 - RNA, 186 nt, synthetic construct
  misc_feature 1..186: note = Single strand oligonucleotide
  source 1..186: mol_type = other RNA
aaggtgggagtggagagagaattcgtggggttcactaatcctcactagctctggccctagaggtgacacccggatatggaaagctccacacggtgagacagccgtaacgggtgctcaacttgggcagagccagccgggagctagccagagttggtggaagccgccagttcactagctgcccacctt

SEQ ID NO: 139 - RNA, 375 nt, synthetic construct
  misc_feature 1..375: note = Single strand oligonucleotide
  source 1..375: mol_type = other RNA
tttgcgaggctagcaactggattctactatataaaaacacagacttggaatcatgtcacccaccaggactctccgagaatccccgtccgaaagacatgaacgggtggaaaaagactctcaatcatcgacgacaagctaacacaaggtatccagtaaggtactctaaaggaggttgatgtgatggagcactgtagaacacgtgctaaactcacatcgactaaagatgagtgttcaaggtcgtcaagtatccgagccaggatacaaacgactcacacatattggtcctataccctaaaggataccctccgaaagggcaattcggacagcaaaagctgtgttttcctatatggtagatgtggttgttagccacgcaaa

SEQ ID NO: 140 - RNA, 99 nt, synthetic construct
  misc_feature 1..99: note = Single strand oligonucleotide
  source 1..99: mol_type = other RNA
tcagcgtatgtagtaggctctgcttcatcaagtggtcttgtgttcaaattcagctccaagcacatggtcaactggattctactatatatgtgaatataa

SEQ ID NO: 141 - RNA, 375 nt, synthetic construct
  misc_feature 1..375: note = Single strand oligonucleotide
  source 1..375: mol_type = other RNA
cttgcgaggtcagcgtgtccatccaacttatgtaatgctcccatgcaatgtttgtccaccccagaggccttattttaaagctgatgctaccagggcaaaaaatggtgaaaaacccatctagacaattccgatatccaacttcaaggcttccaggtagtatcacgaaacgtgaaacacgtgaaggatctttgcagagcatgggataaaatcacgtgttttggtctggagtttccaatgacatcaagtcgcagagccatgcgacaaatgttacagataaattgcagtgaatacgacaaggaagccctcctgacggttaatcagggcggcccgtagggggtattccattcgggctcttatgcgcgttgaccacgcaat

SEQ ID NO: 142 - RNA, 375 nt, synthetic construct
  misc_feature 1..375: note = Single strand oligonucleotide
  source 1..375: mol_type = other RNA
aaagagagggattcgtggggttcactaatccgttagcctcacaccccttattccctcacctctcatgccttagaatgaagcaccagtcactagagggaaaaaaggcgaaaaacccttccaaacaatagtgagcaccaaacccaaggtaagcagctggtagcttgaagcaagttatacggggcggaaaacagaagatctcgttttaaacccccgtataagaaccaagagcgctcactggccccaagtttctgagccaagaaacaagggtcacagctgtgttgttgctactacaagaagtttacccaccagaaggccaatctggttagcacggggtggggctaccacgggttagtaagtctgcggatcccactcttc

SEQ ID NO: 143 - RNA, 375 nt, synthetic construct
  misc_feature 1..375: note = Single strand oligonucleotide
  source 1..375: mol_type = other RNA
attgtgaggccaaacagaagattaaggggttgtctaccgctcacctattactgtcacacctagcatgcccagaacctgtgcaccctccacaagtgatagcaatggcgaaaaaaccatccaaccaacagtgctccagaaaaccaaggcttccaggacggatctctaagggagacaatagggtgggatatatgaagatcattgtataaaggtcctgttgtgccagtaggttccacaccctctccaataggctgagccaagcttaaagagaggccgacggaacgtatacaattcattaaggaagcccactagtagcgcaagctagtcaccagagggagcggtagccacaattccttatgttttgtttggtcacacaaa

SEQ ID NO: 144 - RNA, 125 nt, synthetic construct
  misc_feature 1..125: note = Single strand oligonucleotide
  source 1..125: mol_type = other RNA
cactgtgcggtctctaatacgactgttcagagtgatgaaccggtttccgcgcggaacggaggagcgggcgccggcgccgaattggatcgttcctctgagcagctgtatcggaggccgcggtgaac

SEQ ID NO: 145 - RNA, 375 nt, synthetic construct
  misc_feature 1..375: note = Single strand oligonucleotide
  source 1..375: mol_type = other RNA
tttgagaggccaggaaggatattacgatgatgaatcccacatacttgattgttactcaccttgcaagcccaccgtgtgagctcccaccattagagtaactgtaggggaaaaaacctagacttcaactgaccttcacaaacacaaggtttccagtcggtatcactaacggtgataaaagtttcggtaccttctagaagatgggtaaaaaaaacttttattgatttagagcaaacactttatgcaatggtcagagccatgaccaaatatggacagacttgttgtaacaaatacggaaaggaaacccaccacaagggcaatgtggtaagcaaaagatgtgggatcctcattgtcgtggctccttcttggtcactcaac

SEQ ID NO: 146 - RNA, 375 nt, synthetic construct
  misc_feature 1..375: note = Single strand oligonucleotide
  source 1..375: mol_type = other RNA
gatgagaggtttggaggggtaatgccattgcgttgcacacatacttccactttgcccacccctcatggcgccgacggcaccaccagcaaatagggcaaacagagaccaaaaattcttatacacaaacactcgcggcaaatacaaggtaaccagtgagggcgaccaaaggtttgtctagcgagggtactcggcagagctagggtaaaagttgctggacacgtaactgacatcgaaagatctgcaagtgtctgagccaagacataatagatccggaggatgtgggtccagctcccaaaggttacccaccaggaggacaacttggtcatcataagatgtgtgcgccatgtggtggcttctctttccaaaccactcata

SEQ ID NO: 147 - RNA, 375 nt, synthetic construct
  misc_feature 1..375: note = Single strand oligonucleotide
  source 1..375: mol_type = other RNA
aacgagaggtatgataaaacatgcttatgaatgaaatcactcactgcgcccattcccaacccggatgccttatgatgaagcacgcgattcgagggaatgaattagggataaagctaactacgcaaactaataccacaaagtcaaggttgccagaggggaacacgaaccgtgataagagaggaggctcgccgtagaacgcgtgggaaaacttctcttatgagacttgtgagaacaattaccacaagatcccgagccagggatcaatggtgaccgagtcttaggagcaattccactaaggtaacccacccgaagggcaattgggtaatcaacaggagtggtttgccatttgtaagatcttttgtcgtatcactcgtc

SEQ ID NO: 148 - RNA, 111 nt, synthetic construct
  misc_feature 1..111: note = Single strand oligonucleotide
  source 1..111: mol_type = other RNA
catgtttggatactagttaatattaatgtttaaagctactctacaaaaaatatatgatatatttccggtagagtagctttaaatattgatattggctagtatccaaacaga

SEQ ID NO: 149 - RNA, 375 nt, synthetic construct
  misc_feature 1..375: note = Single strand oligonucleotide
  source 1..375: mol_type = other RNA
tttgggaggtctagttaatattaatgtttaatttaatctcagactattatttgtcacaccactaatgcctgaggctcaagcaccagaaataagtgacaacattggtgtaaaatccagcctcccaagtcaaatcgaacaattcaaggctgccagatagtctcactaagggtggttaaagcgagggtggcgtgtagaacaccgtcaaaaatcgctttagcgcgtcgagatttcgcaatatcttcaagtctctgagccaagagataaaagatacccaagaaatgtcgttaagaccataaggcagcccacctgaagggcaatcaggttatcattagctggggttgccagttgtgctccacgttgagtggatcacccaac

SEQ ID NO: 150 - RNA, 375 nt, synthetic construct
  misc_feature 1..375: note = Single strand oligonucleotide
  source 1..375: mol_type = other RNA
gacgtgaggtttggaggggtaatgccattgcgtaaacccccgactcagaattctcccaccgcacagcgcaagctccttacgtcctgacaagagggagaacattggggaaaaagccagccacacaagtaactaaagcaaaatgaaggtgtccagaaacttcccccaagggggtaaaacgtgtggggttgccgaagatcgtgcgacaaagacacgttttagggtttcgagcgcgcaattactccaagtgactgagccaagttataagagtgaccgaagcgttggaatgaggagcttaggggcacccgcctggagggcaaccgggcaagcaggagcgggggtttccacgcggtggcttctccctctgaatcacacgtg

SEQ ID NO: 151 - RNA, 375 nt, synthetic construct
  misc_feature 1..375: note = Single strand oligonucleotide
  source 1..375: mol_type = other RNA
gtagcgaggtcgaatcttgtttataacttaagtttgtcgctaacctggcgcagtctcacctcaccagccatagactatagctcctgtcataagagattgaaatggttaaaaacccatacatacaaatctgatacccaaactcaaggattccaggaagtatggccaatggtcaattcagtcatgggtcacagcagagctcgtggcaaactgactggattacacttaggtttcccagtcacgtcaactgtccgagccaggacagaaacgtgaggcacgaagcgtaacggatacatcaaggaatccctcctgaaggacaattagggcaacaaaggtagcggcagccacttgggttgaaccgaggttcgatcacgctag

SEQ ID NO: 152 - RNA, 102 nt, synthetic construct
  misc_feature 1..102: note = Single strand oligonucleotide
  source 1..102: mol_type = other RNA
gtgaagccactggatggatcggttcaatcctctcttccggaatcgttgttgaacataagaacctagaatcgaggattaaacttatttatttagcgctcactc

SEQ ID NO: 153 - RNA, 375 nt, synthetic construct
  misc_feature 1..375: note = Single strand oligonucleotide
  source 1..375: mol_type = other RNA
gttgtgagggcgaatcttgtttataacttaactaaatcacccactcaagtctgcgtcaccactcatattttaggctaaggtaccaggccatagacgtagtaatggagaaaaaaccatcgaatcaaaactaccctacaaaaccaaggtttccgatgagtatcactacgagtgaaaatagctggggagtttagaagatctggggctaaaacggctgttttgccctttgagtatacattgtctgcaagagtctgagccaagactcaacagataccgagatactggaatgaatacccaaaggaaaccctccctaagggcaataggggcaccacgaggggtggtttccagttgggttgggtgaagattcgctcacgcaaa

SEQ ID NO: 154 - RNA, 375 nt, synthetic construct
  misc_feature 1..375: note = Single strand oligonucleotide
  source 1..375: mol_type = other RNA
gatgtgagtaattcgtggggttcactaatccggttaccacagacagcctaatgacacaccacacatgccaagccgcttagcacctgccacaagtgttataaacgccgaaaaatgcgtcaacacaaccatttgaaccaaagacaaggtacccagcaagtatctccaaaggaggaataagtgagggtgtccgagagactcggggcaaaaattacttgttcagcgttggtgttcgcattctcaccaagctcctgagccaaggagcaagtgagactcaggaacagtaaaaaatacctgaagggtaccctccacgaggacaacgtgggcatgacctgctgtggtagctccgggttggtaagtctgcggattacacgcata

SEQ ID NO: 155 - RNA, 367 nt, synthetic construct
  misc_feature 1..367: note = Single strand oligonucleotide
  source 1..367: mol_type = other RNA
agtaccgagggaaacgagatgctgctcatataatggcggatagccccaacttcacaactatcgggccttccggtgcacgagcggaggttcttagagctagcaagtccctcggtaagattccacagcctgtgggacagctactcacagggccctaccggacagctggaagtagacttggtatgcctgacgtaacagaccagttactcagacagccaaatccatatcgtacaaatgataggaaagagggatcttaccgagggaaccaattaactctaagaaccctcactaagtgaccgggagagctggtagttatggattgagggctaaaagctattgtatgggtagcattctgtttccctcgatactg

SEQ ID NO: 156 - RNA, 87 nt, synthetic construct
  misc_feature 1..87: note = Single strand oligonucleotide
  source 1..87: mol_type = other RNA
cacgtcagcacgacaggatgccaatgcaaaaaaacgtgtccttgagggcgcgtttttttgcattggcatcctgtcacgctgacgtgg

SEQ ID NO: 157 - RNA, 168 nt, synthetic construct
  misc_feature 1..168: note = Single strand oligonucleotide
  source 1..168: mol_type = other RNA
ggaggacaacatgccaatgcaaaaaaagttttggtagaaggagacggtacgcagagcagaagaggaacggtggacttaactgagaccatcttctcctagataagatctcacaaacccctctccatgcagatctactaaggatctgctgcgttggtgtgttgtcctccc

SEQ ID NO: 158 - RNA, 372 nt, synthetic construct
  misc_feature 1..372: note = Single strand oligonucleotide
  source 1..372: mol_type = other RNA
gtggacggtcattcgtggggttcactaatccaaaagaagaccgcagatccaaaggacaagttaagggccatgttgcccctagacacagaggatggcccggcggtgtcactgggttaggggaggacctctgggcgaaggaaatcagagcacacggcctggctctatcttgaacccgtgcatagtaaacggcagatgtgggtaagaacaccacattatgcacgggttggagacggacccaggccaaaattcggaacatggctcccctaacctggtgacaccgccaggccataggggcaaaacatgacccttaattcatctattgggtccgcgagctccttacggattagtgaactttgcggttgaccgtgcacc

SEQ ID NO: 159 - RNA, 367 nt, synthetic construct
  misc_feature 1..367: note = Single strand oligonucleotide
  source 1..367: mol_type = other RNA
cgtaccacggtggcagggagaagagaacgataattgcaaattgcccctgcgtctcgcattacgggctaaactgttccggctagtagggacaatgagctagcaagtctatctgtggttagaaacaccctgtactacaggcaagtacgggaccagacatggcagcgtgactcagacggggcacgccagtagtttcagcccagagacactgacggccccaacgacataaaacaatttatgcgtaatattctaactacagatagaaccaactaactcattgttcccacgagaacggatagtttgagccgtggtgcaagaccagagggcaaaaggcaattgtcgttctctttttgttgccgccgtgatacga

SEQ ID NO: 160 - RNA, 101 nt, synthetic construct
  misc_feature 1..101: note = Single strand oligonucleotide
  source 1..101: mol_type = other RNA
gttgcaaacagaagattaaggggttagatgatggcgtgcaatcaggagtcttgaagcaatgcatcccactcctttggtctcttgatgcttctgttttcacc

SEQ ID NO: 161 - RNA, 214 nt, synthetic construct
  misc_feature 1..214: note = Single strand oligonucleotide
  source 1..214: mol_type = other RNA
ggctgtcggcctcgctaggtatgtttaacaatgttatgccaagcggtgggccaaaaactcgaaaaacaacccctttgaggagtaggcgctgcagtcaaaccctgtactccccgacgggatctccgatgagatcggagacaaaacacgggtctaaaaccgagcatagacaccagggcacaatattgttaagtgtccttggtggcaccagccgccc

SEQ ID NO: 162 - RNA, 367 nt, synthetic construct
  misc_feature 1..367: note = Single strand oligonucleotide
  source 1..367: mol_type = other RNA
tgggccaaggtattcgtggggttcactaatcctcggcacaacgccccttctccgctgaaaaccggcgtaacattacctgtacgcatgcccatagagcgagccagccgcactgtctctacgaacagccagttttacagctaaaaactgggccccatccggcagcggaaggaataccaggcatgccgctaggaacagtctagttccagcgacagcctgaacacgttcatacaagtgaattgtaatatcgtagagacagtgcggaccaactccctctatgggcgcgcctaaaaggagtgttacagcggttttcgatggaaagagggcgtaaagctgagggttggtgagtcctttggataccttgactcat

SEQ ID NO: 163 - RNA, 367 nt, synthetic construct
  misc_feature 1..367: note = Single strand oligonucleotide
  source 1..367: mol_type = other RNA
ggtaccatggtgcacagcagtatttgtgtatatgtgccgaacgccccctcatctcgtggcaggggcaaatcattcagaggtcgtatggtctctgtgccagccagcctttcagccgtcctaaataaccaggttgacagtcacaatctgggcaccactcgtcagcgagaggaacacggggtatgccaatgttgacaggaaagtcagattgccggccctaacaacttcttacaaaagaagtgtaagattaggacggctgaaaggaccaattgacgcggggaccacacaataatctaatgatttagccctgccacaaggtaggagggcgtaatgcacatgtacacgaatactggtgtgcaccatgatacca

SEQ ID NO: 164 - RNA, 101 nt, synthetic construct
  misc_feature 1..101: note = Single strand oligonucleotide
  source 1..101: mol_type = other RNA
gctgttgggtggagttccagagtaaagatgagggagtgcaattaggagtcttgaagcaatgcatccccgtcctttttgctctggaatcttcattcatcacc

SEQ ID NO: 165 - RNA, 148 nt, synthetic construct
  misc_feature 1..148: note = Single strand oligonucleotide
  source 1..148: mol_type = other RNA
agaggagcccctggtcgatcatggtacgcaatgaacgtcgtcatgtatcgacctaaggacaatcggcagtgattaactttcttaaacagacccgagcatgacacatggcgaccctcatagtgtgcttgggtccgttacggactccgac

SEQ ID NO: 166 - RNA, 372 nt, synthetic construct
  misc_feature 1..372: note = Single strand oligonucleotide
  source 1..372: mol_type = other RNA
ggggtcggccattcgtggggttcactaatccaacgtgtgaccggcggggcggagctcacatatagtgcctggtagcctcaagagtcggaggtttgccctactgtcggtcagtgtttgggggggacctccttacgaaggaaaaaggagcaagcggccaggctgtataaagaacccttgggattaacatggcactagcgggtaagaacaccgctaatttcaagggttgtttacataccttggccaaaagaccaaacacatccccccaaacgttgactggcggtgaggcagttgaggctaaaccagacgctatatgcaagcactgtcccctcgagcagacgacgggttggtgggtcctgtgattggccgagcccc

SEQ ID NO: 167 - RNA, 206 nt, synthetic construct
  misc_feature 1..206: note = Single strand oligonucleotide
  source 1..206: mol_type = other RNA
gggtgccgaagcaatccgaggctttttacttgggtgcttggtcggcagggaatcaaactctcgcgtgaattatgccgttgtcgtcgccctatctcgttccggggagccctaaaacctgactaaataattatagccttcaatgtgatgagttgtgatgatagccgaaccaagggcctgatcaagaagttccggacatttcggcaccc

SEQ ID NO: 168 - RNA, 104 nt, synthetic construct
  misc_feature 1..104: note = Single strand oligonucleotide
  source 1..104: mol_type = other RNA
ggcgcgagctaccacaagatgtacggcaaacggccgggcgtgacggcaccggcggtcgtgccgtcgcggccgcttgctgtatgatctttgtggtagccggtgcc

SEQ ID NO: 169 - RNA, 139 nt, synthetic construct
  misc_feature 1..139: note = Single strand oligonucleotide
  source 1..139: mol_type = other RNA
aaggtccggaaccgaacggcaagagagctcggcaagggcctgatcaagaactcttggatctcaagccaaggagtgggtggctgatgggggttcttggttctgtccttatcgagcgggccaagttcacgctatcagcctt

SEQ ID NO: 170 - RNA, 206 nt, synthetic construct
  misc_feature 1..206: note = Single strand oligonucleotide
  source 1..206: mol_type = other RNA
gggggcccatccaatctgaggattcgtggggttcactaatcccccctaaaagtgactcacacgtccgggcgagccttttcgcgatgaccgatctcgagccggtgttcacgcaaaaacatcccacgaaagcttgaatccagggtgtagtgagctcacgtctggggagggttgatgagccatgcgggtccccagacagatgggccccc

SEQ ID NO: 171 - RNA, 206 nt, synthetic construct
  misc_feature 1..206: note = Single strand oligonucleotide
  source 1..206: mol_type = other RNA
gggagccccgccaagatgaggatccgaatttatgtaccttatcgtcacccgattacgctcacgggagtcgccagcttgactatttcaccaagccagatcgagtgatcgcgaaaatcgaaacgaagtcactggccagacattccgtagagcgctgatgccggatgaatagggagcatgatctcggatccccatccagcggggctccc

SEQ ID NO: 172 - RNA, 99 nt, synthetic construct
  misc_feature 1..99: note = Single strand oligonucleotide
  source 1..99: mol_type = other RNA
ggaatggggctggctgtcacgtcgacactcatctagagcaacaaacttctgcgagaggttgcctatgatggatgttgatgttacagccaactttattcc

SEQ ID NO: 173 - RNA, 165 nt, synthetic construct
  misc_feature 1..165: note = Single strand oligonucleotide
  source 1..165: mol_type = other RNA
tgggggcggggcgggtacgggaacacgtaagctgtgatcgttgatgttacagccaactttaagggtgcggcgggcagtactcagcgggggttcttcctccacccccggggatggtcggatcgagggaacatcgcacgtcgcccccgcccgcaacccccgccccta

SEQ ID NO: 174 - RNA, 206 nt, synthetic construct
  misc_feature 1..206: note = Single strand oligonucleotide
  source 1..206: mol_type = other RNA
gggagccagtgcaatctgaggattcgtggggttcactaatcccctcgacgaaatgctcacacctacttacgcttcggcgctagatcgcccatccaggtcccctgaccacgcaaaaggatcagaagcgcaagtgaatgacgtgggtggtgagacgttgacggaggagggttgatgagtcaaacgagtccccagacacactggctccc

SEQ ID NO: 175 - RNA, 206 nt, synthetic construct
  misc_feature 1..206: note = Single strand oligonucleotide
  source 1..206: mol_type = other RNA
gggagcctatccaaaaggattgggattatgctggggtttggcctacttcgagttaggcacacgggtggcatcccgtagacgggttagccgcgaacgattgtccaatccctaaactgtagcaggcgtctgggatcagccaacccgtcgtgctgtgacgtctgtagagctaaagtttagttaaatcttaacccttcagatgggctccc

SEQ ID NO: 176 - RNA, 165 nt, synthetic construct
  misc_feature 1..165: note = Single strand oligonucleotide
  source 1..165: mol_type = other RNA
ttggggcggggcgggatcgagcatatgaaaggcgagattgaggtctggatagagtttgcacacggcgcggcggccagccatcggcgggatgcctgcccccgccgcgtgcaagctgctattccagattttatctctcatcactccctcccgcaccccccgccccga

SEQ ID NO: 177 - RNA, 139 nt, synthetic construct
  misc_feature 1..139: note = Single strand oligonucleotide
  source 1..139: mol_type = other RNA
aaggactcgtacaggccggctagagagcgtaaacttttcatattgttttactcttggggcacattccttggcgtgataggaggccgggggtggggcggtctgaggagatgacgcgtgccaaggcccctcgcccctcctt

SEQ ID NO: 178 - RNA, 206 nt, synthetic construct
  misc_feature 1..206: note = Single strand oligonucleotide
  source 1..206: mol_type = other RNA
gggagccgagccaatcagaggttggaggggtaatgccattgccgacgagtatccaccggctcgtgtgatcgctccatagctggtctcccgaggcagtgcgcgggcacgaacaaaacaggcagaagctagagtgcagtcaacgtgatgttggatggggaaggtcgagcgatgacgttgcaacttcaacccctgacagttcggctccc

SEQ ID NO: 179 - RNA, 292 nt, synthetic construct
  misc_feature 1..292: note = Single strand oligonucleotide
  source 1..292: mol_type = other RNA
gaaccaatgcggaagataccaatgcacggtcgcatgtaaaattgggcagttggtcagttttgttggggtgaagtcttgccccagttagccgggaggaaagatattacacccgactcattctgacgaacgcctcaggtagtatgcaacctgatactgaggtgaacggcagaatgatgcgggtgtatatatccacctccgctaactgggcgaagacttcactccaacataacggacccgctgtcaccggcccatacccgacgtattgggctctttggtaacagtggtcgggttc

SEQ ID NO: 180 - RNA, 84 nt, synthetic construct
  misc_feature 1..84: note = Single strand oligonucleotide
  source 1..84: mol_type = other RNA
ataccgagataagcagaggaagaataattaggtgctaattggaagctgcacctacattcctttttatctgtttagttttggtag

SEQ ID NO: 181 - RNA, 199 nt, synthetic construct
  misc_feature 1..199: note = Single strand oligonucleotide
  source 1..199: mol_type = other RNA
agggcgtaccaggaggtatgaagcctcgcatgggagatctgcgaggtattggggtagacttcgtaaaaggcgcaaacaagctcgcacaaaagcgtgaggatgatgtagctaaagatgcataattcaacagcgattcacgcacccagggtgattcgtggacctcttgttgagagctgtactttggatagtaagacgccct

SEQ ID NO: 182 - RNA, 292 nt, synthetic construct
  misc_feature 1..292: note = Single strand oligonucleotide
  source 1..292: mol_type = other RNA
cgccccatgagttagaaacgagttcggggccaaagttctaacacgtaggttggaaagggtttggtgaaccttgcggacacggaggtcgtggtcggaggagaattatcgcccgacgcttgcccgctaacccgtgaggtgccaggaaacgcgatactcacgggaaggagggtaggttgcgggcgataaattcggtccggacgacctccgcattcgtggggttcactaatcccgttcctacctaccccggtcactgaatggcgaacttggttctaaattacaaggagaacgggtg

SEQ ID NO: 183 - RNA, 292 nt, synthetic construct
  misc_feature 1..292: note = Single strand oligonucleotide
  source 1..292: mol_type = other RNA
gccccactaagcttgaagccattcccgcgtctcgggagaaaagcgggcgtgggatatttagttgtgctgacatgtctagcaacggatgacgaaggttaagaaatatcgcccgactcgtcctcacaaacgccagagcacgaacgcaaccgtacactctggcgaatggggggacgaagcgggcgataatttcacgcctttcatccgttgagggacatgttagcataataaaaagtccagcgccctacggtgcccacaagacggaatggagtcgagattaaaatgggacgggggc

SEQ ID NO: 184 - RNA, 91 nt, synthetic construct
  misc_feature 1..91: note = Single strand oligonucleotide
  source 1..91: mol_type = other RNA
aaggcttgctactgtggagcaatcattctgaacgatagagaaaatgggatttaatttatcggtcagaatgatactccacaatactaagcct

SEQ ID NO: 185 - RNA, 195 nt, synthetic construct
  misc_feature 1..195: note = Single strand oligonucleotide
  source 1..195: mol_type = other RNA
ggagggtacctagccggctgtaacttgtggactatcggcggggtaatggggggcaactagctgccgacccattcctccatcccgtccgtggctacgagagggtgggcgcgacttggaatgagggaatggagcgggagatagttggagtccccgcccgtgcctatccttttcaggttgtagcctctgatgagctct

SEQ ID NO: 186 - RNA, 292 nt, synthetic construct
  misc_feature 1..292: note = Single strand oligonucleotide
  source 1..292: mol_type = other RNA
gccccactctgacagagaccatgtcgcgcaatatgaacaaacggggctgagggaggtggcttggtggaccttgcgaacccataggtagacgagggtaccgaaaagacgcccgactctggcatacaaacccggttggatccaagaaatgataaacgaccgggaatgggtgccagaagcgggtgttattttctcgcccttctatctgtgcattcgtggggttcactaatccagctccatcagccgacagcatcgccaattggacatgggttctgtaggactaagtgcagggggc

SEQ ID NO: 187 - RNA, 292 nt, synthetic construct
  misc_feature 1..292: note = Single strand oligonucleotide
  source 1..292: mol_type = other RNA
ggccccgaggtcttgtgcccaaagacatgtctgagactccagggggaagtgggtaatttagtttaaggtgtatgccactctcaggtaggcgaaggttaggattgcccccccgacgcaggcttacaaacgcggatgggttgacggaagaacagacatccgtgaatgagagtctgtcacgggggggacaatcgcaccttcctatctgagcttggcatatattttaaataaaactaccaactttcgaagacttctgaaagactttttggcggcaggttctaaaggtgagcgggtc

SEQ ID NO: 188 - RNA, 84 nt, synthetic construct
  misc_feature 1..84: note = Single strand oligonucleotide
  source 1..84: mol_type = other RNA
ttgggatatgttaggaaataagaaaatcgaggtgctaattggaagctgcaccttaggtcctcttgattcctagccgtatctcat

SEQ ID NO: 189 - RNA, 199 nt, synthetic construct
  misc_feature 1..199: note = Single strand oligonucleotide
  source 1..199: mol_type = other RNA
agggctttcacgggcttcgtaggccttgctaagagattttttaaaattaactggatgacttagaaaattacgcaaacaagtagaaataaacgcatgtgtggtgcgttgccaaagaagtgttccacaaaagatacgcatgctccatcttagtttgggggactttcttacgagagcccgaagctagagggagagaagccct

SEQ ID NO: 190 - RNA, 292 nt, synthetic construct
  misc_feature 1..292: note = Single strand oligonucleotide
  source 1..292: mol_type = other RNA
gtccccaaccagttgtagccaattcggatgctgatggacaacacgtaagaaggatgtggcttggtgagctccgcggacccgtaggtttttgtgggagttgatcgtgatcccgactcatgcacgcaaacgcaacgggctgtacgaaatcagagaccgttgcgaatgagtgtatgacgcgggattgtcgatctatcccaggaacctgcgcattcgtggggttcactaatccgagtccgtcttacctcgccgcgtaaaagtagaattgggggcaacgggtatagacgagaggggc

SEQ ID NO: 191 - RNA, 237 nt, synthetic construct
  misc_feature 1..237: note = Single strand oligonucleotide
  source 1..237: mol_type = other RNA
ctgacccgtacatatcgagagttgtctggcccgccacagcccctagcaaatctcgtcgcggagccacgcgtagtatggtcgcgagccacgcccgaagctaaaccagaagcaccgaaggctacgcgtcccttatacactgtaacggcaccccgaacacgggaagagggctccgaaggccgacgagatttcctaggggctgtggcgggttggacaactttggatatgtgccggtcaggg

SEQ ID NO: 192 - RNA, 64 nt, synthetic construct
  misc_feature 1..64: note = Single strand oligonucleotide
  source 1..64: mol_type = other RNA
cgctgcggagctcaagaagaaccctcttcgaataatccagggttcttttagggctttgcggtat

SEQ ID NO: 193 - RNA, 110 nt, synthetic construct
  misc_feature 1..110: note = Single strand oligonucleotide
  source 1..110: mol_type = other RNA
aaagttaggggagcactagggttgattggtcgtcacagttagatctttctgaccctagagacgaaaaagctgtggcgggttggacaacttcatacctcccaaccatagcg

SEQ ID NO: 194 - RNA, 237 nt, synthetic construct
  misc_feature 1..237: note = Single strand oligonucleotide
  source 1..237: mol_type = other RNA
cgggacagttcattgcgggggttggtggaccccacgagttcccaagcaggagtaggcgcagagtagcttgacgcaaggtaccggcccacggacgacctcaaacagtagagacctaacgcgtcaggctcctatggaacccatacgaaacccccagcaggggaagagtactctgtagaccgtctactcctccttgggaattcgtggggttcactaatcccggcagtgagccgtcccggg

SEQ ID NO: 195 - RNA, 237 nt, synthetic construct
  misc_feature 1..237: note = Single strand oligonucleotide
  source 1..237: mol_type = other RNA
ctgaaccgatcgcaacggagtggtatcattcactgcctgtcactttcatattgtggcgcaggtctgcctgtggcatgggcgtgctccacacagaattcataacctcttgatccaaacgctgcgggtgacgcattgaaaatgtttctggcccgaactcggggaaaaggacctggaggctgtcgcaatatcaaagtgacaggcggtgaatgatgttattcggttgcgatccgttcaggg

SEQ ID NO: 196 - RNA, 64 nt, synthetic construct
  misc_feature 1..64: note = Single strand oligonucleotide
  source 1..64: mol_type = other RNA
accggaagaggtaaatgaattacctttccgaataatccaggtggtttatatgccttttccggat

SEQ ID NO: 197 - RNA, 110 nt, synthetic construct
  misc_feature 1..110: note = Single strand oligonucleotide
  source 1..110: mol_type = other RNA
aaggcgggggaagcatctggacgccggaagaggtaaatgaagatcttccacattggtgggacgaaaatcgtttgctatgtatcatcgtcccataacttccaattaggaca

SEQ ID NO: 198 - RNA, 237 nt, synthetic construct
  misc_feature 1..237: note = Single strand oligonucleotide
  source 1..237: mol_type = other RNA
ctgatcagagcattgcgagggttggtggaccccgcggatacactctctgtcaagggccctgagtcgcttggcggatggttcgcagcccgcgcgtaggacaaactggagtcaccaaacccgccaggtccataagcgacgtttacggaaccccaagcatgggcagaggactcaggaggcggtccttgatacagagtgtattcgtggggttcactaatcctggcaatgctccggtcaggg

SEQ ID NO: 199 - RNA, 237 nt, synthetic construct
  misc_feature 1..237: note = Single strand oligonucleotide
  source 1..237: mol_type = other RNA
cagaacagatccggacggttgtgttgtgcttttgctggaacactagcttttgtaggcccagcgttccaaggcgcaaggtgacacgctctgtacgaacctaaacccaaaggacctaacgcgtcttggagactctagactagggccttattgcaagcatgcgcggacgacgctgaagacggcctatagaacctagtgttccagcaaaagtacagcacaacggtccggatccgttctggg

SEQ ID NO: 200 - RNA, 52 nt, synthetic construct
  misc_feature 1..52: note = Single strand oligonucleotide
  source 1..52: mol_type = other RNA
tttggtcccatgttggagctggtcgcacccagctccacatggaagacttacc

SEQ ID NO: 201 - RNA, 118 nt, synthetic construct
  misc_feature 1..118: note = Single strand oligonucleotide
  source 1..118: mol_type = other RNA
aaacaggaaatgggaagcggcagaaatgccattctccgtttacgttcttcgacgcttcccgctttcctgggacgtgagtagggaatagtgtttttcccacttctcaagaccgacccgc

SEQ ID NO: 202 - RNA, 237 nt, synthetic construct
  misc_feature 1..237: note = Single strand oligonucleotide
  source 1..237: mol_type = other RNA
ccgagccgatcatgtcgcggattggtgagccccacgggtgcacctacgtcatttgtcgctgagtaacatgctgcaagtgacgcgccctgcggatattacaaactagagtatactaacgcggcatgttcggaagcaacgcttcagaaaccccgatcacgggcagagtactcgggagcccggcaaatgatctaggtgcattcgtggggttcactaatccgggatatggtccgctcgggg

SEQ ID NO: 203 - RNA, 193 nt, synthetic construct
  misc_feature 1..193: note = Single strand oligonucleotide
  source 1..193: mol_type = other RNA
ggtggtgtgtcaaaggttcgctgcctctgcatctaccctctaattgccatatgagaccctaaactacccaagccactggaataaaaatgaggccgccgttccaaccaaaacgataacataacaaatgttattacaagggtgaaaagggcgaaggggggtgggtgcacgggcgacgcaccggacgcaccgccac

SEQ ID NO: 204 - RNA, 111 nt, synthetic construct
  misc_feature 1..111: note = Single strand oligonucleotide
  source 1..111: mol_type = other RNA
gccgtgcacccactagcagttacagtgttcatgaaatccttaacttccttccctgtcgggaggatagataggggtttttgggtgctgtacctgttgttgggatcacgaaga

SEQ ID NO: 205 - RNA, 138 nt, synthetic construct
  misc_feature 1..138: note = Single strand oligonucleotide
  source 1..138: mol_type = other RNA
gaacgccgcagcgttaaaagctgcctctgcctctgcatctatcttcggttagaaggtgaaggggggcgccaagaccgcggttctaaccctagctaggtgtagcccgagggagccagctcctggcggcttggggcccca

SEQ ID NO: 206 - RNA, 193 nt, synthetic construct
  misc_feature 1..193: note = Single strand oligonucleotide
  source 1..193: mol_type = other RNA
gcaggcgtcgccaggggctgttggaggggtaatgccattgcaatggctataagggactgaatactaccgagagcgcggatgccaaacccaagagacagcatctaagacaacgaagaaccaataaggttctttaaatcggtaaaaagagccaagcggtggtattgccattccattaacccagtggcgcctgcac

SEQ ID NO: 207 - RNA, 351 nt, synthetic construct
  misc_feature 1..351: note = Single strand oligonucleotide
  source 1..351: mol_type = other RNA
caggggagtgaagcgagtgcatcttcattcatcgaagcatgcaccttcacattcggaaaaatccccacggctggaaccgtccacccaacactaggcagagtcgagcccgcgcggtaccaaatctgggttggagaataagtcccgggggtagccctccagagggggcccaaggccacatgagggatgcgtgcaagggagggacacagcatggaccggcctgactactattgaaagccgaacgtggatcagagtcggccgaaggggcagaatctacggcccgaatcttgatccaaaccccatagagtcagggcgccatgggtgggtgaaatccactcgcttcacacccccaca

SEQ ID NO: 208 - RNA, 93 nt, synthetic construct
  misc_feature 1..93: note = Single strand oligonucleotide
  source 1..93: mol_type = other RNA
aaaaccaagctggtgctataatgcacaataaggatcaatcagcacctggatctgagattcgagttgtgcgttggcgcacccacgtggatccta

SEQ ID NO: 209 - RNA, 351 nt, synthetic construct
  misc_feature 1..351: note = Single strand oligonucleotide
  source 1..351: mol_type = other RNA
gctgggaggtcagcgtacaactcatgaatatttgaatctcccaccatcccgaacctctctgaccccatgcccggtatcgaccaccgatccacgaagacagaccaggccgcccgattcccaatttgtgatcgataaatgggctcggggttttcccttagcagggggacccagtccctaggatggatgggggaatgggtggtaaaagccagggctaggtctgtaatctatacaaaccccaacggggctagtagtcagctgaggggggaaaactcatagcctgaatacctagcctaacaccatagttacagggctccctgaggtgttcatatgtgtgcgttgatcacccaccca

SEQ ID NO: 210 - RNA, 351 nt, synthetic construct
  misc_feature 1..351: note = Single strand oligonucleotide
  source 1..351: mol_type = other RNA
tttgagaggcttggaggggtaatgccattgcgtgaaacatcaaccggtcccagcatcgaatggcgcaagtcagggcccgcccaccgttcctttgcgatactccaggccgcccgatagccaggcaaagaacgagaaattggcacgcgtcacgtcccaactgggagttgccacgacgaagaccggattggtgtaaggggggcagagaacagggtggtgaccttccatcatctaaagactaacggggtatcgactcagctgaggtggtagaaaccacagcctgaatcagatacctaacgacatggggagggtgacccctgacgtggtggcttctctttccgagccactcacgca

SEQ ID NO: 211 - RNA, 351 nt, synthetic construct
  misc_feature 1..351: note = Single strand oligonucleotide
  source 1..351: mol_type = other RNA
gatgagaggtatgggttttgagacaaaatttgagaaccgaaccccatcccaccgataaagggaccgaagtcggctacatagcaccgtctctacagaatagtccaggccgcccgtagcccagctgtaagacgagaacatgatgccggtccgcccccatttggggagatcgaatctctaggatggaggttcggacgggcgacagagatggcggtgaggacgggccaccagtaaaaatccaacggggcgttaagttagcgtagtgggcagaatgcaacgcctaagttcaatgcctaacaccttggggtttgtgctccgcctcaagttttgctgggagcccgtaccactcacgca

SEQ ID NO: 212 - RNA, 91 nt, synthetic construct
  misc_feature 1..91: note = Single strand oligonucleotide
  source 1..91: mol_type = other RNA
cagtagcagttggcgccatgatcaagaagaaaacttccctaaacaagccaaggaaagatcttttcttgtcgtggcctcacctgttgatggg

SEQ ID NO: 213 - RNA, 351 nt, synthetic construct
  misc_feature 1..351: note = Single strand oligonucleotide
  source 1..351: mol_type = other RNA
atcgagagtgcagattcttcttggcgaattagtgaagctaacaccttctcagccaccacagaccccaggccgggacccgtccaccgatccaccacaagagcccaggccgcccgtcgccaacgtggtgatcgagaattcggcgcggggtcaaattccgacgaatggttcgaagcccaaggagggatgttagcaagggcggtaaataacatggtgagggtcgtttgcaattttaactctaacgtggttaatatttcgctgaggaggtagaatccacagccgaactacttaaccgaactccattgagatgacgttccatgactagttcgggctggggatctgtacactcgctca

SEQ ID NO: 214 - RNA, 351 nt, synthetic construct
  misc_feature 1..351: note = Single strand oligonucleotide
  source 1..351: mol_type = other RNA
aaagagaggaattcgtggggttcactaatccttaaaacttccacctacccgaacatctctgtctcggcgtctggatacgtccactgagcctgatcgaaagaccagaccgctcgcttcccaagatcagctcaccacggaggctgcgaggctagtcaaacgtgacggacccagtccacaggtgggatgggggtatacgtgttatagagattggagttgcctgtctgcgaccaaaatccaaacgcgggttttcggcagctgagggggaacaacccatagcctgcagacaaacccgaacgccgtcgagatagggaaccaataggggttagtaggtccgcgaatttcactctcgca

SEQ ID NO: 215 - RNA, 351 nt, synthetic construct
  misc_feature 1..351: note = Single strand oligonucleotide
  source 1..351: mol_type = other RNA
aacccgagttcagaatctcacttgagattctgtgaacgatgcacctcgctcccctattccgaccccaggaccggacccgtccaccgtccacggaataaaggccactccgcagaacccccactttcgggacgacaaatggtcacggggtctctggtcattaccgtgtatgttgcaattgtggggatgcgttgagaataggcaaataccctggcggtgtgtgtttcagatcacaaagcgaacggggttctgagtgtgccgagggggcataactcatggccacagtcaagaacctaaccccatttagatacggacccaggatagggtctcggcggggttttgaacacgggcgca

SEQ ID NO: 216 - RNA, 100 nt, synthetic construct
  misc_feature 1..100: note = Single strand oligonucleotide
  source 1..100: mol_type = other RNA
aacagttgagtggggctctgtctgcgtctagaccactcgcaccaagtacagacataatctgaatgatctaggcgcagattccaccccatccaactcaaca

SEQ ID NO: 217 - RNA, 351 nt, synthetic construct
  misc_feature 1..351: note = Single strand oligonucleotide
  source 1..351: mol_type = other RNA
tatggcagggcagaatctcacttgagattctgtgaaacaaacaccagctcattctgttcaaaccccacgtctggtaacgaccaccggaccgtgatcacagttcaggccgcccgatacccaagtcgcgtccgaaaagtaggcccggggttggcgctccaaagcgggaccgagtcctaaggttggatgtttgtaagggtcggatacatcagggtgagggctgtctgctttaaaaaacccaacgcgggtttgtcagtgctgagtcgggacaagacacagccattagcaaaacccgaacgcgaaagagatagcgttccctgatagggtcttggcgatgtatggttcagtcacgca

SEQ ID NO: 218 - RNA, 351 nt, synthetic construct
  misc_feature 1..351: note = Single strand oligonucleotide
  source 1..351: mol_type = other RNA
gatgagagctattcgtggggttcactaatccctgaatcacacacctgccctcccagtcccgccccctagtcagatgacgatcccggccgcttgatcatagcccaacccgcgtgcagcccacgtcgacggtcaaaacttggcacgggggctccgcccgatggcggggttcaacccgcaggcaggatgtgtgaatagggaggaagcaccacggcgagggctgtattcttgataaaaccaaacctggagtgcacttggcggagttggaagaaagcattgcccaattgccactccgaacgcccaagatatagtgctccgtgaggggttagtaagcctgcgagtagcactcacaca

SEQ ID NO: 219 - RNA, 351 nt, synthetic construct
  misc_feature 1..351: note = Single strand oligonucleotide
  source 1..351: mol_type = other RNA
ccggggacgtcacctgtggcttctaccgcaagagaactgcaatccgtatcacgccggaataaccacaggtttgggagcgcccaccgtccagctgttaaagtccaggccgcccgtcgcccatgcagcggacgaaaattaagcacgtggttcgcccttactagggggctcgaagccataatacggaattgcggacggggggcacatatcacggaacggccctacggcgttgagaacactaaggcggtacttgataagcccagtgggaaaaatgcagggccttaccacggtaccgaaagcaaatgcgtaggggcgccgtgttttgtggtaaactcacgggtgatgaccctccca

SEQ ID NO: 220 - RNA, 99 nt, synthetic construct
  misc_feature 1..99: note = Single strand oligonucleotide
  source 1..99: mol_type = other RNA
gtttttagaggtgaatgctggatggctcgtcgcattcacataagaagaagaaatataagtgaaggctgcgaatcgctcttcgtttatctctaatataca

SEQ ID NO: 221 - RNA, 351 nt, synthetic construct
  misc_feature 1..351: note = Single strand oligonucleotide
  source 1..351: mol_type = other RNA
cacgtgaggtccaagccaccatccaagccaagcgaaacaaacacaagcccattagccccttaccccacggctgggtccgcccacagttccactacgaaaggacaggcagcccggaccccaagtagtgaactagaataagtcccggggtacccgcgtatacgcgtgatctaatcagcaggtttgatgtttgtaagggaggtatacaacttggcggttgagttccactgttctaacactatcggggtcctaattcagccgatggggaaaaattaacggcctgatttcaggacccaacgtcacagggagtttgacccagggcttggtttccttgtggcttggatcacgcgcgca

SEQ ID NO: 222 - RNA, 351 nt, synthetic construct
  misc_feature 1..351: note = Single strand oligonucleotide
  source 1..351: mol_type = other RNA
agtgtgaggaattcgtggggttcactaatccgggaaactgtcgccttcacttactgcccgtaccccaagtcaggaacagtccacagggccagtgcgaaagaccatgccgccagcctcccaggcattgccctacaacgtggccaggggtaaagactatcgagttgggcccagcccctatgaaggatggcagtcaaggcaggaaatacacgggaggcgtccgcaccatgtcccaatacgaacggggttcatcatacgctgagccggtagaaggcatagccgtaggactgaacctaacaccacgtgtgtgggggtcccgttcgggttggtaagtctgcgaattccacacaatca

SEQ ID NO: 223 - RNA, 213 nt, synthetic construct
  misc_feature 1..213: note = Single strand oligonucleotide
  source 1..213: mol_type = other RNA
tgattgtgtggcttgaggtccggggtcagcgtcccccgctccgacccgaactagaagtgagtagaagctagagtgaacggtaccacacagacccgcatctcggaccaaccctccgcctgcaaccccttccaccccgacccccctctcgaggtccgagacagggcggtgggagacgagggggcaagaactccgtactttatttcacgtaaccaa

SEQ ID NO: 224 - RNA, 100 nt, synthetic construct
  misc_feature 1..100: note = Single strand oligonucleotide
  source 1..100: mol_type = other RNA
aggatttgcctgggctacagtatggcactcacctagagcaacgacagcttgggagggttgcctataaggtggatgccgtactttatttcacgtaaatctc

SEQ ID NO: 225 - RNA, 167 nt, synthetic construct
  misc_feature 1..167: note = Single strand oligonucleotide
  source 1..167: mol_type = other RNA
cggcggccagaatgcgcgcaaaaaaaaaaaaaaaaaaaaagcggcggccggactgcacctgtggacggccgccccaaagcgacggagagacgggttcctaaccaaggtgtaggagcagccgccggcgcttttttttagtagcttttctcttgtcagtctctcctcca

SEQ ID NO: 226 - RNA, 213 nt, synthetic construct
  misc_feature 1..213: note = Single strand oligonucleotide
  source 1..213: mol_type = other RNA
tgaggattcgtggggttcactaatcccagacgccgccgctcccccctgagcagggacatcgcccagacctgaggcaacgcggcgccaccaaccacccactcgttcccgcactgaaacatccaccacaacgtacgtagccccccccacacggagcgagttctggcgggcggagacgagcggcggagagggttgatgagccgagtggatccccaa

SEQ ID NO: 227 - RNA, 213 nt, synthetic construct
  misc_feature 1..213: note = Single strand oligonucleotide
  source 1..213: mol_type = other RNA
cgattttttatgctttttggttcggtcagccgccgccgcgccagcccacctcgtacccccagaccacacgacgcccacgcagcggcaacaaggcccagcccgagcccccgcaccctcgtcttccacatcaaccgcagccccccaccccaggctcgggcccgcccggtcggtgacgagcggcgtagaaccgaggcaaagataataaaaaaccga

SEQ ID NO: 228 - RNA, 76 nt, synthetic construct
  misc_feature 1..76: note = Single strand oligonucleotide
  source 1..76: mol_type = other RNA
tggttatgacgagcgtgccgtacgagccacggctgctgctgcgccgccgcgggctacggcactcttttcataatta

SEQ ID NO: 229 - RNA, 155 nt, synthetic construct
  misc_feature 1..155: note = Single strand oligonucleotide
  source 1..155: mol_type = other RNA
atggccgccggcttagagagtgaaaaaatatatttctgcaaaaggcccaccggccgtgggcggcagaatgccgatttctgcgcctgggagagaggggcccgcgcagcgataaagcttttgtgggtgtgtatttttacaaccctctctagcctaca

SEQ ID NO: 230 - RNA, 213 nt, synthetic construct
  misc_feature 1..213: note = Single strand oligonucleotide
  source 1..213: mol_type = other RNA
cgaggattcgtggggttcactaatccccgccgcctccgcgccgcccggccgcttactcccttcatccaagcacatgacgcgaaatgacccactccaagccctaccccacgaacctgcgacgactgctacaccctccgacccccccccccgggtagggcccgagaggggggtgacgagaggtgaacaggattgatgagccggatgagtccccga

SEQ ID NO: 231 - RNA, 213 nt, synthetic construct
  misc_feature 1..213: note = Single strand oligonucleotide
  source 1..213: mol_type = other RNA
acattggttttactgagtccgcggggcagagcccgccgccatgaagatccagtataacccatctagctactcgagtaccctgcctccccgacaccaagctcgacccctcgctccaacgtaaacaacagatcccccagccccccccacctgggtcgagcccgtgctttggtggacgagcgggcgagatctcgcagatttacaaaaactaacgta

SEQ ID NO: 232 - RNA, 74 nt, synthetic construct
  misc_feature 1..74: note = Single strand oligonucleotide
  source 1..74: mol_type = other RNA
gtgcaggattgtgagggagcatttccccagctcgatcattgcatgattagaagagtttcttcatgttcctgcat

SEQ ID NO: 233 - RNA, 213 nt, synthetic construct
  misc_feature 1..213: note = Single strand oligonucleotide
  source 1..213: mol_type = other RNA
tgagggtgctcgatcacggccctgggccaacgccgccgctcccgccccctatcgcctctccacacaccgatcagcaacacgccgctacccactcctaacgcgagcccacccgtccacttcttcttcaaccgccgcgaccccccccccccggctcgtgtccgagcggtcggagacgagtggtggagatccaggattgtgagggagcatttccaa

SEQ ID NO: 234 - RNA, 213 nt, synthetic construct
  misc_feature 1..213: note = Single strand oligonucleotide
  source 1..213: mol_type = other RNA
agaggattcgtggggttcactaatccccgcctccgccgctccaaccaatagctacaggtcgagaatatagcatgctaaccgccgcaaccaacgcccatctcgtgccagccccacaccatgccctcctacagcctcagacaccccaacagggtacgagaccgcgcggtcggagacgagcggagcaaaggattgatgagtcagatggatccccta

SEQ ID NO: 235 - DNA, 23 bp, synthetic construct
  misc_feature 1..23: note = Single strand oligonucleotide
  source 1..23: mol_type = other DNA
agaaataggcaagtcatccttgg

SEQ ID NO: 236 - DNA, 23 bp, synthetic construct
  misc_feature 1..23: note = Single strand oligonucleotide
  source 1..23: mol_type = other DNA
agaagagagtgagcacacaaagg

SEQ ID NO: 237 - DNA, 23 bp, synthetic construct
  misc_feature 1..23: note = Single strand oligonucleotide
  source 1..23: mol_type = other DNA
agaaataggcaagtcatccttgg

SEQ ID NO: 238 - DNA, 23 bp, synthetic construct
  misc_feature 1..23: note = Single strand oligonucleotide
  source 1..23: mol_type = other DNA
agaaataggcaagtcatccttgg

SEQ ID NO: 239 - DNA, 23 bp, synthetic construct
  misc_feature 1..23: note = Single strand oligonucleotide
  source 1..23: mol_type = other DNA
agaaataggcaagtcatccttgg

SEQ ID NO: 240 - DNA, 23 bp, synthetic construct
  misc_feature 1..23: note = Single strand oligonucleotide
  source 1..23: mol_type = other DNA
agaagagagtgagcacacaaagg

SEQ ID NO: 241 - DNA, 23 bp, synthetic construct
  misc_feature 1..23: note = Single strand oligonucleotide
  source 1..23: mol_type = other DNA
acagaagatagagagcactaagg

SEQ ID NO: 242 - DNA, 23 bp, synthetic construct
  misc_feature 1..23: note = Single strand oligonucleotide
  source 1..23: mol_type = other DNA
agaaataggcaagtcatccttgg

SEQ ID NO: 243 - DNA, 23 bp, synthetic construct
  misc_feature 1..23: note = Single strand oligonucleotide
  source 1..23: mol_type = other DNA
agaaataggcaagtcatccttgg

SEQ ID NO: 244 - DNA, 23 bp, synthetic construct
  misc_feature 1..23: note = Single strand oligonucleotide
  source 1..23: mol_type = other DNA
aagaatctgtaaagctcaggagg

SEQ ID NO: 245 - DNA, 23 bp, synthetic construct
  misc_feature 1..23: note = Single strand oligonucleotide
  source 1..23: mol_type = other DNA
acagaagatagagagcactaagg

SEQ ID NO: 246 - DNA, 23 bp, synthetic construct
  misc_feature 1..23: note = Single strand oligonucleotide
  source 1..23: mol_type = other DNA
agaaataggcaagtcatccttgg

SEQ ID NO: 247 - DNA, 23 bp, synthetic construct
  misc_feature 1..23: note = Single strand oligonucleotide
  source 1..23: mol_type = other DNA
cgtagatgaatggttccatcagg

SEQ ID NO: 248 - DNA, 23 bp, synthetic construct
  misc_feature 1..23: note = Single strand oligonucleotide
  source 1..23: mol_type = other DNA
aaaaatgacgctgacagaagagg

SEQ ID NO: 249 - DNA, 23 bp, synthetic construct
  misc_feature 1..23: note = Single strand oligonucleotide
  source 1..23: mol_type = other DNA
cgtagatgaatggttccatcagg

SEQ ID NO: 250 - DNA, 23 bp, synthetic construct
  misc_feature 1..23: note = Single strand oligonucleotide
  source 1..23: mol_type = other DNA
cgtagatgaatggttccatcagg

SEQ ID NO: 251 - DNA, 23 bp, synthetic construct
  misc_feature 1..23: note = Single strand oligonucleotide
  source 1..23: mol_type = other DNA
aaggtgccgcggtcatgatgggg

SEQ ID NO: 252 - DNA, 23 bp, synthetic construct
  misc_feature 1..23: note = Single strand oligonucleotide
  source 1..23: mol_type = other DNA
aatcttagggatgggaggtgtgg

SEQ ID NO: 253 - DNA, 23 bp, synthetic construct
  misc_feature 1..23: note = Single strand oligonucleotide
  source 1..23: mol_type = other DNA
aggagtgaacctgagaacagagg

SEQ ID NO: 254 - DNA, 23 bp, synthetic construct
  misc_feature 1..23: note = Single strand oligonucleotide
  source 1..23: mol_type = other DNA
aaggtgccgcggtcatgatgggg

SEQ ID NO: 255 - DNA, 23 bp, synthetic construct
  misc_feature 1..23: note = Single strand oligonucleotide
  source 1..23: mol_type = other DNA
cgtagatgaatggttccatcagg

SEQ ID NO: 256 - DNA, 23 bp, synthetic construct
  misc_feature 1..23: note = Single strand oligonucleotide
  source 1..23: mol_type = other DNA
agtggcacgtgatattggcacgg

SEQ ID NO: 257 - DNA, 23 bp, synthetic construct
  misc_feature 1..23: note = Single strand oligonucleotide
  source 1..23: mol_type = other DNA
cgtagatgaatggttccatcagg

SEQ ID NO: 258 - DNA, 23 bp, synthetic construct
  misc_feature 1..23: note = Single strand oligonucleotide
  source 1..23: mol_type = other DNA
cgtagatgaatggttccatcagg

SEQ ID NO: 259 - DNA, 23 bp, synthetic construct
  misc_feature 1..23: note = Single strand oligonucleotide
  source 1..23: mol_type = other DNA
catcatcaagattctcatcctgg

SEQ ID NO: 260 - DNA, 23 bp, synthetic construct
  misc_feature 1..23: note = Single strand oligonucleotide
  source 1..23: mol_type = other DNA
catcatcaagattctcatcctgg

SEQ ID NO: 261 - DNA, 23 bp, synthetic construct
  misc_feature 1..23: note = Single strand oligonucleotide
  source 1..23: mol_type = other DNA
catcatcaagattctcatcctgg

SEQ ID NO: 262 - DNA, 23 bp, synthetic construct
  misc_feature 1..23: note = Single strand oligonucleotide
  source 1..23: mol_type = other DNA
catcatcaagattctcatcctgg

SEQ ID NO: 263 - DNA, 23 bp, synthetic construct
  misc_feature 1..23: note = Single strand oligonucleotide
  source 1..23: mol_type = other DNA
ccgctgcttgtatttcccattgg

SEQ ID NO: 264 - DNA, 23 bp, synthetic construct
  misc_feature 1..23: note = Single strand oligonucleotide
  source 1..23: mol_type = other DNA
caaatggcatacagggagccagg

SEQ ID NO: 265 - DNA, 23 bp, synthetic construct
  misc_feature 1..23: note = Single strand oligonucleotide
  source 1..23: mol_type = other DNA
ctgaatgatgatctcggaccagg

SEQ ID NO: 266 - DNA, 23 bp, synthetic construct
  misc_feature 1..23: note = Single strand oligonucleotide
  source 1..23: mol_type = other DNA
aaggtgtgagttgagcaagatgg

SEQ ID NO: 267 - DNA, 23 bp, synthetic construct
  misc_feature 1..23: note = Single strand oligonucleotide
  source 1..23: mol_type = other DNA
ccgctgcttgtatttcccattgg

SEQ ID NO: 268 - DNA, 23 bp, synthetic construct
  misc_feature 1..23: note = Single strand oligonucleotide
  source 1..23: mol_type = other DNA
ctggcagcttcaactattggagg

SEQ ID NO: 269 - DNA, 23 bp, synthetic construct
  misc_feature 1..23: note = Single strand oligonucleotide
  source 1..23: mol_type = other DNA
ggaggcaggcaagtcatccttgg

SEQ ID NO: 270 - DNA, 23 bp, synthetic construct
  misc_feature 1..23: note = Single strand oligonucleotide
  source 1..23: mol_type = other DNA
aaggtgtgagttgagcaagatgg

SEQ ID NO: 271 - DNA, 23 bp, synthetic construct
  misc_feature 1..23: note = Single strand oligonucleotide
  source 1..23: mol_type = other DNA
tgtgatttaccagatcatgctgg

SEQ ID NO: 272 - DNA, 23 bp, synthetic construct
  misc_feature 1..23: note = Single strand oligonucleotide
  source 1..23: mol_type = other DNA
cctcaagcacatggttaaccagg

SEQ ID NO: 273 - DNA, 23 bp, synthetic construct
  misc_feature 1..23: note = Single strand oligonucleotide
  source 1..23: mol_type = other DNA
tgtgatttaccagatcatgctgg

SEQ ID NO: 274 - DNA, 23 bp, synthetic construct
  misc_feature 1..23: note = Single strand oligonucleotide
  source 1..23: mol_type = other DNA
tgtgatttaccagatcatgctgg

SEQ ID NO: 275 - DNA, 23 bp, synthetic construct
  misc_feature 1..23: note = Single strand oligonucleotide
  source 1..23: mol_type = other DNA
tgtgatttaccagatcatgctgg

SEQ ID NO: 276 - DNA, 23 bp, synthetic construct
  misc_feature 1..23: note = Single strand oligonucleotide
  source 1..23: mol_type = other DNA
cctccattccgcgcgaaaaccgg

SEQ ID NO: 277 - DNA, 23 bp, synthetic construct
  misc_feature 1..23: note = Single strand oligonucleotide
  source 1..23: mol_type = other DNA
tgtgatttaccagatcatgctgg

SEQ ID NO: 278 - DNA, 23 bp, synthetic construct
  misc_feature 1..23: note = Single strand oligonucleotide
  source 1..23: mol_type = other DNA
tgtgatttaccagatcatgctgg

SEQ ID NO: 279 - DNA, 23 bp, synthetic construct
  misc_feature 1..23: note = Single strand oligonucleotide
  source 1..23: mol_type = other DNA
tgtgatttaccagatcatgctgg

SEQ ID NO: 280 - DNA, 23 bp, synthetic construct
  misc_feature 1..23: note = Single strand oligonucleotide
  source 1..23: mol_type = other DNA
gaagctctcaaaagagcttatgg

SEQ ID NO: 281 - DNA, 23 bp, synthetic construct
  misc_feature 1..23: note = Single strand oligonucleotide
  source 1..23: mol_type = other DNA
tgtgatttaccagatcatgctgg

SEQ ID NO: 282 - DNA, 23 bp, synthetic construct
  misc_feature 1..23: note = Single strand oligonucleotide
  source 1..23: mol_type = other DNA
tgtgatttaccagatcatgctgg

SEQ ID NO: 283 - DNA, 23 bp, synthetic construct
  misc_feature 1..23: note = Single strand oligonucleotide
  source 1..23: mol_type = other DNA
tgtgatttaccagatcatgctgg

SEQ ID NO: 284 - DNA, 23 bp, synthetic construct
  misc_feature 1..23: note = Single strand oligonucleotide
  source 1..23: mol_type = other DNA
gaggtttatcgatcgattcatgg

SEQ ID NO: 285 - DNA, 23 bp, synthetic construct
  misc_feature 1..23: note = Single strand oligonucleotide
  source 1..23: mol_type = other DNA
tgtgatttaccagatcatgctgg

SEQ ID NO: 286 - DNA, 23 bp, synthetic construct
  misc_feature 1..23: note = Single strand oligonucleotide
  source 1..23: mol_type = other DNA
tgtgatttaccagatcatgctgg

SEQ ID NO: 287 - DNA, 23 bp, synthetic construct
  misc_feature 1..23: note = Single strand oligonucleotide
  source 1..23: mol_type = other DNA
agcaatccgactctcaatacagg

SEQ ID NO: 288 - DNA, 23 bp, synthetic construct
  misc_feature 1..23: note = Single strand oligonucleotide
  source 1..23: mol_type = other DNA
gcacgacttgctgacgtggaagg

SEQ ID NO: 289 - DNA, 23 bp, synthetic construct
  misc_feature 1..23: note = Single strand oligonucleotide
  source 1..23: mol_type = other DNA
ggacaactaagtgaagaaagagg

SEQ ID NO: 290 - DNA, 23 bp, synthetic construct
  misc_feature 1..23: note = Single strand oligonucleotide
  source 1..23: mol_type = other DNA
ggtctcggatcttcaaacggtgg

SEQ ID NO: 291 - DNA, 23 bp, synthetic construct
  misc_feature 1..23: note = Single strand oligonucleotide
  source 1..23: mol_type = other DNA
agcaatccgactctcaatacagg

SEQ ID NO: 292 - DNA, 23 bp, synthetic construct
  misc_feature 1..23: note = Single strand oligonucleotide
  source 1..23: mol_type = other DNA
aagatagagagcacagatgatgg

SEQ ID NO: 293 - DNA, 23 bp, synthetic construct
  misc_feature 1..23: note = Single strand oligonucleotide
  source 1..23: mol_type = other DNA
accggaccctctaacagtggagg

SEQ ID NO: 294 - DNA, 23 bp, synthetic construct
  misc_feature 1..23: note = Single strand oligonucleotide
  source 1..23: mol_type = other DNA
agcaatccgactctcaatacagg

SEQ ID NO: 295 - DNA, 23 bp, synthetic construct
  misc_feature 1..23: note = Single strand oligonucleotide
  source 1..23: mol_type = other DNA
agcaatccgactctcaatacagg

SEQ ID NO: 296 - DNA, 23 bp, synthetic construct
  misc_feature 1..23: note = Single strand oligonucleotide
  source 1..23: mol_type = other DNA
aagatagagagcacagatgatgg

SEQ ID NO: 297 - DNA, 23 bp, synthetic construct
  misc_feature 1..23: note = Single strand oligonucleotide
  source 1..23: mol_type = other DNA
caacattctagccctacttctgg

SEQ ID NO: 298 - DNA, 23 bp, synthetic construct
  misc_feature 1..23: note = Single strand oligonucleotide
  source 1..23: mol_type = other DNA
ggtctcggatcttcaaacggtgg

SEQ ID NO: 299 - DNA, 23 bp, synthetic construct
  misc_feature 1..23: note = Single strand oligonucleotide
  source 1..23: mol_type = other DNA
aggggaatgttgtctggctcggg

SEQ ID NO: 300 - DNA, 23 bp, synthetic construct
  misc_feature 1..23: note = Single strand oligonucleotide
  source 1..23: mol_type = other DNA
gacagaagagagtgagcacacgg

SEQ ID NO: 301 - DNA, 23 bp, synthetic construct
  misc_feature 1..23: note = Single strand oligonucleotide
  source 1..23: mol_type = other DNA
catggattgggctacatgagtgg

SEQ ID NO: 302 - DNA, 23 bp, synthetic construct
  misc_feature 1..23: note = Single strand oligonucleotide
  source 1..23: mol_type = other DNA
aggggaatgttgtctggctcggg

SEQ ID NO: 303 - DNA, 23 bp, synthetic construct
  misc_feature 1..23: note = Single strand oligonucleotide
  source 1..23: mol_type = other DNA
aggggaatgttgtctggctcggg

SEQ ID NO: 304 - DNA, 23 bp, synthetic construct
  misc_feature 1..23: note = Single strand oligonucleotide
  source 1..23: mol_type = other DNA
agtttgttgctctagatgagtgg

SEQ ID NO: 305 - DNA, 23 bp, synthetic construct
  misc_feature 1..23: note = Single strand oligonucleotide
  source 1..23: mol_type = other DNA
aaagaggagcgagcacgcggcgg

SEQ ID NO: 306 - DNA, 23 bp, synthetic construct
  misc_feature 1..23: note = Single strand oligonucleotide
  source 1..23: mol_type = other DNA
aggggaatgttgtctggctcggg

SEQ ID NO: 307 - DNA, 23 bp, synthetic construct
  misc_feature 1..23: note = Single strand oligonucleotide
  source 1..23: mol_type = other DNA
aggggaatgttgtctggctcggg

SEQ ID NO: 308 - DNA, 23 bp, synthetic construct
  misc_feature 1..23: note = Single strand oligonucleotide
  source 1..23: mol_type = other DNA
tgagagatgctgacagaaagagg

SEQ ID NO: 309 - DNA, 23 bp, synthetic construct
  misc_feature 1..23: note = Single strand oligonucleotide
  source 1..23: mol_type = other DNA
catggattgggctacatgagtgg

SEQ ID NO: 310 - DNA, 23 bp, synthetic construct
  misc_feature 1..23: note = Single strand oligonucleotide
  source 1..23: mol_type = other DNA
aggggaatgttgtctggctcggg

SEQ ID NO: 311 - DNA, 23 bp, synthetic construct
  misc_feature 1..23: note = Single strand oligonucleotide
  source 1..23: mol_type = other DNA
ttatctgtgcttggactgaaggg

SEQ ID NO: 312 - DNA, 23 bp, synthetic construct
  misc_feature 1..23: note = Single strand oligonucleotide
  source 1..23: mol_type = other DNA
gatagagagcacgaataatgagg

SEQ ID NO: 313 - DNA, 23 bp, synthetic construct
  misc_feature 1..23: note = Single strand oligonucleotide
  source 1..23: mol_type = other DNA
aagccttgctgaagtgtttgggg

SEQ ID NO: 314 - DNA, 23 bp, synthetic construct
  misc_feature 1..23: note = Single strand oligonucleotide
  source 1..23: mol_type = other DNA
ttatctgtgcttggactgaaggg

SEQ ID NO: 315 - DNA, 23 bp, synthetic construct
  misc_feature 1..23: note = Single strand oligonucleotide
  source 1..23: mol_type = other DNA
ttatctgtgcttggactgaaggg

SEQ ID NO: 316 - DNA, 23 bp, synthetic construct
  misc_feature 1..23: note = Single strand oligonucleotide
  source 1..23: mol_type = other DNA
ctacgcaggagagatgatgctgg

SEQ ID NO: 317 - DNA, 23 bp, synthetic construct
  misc_feature 1..23: note = Single strand oligonucleotide
  source 1..23: mol_type = other DNA
agctcccttcagtccaagcaagg

SEQ ID NO: 318 - DNA, 23 bp, synthetic construct
  misc_feature 1..23: note = Single strand oligonucleotide
  source 1..23: mol_type = other DNA
ttatctgtgcttggactgaaggg

SEQ ID NO: 319 - DNA, 23 bp, synthetic construct
  misc_feature 1..23: note = Single strand oligonucleotide
  source 1..23: mol_type = other DNA
ttatctgtgcttggactgaaggg

SEQ ID NO: 320 - DNA, 23 bp, synthetic construct
  misc_feature 1..23: note = Single strand oligonucleotide
  source 1..23: mol_type = other DNA
gatagagagcacgaataatgagg

SEQ ID NO: 321 - DNA, 23 bp, synthetic construct
  misc_feature 1..23: note = Single strand oligonucleotide
  source 1..23: mol_type = other DNA
aagccttgctgaagtgtttgggg

SEQ ID NO: 322 - DNA, 23 bp, synthetic construct
  misc_feature 1..23: note = Single strand oligonucleotide
  source 1..23: mol_type = other DNA
ttatctgtgcttggactgaaggg

SEQ ID NO: 323 - DNA, 23 bp, synthetic construct
  misc_feature 1..23: note = Single strand oligonucleotide
  source 1..23: mol_type = other DNA
ggaagggagaatatccaggatgg

SEQ ID NO: 324 - DNA, 23 bp, synthetic construct
  misc_feature 1..23: note = Single strand oligonucleotide
  source 1..23: mol_type = other DNA
acaccctggattattcgaaaagg

SEQ ID NO: 325 - DNA, 23 bp, synthetic construct
  misc_feature 1..23: note = Single strand oligonucleotide
  source 1..23: mol_type = other DNA
gatatgggcatgggcggtgtagg

SEQ ID NO: 326 - DNA, 23 bp, synthetic construct
  misc_feature 1..23: note = Single strand oligonucleotide
  source 1..23: mol_type = other DNA
ggaagggagaatatccaggatgg

SEQ ID NO: 327 - DNA, 23 bp, synthetic construct
  misc_feature 1..23: note = Single strand oligonucleotide
  source 1..23: mol_type = other DNA
ggaagggagaatatccaggatgg

SEQ ID NO: 328 - DNA, 23 bp, synthetic construct
  misc_feature 1..23: note = Single strand oligonucleotide
  source 1..23: mol_type = other DNA
acaccctggattattcgaaaagg

SEQ ID NO: 329 - DNA, 23 bp, synthetic construct
  misc_feature 1..23: note = Single strand oligonucleotide
  source 1..23: mol_type = other DNA
gatatgggcatgggcggtgtagg

SEQ ID NO: 330 - DNA, 23 bp, synthetic construct
  misc_feature 1..23: note = Single strand oligonucleotide
  source 1..23: mol_type = other DNA
ggaagggagaatatccaggatgg

SEQ ID NO: 331 - DNA, 23 bp, synthetic construct
  misc_feature 1..23: note = Single strand oligonucleotide
  source 1..23: mol_type = other DNA
ggaagggagaatatccaggatgg

SEQ ID NO: 332 - DNA, 23 bp, synthetic construct
  misc_feature 1..23: note = Single strand oligonucleotide
  source 1..23: mol_type = other DNA
cgcaccgggcttgcctagaacgg

SEQ ID NO: 333 - DNA, 23 bp, synthetic construct
  misc_feature 1..23: note = Single strand oligonucleotide
  source 1..23: mol_type = other DNA
gagagacggaattgagaagaggg

SEQ ID NO: 334 - DNA, 23 bp, synthetic construct
  misc_feature 1..23: note = Single strand oligonucleotide
  source 1..23: mol_type = other DNA
ggaagggagaatatccaggatgg

SEQ ID NO: 335 - DNA, 23 bp, synthetic construct
  misc_feature 1..23: note = Single strand oligonucleotide
  source 1..23: mol_type = other DNA
gaggctatgtgctgcagccaagg

SEQ ID NO: 336 - DNA, 23 bp, synthetic construct
  misc_feature 1..23: note = Single strand oligonucleotide
  source 1..23: mol_type = other DNA
gaaggaagtttagatcatgctgg

SEQ ID NO: 337 - DNA, 23 bp, synthetic construct
  misc_feature 1..23: note = Single strand oligonucleotide
  source 1..23: mol_type = other DNA
ctgccagcatgatctatctttgg

SEQ ID NO: 338 - DNA, 23 bp, synthetic construct
  misc_feature 1..23: note = Single strand oligonucleotide
  source 1..23: mol_type = other DNA
gaggctatgtgctgcagccaagg

SEQ ID NO: 339 - DNA, 23 bp, synthetic construct
  misc_feature 1..23: note = Single strand oligonucleotide
  source 1..23: mol_type = other DNA
ttgaagctgccagcatgatctgg

SEQ ID NO: 340 - DNA, 23 bp, synthetic construct
  misc_feature 1..23: note = Single strand oligonucleotide
  source 1..23: mol_type = other DNA
taaaccatgctggagaagcaggg

SEQ ID NO: 341 - DNA, 23 bp, synthetic construct
  misc_feature 1..23: note = Single strand oligonucleotide
  source 1..23: mol_type = other DNA
ttgaagctgccagcatgatctgg

SEQ ID NO: 342 - DNA, 23 bp, synthetic construct
  misc_feature 1..23: note = Single strand oligonucleotide
  source 1..23: mol_type = other DNA
ttgaagctgccagcatgatctgg

SEQ ID NO: 343 - DNA, 23 bp, synthetic construct
  misc_feature 1..23: note = Single strand oligonucleotide
  source 1..23: mol_type = other DNA
ttgaagctgccagcatgatctgg

SEQ ID NO: 344 - DNA, 23 bp, synthetic construct
  misc_feature 1..23: note = Single strand oligonucleotide
  source 1..23: mol_type = other DNA
tagggaagttgagatcatgctgg

SEQ ID NO: 345 - DNA, 23 bp, synthetic construct
  misc_feature 1..23: note = Single strand oligonucleotide
  source 1..23: mol_type = other DNA
ttgaagctgccagcatgatctgg

SEQ ID NO: 346 - DNA, 23 bp, synthetic construct
  misc_feature 1..23: note = Single strand oligonucleotide
  source 1..23: mol_type = other DNA
ttgaagctgccagcatgatctgg

SEQ ID NO: 347 - DNA, 23 bp, synthetic construct
  misc_feature 1..23: note = Single strand oligonucleotide
  source 1..23: mol_type = other DNA
ttgaagctgccagcatgatctgg

SEQ ID NO: 348 - DNA, 23 bp, synthetic construct
  misc_feature 1..23: note = Single strand oligonucleotide
  source 1..23: mol_type = other DNA
aatctgaatgatctcggaccagg

SEQ ID NO: 349 - DNA, 23 bp, synthetic construct
  misc_feature 1..23: note = Single strand oligonucleotide
  source 1..23: mol_type = other DNA
ttgaagctgccagcatgatctgg

SEQ ID NO: 350 - DNA, 23 bp, synthetic construct
  misc_feature 1..23: note = Single strand oligonucleotide
  source 1..23: mol_type = other DNA
ttgaagctgccagcatgatctgg

SEQ ID NO: 351 - DNA, 23 bp, synthetic construct
  misc_feature 1..23: note = Single strand oligonucleotide
  source 1..23: mol_type = other DNA
ttgaagctgccagcatgatctgg

SEQ ID NO: 352 - DNA, 23 bp, synthetic construct
  misc_feature 1..23: note = Single strand oligonucleotide
  source 1..23: mol_type = other DNA
ataagtgaaggtgtcggaccagg

SEQ ID NO: 353 - DNA, 23 bp, synthetic construct
  misc_feature 1..23: note = Single strand oligonucleotide
  source 1..23: mol_type = other DNA
ttgaagctgccagcatgatctgg

SEQ ID NO: 354 - DNA, 23 bp, synthetic construct
  misc_feature 1..23: note = Single strand oligonucleotide
  source 1..23: mol_type = other DNA
ttgaagctgccagcatgatctgg

SEQ ID NO: 355 - DNA, 23 bp, synthetic construct
  misc_feature 1..23: note = Single strand oligonucleotide
  source 1..23: mol_type = other DNA
aggggaatgttgtctggctcggg

SEQ ID NO: 356 - DNA, 23 bp, synthetic construct
  misc_feature 1..23: note = Single strand oligonucleotide
  source 1..23: mol_type = other DNA
agggttgcctataagatggatgg

SEQ ID NO: 357 - DNA, 23 bp, synthetic construct
  misc_feature 1..23: note = Single strand oligonucleotide
  source 1..23: mol_type = other DNA
agaagagagtgagcacgcatcgg

SEQ ID NO: 358 - DNA, 23 bp, synthetic construct
  misc_feature 1..23: note = Single strand oligonucleotide
  source 1..23: mol_type = other DNA
aggggaatgttgtctggctcggg

SEQ ID NO: 359 - DNA, 23 bp, synthetic construct
  misc_feature 1..23: note = Single strand oligonucleotide
  source 1..23: mol_type = other DNA
aggggaatgttgtctggctcggg

SEQ ID NO: 360 - DNA, 23 bp, synthetic construct
  misc_feature 1..23: note = Single strand oligonucleotide
  source 1..23: mol_type = other DNA
cagccgtggctcgttcggaccgg

SEQ ID NO: 361 - DNA, 23 bp, synthetic construct
  misc_feature 1..23: note = Single strand oligonucleotide
  source 1..23: mol_type = other DNA
agaaggttgtgatattggcacgg

SEQ ID NO: 362 - DNA, 23 bp, synthetic construct
  misc_feature 1..23: note = Single strand oligonucleotide
  source 1..23: mol_type = other DNA
aggggaatgttgtctggctcggg

SEQ ID NO: 363 - DNA, 23 bp, synthetic construct
  misc_feature 1..23: note = Single strand oligonucleotide
  source 1..23: mol_type = other DNA
aggggaatgttgtctggctcggg

SEQ ID NO: 364 - DNA, 23 bp, synthetic construct
  misc_feature 1..23: note = Single strand oligonucleotide
  source 1..23: mol_type = other DNA
gctggggatgtgaatcttgatgg

SEQ ID NO: 365 - DNA, 23 bp, synthetic construct
  misc_feature 1..23: note = Single strand oligonucleotide
  source 1..23: mol_type = other DNA
aggggaatgttgtctggctcggg

SEQ ID NO: 366 - DNA, 23 bp, synthetic construct
  misc_feature 1..23: note = Single strand oligonucleotide
  source 1..23: mol_type = other DNA
aggggaatgttgtctggctcggg

SEQ ID NO: 367 - DNA, 22 bp, synthetic construct
  misc_feature 1..22: note = Single strand oligonucleotide
  source 1..22: mol_type = other DNA
gttgagagtgttggagaaggag

SEQ ID NO: 368 - DNA, 21 bp, synthetic construct
  misc_feature 1..21: note = Single strand oligonucleotide
  source 1..21: mol_type = other DNA
ctcggtgttgatcctgagaag

SEQ ID NO: 369 - DNA, 20 bp, synthetic construct
  misc_feature 1..20: note = Single strand oligonucleotide
  source 1..20: mol_type = other DNA
gtactgctggtcctttgcag

SEQ ID NO: 370 - DNA, 20 bp, synthetic construct
  misc_feature 1..20: note = Single strand oligonucleotide
  source 1..20: mol_type = other DNA
aggagcactacggaaggatg

SEQ ID NO: 371 - DNA, 19 bp, synthetic construct
  misc_feature 1..19: note = Single strand oligonucleotide
  source 1..19: mol_type = other DNA
acaccctgggaattggttt

SEQ ID NO: 372 - DNA, 20 bp, synthetic construct
  misc_feature 1..20: note = Single strand oligonucleotide
  source 1..20: mol_type = other DNA
gtatgcgccaataagaccac

SEQ ID NO: 373 - DNA, 95 bp, synthetic construct
  misc_feature 1..95: note = Single strand oligonucleotide
  source 1..95: mol_type = other DNA
accagaagcgaacatctctttttagtgttgttttgttacgacaaagtagagcttttgtaggcattgggttgctttagtttcttctcctgcccttc

SEQ ID NO: 374 - DNA, 22 bp, synthetic construct
  misc_feature 1..22: note = Single strand oligonucleotide
  source 1..22: mol_type = other DNA
ttcgcttgcagagagaaatcac

SEQ ID NO: 375 - DNA, 95 bp, synthetic construct
  misc_feature 1..95: note = Single strand oligonucleotide
  source 1..95: mol_type = other DNA
aacttcttcagcacgacgaagtacaccagccccatcgccgacttcgtgacgtgcggcatccagtcccaccagtgctcccacccggtcccgtgccc

SEQ ID NO: 376 - DNA, 21 bp, synthetic construct
  misc_feature 1..21: note = Single strand oligonucleotide
  source 1..21: mol_type = other DNA
aagtcgtgctgcttcatgtgg

SEQ ID NO: 377 - DNA, 95 bp, synthetic construct
  misc_feature 1..95: note = Single strand oligonucleotide
  source 1..95: mol_type = other DNA
acaacatcaacatgaggtcgaacacggggtcctacaacggcaggaggaacttcagctacgggaagtcgagctacgccaagtggtcccacagcggg

SEQ ID NO: 378 - DNA, 21 bp, synthetic construct
  misc_feature 1..21: note = Single strand oligonucleotide
  source 1..21: mol_type = other DNA
agttgtactccagcttgtgcc

SEQ ID NO: 379 - DNA, 95 bp, synthetic construct
  misc_feature 1..95: note = Single strand oligonucleotide
  source 1..95: mol_type = other DNA
aaccttataggtgtgtttgatggacgtttcctggtcgtcatgaggaggaggaacaagaacagaattcgcgaactcttcacccttgggatttcgat

SEQ ID NO: 380 - DNA, 21 bp, synthetic construct
  misc_feature 1..21: note = Single strand oligonucleotide
  source 1..21: mol_type = other DNA
tatccacacaaactacctgca

SEQ ID NO: 381 - DNA, 95 bp, synthetic construct
  misc_feature 1..95: note = Single strand oligonucleotide
  source 1..95: mol_type = other DNA
acgtcaactgttaggtcggttaggtcgtggtcgttaatgttgaaagtttccgaatcgtcctgctcctcgtgatgccttcctacgtctatttgatc

SEQ ID NO: 382 - DNA, 21 bp, synthetic construct
  misc_feature 1..21: note = Single strand oligonucleotide
  source 1..21: mol_type = other DNA
tgacaatccagccaatccagc

SEQ ID NO: 383 - DNA, 94 bp, synthetic construct
  misc_feature 1..94: note = Single strand oligonucleotide
  source 1..94: mol_type = other DNA
gaggccatttctagccgttgtgtactagaggaccgacttctagtcagtgaggaagaggttgtgagagttgttagggaggtcgaagtaccggctt

SEQ ID NO: 384 - DNA, 21 bp, synthetic construct
  misc_feature 1..21: note = Single strand oligonucleotide
  source 1..21: mol_type = other DNA
taaagatcggcaacacatgat

SEQ ID NO: 385 - DNA, 94 bp, synthetic construct
  misc_feature 1..94: note = Single strand oligonucleotide
  source 1..94: mol_type = other DNA
tgtgaaactggaaagaacccaaatcggtgtaagtttcaacgaggattgggtcatctgtttggtgttgactgttatgtctggaacagttctcctc

SEQ ID NO: 386 - DNA, 21 bp, synthetic construct
  misc_feature 1..21: note = Single strand oligonucleotide
  source 1..21: mol_type = other DNA
tgacctttcttgggtttagcc

SEQ ID NO: 387 - DNA, 87 bp, synthetic construct
  misc_feature 1..87: note = Single strand oligonucleotide
  source 1..87: mol_type = other DNA
ctgattttcgagtctatcctattgtggcgaaatagtaactttgaccttacggcttctttttgagttacagagtcgtgtcgcctaggt

SEQ ID NO: 388 - DNA, 21 bp, synthetic construct
  misc_feature 1..21: note = Single strand oligonucleotide
  source 1..21: mol_type = other DNA
aagctcaggagggatagcgcc

SEQ ID NO: 389 - DNA, 87 bp, synthetic construct
  misc_feature 1..87: note = Single strand oligonucleotide
  source 1..87: mol_type = other DNA
aacttcttcagcacgacgaagtacaccagccccatcgccgacttcgtgacgtgcggcatccagtcccaccagtgctcccacccggtc

SEQ ID NO: 390 - DNA, 21 bp, synthetic construct
  misc_feature 1..21: note = Single strand oligonucleotide
  source 1..21: mol_type = other DNA
aagtcgtgctgcttcatgtgg

SEQ ID NO: 391 - DNA, 87 bp, synthetic construct
  misc_feature 1..87: note = Single strand oligonucleotide
  source 1..87: mol_type = other DNA
acaacatcaacatgaggtcgaacacggggtcctacaacggcaggaggaacttcagctacgggaagtcgagctacgccaagtggtccc

SEQ ID NO: 392 - DNA, 21 bp, synthetic construct
  misc_feature 1..21: note = Single strand oligonucleotide
  source 1..21: mol_type = other DNA
agttgtactccagcttgtgcc

SEQ ID NO: 393 - DNA, 87 bp, synthetic construct
  misc_feature 1..87: note = Single strand oligonucleotide
  source 1..87: mol_type = other DNA
aaccttataggtgtgtttgatggacgtttcctggtcgtcatgaggaggaggaacaagaacagaattcgcgaactcttcacccttggg

SEQ ID NO: 394 - DNA, 21 bp, synthetic construct
  misc_feature 1..21: note = Single strand oligonucleotide
  source 1..21: mol_type = other DNA
tatccacacaaactacctgca

SEQ ID NO: 395 - DNA, 87 bp, synthetic construct
  misc_feature 1..87: note = Single strand oligonucleotide
  source 1..87: mol_type = other DNA
acgtcaactgttaggtcggttaggtcgtggtcgttaatgttgaaagtttccgaatcgtcctgctcctcgtgatgccttcctacgtct

SEQ ID NO: 396 - DNA, 21 bp, synthetic construct
  misc_feature 1..21: note = Single strand oligonucleotide
  source 1..21: mol_type = other DNA
tgacaatccagccaatccagc

SEQ ID NO: 397 - DNA, 87 bp, synthetic construct
  misc_feature 1..87: note = Single strand oligonucleotide
  source 1..87: mol_type = other DNA
gaggccatttctagccgttgtgtactagaggaccgacttctagtcagtgaggaagaggttgtgagagttgttagggaggtcgaagta

SEQ ID NO: 398 - DNA, 21 bp, synthetic construct
  misc_feature 1..21: note = Single strand oligonucleotide
  source 1..21: mol_type = other DNA
taaagatcggcaacacatgat

SEQ ID NO: 399 - DNA, 89 bp, synthetic construct
  misc_feature 1..89: note = Single strand oligonucleotide
  source 1..89: mol_type = other DNA
tgtgaaactggaaagaacccaaatcggtgtaagtttcaacgaggattgggtcatctgtttggtgttgactgttatgtctggaacagttc

SEQ ID NO: 400 - DNA, 21 bp, synthetic construct
  misc_feature 1..21: note = Single strand oligonucleotide
  source 1..21: mol_type = other DNA
tgacctttcttgggtttagcc

SEQ ID NO: 401 - DNA, 416 bp, synthetic construct
  misc_feature 1..416: note = Single strand oligonucleotide
  source 1..416: mol_type = other DNA
aaaacgtaataagttctttttgtgtgtgtctgcaggcaatatcaaaaacataaccatcatgatgtatagagactgtcgggtccattgtgaggagacattcagtttctctttaaaactccttcattgaaatagtccggtgttatccctacctgagcttagttttttttttttaattttttttctgtcctattgaattattctattctcttgtccatgttcgacccatccctttcaaagtatctcaaccttctatcgttttaaagactctctcctatctctttttggtgttgagtatgtgtgtatctctactcctagttcatttgaatcagtttttctaccttgtctatccctcctgagctaatgtttgcatcttcttgttggtcattgatgtatggttgatataaattccaaataaa

SEQ ID NO: 402 - DNA, 416 bp, synthetic construct
  misc_feature 1..416: note = Single strand oligonucleotide
  source 1..416: mol_type = other DNA
aaaacgtaataagttctttttgtgtgtgtctgcaggcaatatcaaaaacataaccatcatgatgtatagagactgtcgggtccattgtgaggagacattcagtttctctttaaaactccttcattgaaatagtccggtgttatccctacctgagcttagttttttttttttaattttttttctgtcctattgaattattctattggtcacttgaccgccatgacatccctttcaaagtatctcaaccttctatcgttttaaagactctctcctatctctttttggtgttgagtatgtgtgtatctctactcctagttcatttgaatcagtttttctaccttgtctatccctcctgagctaatgtttgcatcttcttgttggtcattgatgtatggttgatataaattccaaataaa

SEQ ID NO: 403 - DNA, 416 bp, synthetic construct
  misc_feature 1..416: note = Single strand oligonucleotide
  source 1..416: mol_type = other DNA
aaaacgtaataagttctttttgtgtgtgtctgcaggcaatatcaaaaacataaccatcatgatgtatagagactgtcgggtccattgtgaggagacattcagtttctctttaaaactccttcattgaaatagtccggtgttatccctacctgagcttagttttttttttttaattttttttctgtcctattgaattattctattttcttgaccttgtaagacccatccctttcaaagtatctcaaccttctatcgttttaaagactctctcctatctctttttggtgttgagtatgtgtgtatctctactcctagttcatttgaatcagtttttctaccttgtctatccctcctgagctaatgtttgcatcttcttgttggtcattgatgtatggttgatataaattccaaataaa

SEQ ID NO: 404 - DNA, 107 bp, synthetic construct; identical to SEQ ID NO: 64
  misc_feature 1..107: note = Single strand oligonucleotide
  source 1..107: mol_type = other DNA

SEQ ID NO: 405 - DNA, 107 bp, synthetic construct; identical to SEQ ID NO: 65
  misc_feature 1..107: note = Single strand oligonucleotide
  source 1..107: mol_type = other DNA

SEQ ID NO: 406 - DNA, 107 bp, synthetic construct; identical to SEQ ID NO: 66
  misc_feature 1..107: note = Single strand oligonucleotide
  source 1..107: mol_type = other DNA

SEQ ID NO: 407 - DNA, 107 bp, synthetic construct; identical to SEQ ID NO: 67
  misc_feature 1..107: note = Single strand oligonucleotide
  source 1..107: mol_type = other DNA

SEQ ID NO: 408 - DNA, 107 bp, synthetic construct; identical to SEQ ID NO: 68
  misc_feature 1..107: note = Single strand oligonucleotide
  source 1..107: mol_type = other DNA

SEQ ID NO: 409 - DNA, 107 bp, synthetic construct; identical to SEQ ID NO: 69
  misc_feature 1..107: note = Single strand oligonucleotide
  source 1..107: mol_type = other DNA

SEQ ID NO: 410 - DNA, 107 bp, synthetic construct; identical to SEQ ID NO: 70
  misc_feature 1..107: note = Single strand oligonucleotide
  source 1..107: mol_type = other DNA

SEQ ID NO: 411 - DNA, 102 bp, synthetic construct; identical to SEQ ID NO: 57
  misc_feature 1..102: note = Single strand oligonucleotide
  source 1..102: mol_type = other DNA

SEQ ID NO: 412 - DNA, 102 bp, synthetic construct; identical to SEQ ID NO: 58
  misc_feature 1..102: note = Single strand oligonucleotide
  source 1..102: mol_type = other DNA

SEQ ID NO: 413 - DNA, 102 bp, synthetic construct; identical to SEQ ID NO: 59
  misc_feature 1..102: note = Single strand oligonucleotide
  source 1..102: mol_type = other DNA

SEQ ID NO: 414 - DNA, 102 bp, synthetic construct; identical to SEQ ID NO: 60
  misc_feature 1..102: note = Single strand oligonucleotide
  source 1..102: mol_type = other DNA

SEQ ID NO: 415 - DNA, 102 bp, synthetic construct; identical to SEQ ID NO: 61
  misc_feature 1..102: note = Single strand oligonucleotide
  source 1..102: mol_type = other DNA

SEQ ID NO: 416 - DNA, 102 bp, synthetic construct; identical to SEQ ID NO: 62
  misc_feature 1..102: note = Single strand oligonucleotide
  source 1..102: mol_type = other DNA

SEQ ID NO: 417 - DNA, 102 bp, synthetic construct; identical to SEQ ID NO: 63
  misc_feature 1..102: note = Single strand oligonucleotide
  source 1..102: mol_type = other DNA

(19) United States
(12) Patent Application Publication
(10) Pub. No.: US 2026/0009031 A1
(43) Pub. Date: Jan. 8, 2026

(54) MODIFYING THE SPECIFICITY OF PLANT NON-CODING RNA MOLECULES FOR SILENCING GENE EXPRESSION

(71) Applicant: Tropic Biosciences UK Limited, Norwich (GB)

(72) Inventors: Eyal MAORI, Cambridge (GB); Yaron GALANTY, Cambridge (GB); Cristina PIGNOCCHI, Norwich (GB); Angela CHAPARRO GARCIA, Norwich (GB); Ofir Meir, Norwich (GB)

(73) Assignee: Tropic Biosciences UK Limited, Norwich (GB)

(21) Appl. No.: 19/210,663

(22) Filed: May 16, 2025

Related U.S. Application Data
(63) Continuation of application No. 18/152,718, filed on Jan. 10, 2023, now Pat. No. 12,331,295.
Application No. 18/152,718 is a division of application No. 16/648,748, filed on Mar. 19, 2020, now Pat. No. 11,555,199.

(51) Int. Cl. C12N 15/113; C12N 9/22; C12N 15/82; C12N 15/85; C12N 15/90

(52) U.S. Cl. CPC C12N 15/113; C12N 9/22; C12N 15/8213; C12N 15/8279; C12N 15/85; C12N 15/902; C12N 15/8218; C12N 2310/11; C12N 2310/141; C12N 2310/20

(57) ABSTRACT

A method of modifying a gene encoding or processed into a non-coding RNA molecule having no RNA silencing activity in a plant cell is disclosed. The method comprising introducing into the plant cell a DNA editing agent conferring a silencing specificity of the non-coding RNA molecule towards a target RNA of interest. A method of modifying a gene encoding or processed into a RNA silencing molecule in a plant cell is also disclosed. The method comprising introducing into the plant cell a DNA editing agent which redirects the silencing specificity of the non-coding RNA molecule towards a target RNA of interest. Plant cells, plant seeds, plants, and methods of generating plants are also disclosed.

### RELATED APPLICATIONS

This application is a continuation application of U.S. patent application Ser. No. 18/152,718, filed on Jan. 10, 2023, which is a divisional application of U.S. patent application Ser. No. 16/648,748, filed on Mar. 19, 2020, now issued as U.S. Pat. No. 11,555,199, which is a National Phase of PCT Application No. PCT/IB2018/057160, having an International filing date of Sep. 18, 2018, which claims the benefit of priority of Great Britain Patent Application No. 1719516.5 filed on Nov. 23, 2017, and Great Britain Patent Application Nos. 1715116.8 and 1715113.5 both filed on Sep. 19, 2017. The contents of the above applications are all incorporated by reference as if fully set forth herein in their entirety.

### REFERENCE TO AN ELECTRONIC SEQUENCE LISTING

The contents of the electronic sequence listing (251502011902seqlist.xml; Size: 672,147 bytes; and Date of Creation: Mar. 14, 2025) is herein incorporated by reference in its entirety.

### FIELD AND BACKGROUND OF THE INVENTION

The present invention, in some embodiments thereof, relates to modifying genes that encode or are processed into non-coding RNA molecules, including RNA silencing molecules and, more particularly, but not exclusively, to the use of same for silencing endogenous or exogenous target gene-expression of interest in plants.

RNA silencing or RNA interference (RNAi), the endogenous co- or post-transcriptional genetic regulatory mechanism in which RNA molecules inhibit gene expression or translation, is generally mediated by non-coding RNA molecules including microRNAs (miRNAs), small interfering RNAs (siRNAs), trans-acting siRNA (tasiRNA), piwi-interacting RNAs (piRNA), antisense RNA, etc. Recently, additional non-coding RNAs have been implicated to harbour a RNA silencing activity including transfer RNA (tRNA), small nuclear RNA (snoRNA), small nucleolar RNA (snoRNA) and repeats-derived RNA. These canonical and non-canonical RNA silencing molecules differ in their substrates, biogenesis, effector proteins and modes of target down regulation.

Moreover, Argonaute proteins, in complex with small RNAs, form the core of the RNA-induced silencing complex (RISC), the RNA-interference (RNAi) effector complex. The Argonaute superfamily segregates into two clades, termed Ago and Piwi. Ago proteins (e.g. Ago1 and Ago2) typically complex with miRNAs and siRNAs, while Piwi proteins (e.g. Piwi, Ago3 and Aubergine (Aub)) typically complex with piRNA.

Small interfering RNAs (siRNAs) are double-stranded RNA molecules of 20-25 nucleotides (nt) in length, which interfere with the expression of specific genes with complementary nucleotide sequences by degrading their transcript during or after transcription resulting in no translation.

MicroRNAs (miRNAs) are small endogenous non-coding RNAs (ncRNAs) of 20 to 24 nt in length, originating from long self-complementary precursors. Mature miRNAs regulate gene expression in two ways; (i) by inhibiting translation or (ii) by degrading coding mRNAs by perfect or near-perfect complement with the target transcript. The majority of plant target mRNAs contain a single miRNA-complementary site, which results in the target mRNAs being cleaved and degraded by the RNA silencing molecule and RNA decay machinery.

Piwi-interacting RNAs (piRNAs) are small non-coding RNAs which are the product of long single stranded precursor molecules, and which are generated without a dicing step. piRNAs are typically 26 to 31 nt in length and are mostly antisense. piRNAs form RNA-protein complexes through interactions with Piwi proteins. Antisense piRNAs are typically loaded into Piwi or Aub.

Transacting siRNA (tasiRNA) are a class of small interfering RNA (siRNA) that repress gene expression through post-transcriptional gene silencing. Their biogenesis is primed by association of miRNAs to tasiRNA precursors, which recruits RNA-dependent RNA-polymerases (RdRp) that synthesize dsRNA from the tasiRNA precursor template. Next, such dsRNA is processed by DICER-LIKE 4 (DCL4) into about 21-nucleotide “phased” intervals mature tasiRNAs.

Recent advances in genome editing techniques have made it possible to alter DNA sequences in living cells. By editing only a few of the billions of nucleotides in the cells of plants, these new techniques might be the most effective way to get crops to grow better in harsh climates (crop performance and abiotic stress) and enhance resistance to biotic stress (insects, viruses, bacteria, beetles, nematodes etc.). There are limited approaches to achieve resistance to pests using genome editing technologies such as CRISPR/Cas9: plant susceptible genes knock-out (such as the well-known MLO genes), by introduction of stop codons, frame shifts, insertions, deletions etc.; or up regulation of resistance genes, like R genes, by modification of regulatory elements like promoters, microRNA binding sites etc. Nevertheless, approaches that target specifically the pathogen are limited to transgenic CRISPR applications.

Previous work on genome editing of RNA molecules in various organisms (e.g. murine, human, shrimp, plants), focused on knocking-out miRNA activity or changing their binding site in target RNAs, for example:

Zhao et al., [Zhao et al., Scientific Reports (2014) 4:3943] provided a miRNA inhibition strategy employing the CRISPR system in murine cells. Zhao used a specifically designed gRNAs to cut a miRNA gene at a single site by Cas9, resulting in knockdown of the miRNA in murine cells.

Jiang et al. [Jiang et al., RNA Biology (2014) 11 (10): 1243-9] used CRISPR/Cas9 to deplete human miR-93 from a cluster by targeting its 5′ region in HeLa cells. Various small indels were induced in the targeted region containing the Drosha processing site (i.e. the position at which Drosha, a double-stranded RNA-specific RNase III enzyme, binds, cleaves and thereby processes primary miRNAs (pri-miRNAs) into pre-miRNA in the nucleus of a host cell) and seed sequences (i.e. the conserved heptametrical sequences which are essential for the binding of the miRNA to mRNA, typically situated at positions 2-7 from the miRNA 5′-end). According to Jiang et al. even a single nucleotide deletion led to complete knockout of the target miRNA with high specificity.

With regard to plant genome editing, Bortesi and Fischer [Bortesi and Fischer, Biotechnology Advances (2015) 33: 41-52] discussed the use of CRISPR-Cas9 technology in plants compared to ZFNs and TALENs, and Basak and Nithin [Basak and Nithin, Front Plant Sci. (2015) 6: 1001] demonstrated the use of CRISPR-Cas9 technology for knockdown of protein-coding genes in model plants such as Arabidopsis and tobacco and crops like wheat, maize, and rice.

In addition to disruption of miRNA activity or target binding sites, gene silencing using artificial microRNAs (amiRNAs)-mediated gene silencing of endogenous and exogenous target genes were used [Tiwari et al. Plant Mol Biol (2014) 86: 1]. Similar to microRNAs, amiRNAs are single-stranded, approximately 21 nt long, and designed by replacing the mature miRNA sequences of duplex within pre-miRNAs [Tiwari et al. (2014) supra]. These amiRNAs are introduced as a transgene within an artificial expression cassette (including a promoter, terminator etc.) [Carbonell et al., Plant Physiology (2014) pp. 113.234989], are processed via small RNA biogenesis and silencing machinery and downregulate target expression. According to Schwab et al. [Schwab et al. The Plant Cell (2006) Vol. 18, 1121-1133], amiRNAs are active when expressed under tissue-specific or inducible promoters and can be used for specific gene silencing in plants, especially when several related, but not identical, target genes need to be downregulated.

Senis et al. [Senis et al., Nucleic Acids Research (2017) Vol. 45(1): e3] disclose engineering of a promoterless anti-viral amiRNA into an endogenous miRNA locus. Specifically, Senis et al. insert a amiRNA precursor transgene (hairpin pri-amiRNA) adjacent to a naturally occurring miRNA gene (e.g. miR122) by homology-directed DNA recombination that is induced by sequence-specific nuclease such as Cas9 or TALEN. This approach uses promoter- and terminator-free amiRNAs by utilizing transcriptionally active DNA that expresses natural miRNA (miR122), that is, the endogenous promoter and terminator drove and regulated the transcription of the inserted amiRNA transgene.

Various DNA-free methods of introducing RNA and/or proteins into cells have been previously described. For example, RNA transfection using electroporation and lipofection has been described in U.S. Patent Application No. 20160289675. Direct delivery of Cas9/gRNA ribonucleoprotein (RNP) complexes to cells by microinjection of Cas9 protein and gRNA complexes was described by Cho [Cho et al., “Heritable gene knockout in Caenorhabditis elegans by direct injection of Cas9-sgRNA ribonucleoproteins,” Genetics (2013) 195:1177-1180]. Delivery of Cas9 protein/gRNA complexes via electroporation was described by Kim [Kim et al., “Highly efficient RNA-guided genome editing in human cells via delivery of purified Cas9 ribonucleoproteins” Genome Res. (2014) 24:1012-1019]. Delivery of Cas9 protein-associated gRNA complexes via liposomes was reported by Zuris [Zuris et al., “Cationic lipid-mediated delivery of proteins enables efficient protein-based genome editing in vitro and in vivo” Nat Biotechnol. (2014) doi: 10.1038/nbt.3081].

### SUMMARY OF THE INVENTION

According to an aspect of some embodiments of the present invention, there is provided a method of modifying a gene encoding or processed into a non-coding RNA molecule having no RNA silencing activity in a plant cell, the method comprising introducing into the plant cell a DNA editing agent conferring a silencing specificity of the non-coding RNA molecule towards a target RNA of interest, thereby modifying the gene encoding or processed into the non-coding RNA molecule.

According to an aspect of some embodiments of the present invention, there is provided a method of modifying a gene encoding or processed into a non-coding RNA molecule having no RNA silencing activity in a plant cell, the method comprising introducing into the plant cell a DNA editing agent conferring a silencing specificity of the non-coding RNA molecule towards a target RNA of interest.

According to an aspect of some embodiments of the present invention, there is provided a method of modifying a gene encoding or processed into a RNA silencing molecule to a target RNA in a plant cell, the method comprising introducing into the plant cell a DNA editing agent which redirects a silencing specificity of the RNA silencing molecule towards a second target RNA, the target RNA and the second target RNA being distinct, thereby modifying the gene encoding the RNA silencing molecule.

According to an aspect of some embodiments of the present invention, there is provided a method of modifying a gene encoding or processed into a RNA silencing molecule to a target RNA in a plant cell, the method comprising introducing into the plant cell a DNA editing agent which redirects a silencing specificity of the RNA silencing molecule towards a second target RNA, the target RNA and the second target RNA being distinct.

According to an aspect of some embodiments of the present invention, there is provided a plant cell generated according to the method of some embodiments of the invention.

According to an aspect of some embodiments of the present invention, there is provided a plant comprising the plant cell of some embodiments of the invention.

According to an aspect of some embodiments of the present invention, there is provided a method of producing a plant with reduced expression of a target gene, the method comprising: (a) breeding the plant of some embodiments of the invention; and (b) selecting for progeny plants that have reduced expression of the target RNA of interest or the second target RNA, or progeny that comprises a silencing specificity in the non-coding RNA molecule towards a target RNA of interest, and which do not comprise the DNA editing agent, thereby producing the plant with reduced expression of a target gene.

According to an aspect of some embodiments of the present invention, there is provided a method of generating a plant with increased stress tolerance, increased yield, increased growth rate or increased yield quality, the method comprising modifying a gene encoding or processed into a non-coding RNA molecule or into a RNA silencing molecule in a plant cell according to some embodiments of the invention, wherein the target RNA of interest is of a gene of the plant conferring sensitivity to stress, decreased yield, decreased growth rate or decreased yield quality thereby generating the plant.

According to an aspect of some embodiments of the present invention, there is provided a method of generating a pathogen tolerant or resistant plant, the method comprising modifying a gene encoding or processed into a non-coding RNA molecule or into a RNA silencing molecule in a plant cell according to some embodiments of the invention, wherein the target RNA of interest is of a gene of the plant conferring sensitivity to the pathogen, thereby generating the pathogen tolerant or resistant plant.

According to an aspect of some embodiments of the present invention, there is provided a method of generating a pathogen tolerant or resistant plant, the method comprising modifying a gene encoding or processed into a non-coding RNA molecule or into a RNA silencing molecule in a plant cell according to some embodiments of the invention, wherein the target RNA of interest is of a gene of the pathogen, thereby generating the pathogen tolerant or resistant plant.

According to an aspect of some embodiments of the present invention, there is provided a method of generating a pest tolerant or resistant plant, the method comprising modifying a gene encoding or processed into a non-coding RNA molecule or into a RNA silencing molecule in a plant cell according to some embodiments of the invention, wherein the target RNA of interest is of a gene of the pest, thereby generating the pest tolerant or resistant plant.

According to an aspect of some embodiments of the present invention, there is provided a method of generating a pest tolerant or resistant plant, the method comprising modifying a gene encoding or processed into a non-coding RNA molecule or into a RNA silencing molecule in a plant cell according to some embodiments of the invention, wherein the target RNA of interest is of a gene of the plant conferring sensitivity to the pest, thereby generating the pest tolerant or resistant plant.

According to an aspect of some embodiments of the present invention, there is provided a method of generating a herbicide resistant plant, the method comprising modifying a gene encoding or processed into a non-coding RNA molecule or into a RNA silencing molecule in a plant cell according to some embodiments of the invention, wherein the target RNA of interest is of a gene of the plant conferring sensitivity to the herbicide, thereby generating the herbicide resistant plant.

According to an aspect of some embodiments of the present invention, there is provided a plant generated according to the method of some embodiments of the invention.

According to an aspect of some embodiments of the present invention, there is provided a seed of the plant of some embodiments of the invention.

According to some embodiments of the invention, the gene encoding or processed into the non-coding RNA molecule is endogenous to the plant cell.

According to some embodiments of the invention, the gene encoding the RNA silencing molecule is endogenous to the plant cell.

According to some embodiments of the invention, modifying the gene encoding or processed into the non-coding RNA molecule comprises imparting the non-coding RNA molecule with at least 45% complementarity towards the target RNA of interest.

According to some embodiments of the invention, modifying the gene encoding the RNA silencing molecule comprises imparting the RNA silencing molecule with at least 45% complementarity towards the second target RNA.

According to some embodiments of the invention, the silencing specificity of the non-coding RNA molecule is determined by measuring a RNA or protein level of the target RNA of interest.

According to some embodiments of the invention, the silencing specificity of the RNA silencing molecule is determined by measuring a RNA level of the second target RNA.

According to some embodiments of the invention, the silencing specificity of the non-coding RNA molecule or the RNA silencing molecule is determined phenotypically.

According to some embodiments of the invention, determined phenotypically is effected by determination of at least one plant phenotype selected from the group consisting of plant a leaf coloring, a flower coloring, a growth rate, a plant size, a crop yield, a fruit trait, a biotic stress resistance, and an abiotic stress resistance.

According to some embodiments of the invention, the silencing specificity of the non-coding RNA molecule is determined genotypically.

According to some embodiments of the invention, the plant phenotype is determined prior to a plant genotype.

According to some embodiments of the invention, the plant genotype is determined prior to a plant phenotype.

According to some embodiments of the invention, the non-coding RNA molecule or the RNA silencing molecule is processed from a precursor.

According to some embodiments of the invention, the non-coding RNA molecule or the RNA silencing molecule is a RNA interference (RNAi) molecule.

According to some embodiments of the invention, the RNAi molecule is selected from the group consisting of a small interfering RNA (siRNA), a short hairpin RNA (shRNA), a microRNA (miRNA), a Piwi-interacting RNA (piRNA) and trans-acting siRNA (tasiRNA).

According to some embodiments of the invention, the non-coding RNA molecule is selected from the group consisting of a small nuclear RNA (snRNA), a small nucleolar RNA (snoRNA), a long non-coding RNA (lncRNA), a ribosomal RNA (rRNA), transfer RNA (tRNA), a repeat-derived RNA, and a transposable element RNA.

According to some embodiments of the invention, the RNA molecule or RNAi molecule is designed such that a sequence of the RNAi molecule is modified to preserve originality of structure and to be recognized by cellular RNAi factors.

According to some embodiments of the invention, modifying the gene is effected by a modification selected from the group consisting of a deletion, an insertion, a point mutation and a combination thereof.

According to some embodiments of the invention, the modification is in a stem region of the non-coding RNA molecule or the RNA silencing molecule.

According to some embodiments of the invention, the modification is in a loop region of the non-coding RNA molecule or the RNA silencing molecule.

According to some embodiments of the invention, the modification is in a non-structured region of the non-coding RNA molecule or the RNA silencing molecule.

According to some embodiments of the invention, the modification is in a stem region and a loop region of the non-coding RNA molecule or the RNA silencing molecule.

According to some embodiments of the invention, the modification is in a stem region and a loop region and in non-structured region of the non-coding RNA molecule or the RNA silencing molecule.

According to some embodiments of the invention, the modification is an insertion.

According to some embodiments of the invention, the modification is a deletion.

According to some embodiments of the invention, the modification is a point mutation.

According to some embodiments of the invention, the modification comprises a modification of at most 200 nucleotides.

According to some embodiments of the invention, the method further comprises introducing into the plant cell donor oligonucleotides.

According to some embodiments of the invention, the DNA editing agent comprises at least one gRNA operatively linked to a plant expressible promoter.

According to some embodiments of the invention, the DNA editing agent does not comprise an endonuclease.

According to some embodiments of the invention, the DNA editing agent comprises an endonuclease.

According to some embodiments of the invention, the DNA editing agent is of a DNA editing system selected from the group consisting of a meganuclease, a zinc finger nucleases (ZFN), a transcription-activator like effector nuclease (TALEN) and CRISPR.

According to some embodiments of the invention, the endonuclease comprises Cas9.

According to some embodiments of the invention, the DNA editing agent is applied to the cell as DNA, RNA or RNP.

According to some embodiments of the invention, the DNA editing agent is linked to a reporter for monitoring expression in a plant cell.

According to some embodiments of the invention, the reporter is a fluorescent protein.

According to some embodiments of the invention, the target RNA of interest or the second target RNA is endogenous to the plant cell.

According to some embodiments of the invention, the target RNA of interest or the second target RNA is exogenous to the plant cell.

According to some embodiments of the invention, the plant cell is a protoplast.

According to some embodiments of the invention, the breeding comprises crossing or selfing.

According to some embodiments of the invention, the plant is non-genetically modified (non-GMO).

According to some embodiments of the invention, the plant is selected from the group consisting of a crop, a flower and a tree.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an embodiment flow chart of Genome Editing Induced Gene Silencing (GEiGS) replacement of endogenous miRNA with siRNA targeting the PDS gene, hence inducing gene silencing of the endogenous PDS gene. To introduce the modification, a 2-component system is being used. First, a CRISPR/CAS9 system, in a GFP containing vector, generates a cleavage in the chosen loci, through designed specific guide RNAs to promote homologous DNA repair (HDR) in the site. Second, A DONOR sequence, with the desired modification of the miRNA sequence, to target the newly assigned genes, is introduced as a template for the HDR. This system is being used in protoplast transformation, enriched by FACS due to the GFP signal in the CRISPR/CAS9 vector, recovered, and regenerated to plants.

FIGS. 2A-C are photographs illustrating that silencing of the PDS gene causes photobleaching. Silencing of the PDS gene in Nicotiana (FIGS. 2A-B) and Arabidopsis (FIG. 2C) plants causes photobleaching in N. benthamiana (FIG. 2B) and Arabidopsis (FIG. 2C, right side). Photographs were taken 3½ weeks after PDS silencing.

FIG. 3A-D are photographs of knock down of GFP expression levels in Arabidopsis using GEiGS. Arabidopsis protoplasts expressing GFP are illustrated as control (FIGS. 3A-B) compared to protoplasts edited using GEiGS to express GFP siRNA (FIGS. 3C-D). Of note, GEiGS protoplasts or plants are silenced for expression of GFP protein.

FIG. 4 is an embodiment flow chart of GEiGS replacement of endogenous miRNA with siRNA targeting GFP, generating Arabidopsis plants with active RNAi against GFP. To introduce the modification, a CRISPR/CAS9 system, in a RFP containing vector, generates a cleavage in the chosen loci, through designed specific guide RNAs to promote homologous DNA repair (HDR) in the site. Second, A DONOR sequence, with the desired modification of the miRNA sequence, to target the GFP gene, is introduced as a template for the HDR. This system is being used in GFP expressing protoplasts. Enrichment of putatively modified cells by FACS due to the RFP signal in the CRISPR/CAS9 vector, is being carried out and recovered. Regenerated plants are being analysed for intensity of GFP signal.

FIG. 5 is an embodiment flow chart of GEiGS replacement of endogenous miRNA with siRNA targeting GFP, generating Arabidopsis plants with GEiGS-directed RNAi against GFP. Of note, GEiGS plants are silenced for GFP expression after plant transformation. RFP is being used for the enrichment of cells with transient presence of CRISPR/CAS9 vector.

FIG. 6 is an embodiment flow chart of GEiGS replacement of endogenous miRNA with siRNA targeting GFP, generating plants resistant to viral infection e.g. TMV infection (i.e. exogenous gene). RFP is being used for the enrichment of cells with transient presence of CRISPR/CAS9 vector.

FIG. 7 is a photograph of lodging banana plants suffering from Toppling Disease caused by the burrowing nematode, Radopholus similis.

FIG. 8 is a table illustrating the occurrence of Radopholus similis and Pratylenchus coffeae on different crops in Tay Nguyen area.

FIG. 9 is an embodiment flow chart of computational pipeline to generate GEiGS templates. The computational GEiGS pipeline applies biological metadata and enables an automatic generation of GEiGS DNA donor templates that are used to minimally edit endogenous non-coding RNA genes (e.g. miRNA genes), leading to a new gain of function, i.e. redirection of their silencing capacity to target gene expression of interest.

FIG. 10 is an embodiment flow chart illustrating design of resistant plant to pests targeting any desired exogenous pest gene. GEiGS replacement of endogenous miRNA with siRNA targeting pathogen/pest essential gene, generating plants resistant to pathogen/pest infection.

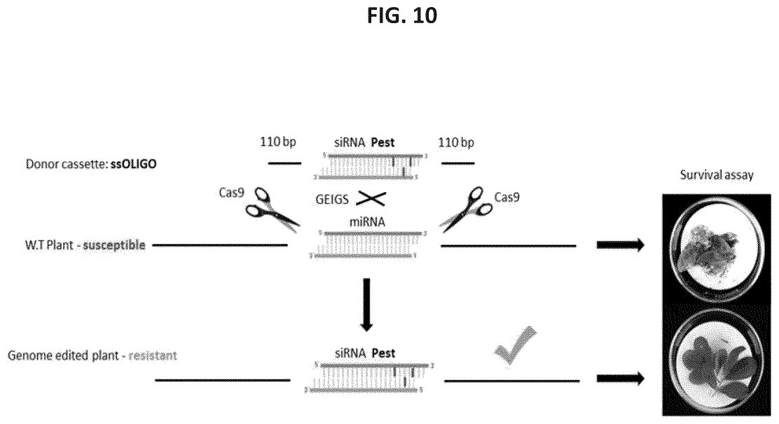

FIG. 11 is an embodiment drawing illustrating the main stages required to design RNA silencing molecule and with minimally edited miRNA gene bases.

FIGS. 12A-G illustrate primary transcripts of miR-390 and modified miR390-structure and targeted sequences. Secondary structure representation of primary transcripts of miR390, and its modified versions—(FIG. 12A) wild type; (FIGS. 12B-C) modified version to target GFP; (FIGS. 12D-E) modified version to target AtPDS3; (FIGS. 12F-G) modified version to target AtADH1. Mature miRNA/siRNAs are outlined in red, exhibiting structure conservation through design. The regions targeted for manipulation by CRISPR/CAS9 system are outlined in purple and the NGG sequence is highlighted in yellow (FIG. 12A).

FIGS. 13A-G illustrate primary transcripts of miR-173 and modified miR173-structure and targeted sequences. Secondary structure representation of primary transcripts of miR173, and its modified versions—(FIG. 13A) wild type; (FIGS. 13B-C) modified version to target GFP; (FIG. 13D-E) modified version to target AtPDS3; (FIG. 13F-G) modified version to target AtADH1. Mature miRNA/siRNAs are outlined in red, exhibiting structure conservation through design. The regions targeted for manipulation by CRISPR/CAS9 system are outlined in purple and the NGG sequence is highlighted in yellow (FIG. 13A).

FIG. 13H illustrates embodiment examples of GEiGS oligo designs in which the precursor structure does not play a role in the biogenesis, hence, it is not required to be maintained. Design based on the Brassica rapa bnTAS3B tasiRNA. From top to bottom: wild-type tasiRNA, GEiGS design with minimal sequence changes, and GEiGS design with maximal sequence changes. The selections of non-coding RNA precursors that give rise to mature small RNA molecules are highlighted in green. Sequence differences between the GEiGS oligos and the wild type sequence are highlighted in red. Of note, tasiRNA biogenesis, unlike miRNAs and tRNAs, does not rely on the precursor secondary structure.

FIGS. 14A-D illustrate gene targeting by miR-173 and its modified versions. (FIG. 14A) Wild type miR-173 target the TAS1c transcript by sequence complementarity of the mature miRNA to a sequence in the gene (in red). The newly modified miRNAs (SWAPs 1, 2, 3, 4, 9 and 10) were designed to target (FIG. 14B) GFP, (FIG. 14C) AtPDS3 and (FIG. 14D) AtADH1 by sequence complementarity to their sequence (in red). Modified nucleotide from wt sequence, are written in lowercase.

FIGS. 15A-D illustrate gene targeting by miR-390 and its modified versions. (FIG. 15A) Wild type miR-390 target the TAS3 transcript by sequence complementarity of the mature miRNA to a sequence in the gene (in red). The newly modified miRNAs (SWAPs 5, 6, 7, 8, 11 and 12) were designed to target (FIG. 15B) GFP, (FIG. 15C) AtPDS3 and (FIG. 15D) AtADH1 by sequence complementarity to their sequence (in red). Modified nucleotide from wt sequence, are written in lowercase.

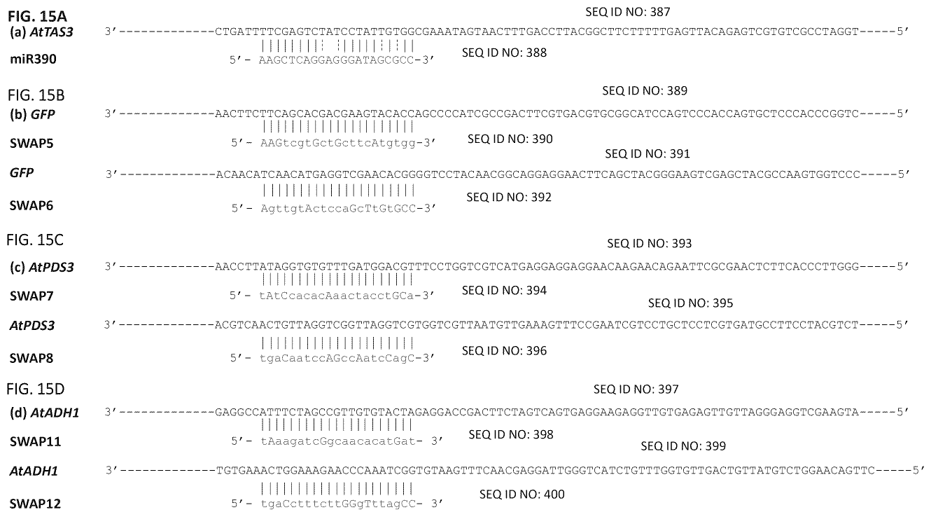

FIG. 16 illustrates PDS3 Phenotype/Genotype: bleached phenotype plants were selected and genotyped through internal amplicon PCR followed by restriction digest analysis with BtsαI (NEB) in order to verify donor presence vs. wild type sequence. Lane 1: Treated plants with NO DONOR, restricted, Lanes 2-4: PDS3 treated plants containing DONOR restricted, Lane 5: Positive plasmid DONOR control unrestricted, Lane 6: Water no template control, Lane 7: Positive Plasmid DONOR restricted, Lane 8: Plants bombarded with negative DONOR restricted, Lane 9: Untreated control plants restricted. Subsequent external PCR amplification of the amplicon was processed and sequenced in order to validate the insertion.

FIG. 17 illustrates ADH1 Phenotype/Genotype: Plants were selected through Allyl alcohol resistance and genotyped through internal amplicon PCR followed by BccI (NEB) restriction digest in order to verify donor presence. Lane 1: Allyl alcohol sensitive control plant restricted, Lane2-4: Allyl alcohol resistant plants containing DONOR restricted, Lane5: Positive plasmid DONOR control unrestricted, Lane 6: no template control, Lane7: Positive Plasmid DONOR restricted, Lane8: Plant bombarded with non-specific DONOR restricted, Lane 9: Non Allyl alcohol treated control restricted.

FIG. 18 is a graph illustrating gene expression analysis in miR-173 modified plant targeting AtPDS3 transcript. Analysis of AtPDS3 expression was carried out through qRT-PCR, in regenerating bombarded plants with GEiGS #4 and SWAP3 compared to plants bombarded with GEiGS #5 and SWAP1 and 2 (GFP). Of note, a reduction of 82% in gene expression level, on the average, was observed, when miR-173 was modified to target AtPDS3, compared to control plants (Error bars present SD; p-value <0.01 calculated on Ct values).

FIG. 19 is a graph illustrating gene expression analysis in miR-390 modified plant targeting AtPDS3 transcript. Analysis of AtADH1 expression was carried out through qRT-PCR, in regenerating bombarded plants with GEiGS #1 and SWAP11, compared to plants bombarded with GEiGS #5 and SWAP1 and 2 (GFP). Of note, a reduction of 82% in gene expression level, on the average, was observed, when miR-390 was modified to target AtADH1, compared to control plants (Error bars represent SD; p-value <0.01 calculated on Ct values).

### DESCRIPTION OF SPECIFIC EMBODIMENTS OF THE INVENTION

The present invention, in some embodiments thereof, relates to modifying genes that encode or are processed into non-coding RNA molecules, including RNA silencing molecules and, more particularly, but not exclusively, to the use of same for silencing endogenous or exogenous target RNA of interest in plants.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the examples. The invention is capable of other embodiments or of being practiced or carried out in various ways. Also, it is to be understood that the phraseology and terminology employed herein is for the purpose of description and should not be regarded as limiting.

Previous work on genome editing of RNA molecules in various organisms (e.g. murine, human, plants), focused on disruption of miRNA activity or target binding sites using transgenesis. Genome editing in plants has further concentrated on the use of CRISPR-Cas9 technology, ZFNs and TALENs, for knockdown of genes or insertions in model plants. Furthermore, gene silencing in plants using artificial microRNAs transgenes to silence endogenous and exogenous target genes were described [Molnar A et al. Plant J. (2009) 58(1):165-74. doi: 10.1111/j.1365-313X.2008.03767.x. Epub 2009 Jan. 19; Borges and Martienssen, Nature Reviews Molecular Cell Biology|AOP, published online 4 Nov. 2015; doi:10.1038/nrm4085]. The artificial miRNAs transgenes are introduced into plant cells within an artificial expression cassette (including a promoter, terminator, selection marker, etc.) and downregulate target expression.

While reducing the present invention to practice, the present inventors have devised a gene editing technology directed to non-coding RNA molecules (e.g. endogenous) designed to target and interfere with a non-natural target gene of interest (endogenous or exogenous to the plant cell). The gene editing technology described herein does not implement the classical molecular genetic and transgenic tools comprising expression cassettes that have a promoter, terminator, selection marker.

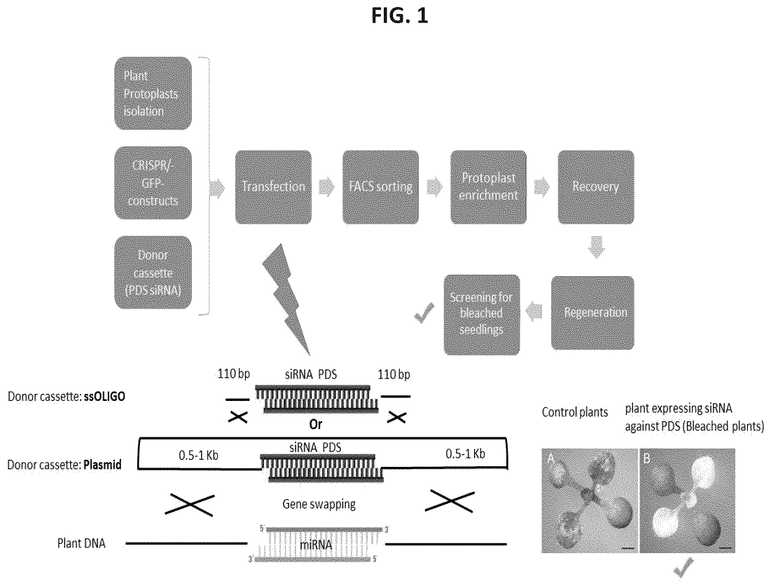

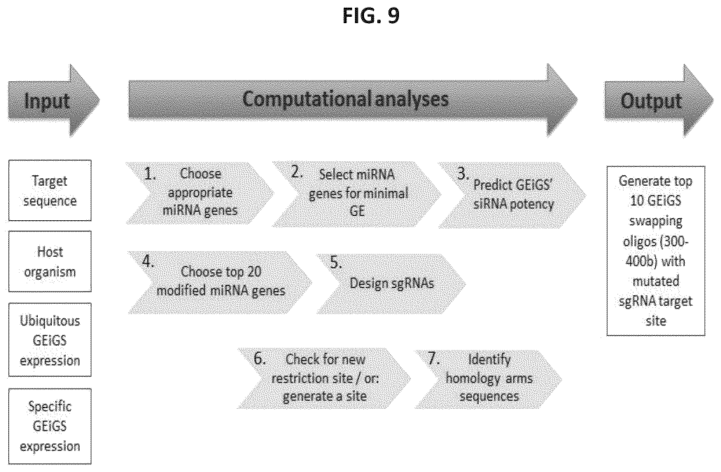

As is shown herein below and in the examples section which follows, the present inventors have designed a Genome Editing Induced Gene Silencing (GEiGS) platform capable of utilizing a plant cell's endogenous non-coding RNA molecules including e.g. RNA silencing molecules (e.g. siRNA, miRNA, piRNA, tasiRNA, tRNA, rRNA, antisense RNA, snRNA, snoRNA etc.) and modifying them to target and down regulate any RNA target of interest (see Exemplary flowchart in FIG. 1). Using GEiGS, the present method enables screening of potential non-coding RNA molecules, editing nucleotides in these endogenous RNA molecules, and thereby redirecting their specificity to effectively and specifically target and down regulate any RNA of interest including, endogenous and/or exogenous RNA encoded by pathogens and pests (see Exemplary flowchart in FIG. 9). Taken together, GEiGS can be utilized as a novel non-GMO technology for increasing crop yield, crop growth rate, crop quality as well as for crop protection against stress, pathogens, pests and herbicides.

Thus, according to an aspect of the invention there is provided a method of modifying a gene encoding or processed into a non-coding RNA molecule having no RNA silencing activity in a plant cell, the method comprising introducing into the plant cell a DNA editing agent conferring a silencing specificity of the non-coding RNA molecule towards a target RNA of interest, thereby modifying the gene encoding or processed into the non-coding RNA molecule.

According to another aspect of the invention there is provided a method of modifying a gene encoding or processed into a RNA silencing molecule to a target RNA in a plant cell, the method comprising introducing into the plant cell a DNA editing agent which redirects the specificity of the RNA silencing molecule towards a second target RNA, the target RNA and the second target RNA being distinct, thereby modifying the gene encoding the RNA silencing molecule.

The term “plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that may be useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, banana, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

According to a specific embodiment, the plant is a crop, a flower or a tree.

According to a specific embodiment, the plant is a woody plant species e.g., Actinidia chinensis (Actinidiaceae), Manihotesculenta (Euphorbiaceae), Firiodendron tulipifera (Magnoliaceae), Populus (Salicaceae), Santalum album (Santalaceae), Ulmus (Ulmaceae) and different species of the Rosaceae (Malus, Prunus, Pyrus) and the Rutaceae (Citrus, Microcitrus), Gymnospermae e.g., Picea glauca and Pinus taeda, forest trees (e.g., Betulaceae, Fagaceae, Gymnospermae and tropical tree species), fruit trees, shrubs or herbs, e.g., (banana, cocoa, coconut, coffee, date, grape and tea) and oil palm.

According to a specific embodiment, the plant is of a tropical crop e.g., coffee, macadamia, banana, pineapple, taro, papaya, mango, barley, beans, cassava, chickpea, cocoa (chocolate), cowpea, maize (corn), millet, rice, sorghum, sugarcane, sweet potato, tobacco, taro, tea, yam.

“Grain,” “seed,” or “bean,” refers to a flowering plant's unit of reproduction, capable of developing into another such plant. As used herein, the terms are used synonymously and interchangeably.

According to a specific embodiment, the plant is a plant cell e.g., plant cell in an embryonic cell suspension.

According to a specific embodiment, the plant cell is a protoplast.

The protoplasts are derived from any plant tissue e.g., fruit, flowers, roots, leaves, embryos, embryonic cell suspension, calli or seedling tissue.

As used herein, the term “non-coding RNA molecule” refers to a RNA sequence that is not translated into an amino acid sequence and does not encode a protein.

According to one embodiment, the non-coding RNA molecule is typically subject to the RNA silencing processing mechanism or activity. However, also contemplated herein are a few changes in nucleotides (e.g. for miRNA up to 24 nucleotides) which may elicit a processing mechanism that results in RNA interference or translation inhibition.

According to a specific embodiment, the non-coding RNA molecule is endogenous (naturally occurring, e.g. native) to the cell. It will be appreciated that the non-coding RNA molecule can also be exogenous to the cell (i.e. externally added and which is not naturally occurring in the cell).

According to some embodiments, the non-coding RNA molecule comprises an intrinsic translational inhibition activity.

According to some embodiments, the non-coding RNA molecule comprises an intrinsic RNAi activity.

According to some embodiments, the non-coding RNA molecule does not comprise an intrinsic translational inhibition activity or an intrinsic RNAi activity (i.e. the non-coding RNA molecule does not have an RNA silencing activity).

According to an embodiment of the invention, the non-coding RNA molecule is specific to a target RNA (e.g., a natural target RNA) and does not cross inhibit or silence a second target RNA or target RNA of interest unless designed to do so (as discussed below) exhibiting 100% or less global homology to the target gene, e.g., less than 99%, 98%, 97%, 96%, 95%, 94%, 93%, 92%, 91%, 90%, 89%, 88%, 87%, 86%, 85%, 84%, 83%, 82%, 81% global homology to the target gene; as determined at the RNA or protein level by RT-PCR, Western blot, Immunohistochemistry and/or flow cytometry, sequencing or any other detection methods.

According to one embodiment, the non-coding RNA molecule is a RNA silencing or RNA interference (RNAi) molecule.

The term “RNA silencing” or RNAi refers to a cellular regulatory mechanism in which non-coding RNA molecules (the “RNA silencing molecule” or “RNAi molecule”) mediate, in a sequence specific manner, co- or post-transcriptional inhibition of gene expression or translation.

According to one embodiment, the RNA silencing molecule is capable of mediating RNA repression during transcription (co-transcriptional gene silencing).

According to a specific embodiment, co-transcriptional gene silencing includes epigenetic silencing (e.g. chromatic state that prevents functional gene expression).

According to one embodiment, the RNA silencing molecule is capable of mediating RNA repression after transcription (post-transcriptional gene silencing).

Post-transcriptional gene silencing (PTGS) typically refers to the process (typically occurring in the cell cytoplasm) of degradation or cleavage of messenger RNA (mRNA) molecules which decrease their activity by preventing translation. For example, and as discussed in detail below, a guide strand of a RNA silencing molecule pairs with a complementary sequence in a mRNA molecule and induces cleavage by e.g. Argonaute 2 (Ago2).

Co-transcriptional gene silencing typically refers to inactivation of gene activity (i.e. transcription repression) and typically occurs in the cell nucleus. Such gene activity repression is mediated by epigenetic-related factors, such as e.g. methyl-transferases, that methylate target DNA and histones. Thus, in co-transcriptional gene silencing, the association of a small RNA with a target RNA (small RNA-transcript interaction) destabilizes the target nascent transcript and recruits DNA- and histone-modifying enzymes (i.e. epigenetic factors) that induce chromatin remodeling into a structure that repress gene activity and transcription. Also, in co-transcriptional gene silencing, chromatin-associated long non-coding RNA scaffolds may recruit chromatin-modifying complexes independently of small RNAs. These co-transcriptional silencing mechanisms form RNA surveillance systems that detect and silence inappropriate transcription events, and provide a memory of these events via self-reinforcing epigenetic loops [as described in D. Hoch and D. Moazed, RNA-mediated epigenetic regulation of gene expression, Nat Rev Genet. (2015) 16(2): 71-84].

According to an embodiment of the invention, the RNAi biogenesis/processing machinery generates the RNA silencing molecule.

According to an embodiment of the invention, the RNAi biogenesis/processing machinery generates the RNA silencing molecule, but no specific target has been identified.

According to one embodiment, the non-coding RNA molecule is a capable of inducing RNA interference (RNAi).

Following is a detailed description of non-coding RNA molecules which comprise an intrinsic RNAi activity (e.g. are RNA silencing molecules) that can be used according to specific embodiments of the present invention.

According to one embodiment, the non-coding RNA molecule or the RNA silencing molecule is processed from a precursor.

According to one embodiment, the non-coding RNA molecule or the RNA silencing molecule is processed from a single stranded RNA (ssRNA) precursor.

According to one embodiment, the non-coding RNA molecule or the RNA silencing molecule is processed from a duplex-structured single-stranded RNA precursor.

According to one embodiment, the non-coding RNA molecule or the RNA silencing molecule is processed from a dsRNA precursor (e.g. comprising perfect and imperfect base pairing).

According to one embodiment, the non-coding RNA molecule or the RNA silencing molecule is processed from a non-structured RNA precursor.

According to one embodiment, the non-coding RNA molecule or the RNA silencing molecule is processed from a protein-coding RNA precursor.

According to one embodiment, the non-coding RNA molecule or the RNA silencing molecule is processed from a non-coding RNA precursor.

According to one embodiment, the dsRNA can be derived from two different complementary RNAs, or from a single RNA that folds on itself to form dsRNA.

Perfect and imperfect based paired RNA (i.e. double stranded RNA; dsRNA), siRNA and shRNA—The presence of long dsRNAs in cells stimulates the activity of a ribonuclease III enzyme referred to as dicer. Dicer (also known as endoribonuclease Dicer or helicase with RNase motif) is an enzyme that in plants is typically referred to as Dicer-like (DCL) protein. Different plants have different numbers of DCL genes, thus for example, Arabidopsis genome typically has four DCL genes, rice has eight DCL genes, and maize genome has five DCL genes. Dicer is involved in the processing of the dsRNA into short pieces of dsRNA known as short interfering RNAs (siRNAs). siRNAs derived from dicer activity are typically about 21 to about 23 nucleotides in length and comprise about 19 base pair duplexes with two 3′ nucleotides overhangs.

Accordingly, some embodiments of the invention contemplate modifying a gene encoding a dsRNA to redirect a silencing specificity (including silencing activity) towards a second target RNA (i.e. RNA of interest).

According to one embodiment dsRNA precursors longer than 21 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

The term “siRNA” refers to small inhibitory RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27 mer) instead of a product (21 mer) and that this improves the rate or efficiency of entry of the siRNA duplex into RISC.

It has been found that position, but not the composition, of the 3′-overhang influences potency of a siRNA and asymmetric duplexes having a 3′-overhang on the antisense strand are generally more potent than those with the 3′-overhang on the sense strand (Rose et al., 2005).

The strands of a double-stranded interfering RNA (e.g., a siRNA) may be connected to form a hairpin or stem-loop structure (e.g., a shRNA). Thus, as mentioned, the RNA silencing molecule of some embodiments of the invention may also be a short hairpin RNA (shRNA).

The term short hairpin RNA, “shRNA”, as used herein, refers to a RNA molecule having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

The RNA silencing molecule of some embodiments of the invention need not be limited to those molecules containing only RNA, but further encompasses chemically-modified nucleotides and non-nucleotides.

Various types of siRNAs are contemplated by the present invention, including trans-acting siRNAs (Ta-siRNAs), repeat-associated siRNAs (Ra-siRNAs) and natural-antisense transcript-derived siRNAs (Nat-siRNAs).

According to one embodiment, silencing RNA includes “piRNA” which is a class of Piwi-interacting RNAs of about 26 and 31 nucleotides in length. piRNAs typically form RNA-protein complexes through interactions with Piwi proteins, i.e. antisense piRNAs are typically loaded into Piwi proteins (e.g. Piwi, Ago3 and Aubergine (Aub)).

miRNA—According to another embodiment the RNA silencing molecule may be a miRNA.

The term “microRNA”, “miRNA”, and “miR” are synonymous and refer to a collection of non-coding single-stranded RNA molecules of about 19-24 nucleotides in length, which regulate gene expression. miRNAs are found in a wide range of organisms (e.g. insects, mammals, plants, nematodes) and have been shown to play a role in development, homeostasis, and disease etiology.

Initially the pre-miRNA is present as a long non-perfect double-stranded stem loop RNA that is further processed by Dicer into a siRNA-like duplex, comprising the mature guide strand (miRNA) and a similar-sized fragment known as the passenger strand (miRNA*). The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-8 of the miRNA (referred as “seed sequence”).

A number of studies have looked at the base-pairing requirement between miRNA and its mRNA target for achieving efficient inhibition of translation (reviewed by Bartel 2004, Cell 116-281). Computational studies, analyzing miRNA binding on whole genomes have suggested a specific role for bases 2-8 at the 5′ of the miRNA (also referred to as “seed sequence”) in target binding but the role of the first nucleotide, found usually to be “A” was also recognized (Lewis et al. 2005 Cell 120-15). Similarly, nucleotides 1-7 or 2-8 were used to identify and validate targets by Krek et al. (2005, Nat Genet 37-495). The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

It will be appreciated that the pre-miRNA sequence may comprise from 45-90, 60-80 or 60-70 nucleotides while the pri-miRNA sequence may comprise from 45-30,000, 50-25,000, 100-20,000, 1,000-1,500 or 80-100 nucleotides.

According to one embodiment, the miRNA comprises miR-390a (as set forth in SEQ ID NO: 28).

According to one embodiment, the miRNA comprises miR-173 (as set forth in SEQ ID NO: 29).

Antisense—Antisense is a single stranded RNA designed to prevent or inhibit expression of a gene by specifically hybridizing to its mRNA. Downregulation of a target RNA can be effected using an antisense polynucleotide capable of specifically hybridizing with an mRNA transcript encoding the target RNA.

As mentioned, the non-coding RNA molecule may not comprise a canonical (intrinsic) RNAi activity (e.g. is not a canonical RNA silencing molecule, or its target has not been identified). Such non-coding RNA molecules include the following:

According to one embodiment, the non-coding RNA molecule is a transfer RNA (tRNA). The term “tRNA” refers to a RNA molecule that serves as the physical link between nucleotide sequence of nucleic acids and the amino acid sequence of proteins, formerly referred to as soluble RNA or sRNA. tRNA is typically about 76 to 90 nucleotides in length.

According to one embodiment, the non-coding RNA molecule is a ribosomal RNA (rRNA). The term “rRNA” refers to the RNA component of the ribosome i.e. of either the small ribosomal subunit or the large ribosomal subunit.

According to one embodiment, the non-coding RNA molecule is a small nuclear RNA (snRNA or U-RNA). The terms “sRNA” or “U-RNA” refer to the small RNA molecules found within the splicing speckles and Cajal bodies of the cell nucleus in eukaryotic cells. snRNA is typically about 150 nucleotides in length.

According to one embodiment, the non-coding RNA molecule is a small nucleolar RNA (snoRNA). The term “snoRNA” refers to the class of small RNA molecules that primarily guide chemical modifications of other RNAs, e.g. rRNAs, tRNAs and snRNAs. snoRNA is typically classified into one of two classes: the C/D box snoRNAs are typically about 70-120 nucleotides in length and are associated with methylation, and the H/ACA box snoRNAs are typically about 100-200 nucleotides in length and are associated with pseudouridylation.

Similar to snoRNAs are the scaRNAs (i.e. Small Cajal body RNA genes) which perform a similar role in RNA maturation to snoRNAs, but their targets are spliceosomal snRNAs and they perform site-specific modifications of spliceosomal snRNA precursors (in the Cajal bodies of the nucleus).

According to one embodiment, the non-coding RNA molecule is an extracellular RNA (exRNA). The term “exRNA” refers to RNA species present outside of the cells from which they were transcribed (e.g. exosomal RNA).

According to one embodiment, the non-coding RNA molecule is a long non-coding RNA (incRNA). The term “incRNA” or “long ncRNA” refers to non-protein coding transcripts typically longer than 200 nucleotides.

According to one embodiment, non-limiting examples of non-coding RNA molecules include, but are not limited to, microRNA (miRNA), piwi-interacting RNA (piRNA), short interfering RNA (siRNA), short-hairpin RNA (shRNA), trans-acting siRNA (tasiRNA), small nuclear RNA (snRNA or URNA), small nucleolar RNA (snoRNA), Small Cajal body RNA (scaRNA), transfer RNA (tRNA), ribosomal RNA (rRNA), extracellular RNA (exRNA), repeat-derived RNA, transposable element RNA and long non-coding RNA (lncRNA).

According to one embodiment, non-limiting examples of RNAi molecules include, but are not limited to, small interfering RNA (siRNA), short hairpin RNA (shRNA), microRNA (miRNA), Piwi-interacting RNA (piRNA) and trans-acting siRNA (tasiRNA).

As mentioned above, the methods of some embodiments of the invention are utilized to redirect a silencing activity and/or specificity of the non-coding RNA molecule (or to generate a silencing activity and/or specificity if the non-coding RNA molecule does not have an intrinsic capability to silence a RNA molecule) towards a second target RNA or towards a target RNA of interest.

According to one embodiment, the target RNA and the second target RNA are distinct.

According to one embodiment, the method of modifying a gene encoding or processed into a RNA silencing molecule to a target RNA in a plant cell comprises introducing into the plant cell a DNA editing agent which redirects a silencing activity and/or specificity of the RNA silencing molecule towards a second target RNA, the target RNA and the second target RNA being distinct, thereby modifying the gene encoding the RNA silencing molecule.

As used herein, the term “redirects a silencing specificity” refers to reprogramming the original specificity of the non-coding RNA (e.g. RNA silencing molecule) towards a non-natural target of the non-coding RNA (e.g. RNA silencing molecule). Accordingly, the original specificity of the non-coding RNA is abolished (i.e. loss of function) and the new specificity is towards a RNA target distinct of the natural target (i.e. RNA of interest), i.e., gain of function. It will be appreciated that only gain of function occurs in cases that the non-coding RNA has no silencing activity.

As used herein, the term “target RNA” refers to a RNA sequence naturally bound by a non-coding RNA molecule. Thus, the target RNA is considered by the skilled artisan as a substrate for the non-coding RNA.

As used herein, the term “second target RNA” refers to a RNA sequence (coding or non-coding) not naturally bound by a non-coding RNA molecule. Thus, the second target RNA is not a natural substrate of the non-coding RNA.

As used herein, the term “target RNA of interest” refers to a RNA sequence (coding or non-coding) to be silenced by the designed non-coding RNA molecule.

As used herein, the phrase “silencing a target gene” refers to the absence or observable reduction in the level of mRNA and/or protein products from the target gene (e.g. due to co- and/or post-transcriptional gene silencing). Thus, silencing of a target gene can be by 5%, 10%, 20%, 30%, 40%, 50%, 60%, 70%, 80%, 90%, 95% or 100% as compared to a target gene not targeted by the designed non-coding RNA molecule of the invention.

The consequences of silencing can be confirmed by examination of the outward properties of a plant cell or whole plant or other organism that take up the designed non-coding RNA from the plant or by biochemical techniques (as discussed below).

It will be appreciated that the designed non-coding RNA molecule of some embodiments of the invention can have some off-target specificity effect/s provided that it does not affect an agriculturally valuable trait (e.g., biomass, yield etc.).

According to one embodiment, the second target RNA or target RNA of interest is endogenous to the plant cell. Exemplary endogenous second target RNA or target RNA of interest include, but are not limited to, a product of a gene conferring sensitivity to stress, to infection, to herbicides, or a product of a gene related to plant growth rate, crop yield, as further discussed herein below.

According to one embodiment, the second target RNA or target RNA of interest is exogenous to the plant cell (also referred to herein as heterologous). In such a case, the second target RNA or target RNA of interest is a product of a gene that is not naturally part of the plant genome. Exemplary exogenous second target RNA include, but are not limited to, a product of a gene of a plant pathogen such as, but not limited to, an insect, a virus, a bacteria, a fungi, a nematode, as further discussed herein below. An exogenous target RNA (coding or non-coding) may comprise a nucleic acid sequence which shares sequence identity with an endogenous RNA sequence (e.g. may be partially homologous to an endogenous nucleic acid sequence) of the plant.

The specific binding of an endogenous non-coding RNA molecule with a target RNA can be determined by computational algorithms (such as BLAST) and verified by methods including e.g. Northern blot, In Situ hybridization, QuantiGene Plex Assay etc.

By use of the term “complementarity” or “complementary” is meant that the non-coding RNA molecule (or at least a portion of it that is present in the processed small RNA form, or at least one strand of a double-stranded polynucleotide or portion thereof, or a portion of a single strand polynucleotide) hybridizes under physiological conditions to the target RNA, or a fragment thereof, to effect regulation or function or suppression of the target gene. For example, in some embodiments, a non-coding RNA molecule has 100 percent sequence identity or at least about 30, 40, 45, 50, 55, 60, 65, 70, 75, 80, 83, 84, 85, 86, 87, 88, 89, 90, 91, 92, 93, 94, 95, 96, 97, 98, or 99 percent sequence identity when compared to a sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 150, 200, 300, 400, 500 or more contiguous nucleotides in the target RNA (or family members of a given target gene).

As used herein, a non-coding RNA molecules, or their processed small RNA forms, are said to exhibit “complete complementarity” when every nucleotide of one of the sequences read 5′ to 3′ is complementary to every nucleotide of the other sequence when read 3′ to 5′. A nucleotide sequence that is completely complementary to a reference nucleotide sequence will exhibit a sequence identical to the reverse complement sequence of the reference nucleotide sequence.

Methods for determining sequence complementarity are well known in the art and include, but not limited to, bioinformatics tools which are well known in the art (e.g. BLAST, multiple sequence alignment).

According to one embodiment, if the non-coding RNA molecule is or processed into a siRNA, the complementarity is in the range of 90-100% (e.g. 100%) to its target sequence.

According to one embodiment, if the non-coding RNA molecule is or processed into a miRNA or piRNA the complementarity is in the range of 33-100% to its target sequence.

According to one embodiment, if the non-coding RNA molecule is a miRNA, the seed sequence complementarity (i.e. nucleotides 2-8 from the 5′) is in the range of 85-100% (e.g. 100%) to its target sequence.

According to one embodiment, the non-coding RNA can be further processed into a small RNA form (e.g. pre-miRNA is processed into a mature miRNA). In such a case, homology is measured based on the processed small RNA form (e.g. the mature miRNA sequence).

As used herein, the term “small RNA form” refers to the mature small RNA being capable of hybridizing with a target RNA (or fragment thereof). According to one embodiment, the small RNA form has a silencing activity.

According to one embodiment, the complementarity to the target sequence is at least about 33% of the processed small RNA form (e.g. 33% of the 21-28 nt). Thus, for example, if the non-coding RNA molecule is a miRNA, 33% of the mature miRNA sequence (e.g. 21 nt) comprises seed complementation (e.g. 7 nt out of the 21 nt).

According to one embodiment, the complementarity to the target sequence is at least about 45% of the processed small RNA form (e.g. 45% of the 21-28 nt). Thus, for example, if the non-coding RNA molecule is a miRNA, 45% of the mature miRNA sequence (e.g. 21 nt) comprises seed complementation (e.g. 9-10 nt out of the 21 nt).

According to one embodiment, the non-coding RNA molecule (i.e. prior to modification) is typically selected as one having about 10%, 20%, 30%, 33%, 40%, 50%, 60%, 70%, 80%, 85%, 90%, 95%, 96%, 97%, 98% or up to 99% complementarity towards the sequence of the second target RNA or target RNA of interest.

According to a specific embodiment, the non-coding RNA molecule (i.e. prior to modification) is typically selected as one having no more than 99% complementarity towards the sequence of the second target RNA or target RNA of interest.

According to a specific embodiment, the non-coding RNA molecule (i.e. prior to modification) is typically selected as one having no more than 98% complementarity towards the sequence of the second target RNA or target RNA of interest.

According to a specific embodiment, the non-coding RNA molecule (i.e. prior to modification) is typically selected as one having no more than 97% complementarity towards the sequence of the second target RNA or target RNA of interest.

According to a specific embodiment, the non-coding RNA molecule (i.e. prior to modification) is typically selected as one having no more than 96% complementarity towards the sequence of the second target RNA or target RNA of interest.

According to a specific embodiment, the non-coding RNA molecule (i.e. prior to modification) is typically selected as one having no more than 95% complementarity towards the sequence of the second target RNA or target RNA of interest.

According to a specific embodiment, the non-coding RNA molecule (i.e. prior to modification) is typically selected as one having no more than 94% complementarity towards the sequence of the second target RNA or target RNA of interest.

According to a specific embodiment, the non-coding RNA molecule (i.e. prior to modification) is typically selected as one having no more than 93% complementarity towards the sequence of the second target RNA or target RNA of interest.

According to a specific embodiment, the non-coding RNA molecule (i.e. prior to modification) is typically selected as one having no more than 92% complementarity towards the sequence of the second target RNA or target RNA of interest.

According to a specific embodiment, the non-coding RNA molecule (i.e. prior to modification) is typically selected as one having no more than 91% complementarity towards the sequence of the second target RNA or target RNA of interest.

According to a specific embodiment, the non-coding RNA molecule (i.e. prior to modification) is typically selected as one having no more than 90% complementarity towards the sequence of the second target RNA or target RNA of interest.

According to a specific embodiment, the non-coding RNA molecule (i.e. prior to modification) is typically selected as one having no more than 85% complementarity towards the sequence of the second target RNA or target RNA of interest.

According to a specific embodiment, the non-coding RNA molecule (i.e. prior to modification) is typically selected as one having no more than 50% complementarity towards the sequence of the second target RNA or target RNA of interest.

According to a specific embodiment, the non-coding RNA molecule (i.e. prior to modification) is typically selected as one having no more than 33% complementarity towards the sequence of the second target RNA or target RNA of interest.

According to one embodiment, the non-coding RNA molecule (e.g. RNA silencing molecule) is designed so as to comprise at least about 33%, 40%, 45%, 50%, 55%, 60%, 70%, 80%, 85%, 90%, 91%, 92%, 93%, 94%, 95%, 96%, 97%, 98%, 99% or even 100% complementarity towards the sequence of the second target RNA or target RNA of interest.

According to a specific embodiment, the non-coding RNA molecule (e.g. RNA silencing molecule) is designed so as to comprise a minimum of 33% complementarity towards the second target RNA or target RNA of interest (e.g. 85-100% seed match).

According to a specific embodiment, the non-coding RNA molecule (e.g. RNA silencing molecule) is designed so as to comprise a minimum of 40% complementarity towards the second target RNA or target RNA of interest.

According to a specific embodiment, the non-coding RNA molecule (e.g. RNA silencing molecule) is designed so as to comprise a minimum of 45% complementarity towards the second target RNA or target RNA of interest.

According to a specific embodiment, the non-coding RNA molecule (e.g. RNA silencing molecule) is designed so as to comprise a minimum of 50% complementarity towards the second target RNA or target RNA of interest.

According to a specific embodiment, the non-coding RNA molecule (e.g. RNA silencing molecule) is designed so as to comprise a minimum of 45% complementarity towards the second target RNA or target RNA of interest.

According to a specific embodiment, the non-coding RNA molecule (e.g. RNA silencing molecule) is designed so as to comprise a minimum of 60% complementarity towards the second target RNA or target RNA of interest.

According to a specific embodiment, the non-coding RNA molecule (e.g. RNA silencing molecule) is designed so as to comprise a minimum of 70% complementarity towards the second target RNA or target RNA of interest.

According to a specific embodiment, the non-coding RNA molecule (e.g. RNA silencing molecule) is designed so as to comprise a minimum of 80% complementarity towards the second target RNA or target RNA of interest.

According to a specific embodiment, the non-coding RNA molecule (e.g. RNA silencing molecule) is designed so as to comprise a minimum of 85% complementarity towards the second target RNA or target RNA of interest.

According to a specific embodiment, the non-coding RNA molecule (e.g. RNA silencing molecule) is designed so as to comprise a minimum of 90% complementarity towards the second target RNA or target RNA of interest.

According to a specific embodiment, the non-coding RNA molecule (e.g. RNA silencing molecule) is designed so as to comprise a minimum of 91% complementarity towards the second target RNA or target RNA of interest.

According to a specific embodiment, the non-coding RNA molecule (e.g. RNA silencing molecule) is designed so as to comprise a minimum of 92% complementarity towards the second target RNA or target RNA of interest.

According to a specific embodiment, the non-coding RNA molecule (e.g. RNA silencing molecule) is designed so as to comprise a minimum of 93% complementarity towards the second target RNA or target RNA of interest.

According to a specific embodiment, the non-coding RNA molecule (e.g. RNA silencing molecule) is designed so as to comprise a minimum of 94% complementarity towards the second target RNA or target RNA of interest.

According to a specific embodiment, the non-coding RNA molecule (e.g. RNA silencing molecule) is designed so as to comprise a minimum of 95% complementarity towards the second target RNA or target RNA of interest.

According to a specific embodiment, the non-coding RNA molecule (e.g. RNA silencing molecule) is designed so as to comprise a minimum of 96% complementarity towards the second target RNA or target RNA of interest.

According to a specific embodiment, the non-coding RNA molecule (e.g. RNA silencing molecule) is designed so as to comprise a minimum of 97% complementarity towards the second target RNA or target RNA of interest.

According to a specific embodiment, the non-coding RNA molecule (e.g. RNA silencing molecule) is designed so as to comprise a minimum of 98% complementarity towards the second target RNA or target RNA of interest.

According to a specific embodiment, the non-coding RNA molecule (e.g. RNA silencing molecule) is designed so as to comprise a minimum of 99% complementarity towards the second target RNA or target RNA of interest.

According to a specific embodiment, the non-coding RNA molecule (e.g. RNA silencing molecule) is designed so as to comprise 100% complementarity towards the second target RNA or target RNA of interest.

In order to induce silencing activity and/or specificity of a non-coding RNA molecule or redirect a silencing activity and/or specificity of a non-coding RNA molecule (e.g. RNA silencing molecule) towards a second target RNA or target RNA of interest, the gene encoding a non-coding RNA molecule (e.g. RNA silencing molecule) is modified using a DNA editing agent.

Following is a description of various non-limiting examples of methods and DNA editing agents used to introduce nucleic acid alterations to a gene encoding a non-coding RNA molecule (e.g. RNA silencing molecule) and agents for implementing same that can be used according to specific embodiments of the present disclosure.

Genome Editing using engineered endonucleases—this approach refers to a reverse genetics method using artificially engineered nucleases to typically cut and create specific double-stranded breaks (DSBs) at a desired location(s) in the genome, which are then repaired by cellular endogenous processes such as, homologous recombination (HR) or non-homologous end-joining (NHEJ). NHEJ directly joins the DNA ends in a double-stranded break (DSB) with or without minimal ends trimming, while HR utilizes a homologous donor sequence as a template (i.e. the sister chromatid formed during S-phase) for regenerating/copying the missing DNA sequence at the break site. In order to introduce specific nucleotide modifications to the genomic DNA, a donor DNA repair template containing the desired sequence must be present during HR (exogenously provided single stranded or double stranded DNA).

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks (DSBs), several distinct classes of nucleases have been discovered and bioengineered to date. These include the meganucleases, Zinc finger nucleases (ZFNs), transcription-activator like effector nucleases (TALENs) and CRISPR/Cas9 system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks (DSBs) in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (DSBs) (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fok1. Additionally Fok1 has the advantage of requiring dimerization to have nuclease activity and this means the specificity increases dramatically as each nuclease partner recognizes a unique DNA sequence. To enhance this effect, Fok1 nucleases have been engineered that can only function as heterodimers and have increased catalytic activity. The heterodimer functioning nucleases avoid the possibility of unwanted homodimer activity and thus increase specificity of the double-stranded break (DSB).

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break (DSB). Repair of these double-stranded breaks (DSBs) through the non-homologous end-joining (NHEJ) pathway often results in small deletions or small sequence insertions (Indels). Since each repair made by NHEJ is unique, the use of a single nuclease pair can produce an allelic series with a range of different insertions or deletions at the target site.

In general NHEJ is relatively accurate (about 85% of DSBs in human cells are repaired by NHEJ within about 30 min from detection) in gene editing erroneous NHEJ is relied upon as when the repair is accurate the nuclease will keep cutting until the repair product is mutagenic and the recognition/cut site/PAM motif is gone/mutated or that the transiently introduced nuclease is no longer present.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break (DSB) can be repaired via homologous recombination (HR) to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system and all its variants (also referred to herein as “CRISPR”)—Many bacteria and archea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form a RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821).

It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded breaks (DSBs) in a variety of different species (Cho et al., 2013; Cong et al., 2013; DiCarlo et al., 2013; Hwang et al., 2013a,b; Jinek et al., 2013; Mali et al., 2013).

The CRISPR/Cas system for genome editing contains two distinct components: a gRNA and an endonuclease e.g. Cas9.

The gRNA (also referred to herein as short guide RNA (sgRNA)) is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break (DSB). Just as with ZFNs and TALENs, the double-stranded breaks (DSBs) produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks (DSBs) in the genomic DNA.

A significant advantage of CRISPR/Cas is that the high efficiency of this system is coupled with the ability to easily create synthetic gRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is mostly repaired by single strand break repair mechanism involving proteins such as but not only, PARP (sensor) and XRCC1/LIG III complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas9 nickase are treated as a double-strand break, in what is often referred to as a ‘double nick’ CRISPR system. A double-nick, which is basically non-parallel DSB, can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that are not likely to change the genomic DNA, even though these events are not impossible.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publicly available tools available to help choose and/or design target sequences as well as lists of bioinformatically determined unique gRNAs for different genes in different species such as, but not limited to, the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and a Cas endonuclease (e.g. Cas9) should be expressed or present (e.g., as a ribonucleoprotein complex) in a target cell. The insertion vector can contain both cassettes on a single plasmid or the cassettes are expressed from two separate plasmids. CRISPR plasmids are commercially available such as the px330 plasmid from Addgene (75 Sidney St, Suite 550A⋅Cambridge, MA 02139). Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety. Cas endonucleases that can be used to effect DNA editing with gRNA include, but are not limited to, Cas9, Cpf1 (Zetsche et al., 2015, Cell. 163(3):759-71), C2c1, C2c2, and C2c3 (Shmakov et al., Mol Cell. 2015 Nov. 5; 60(3):385-97).

According to a specific embodiment, the CRISPR comprises a short guide RNA (sgRNA) comprising a nucleic acid sequence selected from the group consisting of SEQ ID NOs: 1-4 or SEQ ID NOs: 235-366.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, introduced into the cells, and positive selection is performed to isolate homologous recombination mediated events. The DNA carrying the homologous sequence can be provided as a plasmid, single or double stranded oligo. These homologous recombinants contain a local duplication that is separated by intervening vector sequence, including the selection cassette. In the second step, targeted clones are subjected to negative selection to identify cells that have lost the selection cassette via intra-chromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After the system components have been introduced to the cell and positive selection applied, HR mediated events could be identified. Next, a second targeting vector that contains a region of homology with the desired mutation is introduced into targeted clones, and negative selection is applied to remove the selection cassette and introduce the mutation. The final allele contains the desired mutation while eliminating unwanted exogenous sequences.

According to a specific embodiment, the DNA editing agent comprises a DNA targeting module (e.g., gRNA).

According to a specific embodiment, the DNA editing agent does not comprise an endonuclease.

According to a specific embodiment, the DNA editing agent comprises a nuclease (e.g. an endonuclease) and a DNA targeting module (e.g., gRNA).

According to a specific embodiment, the DNA editing agent is CRISPR/Cas, e.g. gRNA and Cas9.

According to a specific embodiment, the DNA editing agent is TALEN.

According to a specific embodiment, the DNA editing agent is ZFN.

According to a specific embodiment, the DNA editing agent is meganuclease.

According to one embodiment, the DNA editing agent is linked to a reporter for monitoring expression in a plant cell.

According to one embodiment, the reporter is a fluorescent reporter protein.

The term “a fluorescent protein” refers to a polypeptide that emits fluorescence and is typically detectable by flow cytometry, microscopy or any fluorescent imaging system, therefore can be used as a basis for selection of cells expressing such a protein.

Examples of fluorescent proteins that can be used as reporters are, without being limited to, the Green Fluorescent Protein (GFP), the Blue Fluorescent Protein (BFP) and the red fluorescent proteins (e.g. dsRed, mCherry, RFP). A non-limiting list of fluorescent or other reporters includes proteins detectable by luminescence (e.g. luciferase) or colorimetric assay (e.g. GUS). According to a specific embodiment, the fluorescent reporter is a red fluorescent protein (e.g. dsRed, mCherry, RFP) or GFP.

A review of new classes of fluorescent proteins and applications can be found in Trends in Biochemical Sciences [Rodriguez, Erik A.; Campbell, Robert E.; Lin, John Y.; Lin, Michael Z.; Miyawaki, Atsushi; Palmer, Amy E.; Shu, Xiaokun; Zhang, Jin; Tsien, Roger Y. “The Growing and Glowing Toolbox of Fluorescent and Photoactive Proteins”. Trends in Biochemical Sciences. doi:10.1016/j.tibs.2016.09.010].

According to another embodiment, the reporter is an endogenous gene of a plant. An exemplary reporter is the phytoene desaturase gene (PDS3) which encodes one of the important enzymes in the carotenoid biosynthesis pathway. Its silencing produces an albino/bleached phenotype. Accordingly, plants with reduced expression of PDS3 exhibit reduced chlorophyll levels, up to complete albino and dwarfism. Additional genes which can be used in accordance with the present teachings include, but are not limited to, genes which take part in crop protection. Exemplary genes are described in Table 1B, below.

According to another embodiment, the reporter is an antibiotic selection marker. Examples of antibiotic selection markers that can be used as reporters are, without being limited to, neomycin phosphotransferase II (nptII) and hygromycin phosphotransferase (hpt). Additional marker genes which can be used in accordance with the present teachings include, but are not limited to, gentamycin acetyltransferase (accC3) resistance and bleomycin and phleomycin resistance genes.

It will be appreciated that the enzyme NPTII inactivates by phosphorylation a number of aminoglycoside antibiotics such as kanamycin, neomycin, geneticin (or G418) and paromomycin. Of these, kanamycin, neomycin and paromomycin are used in a diverse range of plant species.

According to another embodiment, the reporter is a toxic selection marker. An exemplary toxic selection marker that can be used as a reporter is, without being limited to, allyl alcohol selection using the Alcohol dehydrogenase (ADH1) gene. ADH1, comprising a group of dehydrogenase enzymes which catalyse the interconversion between alcohols and aldehydes or ketones with the concomitant reduction of NAD+ or NADP+, breaks down alcoholic toxic substances within tissues. Plants harbouring reduced ADH1 expression exhibit increase tolerance to allyl alcohol. Accordingly, plants with reduced ADH1 are resistant to the toxic effect of allyl alcohol.

Regardless of the DNA editing agent used, the method of the invention is employed such that the gene encoding the non-coding RNA molecule (e.g. RNA silencing molecule) is modified by at least one of a deletion, an insertion or a point mutation.

According to one embodiment, the modification is in a structured region of the non-coding RNA molecule or the RNA silencing molecule.

According to one embodiment, the modification is in a stem region of the non-coding RNA molecule or the RNA silencing molecule.

According to one embodiment, the modification is in a loop region of the non-coding RNA molecule or the RNA silencing molecule.

According to one embodiment, the modification is in a stem region and a loop region of the non-coding RNA molecule or the RNA silencing molecule.

According to one embodiment, the modification is in a non-structured region of the non-coding RNA molecule or the RNA silencing molecule.

According to one embodiment, the modification is in a stem region and a loop region and in non-structured region of the non-coding RNA molecule or the RNA silencing molecule.

According to a specific embodiment, the modification comprises a modification of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native non-coding RNA molecule, e.g. RNA silencing molecule).

According to one embodiment, the modification comprises a modification of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides (as compared to the native non-coding RNA molecule, e.g. RNA silencing molecule).

According to one embodiment, the modification can be in a consecutive nucleic acid sequence (e.g. at least 5, 10, 20, 30, 40, 50, 100, 150, 200 bases).

According to one embodiment, the modification can be in a non-consecutive manner, e.g. throughout a 20, 50, 100, 150, 200, 500, 1000 nucleic acid sequence.

According to a specific embodiment, the modification comprises a modification of at most 200 nucleotides.

According to a specific embodiment, the modification comprises a modification of at most 150 nucleotides.

According to a specific embodiment, the modification comprises a modification of at most 100 nucleotides.

According to a specific embodiment, the modification comprises a modification of at most 50 nucleotides.

According to a specific embodiment, the modification comprises a modification of at most 25 nucleotides.

According to a specific embodiment, the modification comprises a modification of at most 20 nucleotides.

According to a specific embodiment, the modification comprises a modification of at most 15 nucleotides.

According to a specific embodiment, the modification comprises a modification of at most 10 nucleotides.

According to a specific embodiment, the modification comprises a modification of at most 5 nucleotides.

According to one embodiment, the modification depends on the structure of the RNA silencing molecule.

Accordingly, when the RNA silencing molecule contains a non-essential structure (i.e. a secondary structure of the RNA silencing molecule which does not play a role in its proper biogenesis and/or function) or is purely dsRNA (i.e. the RNA silencing molecule having a perfect or almost perfect dsRNA), a few modifications (e.g. 20-30 nucleotides, e.g. 1-10 nucleotides, e.g. 5 nucleotides) are introduced in order to redirect the silence specificity of the RNA silencing molecule.

According to another embodiment, when the RNA silencing molecule has an essential structure (i.e. the proper biogenesis and/or activity of the RNA silencing molecule is dependent on its secondary structure), larger modifications (e.g. 10-200 nucleotides, e.g. 50-150 nucleotides, e.g., more than 30 nucleotides and not exceeding 200 nucleotides, 30-200 nucleotides, 35-200 nucleotides, 35-150 nucleotides, 35-100 nucleotides) are introduced in order to redirect the silence specificity of the RNA silencing molecule.

According to one embodiment, the modification is such that the recognition/cut site/PAM motif of the RNA silencing molecule is modified to abolish the original PAM recognition site.

According to a specific embodiment, the modification is in at least 1, 2, 3, 4, 5, 6, 7, 8, 9, 10 or more nucleic acids in a PAM motif.

According to one embodiment, the modification comprises an insertion.

According to a specific embodiment, the insertion comprises an insertion of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native non-coding RNA molecule, e.g. RNA silencing molecule).

According to one embodiment, the insertion comprises an insertion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40,42,44,46,48, 50, 60,70, 80, 90, 100, 110, 120, 130, 140, 150, 160, 170, 180, 190,200 or at most 250 nucleotides (as compared to the native non-coding RNA molecule, e.g. RNA silencing molecule).

According to a specific embodiment, the insertion comprises an insertion of at most 200 nucleotides.

According to a specific embodiment, the insertion comprises an insertion of at most 150 nucleotides.

According to a specific embodiment, the insertion comprises an insertion of at most 100 nucleotides.

According to a specific embodiment, the insertion comprises an insertion of at most 50 nucleotides.

According to a specific embodiment, the insertion comprises an insertion of at most 25 nucleotides.

According to a specific embodiment, the insertion comprises an insertion of at most 20 nucleotides.

According to a specific embodiment, the insertion comprises an insertion of at most 15 nucleotides.

According to a specific embodiment, the insertion comprises an insertion of at most 10 nucleotides.

According to a specific embodiment, the insertion comprises an insertion of at most 5 nucleotides.

According to one embodiment, the modification comprises a deletion.

According to a specific embodiment, the deletion comprises a deletion of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native non-coding RNA molecule, e.g. RNA silencing molecule).

According to one embodiment, the deletion comprises a deletion of at most 1, 2, 3,4, 5, 6, 7, 8, 9, 10, 11, 12, 13, 14, 15, 16, 17, 18, 19, 20, 22, 24, 26, 28, 30, 32, 34, 36, 38, 40,42,44,46,48,50,60,70, 80, 90, 100, 110, 120, 130, 140, 150, 160, 170, 180, 190, 200 or at most 250 nucleotides (as compared to the native non-coding RNA molecule, e.g. RNA silencing molecule).

According to a specific embodiment, the deletion comprises a deletion of at most 200 nucleotides.

According to a specific embodiment, the deletion comprises a deletion of at most 150 nucleotides.

According to a specific embodiment, the deletion comprises a deletion of at most 100 nucleotides.

According to a specific embodiment, the deletion comprises a deletion of at most 50 nucleotides.

According to a specific embodiment, the deletion comprises a deletion of at most 25 nucleotides.

According to a specific embodiment, the deletion comprises a deletion of at most 20 nucleotides.

According to a specific embodiment, the deletion comprises a deletion of at most 15 nucleotides.

According to a specific embodiment, the deletion comprises a deletion of at most 10 nucleotides.

According to a specific embodiment, the deletion comprises a deletion of at most 5 nucleotides.

According to one embodiment, the modification comprises a point mutation.

According to a specific embodiment, the point mutation comprises a point mutation of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native non-coding RNA molecule, e.g. RNA silencing molecule).

According to one embodiment, the point mutation comprises a point mutation in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides (as compared to the native non-coding RNA molecule, e.g. RNA silencing molecule).

According to a specific embodiment, the point mutation comprises a point mutation in at most 200 nucleotides.

According to a specific embodiment, the point mutation comprises a point mutation in at most 150 nucleotides.

According to a specific embodiment, the point mutation comprises a point mutation in at most 100 nucleotides.

According to a specific embodiment, the point mutation comprises a point mutation in at most 50 nucleotides.

According to a specific embodiment, the point mutation comprises a point mutation in at most 25 nucleotides.

According to a specific embodiment, the point mutation comprises a point mutation in at most 20 nucleotides.

According to a specific embodiment, the point mutation comprises a point mutation in at most 15 nucleotides.

According to a specific embodiment, the point mutation comprises a point mutation in at most 10 nucleotides.

According to a specific embodiment, the point mutation comprises a point mutation in at most 5 nucleotides.

According to one embodiment, the modification comprises a combination of any of a deletion, an insertion and/or a point mutation.

According to one embodiment, the modification comprises nucleotide replacement (e.g. nucleotide swapping).

According to a specific embodiment, the swapping comprises swapping of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native non-coding RNA molecule, e.g. RNA silencing molecule).

According to one embodiment, the nucleotide swap comprises a nucleotide replacement in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides (as compared to the native non-coding RNA molecule, e.g. RNA silencing molecule).

According to a specific embodiment, the nucleotide swapping comprises a nucleotide replacement in at most 200 nucleotides.

According to a specific embodiment, the nucleotide swapping comprises a nucleotide replacement in at most 150 nucleotides.

According to a specific embodiment, the nucleotide swapping comprises a nucleotide replacement in at most 100 nucleotides.

According to a specific embodiment, the nucleotide swapping comprises a nucleotide replacement in at most 50 nucleotides.

According to a specific embodiment, the nucleotide swapping comprises a nucleotide replacement in at most 25 nucleotides.

According to a specific embodiment, the nucleotide swapping comprises a nucleotide replacement in at most 20 nucleotides.

According to a specific embodiment, the nucleotide swapping comprises a nucleotide replacement in at most 15 nucleotides.

According to a specific embodiment, the nucleotide swapping comprises a nucleotide replacement in at most 10 nucleotides.

According to a specific embodiment, the nucleotide swapping comprises a nucleotide replacement in at most 5 nucleotides.

According to one embodiment, the gene encoding the non-coding RNA molecule (e.g. RNA silencing molecule) is modified by swapping a sequence of an endogenous RNA silencing molecule (e.g. miRNA) with a RNA silencing sequence of choice (e.g. siRNA).

According to a specific embodiment, the sequence of a siRNA used for gene swapping of an endogenous RNA silencing molecule (e.g. miRNA) comprising a nucleic acid sequence selected from the group consisting of SEQ ID NOs: 5-12 or SEQ ID NOs: 103-234.

According to one embodiment, the guide strand of the non-coding RNA molecule (e.g. RNA silencing molecule such as miRNA precursors (pri/pre-miRNAs) or siRNA precursors (dsRNA)) is modified to preserve originality of structure and keep the same base pairing profile.

According to one embodiment, the passenger strand of the non-coding RNA molecule (e.g. RNA silencing molecule such as miRNA precursors (pri/pre-miRNAs) or siRNA precursors (dsRNA)) is modified to preserve originality of structure and keep the same base pairing profile.

As used herein, the term “originality of structure” refers to the secondary RNA structure (i.e. base pairing profile). Keeping the originality of structure is important for correct and efficient biogenesis/processing of the non-coding RNA (e.g. RNA silencing molecule such as siRNA or miRNA) that is structure- and not purely sequence-dependent.

According to one embodiment, the non-coding RNA (e.g. RNA silencing molecule) is modified in the guide strand (silencing strand) as to comprise about 50-100% complementarity to the target RNA (as discussed above) while the passenger strand is modified to preserve the original (unmodified) non-coding RNA structure.

According to one embodiment, the non-coding RNA (e.g. RNA silencing molecule) is modified such that the seed sequence (e.g. for miRNA nucleotides 2-8 from the 5′ terminal) is complimentary to the target sequence.

According to a specific embodiment, the RNA silencing molecule (i.e. RNAi molecule) is designed such that a sequence of the RNAi molecule is modified to preserve originality of structure and to be recognized by cellular RNAi processing and executing factors.

According to a specific embodiment, the non-coding RNA molecule (i.e. rRNA, tRNA, lncRNA, snoRNA, etc.) is designed such that a sequence of the RNAi molecule is modified to be recognized by cellular RNAi processing and executing factors.

The DNA editing agent of the invention may be introduced into plant cells using DNA delivery methods (e.g. by expression vectors) or using DNA-free methods.

According to one embodiment, the gRNA (or any other DNA recognition module used, dependent on the DNA editing system that is used) can be provided as RNA to the cell.

Thus, it will be appreciated that the present techniques relate to introducing the DNA editing agent using transient DNA or DNA-free methods such as RNA transfection (e.g. mRNA+gRNA transfection), or Ribonucleoprotein (RNP) transfection (e.g. protein-RNA complex transfection, e.g. Cas9/gRNA ribonucleoprotein (RNP) complex transfection).

For example, Cas9 can be introduced as a DNA expression plasmid, in vitro transcript (i.e. RNA), or as a recombinant protein bound to the RNA portion in a ribonucleoprotein particle (RNP). gRNA, for example, can be delivered either as a DNA plasmid or as an in vitro transcript (i.e. RNA).

Any method known in the art for RNA or RNP transfection can be used in accordance with the present teachings, such as, but not limited to microinjection [as described by Cho et al., “Heritable gene knockout in Caenorhabditis elegans by direct injection of Cas9-sgRNA ribonucleoproteins,” Genetics (2013) 195:1177-1180, incorporated herein by reference], electroporation [as described by Kim et al., “Highly efficient RNA-guided genome editing in human cells via delivery of purified Cas9 ribonucleoproteins” Genome Res. (2014) 24:1012-1019, incorporated herein by reference], or lipid-mediated transfection e.g. using liposomes [as described by Zuris et al., “Cationic lipid-mediated delivery of proteins enables efficient protein-based genome editing in vitro and in vivo” Nat Biotechnol. (2014) doi: 10.1038/nbt.3081, incorporated herein by reference]. Additional methods of RNA transfection are described in U.S. Patent Application No. 20160289675, incorporated herein by reference in its entirety.

One advantage of RNA transfection methods of the invention is that RNA transfection is essentially transient and vector-free. A RNA transgene can be delivered to a cell and expressed therein, as a minimal expressing cassette without the need for any additional sequences (e.g. viral sequences).

According to one embodiment, the DNA editing agent of the invention is introduced into the plant cell using expression vectors.

The “expression vector” (also referred to herein as “a nucleic acid construct”, “vector” or “construct”) of some embodiments of the invention includes additional sequences which render this vector suitable for replication in prokaryotes, eukaryotes, or preferably both (e.g., shuttle vectors).

Constructs useful in the methods according to some embodiments of the invention may be constructed using recombinant DNA technology well known to persons skilled in the art. The nucleic acid sequences may be inserted into vectors, which may be commercially available, suitable for transforming into plants and suitable for transient expression of the gene of interest in the transformed cells. The genetic construct can be an expression vector wherein the nucleic acid sequence is operably linked to one or more regulatory sequences allowing expression in the plant cells.

According to one embodiment, in order to express a functional DNA editing agent, in cases where the cleaving module (nuclease) is not an integral part of the DNA recognition unit, the expression vector may encode the cleaving module as well as the DNA recognition unit (e.g. gRNA in the case of CRISPR/Cas).

Alternatively, the cleaving module (nuclease) and the DNA recognition unit (e.g. gRNA) may be cloned into separate expression vectors. In such a case, at least two different expression vectors must be transformed into the same plant cell.

Alternatively, when a nuclease is not utilized (i.e. not administered from an exogenous source to the cell), the DNA recognition unit (e.g. gRNA) may be cloned and expressed using a single expression vector.

Typical expression vectors may also contain a transcription and translation initiation sequence, transcription and translation terminator and optionally a polyadenylation signal.

According to one embodiment, the DNA editing agent comprises a nucleic acid agent encoding at least one DNA recognition unit (e.g. gRNA) operatively linked to a cis-acting regulatory element active in plant cells (e.g., promoter).

According to one embodiment, the nuclease (e.g. endonuclease) and the DNA recognition unit (e.g. gRNA) are encoded from the same expression vector. Such a vector may comprise a single cis-acting regulatory element active in plant cells (e.g., promoter) for expression of both the nuclease and the DNA recognition unit. Alternatively, the nuclease and the DNA recognition unit may each be operably linked to a cis-acting regulatory element active in plant cells (e.g., promoter).

According to one embodiment, the nuclease (e.g. endonuclease) and the DNA recognition unit (e.g. gRNA) are encoded from different expression vectors whereby each is operably linked to a cis-acting regulatory element active in plant cells (e.g., promoter).

As used herein the phrase “plant-expressible” or “active in plant cells” refers to a promoter sequence, including any additional regulatory elements added thereto or contained therein, that is at least capable of inducing, conferring, activating or enhancing expression in a plant cell, tissue or organ, preferably a monocotyledonous or dicotyledonous plant cell, tissue, or organ.

The plant promoter employed can be a constitutive promoter, a tissue specific promoter, an inducible promoter, a chimeric promoter or a developmentally regulated promoter.

Examples of preferred promoters useful for the methods of some embodiments of the invention are presented in Table I, II, III and IV.

TABLE I
Exemplary constitutive promoters for use in the performance of some embodiments of the invention

| Gene Source | Expression Pattern | Reference |
|---|---|---|
| Actin | constitutive | McElroy et al, Plant Cell, 2: 163-171, 1990 |
| CAMV 35S | constitutive | Odell et al, Nature, 313: 810-812, 1985 |
| CaMV 19S | constitutive | Nilsson et al., Physiol. Plant 100: 456-462, 1997 |
| GOS2 | constitutive | de Pater et al, Plant J Nov; 2(6): 837-44, 1992 |
| ubiquitin | constitutive | Christensen et al, Plant Mol. Biol. 18: 675-689, 1992 |
| Rice cyclophilin | constitutive | Bucholz et al, Plant Mol Biol. 25(5): 837-43, 1994 |
| Maize H3 histone | constitutive | Lepetit et al, Mol. Gen. Genet. 231: 276-285, 1992 |
| Actin 2 | constitutive | An et al, Plant J. 10(1); 107121, 1996 |
| CVMV (Cassava Vein Mosaic Virus | constitutive | Lawrenson et al, Gen Biol 16: 258, 2015 |
| U6 (AtU626; TaU6) | constitutive | Lawrenson et al, Gen Biol 16: 258, 2015 |

TABLE II
Exemplary seed-preferred promoters for use in the performance of some embodiments of the invention

| Gene Source | Expression Pattern | Reference |
|---|---|---|
| Seed specific genes | seed | Simon, et al., Plant Mol. Biol. 5. 191, 1985; |
| | | Scofield, et al., J. Biol. Chem. 262: 12202, |
| | | 1987; Baszczynski, et al., Plant Mol. Biol. |
| | | 14: 633, 1990. |
| Brazil Nut albumin | seed | Pearson' et al., Plant Mol. Biol. 18: |
| | | 235-245, 1992. |
| legumin | seed | Ellis, et al. Plant Mol. Biol. 10: 203-214, 1988 |
| Glutelin (rice) | seed | Takaiwa, et al., Mol. Gen. Genet. 208: |
| | | 15-22, 1986; Takaiwa, et al., FEBS Letts. 221: |
| | | 43-47, 1987 |
| Zein | seed | Matzke et al Plant Mol Biol, 143). 323-32 1990 |
| napA | seed | Stalberg, et al, Planta 199: 515-519, 1996 |
| wheat LMW and HMW, | endosperm | Mol Gen Genet 216: 81-90, 1989; NAR 17: 461-2, |
| glutenin-1 | | |
| Wheat SPA | seed | Albanietal, Plant Cell, 9: 171-184, 1997 |
| wheat a, b and g gliadins | endosperm | EMBO3: 1409-15, 1984 |
| Barley ltrl promoter | endosperm | |
| barley B1, C, D hordein | endosperm | Theor Appl Gen 98: 1253-62, 1999; |
| | | Plant J 4: 343-55, 1993; Mol Gen Genet |
| | | 250: 750-60, 1996 |
| Barley DOF | endosperm | Mena et al, The Plant Journal, 116(1): 53-62, 1998 |
| Biz2 | endosperm | EP99106056.7 |
| Synthetic promoter | endosperm | Vicente-Carbajosa et al., Plant J. 13: 629-640, 1998 |
| rice prolamin NRP33 | endosperm | Wu et al, Plant Cell Physiology 39(8) 885-889, 1998 |
| rice-globulin Glb-1 | endosperm | Wu et al, Plant Cell Physiology 398) 885-889, 1998 |
| rice OSH1 | emryo | Sato et al, Proc. Nati. Acad. Sci. USA, 93: 8117-8122 |
| rice alpha-globulin | endosperm | Nakase et al. Plant Mol. Biol. 33: 513-S22, 1997 |
| REB/OHP-1 | | |
| rice ADP-glucose PP | endosperm | Trans Res 6: 157-68, 1997 |
| maize ESR gene family | endosperm | Plant J 12: 235-46, 1997 |
| sorgum gamma-kafirin | endosperm | PMB 32: 1029-35, 1996 |
| KNOX | emryo | Postma-Haarsma ef al, Plant Mol. Biol. 39: 257-71, 1999 |
| rice oleosin | Embryo and aleuton | Wu et at, J. Biochem., 123: 386, 1998 |
| sunflower oleosin | Seed (embryo and dry seed) | Cummins, et al., Plant Mol. Biol. 19: 873-876, 1992 |

TABLE III
Exemplary flower-specific promoters for
use in the performance of the invention

| Gene Source | Expression Pattern | Reference |
|---|---|---|
| AtPRP4 | flowers | www(dot)salus(dot) |
| | | medium(dot)edu/m |
| | | mg/tierney/html |
| chalene | flowers | Van der Meer, et al., |
| synthase (chsA) | | Plant Mol. Biol. |
| | | 15, 95-109, 1990. |
| LAT52 | anther | Twell et al Mol. Gen |
| | | Genet. 217: 240-245 (1989) |
| apetala-3 | flowers | |

TABLE IV
Alternative rice promoters for use
in the performance of the invention

| PRO # | Gene | Expression |
|---|---|---|
| PR00001 | Metallothionein Mte | transfer layer of embryo + |
| | | calli |
| PR00005 | putative beta-amylase | transfer layer of embryo |
| PR00009 | Putative cellulose synthase | Weak in roots |
| PR00012 | lipase (putative) | |
| PR00014 | Transferase (putative) | |
| PR00016 | peptidyl prolyl cis-trans | |
| | isomerase (putative) | |
| PR00019 | unknown | |
| PR00020 | prp protein (putative) | |
| PR00029 | noduline (putative) | |
| PR00058 | Proteinase inhibitor Rgpi9 | seed |
| PR00061 | beta expansine EXPB9 | Weak in young flowers |
| PR00063 | Structural protein | young tissues + calli + |
| | | embryo |
| PR00069 | xylosidase (putative) | |
| PR00075 | Prolamine 10 Kda | strong in endosperm |
| PR00076 | allergen RA2 | strong in endosperm |
| PR00077 | prolamine RP7 | strong in endosperm |
| PR00078 | CBP80 | |
| PR00079 | starch branching enzyme I | |
| PR00080 | Metallothioneine-like ML2 | transfer layer of embryo + |
| | | calli |
| PR00081 | putative caffeoyl-CoA | shoot |
| | 3-0 methyltransferase | |
| PR00087 | prolamine RM9 | strong in endosperm |
| PR00090 | prolamine RP6 | strong in endosperm |
| PR00091 | prolamine RP5 | strong in endosperm |
| PR00092 | allergen RA5 | |
| PR00095 | putative | embryo |
| | methionine aminopeptidase | |
| PR00098 | ras-related GTP binding | |
| | protein | |
| PR00104 | beta expansine EXPB1 | |
| PR00105 | Glycine rich protein | |
| PR00108 | metallothionein like | |
| | protein (putative) | |
| PR00110 | RCc3 strong root | |
| PR00111 | uclacyanin 3-like protein | weak discrimination center/ |
| | | shoot meristem |
| PR00116 | 26S proteasome | very weak meristem |
| | regulatory particle | specific |
| | non-ATPase subunit 11 | |
| PR00117 | putative 40S ribosomal protein | weak in endosperm |
| PR00122 | chlorophyll a/lo-binding | very weak in shoot |
| | protein precursor (Cab27) | |
| PR00123 | putative | Strong leaves |
| | protochlorophyllide reductase | |
| PR00126 | metallothionein RiCMT | strong discrimination |
| | | center shoot meristem |
| PR00129 | GOS2 | Strong constitutive |
| PR00131 | GOS9 | |
| PR00133 | chitinase Cht-3 | very weak meristem |
| | | specific |
| PR00135 | alpha-globulin | Strong in endosperm |
| PR00136 | alanine aminotransferase | Weak in endosperm |
| PR00138 | Cyclin A2 | |
| PR00139 | Cyclin D2 | |
| PR00140 | Cyclin D3 | |
| PR00141 | Cyclophyllin 2 | Shoot and seed |
| PR00146 | sucrose synthase SS1 (barley) | medium constitutive |
| PR00147 | trypsin inhibitor ITR1 (barley) | weak in endosperm |
| PR00149 | ubiquitine 2 with intron | strong constitutive |
| PR00151 | WSI18 | Embryo and stress |
| PR00156 | HVA22 homologue (putative) | |
| PR00157 | EL2 | |
| PR00169 | aquaporine | medium constitutive in |
| | | young plants |
| PR00170 | High mobility group protein | Strong constitutive |
| PR00171 | reversibly glycosylated | weak constitutive |
| | protein RGP1 | |
| PR00173 | cytosolic MDH | shoot |
| PR00175 | RAB21 | Embryo and stress |
| PR00176 | CDPK7 | |
| PR00177 | Cdc2-1 | very weak in meristem |
| PR00197 | sucrose synthase 3 | |
| PRO0198 | OsVP1 | |
| PRO0200 | OSHI | very weak in young |
| | | plant meristem |
| PRO0208 | putative chlorophyllase | |
| PRO0210 | OsNRT1 | |
| PRO0211 | EXP3 | |
| PRO0216 | phosphate transporter OjPT1 | |
| PRO0218 | oleosin 18 kd | aleurone + embryo |
| PRO0219 | ubiquitine 2 without intron | |
| PRO0220 | RFL | |
| PRO0221 | maize UBI delta intron | not detected |
| PRO0223 | glutelin-1 | |
| PRO0224 | fragment of prolamin | |
| | RP6 promoter | |
| PRO0225 | 4xABRE | |
| PRO0226 | glutelin OSGLUA3 | |
| PRO0227 | BLZ-2_short (barley) | |
| PR00228 | BLZ-2_long (barley) | |

The inducible promoter is a promoter induced in a specific plant tissue, by a developmental stage or by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

According to one embodiment the promoter is a pathogen-inducible promoter. These promoters direct the expression of genes in plants following infection with a pathogen such as bacteria, fungi, viruses, nematodes and insects. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol 89:245-254; Uknes et al. (1992) Plant Cell 4:645-656; and Van Loon (1985) Plant Mol. Virol. 4:111-116.

According to one embodiment, when more than one promoter is used in the expression vector, the promoters are identical (e.g., all identical, at least two identical).

According to one embodiment, when more than one promoter is used in the expression vector, the promoters are different (e.g., at least two are different, all are different).

According to one embodiment, the promoter in the expression vector includes, but is not limited to, CaMV 35S, 2×CaMV 35S, CaMV 19S, ubiquitin, AtU626 or TaU6.

According to a specific embodiment, the promoter in the expression vector comprises a 35S promoter.

According to a specific embodiment, the promoter in the expression vector comprises a U6 promoter.

Expression vectors may also comprise transcription and translation initiation sequences, transcription and translation terminator sequences and optionally a polyadenylation signal.

According to a specific embodiment, the expression vector comprises a termination sequence, such as but not limited to, a G7 termination sequence, an AtuNos termination sequence or a CaMV-35S terminator sequence.

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

According to one embodiment, an agrobacterium-free expression method is used to introduce foreign genes into plant cells. According to one embodiment, the agrobacterium-free expression method is transient. According to a specific embodiment, a bombardment method is used to introduce foreign genes into plant cells. According to another specific embodiment, bombardment of a plant root is used to introduce foreign genes into plant cells. An exemplary bombardment method which can be used in accordance with some embodiments of the invention is discussed in the examples section which follows.

Furthermore, various cloning kits or gene synthesis can be used according to the teachings of some embodiments of the invention.

According to one embodiment the nucleic acid construct is a binary vector. Examples for binary vectors are pBIN19, pBI101, pBinAR, pGPTV, pCAMBIA, pBIB-HYG, pBecks, pGreen or pPZP (Hajukiewicz, P. et al., Plant Mol. Biol. 25, 989 (1994), and Hellens et al, Trends in Plant Science 5, 446 (2000)).

Examples of other vectors to be used in other methods of DNA delivery (e.g. transfection, electroporation, bombardment, viral inoculation as discussed below) are: pGE-sgRNA (Zhang et al. Nat. Comms. 2016 7:12697), pJIT163-Ubi-Cas9 (Wang et al. Nat. Biotechnol 2004 32, 947-951), pICH47742::2x35S-5′UTR-hCas9(STOP)-NOST (Belhan et al. Plant Methods 2013 11; 9(1):39), pAHC25 (Christensen, A. H. & P. H. Quail, 1996. Ubiquitin promoter-based vectors for high-level expression of selectable and/or screenable marker genes in monocotyledonous plants. Transgenic Research 5: 213-218), pHBT-sGFP(S65T)-NOS (Sheen et al. Protein phosphatase activity is required for light-inducible gene expression in maize, EMBO J. 12 (9), 3497-3505 (1993).

According to one embodiment, the method of some embodiments of the invention further comprises introducing into the plant cell donor oligonucleotides.

According to one embodiment, when the modification is an insertion, the method further comprises introducing into the plant cell donor oligonucleotides.

According to one embodiment, when the modification is a deletion, the method further comprises introducing into the plant cell donor oligonucleotides.

According to one embodiment, when the modification is a deletion and insertion (e.g. swapping), the method further comprises introducing into the plant cell donor oligonucleotides.

According to one embodiment, when the modification is a point mutation, the method further comprises introducing into the plant cell donor oligonucleotides.

As used herein, the term “donor oligonucleotides” or “donor oligos” refers to exogenous nucleotides, i.e. externally introduced into the plant cell to generate a precise change in the genome. According to one embodiment, the donor oligonucleotides are synthetic.

According to one embodiment, the donor oligos are RNA oligos.

According to one embodiment, the donor oligos are DNA oligos.

According to one embodiment, the donor oligos are synthetic oligos.

According to one embodiment, the donor oligonucleotides comprise single-stranded donor oligonucleotides (ssODN).

According to one embodiment, the donor oligonucleotides comprise double-stranded donor oligonucleotides (dsODN).

According to one embodiment, the donor oligonucleotides comprise double-stranded DNA (dsDNA).

According to one embodiment, the donor oligonucleotides comprise double-stranded DNA-RNA duplex (DNA-RNA duplex).

According to one embodiment, the donor oligonucleotides comprise double-stranded DNA-RNA hybrid

According to one embodiment, the donor oligonucleotides comprise single-stranded DNA-RNA hybrid.

According to one embodiment, the donor oligonucleotides comprise single-stranded DNA (ssDNA).

According to one embodiment, the donor oligonucleotides comprise double-stranded RNA (dsRNA).

According to one embodiment, the donor oligonucleotides comprise single-stranded RNA (ssRNA).

According to one embodiment, the donor oligonucleotides comprise the DNA or RNA sequence for swapping (as discussed above).

According to one embodiment, the donor oligonucleotides are provided in a non-expressed vector format or oligo.

According to one embodiment, the donor oligonucleotides comprise a DNA donor plasmid (e.g. circular or linearized plasmid).

According to one embodiment, the donor oligonucleotides comprise about 50-5000, about 100-5000, about 250-5000, about 500-5000, about 750-5000, about 1000-5000, about 1500-5000, about 2000-5000, about 2500-5000, about 3000-5000, about 4000-5000, about 50-4000, about 100-4000, about 250-4000, about 500-4000, about 750-4000, about 1000-4000, about 1500-4000, about 2000-4000, about 2500-4000, about 3000-4000, about 50-3000, about 100-3000, about 250-3000, about 500-3000, about 750-3000, about 1000-3000, about 1500-3000, about 2000-3000, about 50-2000, about 100-2000, about 250-2000, about 500-2000, about 750-2000, about 1000-2000, about 1500-2000, about 50-1000, about 100-1000, about 250-1000, about 500-1000, about 750-1000, about 50-750, about 150-750, about 250-750, about 500-750, about 50-500, about 150-500, about 200-500, about 250-500, about 350-500, about 50-250, about 150-250, or about 200-250 nucleotides.

According to a specific embodiment, the donor oligonucleotides comprising the ssODN (e.g. ssDNA or ssRNA) comprise about 200-500 nucleotides.

According to a specific embodiment, the donor oligonucleotides comprising the dsODN (e.g. dsDNA or dsRNA) comprise about 250-5000 nucleotides.

According to one embodiment, for gene swapping of an endogenous RNA silencing molecule (e.g. miRNA) with a RNA silencing sequence of choice (e.g. siRNA), the expression vector, ssODN (e.g. ssDNA or ssRNA) or dsODN (e.g. dsDNA or dsRNA) does not have to be expressed in a plant cell and could serve as a non-expressing template. According to a specific embodiment, in such a case only the DNA editing agent (e.g. Cas9/sgRNA modules) need to be expressed if provided in a DNA form.

According to some embodiments, for gene editing of an endogenous non-coding RNA molecule (e.g. RNA silencing molecule) without the use of a nuclease, the DNA editing agent (e.g., gRNA) may be introduced into the eukaryotic cell with our without (e.g. oligonucleotide donor DNA or RNA, as discussed herein).

According to one embodiment, introducing into the plant cell donor oligonucleotides is effected using any of the methods described above (e.g. using the expression vectors or RNP transfection).

According to one embodiment, the gRNA and the DNA donor oligonucleotides are co-introduced into the plant cell (e.g. via bombardment). It will be appreciated that any additional factors (e.g. nuclease) may be co-introduced therewith.

According to one embodiment, the gRNA is introduced into the plant cell prior to the DNA donor oligonucleotides (e.g. within a few minutes or a few hours). It will be appreciated that any additional factors (e.g. nuclease) may be introduced prior to, concomitantly with, or following the gRNA or the DNA donor oligonucleotides.

According to one embodiment, the gRNA is introduced into the plant cell subsequent to the DNA donor oligonucleotides (e.g. within a few minutes or a few hours). It will be appreciated that any additional factors (e.g. nuclease) may be introduced prior to, concomitantly with, or following the gRNA or the DNA donor oligonucleotides.

According to one embodiment, there is provided a composition comprising at least one gRNA and DNA donor oligonucleotides for genome editing.

According to one embodiment, there is provided a composition comprising at least one gRNA, a nuclease (e.g. endonuclease) and DNA donor oligonucleotides for genome editing.

There are various methods of direct DNA transfer into plant cells and the skilled artisan will know which to select. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or gold or tungsten particles, and the microprojectiles are physically accelerated into protoplasts, cells or plant tissues.

Thus, the delivery of nucleic acids may be introduced into a plant cell in embodiments of the invention by any method known to those of skill in the art, including, for example and without limitation: by transformation of protoplasts (See, e.g., U.S. Pat. No. 5,508,184); by desiccation/inhibition-mediated DNA uptake (See, e.g., Potrykus et al. (1985) Mol. Gen. Genet. 199:183-8); by electroporation (See, e.g., U.S. Pat. No. 5,384,253); by agitation with silicon carbide fibers (See, e.g., U.S. Pat. Nos. 5,302,523 and 5,464,765); by Agrobacterium-mediated transformation (See, e.g., U.S. Pat. Nos. 5,563,055, 5,591,616, 5,693,512, 5,824,877, 5,981,840, and 6,384,301); by acceleration of DNA-coated particles (See, e.g., U.S. Pat. Nos. 5,015,580, 5,550,318, 5,538,880, 6,160,208, 6,399,861, and 6,403,865) and by Nanoparticles, nanocarriers and cell penetrating peptides (WO201126644A2; WO2009046384A1; WO2008148223A1) in the methods to deliver DNA, RNA, Peptides and/or proteins or combinations of nucleic acids and peptides into plant cells.

Other methods of transfection include the use of transfection reagents (e.g. Lipofectin, ThermoFisher), dendrimers (Kukowska-Latallo, J. F. et al., 1996, Proc. Natl. Acad. Sci. USA93, 4897-902), cell penetrating peptides (Mae et al., 2005, Internalisation of cell-penetrating peptides into tobacco protoplasts, Biochimica et Biophysica Acta 1669(2):101-7) or polyamines (Zhang and Vinogradov, 2010, Short biodegradable polyamines for gene delivery and transfection of brain capillary endothelial cells, J Control Release, 143(3):359-366).

According to a specific embodiment, for introducing DNA into plant cells (e.g. protoplasts) the method comprises polyethylene glycol (PEG)-mediated DNA uptake. For further details see Karesch et al. (1991) Plant Cell Rep. 9:575-578; Mathur et al. (1995) Plant Cell Rep. 14:221-226; Negrutiu et al. (1987) Plant Cell Mol. Biol. 8:363-373. Plant cells (e.g. protoplasts) are then cultured under conditions that allowed them to grow cell walls, start dividing to form a callus, develop shoots and roots, and regenerate whole plants.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the genetically identical transformed plants.

Micropropagation is a process of growing new generation plants from a single piece of tissue that has been excised from a selected parent plant or cultivar. This process permits the mass reproduction of plants having the desired trait. The new generated plants are genetically identical to, and have all of the characteristics of, the original plant. Micropropagation (or cloning) allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV, TRV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is a RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Regardless of the transformation/infection method employed, the present teachings further select transformed cells comprising a genome editing event.

According to a specific embodiment, selection is carried out such that only cells comprising a successful accurate modification (e.g. swapping, insertion, deletion, point mutation) in the specific locus are selected. Accordingly, cells comprising any event that includes a modification (e.g. an insertion, deletion, point mutation) in an unintended locus are not selected.

According to one embodiment, selection of modified cells can be performed at the phenotypic level, by detection of a molecular event, by detection of a fluorescent reporter, or by growth in the presence of selection (e.g., antibiotic).

According to one embodiment, selection of modified cells is performed by analyzing the biogenesis and occurrence of the newly edited non-coding RNA molecule (e.g. the presence of new miRNA version, the presence of novel edited siRNAs, piRNAs, tasiRNAs etc).

According to one embodiment, selection of modified cells is performed by analyzing the silencing activity and/or specificity of the non-coding RNA molecule (e.g. RNA silencing molecule) towards a second target RNA or target RNA of interest by validating at least one phenotype in the plant or the organism that encode the target RNA, e.g. plant leaf coloring, e.g. partial or complete loss of chlorophyll in leaves and other organs (bleaching), presence/absence of nacrotic patterns, flower coloring, fruit traits (such as shelf life, firmness and flavor), growth rate, plant size (e.g. dwarfism), crop yield, biotic stress resistance (e.g. disease resistance, nematode mortality, beetle's egg laying rate, or other resistant phenotypes associated with any of bacteria, viruses, fungi, parasites, insects, weeds, and cultivated or native plants), abiotic stress resistance (e.g. heat/cold resistance, drought resistance, salt resistance, resistance to allyl alcohol, or resistant to lack of nutrients e.g. Phosphorus (P)).

According to one embodiment, the silencing specificity of the non-coding RNA molecule is determined genotypically, e.g. by expression of a gene or lack of expression.

According to one embodiment, the silencing specificity of the non-coding RNA molecule is determined phenotypically.

According to one embodiment, a phenotype of the plant is determined prior to a genotype.

According to one embodiment, a genotype of the plant is determined prior to a phenotype.

According to one embodiment, selection of modified cells is performed by analyzing the silencing activity and/or specificity of the non-coding RNA molecule (e.g. RNA silencing molecule) towards a second target RNA or target RNA of interest by measuring a RNA level of the second target RNA or target RNA of interest. This can be performed using any method known in the art, e.g. by Northern blotting, Nuclease Protection Assays, In Situ hybridization, or quantitative RT-PCR.

According to one embodiment, selection of modified cells is performed by analyzing plant cells or clones comprising the DNA editing event also referred to herein as “mutation” or “edit”, dependent on the type of editing sought e.g., insertion, deletion, insertion-deletion (Indel), inversion, substitution and combinations thereof.

Methods for detecting sequence alteration are well known in the art and include, but not limited to, DNA and RNA sequencing (e.g., next generation sequencing), electrophoresis, an enzyme-based mismatch detection assay and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based T7 endonuclease, Hetroduplex and Sanger sequencing, or PCR followed by restriction digest to detect appearance or disappearance of unique restriction site/s.

Another method of validating the presence of a DNA editing event e.g., Indels comprises a mismatch cleavage assay that makes use of a structure selective enzyme (e.g. endonuclease) that recognizes and cleaves mismatched DNA.

According to one embodiment, selection of transformed cells is effected by flow cytometry (FACS) selecting transformed cells exhibiting fluorescence emitted by the fluorescent reporter. Following FACS sorting, positively selected pools of transformed plant cells, displaying the fluorescent marker are collected and an aliquot can be used for testing the DNA editing event as discussed above.

In cases where antibiotic selection marker was used, following transformation plant cell clones are cultivated in the presence of selection (e.g., antibiotic) until they develop into colonies i.e., clones and micro-calli. A portion of the cells of the calli are then analyzed (validated) for the DNA editing event, as discussed above.

Thus, according to one embodiment of the invention, the method further comprises validating in the transformed cells complementarity of the endogenous non-coding RNA molecule (e.g. RNA silencing molecule) towards the second target RNA.

As mentioned above, following modification of the gene encoding the non-coding RNA molecule (e.g. RNA silencing molecule), the non-coding RNA molecule (e.g. RNA silencing molecule) comprises at least about 30%, 33%, 40%, 50%, 60%, 70%, 80%, 85%, 90%, 91%, 92%, 93%, 94%, 95%, 96%, 97%, 98%, 99% or even 100% complementarity towards the sequence of the second target RNA or target RNA of interest.

The specific binding of designed non-coding RNA molecule with a target RNA of interest can be determined by any method known in the art, such as by computational algorithms (e.g. BLAST) and verified by methods including e.g. Northern blot, In Situ hybridization, QuantiGene Plex Assay etc.

It will be appreciated that positive clones can be homozygous or heterozygous for the DNA editing event. In case of a heterozygous cell, the cell (e.g., when diploid) may comprise a copy of a modified gene and a copy of a non-modified gene of the non-coding RNA molecule (e.g. RNA silencing molecule). The skilled artisan will select the clone for further culturing/regeneration according to the intended use.

According to one embodiment, when a transient method is desired, clones exhibiting the presence of a DNA editing event as desired are further analyzed and selected for the absence of the DNA editing agent, namely, loss of DNA sequences encoding for the DNA editing agent. This can be done, for example, by analyzing the loss of expression of the DNA editing agent (e.g., at the mRNA, protein) e.g., by fluorescent detection of GFP or q-PCR, HPLC.

According to one embodiment, when a transient method is desired, the cells may be analyzed for the absence of the nucleic acid construct as described herein or portions thereof e.g., nucleic acid sequence encoding the DNA editing agent. This can be affirmed by fluorescent microscopy, q-PCR, FACS, and or any other method such as Southern blot, PCR, sequencing, HPLC).

According to one embodiment, the plant is crossed in order to obtain a plant devoid of the DNA editing agent (e.g. of the endonuclease), as discussed below.

Positive clones may be stored (e.g., cryopreserved).

Alternatively, plant cells (e.g., protoplasts) may be regenerated into whole plants first by growing into a group of plant cells that develops into a callus and then by regeneration of shoots (callogenesis) from the callus using plant tissue culture methods. Growth of protoplasts into callus and regeneration of shoots requires the proper balance of plant growth regulators in the tissue culture medium that must be customized for each species of plant.

Protoplasts may also be used for plant breeding, using a technique called protoplast fusion. Protoplasts from different species are induced to fuse by using an electric field or a solution of polyethylene glycol. This technique may be used to generate somatic hybrids in tissue culture.

Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment. For a thorough review see Maheshwari et al. 1986 Differentiation of Protoplasts and of Transformed Plant Cells: 3-36. Springer-Verlag, Berlin.

The regenerated plants can be subjected to further breeding and selection as the skilled artisan sees fit.

Thus, embodiments of the invention further relate to plants, plant cells and processed product of plants comprising the non-coding RNA molecule (e.g. RNA silencing molecule) capable of silencing a second target RNA generated according to the present teachings.

According to one aspect of the invention, there is provided a method of producing a plant with reduced expression of a target gene, the method comprising: (a) breeding the plant according to some embodiments of the invention and (b) selecting for progeny plants that have reduced expression of the target RNA of interest or the second target RNA, or progeny that comprises a silencing specificity in the non-coding RNA molecule towards a target RNA of interest, and which do not comprise said DNA editing agent, thereby producing the plant with reduced expression of a target gene.

According to one embodiment, breeding comprises crossing or selfing.

The term “crossing” as used herein refers to the fertilization of female plants (or gametes) by male plants (or gametes). The term “gamete” refers to the haploid reproductive cell (egg or sperm) produced in plants by mitosis from a gametophyte and involved in sexual reproduction, during which two gametes of opposite sex fuse to form a diploid zygote. The term generally includes reference to a pollen (including the sperm cell) and an ovule (including the ovum). “crossing” therefore generally refers to the fertilization of ovules of one individual with pollen from another individual, whereas “selfing” refers to the fertilization of ovules of an individual with pollen from the same individual. Crossing is widely used in plant breeding and results in a mix of genomic information between the two plants crossed one chromosome from the mother and one chromosome from the father. This will result in a new combination of genetically inherited traits.

As mentioned above, the plant may be crossed in order to obtain a plant devoid of undesired factors e.g. DNA editing agent (e.g. endonuclease).

According to one embodiment, there is provided a method of generating a plant with increased stress tolerance, increased yield, increased growth rate or increased yield quality, the method comprising modifying a gene encoding or processed into a non-coding RNA molecule or into a RNA silencing in a plant cell according to the method of some embodiments of the invention, wherein the target RNA of interest is of a gene of the plant conferring sensitivity to stress, decreased yield, decreased growth rate or decreased yield quality thereby generating the plant.

The phrase “stress tolerance” as used herein refers to the ability of a plant to endure a biotic or abiotic stress without suffering a substantial alteration in metabolism, growth, productivity and/or viability.

The phrase “abiotic stress” as used herein refers to the exposure of a plant, plant cell, or the like, to a non-living (“abiotic”) physical or chemical agent that has an adverse effect on metabolism, growth, development, propagation, or survival of the plant (collectively, “growth”). An abiotic stress can be imposed on a plant due, for example, to an environmental factor such as water (e.g., flooding, drought, or dehydration), anaerobic conditions (e.g., a lower level of oxygen or high level of CO₂), abnormal osmotic conditions (e.g. osmotic stress), salinity, or temperature (e.g., hot/heat, cold, freezing, or frost), an exposure to pollutants (e.g. heavy metal toxicity), anaerobiosis, nutrient deficiency (e.g., nitrogen deficiency or limited nitrogen), atmospheric pollution or UV irradiation.

The phrase “biotic stress” as used herein refers to the exposure of a plant, plant cell, or the like, to a living (“biotic”) organism that has an adverse effect on metabolism, growth, development, propagation, or survival of the plant (collectively, “growth”). Biotic stress can be caused by, for example, bacteria, viruses, fungi, parasites, beneficial and harmful insects, weeds, and cultivated or native plants.

The phrase “yield” or “plant yield” as used herein refers to increased plant growth (growth rate), increased crop growth, increased biomass, and/or increased plant product production (including grain, fruit, seeds, etc.).

According to one embodiment, in order to generate a plant with increased stress tolerance, increased yield, increased growth rate or increased yield quality, the non-coding RNA molecule is designed to target a RNA of interest being of a gene of the plant conferring sensitivity to stress, decreased yield, decreased growth rate or decreased yield quality.

According to one embodiment, exemplary susceptibility plant genes to be targeted (e.g. knocked out) include, but are not limited to, the susceptibility S-genes, such as those residing at genetic loci known as MLO (Mildew, Locus O).

According to one embodiment, the plants generated by the present method comprise increased stress tolerance, increased yield, increased yield quality, increased growth rate, by at least about 10%, 20%, 30%, 40%, 50%, 60%, 70%, 80%, 90%, 95% or 100% as compared to plants not generated by the present methods.

Any method known in the art for assessing increased stress tolerance may be used in accordance with the present invention. Exemplary methods of assessing increased stress tolerance include, but are not limited to, downregulation of PagSAP1 in poplar for increased salt stress tolerance as described in Yoon, SK., Bae, EK., Lee, H. et al. Trees (2018) 32: 823. www(dot)doi(dot)org/10.1007/s00468-018-1675-2), and increased drought tolerance in tomato by downregulation of SlbZIP38 (Pan Y et al. Genes 2017, 8, 402; doi:10.3390/genes8120402, incorporated herein by reference.

Any method known in the art for assessing increased yield may be used in accordance with the present invention. Exemplary methods of assessing increased yield include, but are not limited to, reduced DST expression in rice as described in Ar-Rafi Md. Faisal, et al, AJPS> Vol. 8 No. 9, August 2017 DOI: 10.4236/ajps.2017.89149; and downregulation of BnFTA in canola resulted in increased yield as described in Wang Y et al., Mol Plant. 2009 January; 2(1): 191-200.doi: 10.1093/mp/ssn088), both incorporated herein by reference.

Any method known in the art for assessing increased growth rate may be used in accordance with the present invention. Exemplary methods of assessing increased growth rate include, but are not limited to, reduced expression of BIG BROTHER in Arabidopsis or GA2-OXIDASE results in enhance growth and biomass as described in Marcelo de Freitas Lima et al. Biotechnology Research and Innovation(2017)1, 14 - - - 25, incorporated herein by reference.

Any method known in the art for assessing increased yield quality may be used in accordance with the present invention. Exemplary methods of assessing increased yield quality include, but are not limited to, down regulation of OsCKX2 in rice results in production of more tillers, more grains, and the grains were heavier as described in Yeh S_Y et al. Rice (N Y). 2015; 8: 36; and reduce OMT levels in many plants, which result in altered lignin accumulation, increase the digestibility of the material for industry purposes as described in Verma S R and Dwivedi U N, South African Journal of Botany Volume 91, March 2014, Pages 107-125, both incorporated herein by reference.

According to one embodiment, the method further enables generation of a plant comprising increased sweetness, increased sugar content, increased flavor, improved ripening control, increased water stress tolerance, increased heat stress tolerance, and increased salt tolerance. One of skill in the art will know how to utilize the methods described herein to choose target RNA sequences for modification.

According to one embodiment, there is provided a method of generating a pathogen tolerant or resistant plant, the method comprising modifying a gene encoding or processed into a non-coding RNA molecule or into a RNA silencing molecule in a plant cell according to the method of some embodiments of the invention, wherein the target RNA of interest is of a gene of the plant conferring sensitivity to the pathogen, thereby generating the pathogen tolerant or resistant plant.

According to one embodiment, there is provided a method of generating a pathogen tolerant or resistant plant, the method comprising modifying a gene encoding or processed into a non-coding RNA molecule or into a RNA silencing molecule in a plant cell according to the method of some embodiments of the invention, wherein the target RNA of interest is of a gene of the pathogen, thereby generating the pathogen tolerant or resistant plant.

According to one embodiment, there is provided a method of generating a pest tolerant or resistant plant, the method comprising modifying a gene encoding or processed into a non-coding RNA molecule or into a RNA silencing molecule in a plant cell according to the method of some embodiments of the invention, wherein the target RNA of interest is of a gene of the pest, thereby generating the pest tolerant or resistant plant.

As used herein the term “pathogen” refers to an organism that negatively affect plants by colonizing, damaging, attacking, or infecting them. Thus, pathogen may affect the growth, development, reproduction, harvest or yield of a plant. This includes organisms that spread disease and/or damage the host and/or compete for host nutrients. Plant pathogens include, but are not limited to, fungi, oomycetes, bacteria, viruses, viroids, virus-like organisms, phytoplasmas, protozoa, nematodes, insects and parasitic plants.

Non-limiting examples of pathogens include, but are not limited to, Roundheaded Borer such as long horned borers; psyllids such as red gum lerp psyllids (Glycaspis brimblecombei), blue gum psyllid, spotted gum lerp psyllids, lemon gum lep psyllids; tortoise beetles; snout beetles; leaf beetles; honey fungus; Thaumastocoris peregrinus; sessile gall wasps (Cynipidae) such as Leptocybe invasa, Ophelimus maskelli and Selitrichodes globules; Foliage-feeding caterpillars such as Omnivorous looper and Orange tortrix; Glassy-winged sharpshooter; and Whiteflies such as Giant whitefly. Other non-limiting examples of pathogens include Aphids such as Chaitophorus spp., Cloudywinged cottonwood and Periphyllus spp.; Armored scales such as Oystershell scale and San Jose scale; Carpenterworm; Clearwing moth borers such as American hornet moth and Western poplar clearwing; Flatheaded borers such as Bronze birch borer and Bronze poplar borer; Foliage-feeding caterpillars such as Fall webworm, Fruit-tree leafroller, Redhumped caterpillar, Satin moth caterpillar, Spiny elm caterpillar, Tent caterpillar, Tussock moths and Western tiger swallowtail; Foliage miners such as Poplar shield bearer; Gall and blister mites such as Cottonwood gall mite; Gall aphids such as Poplar petiolegall aphid; Glassy-winged sharpshooter; Leaf beetles and flea beetles; Mealybugs; Poplar and willow borer; Roundheaded borers; Sawflies; Soft scales such as Black scale, Brown soft scale, Cottony maple scale and European fruit lecanium; Treehoppers such as Buffalo treehopper; and True bugs such as Lace bugs and Lygus bugs.

Other non-limiting examples of viral plant pathogens include, but are not limited to Species: Pea early-browning virus (PEBV), Genus: Tobravirus. Species: Pepper ringspot virus (PepRSV), Genus: Tobravirus. Species: Watermelon mosaic virus (WMV), Genus: Potyvirus and other viruses from the Potyvirus Genus. Species: Tobacco mosaic virus Genus (TMV), Tobamovirus and other viruses from the Tobamovirus Genus. Species: Potato virus X Genus (PVX), Potexvirus and other viruses from the Potexvirus Genus. Thus the present teachings envisage targeting of RNA as well as DNA viruses (e.g. Gemini virus or Bigeminivirus). Geminiviridae viruses which may be targeted include, but are not limited to, Abutilon mosaic bigeminivirus, Ageratum yellow vein bigeminivirus, Bean calico mosaic bigeminivirus, Bean golden mosaic bigeminivirus, Bhendi yellow vein mosaic bigeminivirus, Cassava African mosaic bigeminivirus, Cassava Indian mosaic bigeminivirus, Chino del tomaté bigeminivirus, Cotton leaf crumple bigeminivirus, Cotton leaf curl bigeminivirus, Croton yellow vein mosaic bigeminivirus, Dolichos yellow mosaic bigeminivirus, Euphorbia mosaic bigeminivirus, Horsegram yellow mosaic bigeminivirus, Jatropha mosaic bigeminivirus, Lima bean golden mosaic bigeminivirus, Melon leaf curl bigeminivirus, Mung bean yellow mosaic bigeminivirus, Okra leaf-curl bigeminivirus, Pepper hausteco bigeminivirus, Pepper Texas bigeminivirus, Potato yellow mosaic bigeminivirus, Rhynchosia mosaic bigeminivirus, Serrano golden mosaic bigeminivirus, Squash leaf curl bigeminivirus, Tobacco leaf curl bigeminivirus, Tomato Australian leafcurl bigeminivirus, Tomato golden mosaic bigeminivirus, Tomato Indian leafcurl bigeminivirus, Tomato leaf crumple bigeminivirus, Tomato mottle bigeminivirus, Tomato yellow leaf curl bigeminivirus, Tomato yellow mosaic bigeminivirus, Watermelon chlorotic stunt bigeminivirus and Watermelon curly mottle bigeminivirus.

As used herein the term “pest” refers to an organism which directly or indirectly harms the plant. A direct effect includes, for example, feeding on the plant leaves. Indirect effect includes, for example, transmission of a disease agent (e.g. a virus, bacteria, etc.) to the plant. In the latter case the pest serves as a vector for pathogen transmission. Exemplary pests include, but are not limited to, beetles, psylids, insects, nematodes, snails.

According to one embodiment, the pathogen is a nematode. Exemplary nematodes include, but are not limited to, the burrowing nematode (Radopholus similis), Caenorhabditis elegans, Radopholus arabocoffeae, Pratylenchus coffeae, root-knot nematode (Meloidogyne spp.), cyst nematode (Heterodera and Globodera spp.), root lesion nematode (Pratylenchus spp.), the stem nematode (Ditylenchus dipsaci), the pine wilt nematode (Bursaphelenchus xylophilus), the reniform nematode (Rotylenchulus reniformis), Xiphinema index, Nacobbus aberrans and Aphelenchoides besseyi.

According to one embodiment, the pathogen is a fungus. Exemplary fungi include, but are not limited to, Fusarium oxysporum, Leptosphaeria maculans (Phoma lingam), Sclerotinia sclerotiorum, Pyricularia grisea, Gibberella fujikuroi (Fusarium moniliforme), Magnaporthe oryzae, Botrytis cinereal, Puccinia spp., Fusarium graminearum, Blumeria graminis, Mycosphaerella graminicola, Colletotrichum spp., Ustilago maydis, Melampsora lini, Phakopsora pachyrhizi and Rhizoctonia solani.

According to one embodiment, in order to generate a pathogen resistant or tolerant plant, the non-coding RNA molecule is designed to target a RNA of interest being of a gene of the plant conferring sensitivity to a pathogen.

According to one embodiment, an exemplary plant gene to be targeted includes, but is not limited to, the gene eIF4E which confers sensitivity to viral infection in cucumber.

According to one embodiment, in order to generate a pathogen resistant or tolerant plant, the non-coding RNA molecule is designed to target a RNA of interest being of a gene of the pathogen.

Determination of the plant or pathogen target genes may be achieved using any method known in the art such as by routine bioinformatics analysis.

According to one embodiment, the nematode pathogen gene comprises the Radopholus similis genes Calreticulin13 (CRT) or collagen 5 (col-5).

According to one embodiment, the fungi pathogen gene comprises the Fusarium oxysporum genes FOW2, FRP1, and OPR.

According to one embodiment, the pathogen gene includes, for example, vacuolar ATPase (vATPase), dvssj1 and dvssj2, α-tubulin and snf7.

According to a specific embodiment, when the plant is a Brassica napus (rapeseed), the target RNA of interest includes, but is not limited to, a gene of Leptosphaeria maculans (Phoma lingam) (causing e.g. Phoma stem canker) (e.g. as set forth in GenBank Accession No: AM933613.1); a gene of Flea beetle (Phyllotreta vittula or Chrysomelidae, e.g. as set forth in GenBank Accession No: KT959245.1); or a gene of by Sclerotinia sclerotiorum (causing e.g. Sclerotinia stem rot) (e.g. as set forth in GenBank Accession No: NW_001820833.1).

According to a specific embodiment, when the plant is a Citrus x sinensis (Orange), the target RNA of interest includes, but is not limited to, a gene of Citrus Canker (CCK) (e.g. as set forth in GenBank Accession No: AE008925); a gene of Candidatus Liberibacter spp. (causing e.g. Citrus greening disease) (e.g. as set forth in GenBank Accession No: CP001677.5); or a gene of Armillaria root rot (e.g. as set forth in GenBank Accession No: KY389267.1).

According to a specific embodiment, when the plant is a Elaeis guineensis (Oil palm), the target RNA of interest includes, but is not limited to, a gene of Ganoderma spp. (causing e.g. Basal stem rot (BSR) also known as Ganoderma butt rot) (e.g. as set forth in GenBank Accession No: U56128.1), a gene of Nettle Caterpillar or a gene of any one of Fusarium spp., Phytophthora spp., Pythium spp., Rhizoctonia solani (causing e.g. Root rot).

According to a specific embodiment, when the plant is a Fragaria vesca (Wild strawberry), the target RNA of interest includes, but is not limited to, a gene of Verticillium dahlia (causing e.g. Verticillium Wilt) (e.g. as set forth in GenBank Accession No: DS572713.1); or a gene of Fusarium oxysporum f.sp. fragariae (causing e.g. Fusarium wilt) (e.g. as set forth in GenBank Accession No: KR855868.1);

According to a specific embodiment, when the plant is a Glycine max (Soybean), the target RNA of interest includes, but is not limited to, a gene of P. pachyrhizi (causing e.g. Soybean rust, also known as Asian rust) (e.g. as set forth in GenBank Accession No: DQ026061.1); a gene of Soybean Aphid (e.g. as set forth in GenBank Accession No: KJ451424.1); a gene of Soybean Dwarf Virus (SbDV) (e.g. as set forth in GenBank Accession No: NC_003056.1); or a gene of Green Stink Bug (Acrosternum hilare) (e.g. as set forth in GenBank Accession No: NW_020110722.1).

According to a specific embodiment, when the plant is a Gossypium raimondii (Cotton), the target RNA of interest includes, but is not limited to, a gene of Fusarium oxysporum f.sp. vasinfectum (causing e.g. Fusarium wilt) (e.g. as set forth in GenBank Accession No: JN416614.1); a gene of Soybean Aphid (e.g. as set forth in GenBank Accession No: KJ451424.1); or a gene of Pink bollworm (Pectinophora gossypiella) (e.g. as set forth in GenBank Accession No: KU550964.1).

According to a specific embodiment, when the plant is a Oryza sativa (Rice), the target RNA of interest includes, but is not limited to, a gene of Pyricularia grisea (causing e.g. Rice Blast) (e.g. as set forth in GenBank Accession No: AF027979.1); a gene of Gibberella fujikuroi (Fusarium moniliforme) (causing e.g. Bakanae Disease) (e.g. as set forth in GenBank Accession No: AY862192.1); or a gene of a Stem borer, e.g. Scirpophaga incertulas Walker—Yellow Stem Borer, S. innota Walker—White Stem Borer, Chilo suppressalis Walker—Striped Stem Borer, Sesa-mia inferens Walker—Pink Stem Borer (e.g. as set forth in GenBank Accession No: KF290773.1).

According to a specific embodiment, when the plant is a Solanum lycopersicum (Tomato), the target RNA of interest includes, but is not limited to, a gene of Phytophthora infestans (causing e.g. Late blight) (e.g. as set forth in GenBank Accession No: AY855210.1); a gene of a whitefly Bemisia tabaci (e.g. Gennadius, e.g. as set forth in GenBank Accession No: KX390870.1); or a gene of Tomato yellow leaf curl geminivirus (TYLCV) (e.g. as set forth in GenBank Accession No: LN846610.1).

According to a specific embodiment, when the plant is a Solanum tuberosum (Potato), the target RNA of interest includes, but is not limited to, a gene of Phytophthora infestans (causing e.g. Late Blight) (e.g., as set forth in GenBank Accession No: AY050538.3); a gene of Erwinia spp. (causing e.g. Blackleg and Soft Rot) (e.g. as set forth in GenBank Accession No: CP001654.1); or a gene of Cyst Nematodes (e.g. Globodera pallida and G. rostochiensis) (e.g. as set forth in GenBank Accession No: KF963519.1).

According to a specific embodiment, when the plant is a Theobroma cacao (Cacao), the target RNA of interest includes, but is not limited to, a gene of a gene of basidiomycete Moniliophthora roreri (causing e.g. Frosty Pod Rot) (e.g. as set forth in GenBank Accession No: LATX01001521.1); a gene of Moniliophthora perniciosa (causing e.g. Witches' Broom disease); or a gene of Mirids e.g. Distantiella theobroma and Sahlbergella singularis, Helopeltis spp, Monalonion specie.

According to a specific embodiment, when the plant is a Vitis vinifera (Grape or Grapevine), the target RNA of interest includes, but is not limited to, a gene of closterovirus GVA (causing e.g. Rugose wood disease) (e.g. as set forth in GenBank Accession No: AF007415.2); a gene of Grapevine leafroll virus (e.g. as set forth in GenBank Accession No: FJ436234.1); a gene of Grapevine fanleaf degeneration disease virus (GFLV) (e.g. as set forth in GenBank Accession No: NC_003203.1); or a gene of Grapevine fleck disease (GFkV) (e.g. as set forth in GenBank Accession No: NC_003347.1).

According to a specific embodiment, when the plant is a Zea mays (Maize also referred to as corn), the target RNA of interest includes, but is not limited to, a gene of a Fall Armyworm (e.g. Spodoptera frugiperda) (e.g. as set forth in GenBank Accession No: AJ488181.3); a gene of European corn borer (e.g. as set forth in GenBank Accession No: GU329524.1); or a gene of Northern and western corn rootworms (e.g. as set forth in GenBank Accession No: NM_001039403.1).

According to a specific embodiment, when the plant is a sugarcane, the target RNA of interest includes, but is not limited to, a gene of a Internode Borer (e.g. Chilo Saccharifagus Indicus), a gene of a Xanthomonas Albileneans (causing e.g. Leaf Scald) or a gene of a Sugarcane Yellow Leaf Virus (SCYLV).

According to a specific embodiment, when the plant is a wheat, the target RNA of interest includes, but is not limited to, a gene of a Puccinia striiformis (causing e.g. stripe rust) or a gene of an Aphid.

According to a specific embodiment, when the plant is a barley, the target RNA of interest includes, but is not limited to, a gene of a Puccinia hordei (causing e.g. Leaf rust), a gene of Puccinia striiformis f. sp. Hordei (causing e.g. stripe rust), or a gene of an Aphid.

According to a specific embodiment, when the plant is a sunflower, the target RNA of interest includes, but is not limited to, a gene of a Puccinia helianthi (causing e.g. Rust disease); a gene of Boerema macdonaldii (causing e.g. Phoma black stem); a gene of a Seed weevil (e.g. red and gray), e.g. Smicronyx fulvus (red); Smicronyx sordidus (gray); or a gene of Sclerotinia sclerotiorum (causing e.g. Sclerotinia stalk and head rot disease).

According to a specific embodiment, when the plant is a rubber plant, the target RNA of interest includes, but is not limited to, a gene of a Microcyclus ulei (causing e.g. South American leaf blight (SALB)); a gene of Rigidoporus microporus (causing e.g. White root disease); a gene of Ganoderma pseudoferreum (causing e.g. Red root disease).

According to a specific embodiment, when the plant is an apple plant, the target RNA of interest includes, but is not limited to, a gene of Neonectria ditissima (causing e.g. Apple Canker), a gene of Podosphaera leucotricha (causing e.g. Apple Powdery Mildew), or a gene of Venturia inaequalis (causing e.g. Apple Scab).

Exemplary endogenous non-coding RNA molecules which may be modified to target the RNA of interest (e.g. a gene of a pathogen), exemplary sequences of gRNA (i.e. a DNA editing agent) which may be used to modify the endogenous non-coding RNA molecules, and exemplary nucleotide sequences for redirecting a silencing specificity of the endogenous non-coding RNA molecule towards the target RNA of interest are provided in Table 1B, hereinbelow.

TABLE 1B
Examples of GEiGS oligo designs to generate different traits in various hosts

| Host, trait and miRNA- | | | seq difference | | | sgRNA_ |
|---|---|---|---|---|---|---|
| template | Oligo info | oligo_seq | from wt | Sg_seq | pam_difference | strand |
| Host (bold); | | Sequence of GEIGS | Number of | Sequence of the | Number of nucleotide | |
| Pathogen/pest/disease | | oligo, consisting | nucleotide | CRISPR/cas9 small | changes between the | |
| (italic) | | of the precursor | changes | guide RNA targeting | wild type precursor | |
| | | sequence with its | between | the precursor | and the GEiGS | |
| | | corresponding | the wild type | sequence | sequence | |
| | | mature replaced | precursor and | for swapping- | that fall in the PAM | |
| | | by a siRNA | the GeiGs oligo | SEQ ID NO: | region of the sgRNA | |
| | | targeting the | | | | |
| | | desired molecule- | | | | |
| | | SEQ ID NO: | | | | |
| Brassica napus (rapeseed) | | | | | | |
| AM933613.1/Phoma stem | | | | | | |
| canker (caused by | | | | | | |
| leptosphaeria maculans or | | | | | | |
| phoma lingam-fungal | | | | | | |
| pathogen) | | | | | | |
| bna-MIR169e | Max change/ | 103 | 132 | 235 | 3 | rv |
| | perfect structure/ | | | | | |
| | trait-specific | | | | | |
| | siRNA | | | | | |
| bna-MIR156d | Min change/ | 104 | 25 | 236 | 1 | fw |
| | perfect structure/ | | | | | |
| | trait-specific | | | | | |
| | siRNA | | | | | |
| bna-MIR169e | Max change/ | 105 | 128 | 237 | 3 | rv |
| | altered structure/ | | | | | |
| | trait-specific | | | | | |
| | siRNA | | | | | |
| bna-MIR169e | Max change/ | 106 | 131 | 238 | 3 | rv |
| | perfect structure/ | | | | | |
| | non-specific | | | | | |
| | siRNA | | | | | |
| KT959245.1/Flea beetle | | | | | | |
| (Phyllotreta vittula or | | | | | | |
| Chrysomelidae) | | | | | | |
| bna-MIR169e | Max change/ | 107 | 138 | 239 | 3 | rv |
| | perfect structure/ | | | | | |
| | trait-specific | | | | | |
| | siRNA | | | | | |
| bna-MIR156d | Min change/ | 108 | 29 | 240 | 1 | fw |
| | perfect structure/ | | | | | |
| | trait-specific | | | | | |
| | siRNA | | | | | |
| bna-MIR156b | Max change/ | 109 | 76 | 241 | 1 | fw |
| | altered structure/ | | | | | |
| | trait-specific | | | | | |
| | siRNA | | | | | |
| bna-MIR169e | Max change/ | 110 | 133 | 242 | 3 | rv |
| | perfect structure/ | | | | | |
| | non-specific | | | | | |
| | siRNA | | | | | |
| NW_001820833.1/ | | | | | | |
| Sclerotinia stem rot | | | | | | |
| (caused by Sclerotinia | | | | | | |
| sclerotiorum, a fungal | | | | | | |
| pathogen) | | | | | | |
| bna-MIR169e | Max change/ | 111 | 130 | 243 | 3 | rv |
| | perfect structure/ | | | | | |
| | trait-specific | | | | | |
| | siRNA | | | | | |
| bna-MIR390b | Min change/ | 112 | 23 | 244 | 3 | fw |
| | perfect structure/ | | | | | |
| | trait-specific | | | | | |
| | siRNA | | | | | |
| bna-MIR156b | Max change/ | 113 | 68 | 245 | 1 | fw |
| | altered structure/ | | | | | |
| | trait-specific | | | | | |
| | siRNA | | | | | |
| bna-MIR169e | Max change/ | 114 | 135 | 246 | 3 | rv |
| | perfect structure/ | | | | | |
| | non-specific | | | | | |
| | siRNA | | | | | |
| Citrus x sinensis (Orange) | | | | | | |
| AE008925/Citrus Canker | | | | | | |
| (CCK) | | | | | | |
| csi-MIR167c | Max change/ | 115 | 173 | 247 | 3 | fw |
| | perfect structure/ | | | | | |
| | trait-specific | | | | | |
| | siRNA | | | | | |
| csi-MIR156a | Min change/ | 116 | 24 | 248 | 3 | rv |
| | perfect structure/ | | | | | |
| | trait-specific | | | | | |
| | siRNA | | | | | |
| csi-MIR167c | Max change/ | 117 | 150 | 249 | 3 | fw |
| | altered structure/ | | | | | |
| | trait-specific | | | | | |
| | siRNA | | | | | |
| csi-MIR167c | Max change/ | 118 | 173 | 250 | 2 | fw |
| | perfect structure/ | | | | | |
| | non-specific | | | | | |
| | siRNA | | | | | |
| CP001677.5/Citrus | | | | | | |
| greening disease caused | | | | | | |
| by a vector-transmitted | | | | | | |
| pathogen (Candidatus | | | | | | |
| Liberibacter spp.) | | | | | | |
| csi-MIR164a | Max change/ | 119 | 115 | 251 | 2 | rv |
| | perfect structure/ | | | | | |
| | trait-specific | | | | | |
| | siRNA | | | | | |
| csi-MIR482f | Min change/ | 120 | 22 | 252 | 2 | rv |
| | perfect structure/ | | | | | |
| | trait-specific | | | | | |
| | siRNA | | | | | |
| csi-MIR398a | Max change/ | 121 | 88 | 253 | 3 | fw |
| | altered structure/ | | | | | |
| | trait-specific | | | | | |
| | siRNA | | | | | |
| csi-MIR164a | Max change/ | 122 | 119 | 254 | 3 | rv |
| | perfect structure/ | | | | | |
| | non-specific | | | | | |
| | siRNA | | | | | |
| KY389267.1/Armillaria | | | | | | |
| root rot | | | | | | |
| csi-MIR167c | Max change/ | 123 | 168 | 255 | 3 | fw |
| | perfect structure/ | | | | | |
| | trait-specific | | | | | |
| | siRNA | | | | | |
| csi-MIR171a | Min change/ | 124 | 21 | 256 | 3 | rv |
| | perfect structure/ | | | | | |
| | trait-specific | | | | | |
| | siRNA | | | | | |
| csi-MIR167c | Max change/ | 125 | 144 | 257 | 1 | fw |
| | altered structure/ | | | | | |
| | trait-specific | | | | | |
| | siRNA | | | | | |
| csi-MIR167c | Max change/ | 126 | 182 | 258 | 3 | fw |
| | perfect structure/ | | | | | |
| | non-specific | | | | | |
| | siRNA | | | | | |
| Elaeis guineensis | | | | | | |
| (Oil palm) | | | | | | |
| U56128.1/Basal stem rot | | | | | | |
| (BSR) also known as | | | | | | |
| Ganoderma butt rot | | | | | | |
| (Ganoderma spp.) | | | | | | |
| egu-MIR172c | Max change/ | 127 | 89 | 259 | 1 | rv |
| | perfect structure/ | | | | | |
| | trait-specific | | | | | |
| | siRNA | | | | | |
| egu-MIR172c | Min change/ | 128 | 53 | 260 | 1 | rv |
| | perfect structure/ | | | | | |
| | trait-specific | | | | | |
| | siRNA | | | | | |
| egu-MIR172c | Max change/ | 129 | 74 | 261 | 1 | rv |
| | altered structure/ | | | | | |
| | trait-specific | | | | | |
| | siRNA | | | | | |
| egu-MIR172c | Max change/ | 130 | 94 | 262 | 1 | rv |
| | perfect structure/ | | | | | |
| | non-specific | | | | | |
| | siRNA | | | | | |
| Fragaria vesca (Wild | | | | | | |
| strawberry) | | | | | | |
| DS572713.1/Verticillium | | | | | | |
| Wilt (Verticillium dahlia) | | | | | | |
| fve-MIR159c | Max change/ | 131 | 100 | 263 | 3 | fw |
| | perfect structure/ | | | | | |
| | trait-specific | | | | | |
| | siRNA | | | | | |
| fve-MIR160b | Min change/ | 132 | 22 | 264 | 3 | rv |
| | perfect structure/ | | | | | |
| | trait-specific | | | | | |
| | siRNA | | | | | |
| fve-MIR166a | Max change/ | 133 | 56 | 265 | 3 | fw |
| | altered structure/ | | | | | |
| | trait-specific | | | | | |
| | siRNA | | | | | |
| fve-MIR164b | Max change/ | 134 | 95 | 266 | 3 | fw |
| | perfect structure/ | | | | | |
| | non-specific | | | | | |
| | siRNA | | | | | |
| KR855868.1/Fusarium wilt | | | | | | |
| (Fusarium oxysporum f.sp. | | | | | | |
| fragariae) | | | | | | |
| fve-MIR159c | Max change/ | 135 | 97 | 267 | 3 | fw |
| | perfect structure/ | | | | | |
| | trait-specific | | | | | |
| | siRNA | | | | | |
| fve-MIR167b | Min change/ | 136 | 17 | 268 | 1 | rv |
| | perfect structure/ | | | | | |
| | trait-specific | | | | | |
| | siRNA | | | | | |
| fve-MIR169a | Max change/ | 137 | 69 | 269 | 3 | rv |
| | altered structure/ | | | | | |
| | trait-specific | | | | | |
| | siRNA | | | | | |
| fve-MIR164b | Max change/ | 138 | 94 | 270 | 3 | fw |
| | perfect structure/ | | | | | |
| | non-specific | | | | | |
| | siRNA | | | | | |
| Glycine max (Soybean) | | | | | | |
| DQ026061.1/Soybean rust | | | | | | |
| caused by P. pachyrhizi | | | | | | |
| (also known as Asian rust) | | | | | | |
| gma-MIR167c | Max change/ | 139 | 166 | 271 | 3 | rv |
| | perfect structure/ | | | | | |
| | trait-specific | | | | | |
| | siRNA | | | | | |
| gma-MIR1511 | Min change/ | 140 | 23 | 272 | 3 | fw |
| | perfect structure/ | | | | | |
| | trait-specific | | | | | |
| | siRNA | | | | | |
| gma-MIR167c | Max change/ | 141 | 141 | 273 | 3 | rv |
| | altered structure/ | | | | | |
| | trait-specific | | | | | |
| | siRNA | | | | | |
| gma-MIR167c | Max change/ | 142 | 163 | 274 | 3 | rv |
| | perfect structure/ | | | | | |
| | non-specific | | | | | |
| | siRNA | | | | | |
| KJ451424.1/Soybean Aphid | | | | | | |
| gma-MIR167c | Max change/ | 143 | 163 | 275 | 3 | rv |
| | perfect structure/ | | | | | |
| | trait-specific | | | | | |
| | siRNA | | | | | |
| gma-MIR168a | Min change/ | 144 | 23 | 276 | 0 | rv |
| | perfect structure/ | | | | | |
| | trait-specific | | | | | |
| | siRNA | | | | | |
| gma-MIR167c | Max change/ | 145 | 132 | 277 | 3 | rv |
| | altered structure/ | | | | | |
| | trait-specific | | | | | |
| | siRNA | | | | | |
| gma-MIR167c | Max change/ | 146 | 161 | 278 | 3 | rv |
| | perfect structure/ | | | | | |
| | non-specific | | | | | |
| | siRNA | | | | | |
| NC_003056.1/Soybean | | | | | | |
| Dwarf Virus (SbDV) | | | | | | |
| gma-MIR167c | Max change/ | 147 | 165 | 279 | 3 | rv |
| | perfect structure/ | | | | | |
| | trait-specific | | | | | |
| | siRNA | | | | | |
| gma-MIR1516a | Min change/ | 148 | 23 | 280 | 1 | fw |
| | perfect structure/ | | | | | |
| | trait-specific | | | | | |
| | siRNA | | | | | |
| gma-MIR167c | Max change/ | 149 | 135 | 281 | 3 | rv |
| | altered structure/ | | | | | |
| | trait-specific | | | | | |
| | siRNA | | | | | |
| gma-MIR167c | Max change/ | 150 | 163 | 282 | 3 | rv |
| | perfect structure/ | | | | | |
| | non-specific | | | | | |
| | siRNA | | | | | |
| NW_020110722.1/ | | | | | | |
| Green Stink Bug | | | | | | |
| (Acrosternum hilare) | | | | | | |
| gma-MIR167c | Max change/ | 151 | 158 | 283 | 3 | rv |
| | perfect structure/ | | | | | |
| | trait-specific | | | | | |
| | siRNA | | | | | |
| gma-MIR162a | Min change/ | 152 | 22 | 284 | 1 | rv |
| | perfect structure/ | | | | | |
| | trait-specific | | | | | |
| | siRNA | | | | | |
| gma-MIR167c | Max change/ | 153 | 135 | 285 | 3 | rv |
| | altered structure/ | | | | | |
| | trait-specific | | | | | |
| | siRNA | | | | | |
| gma-MIR167c | Max change/ | 154 | 164 | 286 | 3 | rv |
| | perfect structure/ | | | | | |
| | non-specific | | | | | |
| | siRNA | | | | | |
| Gossypium raimondii | | | | | | |
| (Cotton) | | | | | | |
| JN416614.1/Fusarium wilt | | | | | | |
| (Fusarium oxysporum f.sp. | | | | | | |
| vasinfectum) | | | | | | |
| gra-MIR8637 | Max change/ | 155 | 154 | 287 | 3 | rv |
| | perfect structure/ | | | | | |
| | trait-specific | | | | | |
| | siRNA | | | | | |
| gra-MIR7486e | Min change/ | 156 | 2 | 288 | 3 | fw |
| | perfect structure/ | | | | | |
| | trait-specific | | | | | |
| | siRNA | | | | | |
| gra-MIR8633 | Max change/ | 157 | 58 | 289 | 3 | fw |
| | altered structure/ | | | | | |
| | trait-specific | | | | | |
| | siRNA | | | | | |
| gra-MIR8635 | Max change/ | 158 | 149 | 290 | 3 | fw |
| | perfect structure/ | | | | | |
| | non-specific | | | | | |
| | siRNA | | | | | |
| KJ451424.1/Soybean Aphid | | | | | | |
| gra-MIR8637 | Max change/ | 159 | 153 | 291 | 3 | rv |
| | perfect structure/ | | | | | |
| | trait-specific | | | | | |
| | siRNA | | | | | |
| gra-MIR157a | Min change/ | 160 | 16 | 292 | 1 | fw |
| | perfect structure/ | | | | | |
| | trait-specific | | | | | |
| | siRNA | | | | | |
| gra-MIR8636 | Max change/ | 161 | 62 | 293 | 3 | fw |
| | altered structure/ | | | | | |
| | trait-specific | | | | | |
| | siRNA | | | | | |
| gra-MIR8637 | Max change/ | 162 | 149 | 294 | 3 | rv |
| | perfect structure/ | | | | | |
| | non-specific | | | | | |
| | siRNA | | | | | |
| KU550964.1/Pink bollworm | | | | | | |
| (Pectinophora gossypiella) | | | | | | |
| gra-MIR8637 | Max change/ | 163 | 155 | 295 | 3 | rv |
| | perfect structure/ | | | | | |
| | trait-specific | | | | | |
| | siRNA | | | | | |
| gra-MIR157a | Min change/ | 164 | 21 | 296 | 1 | fw |
| | perfect structure/ | | | | | |
| | trait-specific | | | | | |
| | siRNA | | | | | |
| gra-MIR8644 | Max change/ | 165 | 53 | 297 | 3 | rv |
| | altered structure/ | | | | | |
| | trait-specific | | | | | |
| | siRNA | | | | | |
| gra-MIR8635 | Max change/ | 166 | 154 | 298 | 3 | fw |
| | perfect structure/ | | | | | |
| | non-specific | | | | | |
| | siRNA | | | | | |
| Oryza sativa (Rice) | | | | | | |
| AF027979.1/Rice Blast | | | | | | |
| (fungal disease caused by | | | | | | |
| Pyricularia grisea) | | | | | | |
| osa-MIR166b | Max change/ | 167 | 105 | 299 | 1 | fw |
| | perfect structure/ | | | | | |
| | trait-specific | | | | | |
| | siRNA | | | | | |
| osa-MIR156e | Min change/ | 168 | 2.1 | 300 | 0 | fw |
| | perfect structure/ | | | | | |
| | trait-specific | | | | | |
| | siRNA | | | | | |
| osa-MIR160b | Max change/ | 169 | 59 | 301 | 3 | rv |
| | altered structure/ | | | | | |
| | trait-specific | | | | | |
| | siRNA | | | | | |
| osa-MIR166b | Max change/ | 170 | 100 | 302 | 3 | fw |
| | perfect structure/ | | | | | |
| | non-specific | | | | | |
| | siRNA | | | | | |
| AY862192.1/Bakanae | | | | | | |
| Disease (fungal disease | | | | | | |
| caused by Fusarium | | | | | | |
| moniliforme and Gibberella | | | | | | |
| fujikuroi) | | | | | | |
| osa-MIR166b | Max change/ | 171 | 100 | 303 | 3 | fw |
| | perfect structure/ | | | | | |
| | trait-specific | | | | | |
| | siRNA | | | | | |
| osa-MIR160c | Min change/ | 172 | 22 | 304 | 1 | rv |
| | perfect structure/ | | | | | |
| | trait-specific | | | | | |
| | siRNA | | | | | |
| osa-MIR156j | Max change/ | 173 | 68 | 305 | 1 | rv |
| | altered structure/ | | | | | |
| | trait-specific | | | | | |
| | siRNA | | | | | |
| osa-MIR166b | Max change/ | 174 | 104 | 306 | 3 | fw |
| | perfect structure/ | | | | | |
| | non-specific | | | | | |
| | siRNA | | | | | |
| KF290773.1/Stem borer | | | | | | |
| (Scirpophaga incertulas | | | | | | |
| Walker-Yellow Stem | | | | | | |
| Borer, S. innoto Walker- | | | | | | |
| White Stem Borer, Chilo | | | | | | |
| suppressalis Walker | | | | | | |
| Striped Stem Borer, Sesa- | | | | | | |
| mia inferens Walker-Pink | | | | | | |
| Stem Borer | | | | | | |
| osa-MIR166b | Max change/ | 175 | 100 | 307 | 1 | fw |
| | perfect structure/ | | | | | |
| | trait-specific | | | | | |
| | siRNA | | | | | |
| osa-MIR156j | Min change/ | 176 | 21 | 308 | 3 | rv |
| | perfect structure/ | | | | | |
| | trait-specific | | | | | |
| | siRNA | | | | | |
| osa-MIR160b | Max change/ | 177 | 57 | 309 | 3 | rv |
| | altered structure/ | | | | | |
| | trait-specific | | | | | |
| | siRNA | | | | | |
| osa-MIR166b | Max change/ | 178 | 103 | 310 | 3 | fw |
| | perfect structure/ | | | | | |
| | non-specific | | | | | |
| | siRNA | | | | | |
| Solanum lycopersicum | | | | | | |
| (Tomato) | | | | | | |
| AY855210.1/Late blight | | | | | | |
| (Phytophthora infestans) | | | | | | |
| sly-MIR319b | Max change/ | 179 | 143 | 311 | 3 | fw |
| | perfect structure/ | | | | | |
| | trait-specific | | | | | |
| | siRNA | | | | | |
| sly-MIR156b | Min change/ | 180 | 24 | 312 | 0 | fw |
| | perfect structure/ | | | | | |
| | trait-specific | | | | | |
| | siRNA | | | | | |
| sly-MIR395a | Max change/ | 181 | 75 | 313 | 3 | fw |
| | altered structure/ | | | | | |
| | trait-specific | | | | | |
| | siRNA | | | | | |
| sly-MIR319b | Max change/ | 182 | 145 | 314 | 3 | fw |
| | perfect structure/ | | | | | |
| | non-specific | | | | | |
| | siRNA | | | | | |
| KX390870.1/whitefly | | | | | | |
| Bemisia tabaci (Gennadius) | | | | | | |
| sly-MIR319b | Max change/ | 183 | 144 | 315 | 3 | fw |
| | perfect structure/ | | | | | |
| | trait-specific | | | | | |
| | siRNA | | | | | |
| sly-MIR391 | Min change/ | 184 | 16 | 316 | 3 | fw |
| | perfect structure/ | | | | | |
| | trait-specific | | | | | |
| | siRNA | | | | | |
| sly-MIR319c | Max change/ | 185 | 79 | 317 | 1 | rv |
| | altered structure/ | | | | | |
| | trait-specific | | | | | |
| | siRNA | | | | | |
| sly-MIR319b | Max change/ | 186 | 138 | 318 | 3 | fw |
| | perfect structure/ | | | | | |
| | non-specific | | | | | |
| | siRNA | | | | | |
| LN846610.1/Tomato | | | | | | |
| yellow leaf curl geminivirus | | | | | | |
| (TYLCV) | | | | | | |
| sly-MIR319b | Max change/ | 187 | 141 | 319 | 3 | fw |
| | perfect structure/ | | | | | |
| | trait-specific | | | | | |
| | siRNA | | | | | |
| sly-MIR156b | Min change/ | 188 | 24 | 320 | 0 | fw |
| | perfect structure/ | | | | | |
| | trait-specific | | | | | |
| | siRNA | | | | | |
| sly-MIR395a | Max change/ | 189 | 81 | 321 | 3 | fw |
| | altered structure/ | | | | | |
| | trait-specific | | | | | |
| | siRNA | | | | | |
| sly-MIR319b | Max change/ | 190 | 143 | 322 | 3 | fw |
| | perfect structure/ | | | | | |
| | non-specific | | | | | |
| | siRNA | | | | | |
| Solanum tuberosum | | | | | | |
| (Potato) | | | | | | |
| AY050538.3/Late Blight | | | | | | |
| (Phytophthora infestans) | | | | | | |
| stu-MIR6022 | Max change/ | 191 | 110 | 323 | 1 | fw |
| | perfect structure/ | | | | | |
| | trait-specific | | | | | |
| | siRNA | | | | | |
| stu-MIR7988 | Min change/ | 192 | 19 | 324 | 1 | rv |
| | perfect structure/ | | | | | |
| | trait-specific | | | | | |
| | siRNA | | | | | |
| stu-MIR482d | Max change/ | 193 | 50 | 325 | 3 | rv |
| | altered structure/ | | | | | |
| | trait-specific | | | | | |
| | siRNA | | | | | |
| stu-MIR6022 | Max change/ | 194 | 106 | 326 | 1 | fw |
| | perfect structure/ | | | | | |
| | non-specific | | | | | |
| | siRNA | | | | | |
| CP001654.1/Blackleg and | | | | | | |
| Soft Rot (Erwinia spp.) | | | | | | |
| stu-MIR6022 | Max change/ | 195 | 110 | 327 | 1 | fw |
| | perfect structure/ | | | | | |
| | trait-specific | | | | | |
| | siRNA | | | | | |
| stu-MIR7988 | Min change/ | 196 | 16 | 328 | 1 | rv |
| | perfect structure/ | | | | | |
| | trait-specific | | | | | |
| | siRNA | | | | | |
| stu-MIR482d | Max change/ | 197 | 50 | 329 | 2 | rv |
| | altered structure/ | | | | | |
| | trait-specific | | | | | |
| | siRNA | | | | | |
| stu-MIR6022 | Max change/ | 198 | 104 | 330 | 1 | fw |
| | perfect structure/ | | | | | |
| | non-specific | | | | | |
| | siRNA | | | | | |
| KF963519.1/Cyst | | | | | | |
| Nematodes (Globodera | | | | | | |
| pallida and G. rostochiensis) | | | | | | |
| stu-MIR6022 | Max change/ | 199 | 107 | 331 | 1 | fw |
| | perfect structure/ | | | | | |
| | trait-specific | | | | | |
| | siRNA | | | | | |
| stu-MIR7985 | Min change/ | 200 | 20 | 332 | 3 | fw |
| | perfect structure/ | | | | | |
| | trait-specific | | | | | |
| | siRNA | | | | | |
| stu-MIR6024 | Max change/ | 201 | 49 | 333 | 0 | rv |
| | altered structure/ | | | | | |
| | trait-specific | | | | | |
| | siRNA | | | | | |
| stu-MIR6022 | Max change/ | 202 | 107 | 334 | 1 | fw |
| | perfect structure/ | | | | | |
| | non-specific | | | | | |
| | siRNA | | | | | |
| Theobroma cacao (Cacao) | | | | | | |
| LATX01001521.1/Frosty | | | | | | |
| Pod Rot caused by the | | | | | | |
| basidiomycete | | | | | | |
| Moniliophthora roreri | | | | | | |
| tcc-MIR169b | Max change/ | 203 | 100 | 335 | 3 | fw |
| | perfect structure/ | | | | | |
| | trait-specific | | | | | |
| | siRNA | | | | | |
| tcc-MIR167a | Min change/ | 204 | 25 | 336 | 3 | rv |
| | perfect structure/ | | | | | |
| | trait-specific | | | | | |
| | siRNA | | | | | |
| tcc-MIR167b | Max change/ | 205 | 52 | 337 | 1 | fw |
| | altered structure/ | | | | | |
| | trait-specific | | | | | |
| | siRNA | | | | | |
| tcc-MIR169b | Max change/ | 206 | 104 | 338 | 3 | fw |
| | perfect structure/ | | | | | |
| | non-specific | | | | | |
| | siRNA | | | | | |
| Vitis vinifera (Grape) | | | | | | |
| AF007415.2/Rugose wood | | | | | | |
| disease (closterovirus GVA) | | | | | | |
| vvi-MIR167a | Max change/ | 207 | 154 | 339 | 3 | fw |
| | perfect structure/ | | | | | |
| | trait-specific | | | | | |
| | siRNA | | | | | |
| vvi-MIR164b | Min change/ | 208 | 22 | 340 | 3 | fw |
| | perfect structure/ | | | | | |
| | trait-specific | | | | | |
| | siRNA | | | | | |
| vvi-MIR167a | Max change/ | 209 | 108 | 341 | 3 | fw |
| | altered structure/ | | | | | |
| | trait-specific | | | | | |
| | siRNA | | | | | |
| vvi-MIR167a | Max change/ | 210 | 153 | 342 | 2 | fw |
| | perfect structure/ | | | | | |
| | non-specific | | | | | |
| | siRNA | | | | | |
| FJ436234.1/Grapevine | | | | | | |
| leafroll virus | | | | | | |
| vvi-MIR167a | Max change/ | 211 | 152 | 343 | 2 | fw |
| | perfect structure/ | | | | | |
| | trait-specific | | | | | |
| | siRNA | | | | | |
| vvi-MIR167c | Min change/ | 212 | 20 | 344 | 3 | rv |
| | perfect structure/ | | | | | |
| | trait-specific | | | | | |
| | siRNA | | | | | |
| vvi-MIR167a | Max change/ | 213 | 118 | 345 | 2 | fw |
| | altered structure/ | | | | | |
| | trait-specific | | | | | |
| | siRNA | | | | | |
| vvi-MIR167a | Max change/ | 214 | 152 | 346 | 3 | fw |
| | perfect structure/ | | | | | |
| | non-specific | | | | | |
| | siRNA | | | | | |
| NC_003203.1/Grapevine | | | | | | |
| fanleaf degeneration disease | | | | | | |
| virus (GFLV)-a nepovirus | | | | | | |
| vvi-MIR167a | Max change/ | 215 | 158 | 347 | 2 | fw |
| | perfect structure/ | | | | | |
| | trait-specific | | | | | |
| | siRNA | | | | | |
| vvi-MIR166h | Min change/ | 216 | 19 | 348 | 3 | fw |
| | perfect structure/ | | | | | |
| | trait-specific | | | | | |
| | siRNA | | | | | |
| vvi-MIR167a | Max change/ | 217 | 118 | 349 | 2. | fw |
| | altered structure/ | | | | | |
| | trait-specific | | | | | |
| | siRNA | | | | | |
| Vvi-MIR167a | Max change/ | 218 | 156 | 350 | 3 | fw |
| | perfect structure/ | | | | | |
| | non-specific | | | | | |
| | siRNA | | | | | |
| NC_003347.1/Grapevine | | | | | | |
| fleck disease (GFkV) | | | | | | |
| vvi-MIR167a | Max change/ | 219 | 161 | 351 | 2 | fw |
| | perfect structure/ | | | | | |
| | trait-specific | | | | | |
| | siRNA | | | | | |
| vvi-MIR166b | Min change/ | 220 | 19 | 352 | 3 | fw |
| | perfect structure/ | | | | | |
| | trait-specific | | | | | |
| | siRNA | | | | | |
| vvi-MIR167a | Max change/ | 221 | 127 | 353 | 2 | fw |
| | altered structure/ | | | | | |
| | trait-specific | | | | | |
| | siRNA | | | | | |
| vvi-MIR167a | Max change/ | 222 | 157 | 354 | 2 | fw |
| | perfect structure/ | | | | | |
| | non-specific | | | | | |
| | siRNA | | | | | |
| Zea mays (Maize) | | | | | | |
| AJ488181.3/Foll | | | | | | |
| Armyworm (Spodoptera | | | | | | |
| frugiperda) | | | | | | |
| zma-MIR166a | Max change/ | 223 | 100 | 355 | 0 | fw |
| | perfect structure/ | | | | | |
| | trait-specific | | | | | |
| | siRNA | | | | | |
| zma-MIR160c | Min change/ | 224 | 20 | 356 | 2 | fw |
| | perfect structure/ | | | | | |
| | trait-specific | | | | | |
| | siRNA | | | | | |
| zma-MIR156f | Max change/ | 225 | 64 | 357 | 0 | fw |
| | altered structure/ | | | | | |
| | trait-specific | | | | | |
| | siRNA | | | | | |
| zma-MIR166a | Max change/ | 226 | 101 | 358 | 3 | fw |
| | perfect structure/ | | | | | |
| | non-specific | | | | | |
| | siRNA | | | | | |
| GU329524.1/European | | | | | | |
| corn borer | | | | | | |
| zma-MIR166a | Max change/ | 227 | 103 | 359 | 1 | fw |
| | perfect structure/ | | | | | |
| | trait-specific | | | | | |
| | siRNA | | | | | |
| zma-MIR166h | Min change/ | 228 | 20 | 360 | 1 | rv |
| | perfect structure/ | | | | | |
| | trait-specific | | | | | |
| | siRNA | | | | | |
| zma-MIR171f | Max change/ | 229 | 62. | 361 | 3 | rv |
| | altered structure/ | | | | | |
| | trait-specific | | | | | |
| | siRNA | | | | | |
| zma-MIR166a | Max change/ | 230 | 106 | 362 | 3 | fw |
| | perfect structure/ | | | | | |
| | non-specific | | | | | |
| | siRNA | | | | | |
| NM_001039403.1/ | | | | | | |
| Northern and wertern | | | | | | |
| corn rootworms | | | | | | |
| zma-MIR166a | Max change/ | 231 | 107 | 363 | 0 | fw |
| | perfect structure/ | | | | | |
| | trait-specific | | | | | |
| | siRNA | | | | | |
| zma-MIR172d | Min change/ | 232 | 20 | 364 | 3 | rw |
| | perfect structure/ | | | | | |
| | trait-specific | | | | | |
| | siRNA | | | | | |
| zma-MIR166a | Max change/ | 233 | 73 | 365 | 1 | fw |
| | altered structure/ | | | | | |
| | trait-specific | | | | | |
| | siRNA | | | | | |
| zma-MIR166a | Max change/ | 234 | 105 | 366 | 3 | fw |
| | perfect structure/ | | | | | |
| | non-specific | | | | | |
| | siRNA | | | | | |

Table 1B provides example GeiGS oligos designed against a variety of targets in several host organisms. For each host-target combination, four oligos are provided: minimum sequence changes with matching structure and efficient siRNA; maximum sequence changes with matching structure and efficient siRNA; maximum sequence changes and non-matching structure and efficient siRNA; and maximum sequence changes with matching structure and inefficient siRNA.

Table 1B provides example GeiGS oligos designed against a variety of targets in several host organisms. For each host-target combination, four oligos are provided: minimum sequence changes with matching structure and efficient siRNA: maximum sequence changes with matching structure and efficient siRNA; maximum sequence changes and non-matching structure and efficient siRNA; and maximum sequence changes with matching structure and inefficient siRNA.

According to one embodiment, the plants generated by the present method are more resistant or tolerant to pathogens by at least about 10%, 20%, 30%, 40%, 50%, 60%, 70%, 80%, 90%, 95% or 100% as compared to plants not generated by the present methods (i.e. as compared to wild type plants).

Any method known in the art for assessing tolerance or resistance to a pathogen of a plant may be used in accordance with the present invention. Exemplary methods include, but are not limited to, reducing MYB46 expression in Arabidopsis which results in enhance resistance to Botrytis cinereal as described in Ramírez V1, García-Andrade J, Vera P., Plant Signal Behav. 2011 June; 6(6):911-3. Epub 2011 Jun. 1; or downregulation of HCT in alfalfa promotes activation of defense response in the plant as described in Gallego-Giraldo L. et al. New Phytologist (2011) 190: 627-639 doi: 10.1111/j.1469-8137.2010.03621.x), both incorporated herein by reference.

According to one embodiment, there is provided a method of generating a herbicide resistant plant, the method comprising modifying a gene encoding or processed into a non-coding RNA molecule or into a RNA silencing molecule in a plant cell according to the methods of some embodiments of the invention, wherein the target RNA of interest is of a gene of the plant conferring sensitivity to the herbicide, thereby generating the herbicide resistant plant.

According to one embodiment, the herbicides target pathways that reside within plastids (e.g. within the chloroplast).

Thus to generate herbicide resistant plants, the non-coding RNA molecule is designed to target a RNA of interest including, but not limited to, the chloroplast gene psbA (which codes for the photosynthetic quinone-binding membrane protein Q_{B}, the target of the herbicide atrazine) and the gene for EPSP synthase (a nuclear protein, however, its overexpression or accumulation in the chloroplast enables plant resistance to the herbicide glyphosate as it increases the rate of transcription of EPSPs as well as by a reduced turnover of the enzyme).

According to one embodiment, the plants generated by the present method are more resistant to herbicides by at least about 10%, 20%, 30%, 40%, 50%, 60%, 70%, 80%, 90%, 95% or 100% as compared to plants not generated by the present methods.

According to one embodiment, there is provided a plant generated according to the method of some embodiments of the invention.

According to one embodiment, plant is non-genetically modified (non-GMO).

According to one embodiment, there is provided a seed of the plant generated according to the method of some embodiments of the invention.

Designing GEiGS with minimal nucleotide modifications/edits in the endogenous non-coding RNA can be achieved using in silico methods, which are based on bioinformatics tools that are well known to the skilled artisan.

According to one embodiment, such a method is effected as follows:

The following information should be available: a) Target sequence to be silenced by Gene Editing induced Gene Silencing (GEiGS) (“target”); b) Choosing whether the GEiGS (i.e. the non-coding RNA with modified silencing activity and/or specificity) would be expressed ubiquitously (e.g. constitutively) or specifically (e.g. expression specific to a certain tissue, developmental stage, stress, heat/cold shock etc.).

Submitting this information to publicly or inhouse available miRNA datasets (e.g. small RNA sequencing, genomic sequences, microarrays etc.) so as to filter (i.e. elect) only relevant miRNAs that match the input criteria: miRNAs that are expressed according to the requirement(s) described above, such as miRbase (Kozommara and Griffiths-Jones (2014)), tasRNAdb (Zhang Changqing, et al. (2013)) and mirEx 2.0 (Zielezinski, Andrzej et al. “mirEX 2.0—an Integrated Environment for Expression Profiling of Plant microRNAs.” BMC Plant Biology 15 (2015): 144. PMC. Web. 15 Sep. 2018).

Using publically available tools, a list of potent target-specific siRNA sequences may be generated. The miRNAs may be aligned against the potent siRNA sequences and the most homologous miRNAs may be elected. Filtered miRNAs may have a similar sequence in the same orientation like the potent siRNAs.

Modifying the naturally mature miRNAs sequences, which are scored to have high homology to target-specific potent siRNAs, to perfectly match the target's sequence. This modification may occur in one mature miRNA strand with the highest target homology (e.g. could be either the original miRNA guide or passenger strand). Such 100% complementary to the target can potentially turn the miRNA sequence into a siRNA.

Minimal GE may be achieved by filtering miRNA sequences with naturally occurring high homology (reverse complement) to the target.

Using the primary modified miRNA genes to generate ssDNA oligos (e.g. 200-500 nt ssDNA long) and dsDNA fragments (e.g. 250-5000 nt dsDNA fragments only or cloned within plasmids) based on the genomic DNA sequences that flank the modified miRNA precursor sequence (pre-miRNA). The modified miRNA's guide strand (silencing strand) sequence may be designed to be 100% complementary to the target.

Modifying the sequence of the other miRNA gene region to preserve the original (unmodified) miRNA precursor and mature structure, through keeping the same base pairing profile.

Designing sgRNAs to specifically target the original unmodified miRNA gene (specific to the genomic miRNA loci), and not the modified version (i.e. the oligo/fragment sequences).

Analyzing the comparative restriction enzyme site between the modified and the original miRNA gene and summarizing the differential restriction sites. Such a detection system is based on PCR that is followed by restriction enzyme digestion and gel electrophoresis.

Validating as discussed in detail above.

Examining the targeting of the non-coding RNA towards other targets (e.g. “off target effect”), using in silico methods, when the endogenous non-coding RNA (e.g. miRNA) comprises naturally occurring high homology with the target (e.g. 60-90%), so as to obtain specific silencing of the target of interest.

Minimally modifying the endogenous non-coding RNA (e.g. miRNA) to boost its potency to silence the target of interest.

Validating GEiGS outcome of the primary minimally edited miRNA genes to generate candidate refined minimally edited miRNAs. An experimentally effective primary GEiGS outcome (the primary minimally edited miRNA genes) is considered as a miRNA(s) with a guide or passenger strand that is modified to match the target by 100%.

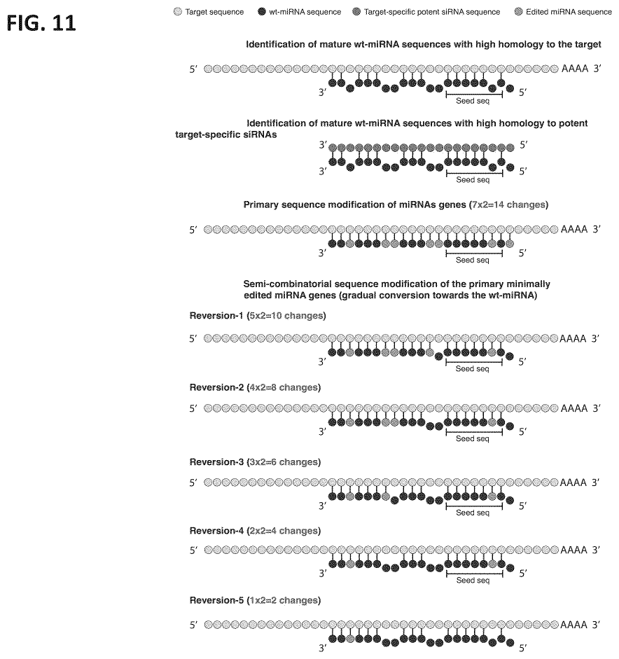

Generating several guide or passenger strand sequences that are gradually reverted back into the original sequence (as illustrated in FIG. 11).

Keeping the seed sequence in a way that there are at least 5 matches out of the seven seed nucleotides (nucleotides 2-8 from the 5′ terminus).

Testing the various candidate ‘refined minimally edited miRNA genes’ for target silencing efficiency. Choosing the gene GE-mediated knock-in that provides the highest silencing with the minimal miRNA sequence modification.

Testing potential “off target effects” of refined minimally edited miRNA candidates. A significant prediction for “off target effects” affects the final evaluation of the refined minimally edited miRNA genes.

Testing the less refined minimally edited miRNA gene candidates based on the experimental validation.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NOs: 1-4 are expressed in a DNA sequence format (e.g., reciting T for thymine), but it can refer to either a DNA sequence that corresponds to an gRNA nucleic acid sequence, or the RNA sequence of a RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

### Arabidopsis Cell Culture

Arabidopsis thaliana (ecotype Landsberg erecta) cell cultures were maintained in 100 mL of liquid growth medium (4.4 g/L Murashige and Skoog (MS) salts with vitamins [Duchefa, Haarlem, The Netherlands], 30 g/L sucrose, 0.5 mg/L 1-Naphthaleneacetic acid (NAA) and 0.5 mg/L 6-Benzylaminopurine (BAP) at 25° C., 16 hour photoperiod and gentle agitation (100 rpm). Every week 6 ml of culture was transferred to fresh medium.

### Plant Growth

Arabidopsis thaliana (ecotype Colombia-0) seedlings were surface sterilized and grown on plates containing MS medium supplemented with 0.8 g/L agar at 20° C. in 16 hour photoperiod.

### Stable Transformation of Arabidopsis Cell Culture

Agrobacterium carrying the pK7WGF2 plasmid were grown in LB medium supplemented with 100 mg/L spectinomycin at 28° C. to an OD of 0.8. Bacteria were collected by centrifugation and resuspended in the same amount of plant cell culture medium. Four days after transfer to fresh medium, 4 ml of Arabidopsis cells were incubated with 0.1 mL of the Agrobacterium suspension in a Petri dish at 25° C. in the dark with gentle agitation (130 rpm). After 48 hours, the cells were collected by centrifugation and washed five times with cell culture medium to remove most of the bacteria. Finally, cells were resuspended in 2 ml of cell culture medium and plates onto a petri dish containing cell culture medium supplemented with 0.4% Phytagel, 500 mg/L timenten and 50 mg/L kanamycin. The dishes were stored at 25° C. in the dark until calli formation was observed, usually after 2 or 3 weeks

### Banana Embryogenic Calli:

Banana embryogenic callus is developed from an initial explant such as immature male flowers or shoot tip as described by Ma [Ma S. S., Proceedings of Symposium on Tissue culture of horticultural crops, Taipei, Taiwan, 8-9 Mar. 1988, pp. 181-188] and Schoofs [Schoofs H., The origin of embryogenic cells in Musa. PhD thesis, KULeuven, Belgium (1997)]. Embryogenic cell suspensions are initiated from freshly developed highly embryogenic calli in liquid medium. 80% of the medium is refreshed every 12-14 days until the initiated cell suspension is fully established (6-9 months).

### Coffee Embryonic Calli:

Coffee embryonic calli is obtained as previously described [Etienne, H., Protocol for somatic embryogenesis in woody plants (2005) Springer. p. 167-1795]. Briefly, young leaves are surface sterilized, cut into 1 cm² pieces and placed on half strength semi solid MS medium supplemented with 2.26 μM 2,4-dichlorophenoxyacetic acid (2,4-D), 4.92 μM indole-3-butyric acid (IBA) and 9.84 μM isopentenyladenine (iP) for one month. Explants are then transferred to half strength semisolid MS medium containing 4.52 μM 2,4-D and 17.76 μM 6-benzylaminopurine (6-BAP) for 6 to 8 months until regeneration of embryogenic calli. Embryogenic calli are maintained on MS media supplemented with 5 μM 6-BAP.

Cell suspension cultures are generated from embryogenic calli as previously described [Acuna, J. R. and M. de Pena, Plant Cell Reports (1991) 10(6): p. 345-348]. Embryogenic calli (30 g/1) are placed in liquid MS medium supplemented with 13.32 μM 6-BAP. Flasks are placed in a shaking incubator (110 rpm) at 28° C. The cell suspension is subcultured/passaged every two to four weeks until fully established. Cell suspension cultures are maintained in liquid MS medium with 4.44 μM 6-BAP.

### Computational Pipeline to Generate GEiGS Templates

The computational GEiGS pipeline applies biological metadata and enables an automatic generation of GEiGS DNA templates that are used to minimally edit non-coding RNA genes (e.g. miRNA genes), leading to a new gain of function. i.e. redirection of their silencing capacity to target sequence of interest.

As illustrated in FIG. 9, the pipeline starts with filling and submitting input: a) target sequence to be silenced by GEiGS; b) the host organism to be gene edited and to express the GEiGS; c) one can choose whether the GEiGS would be expressed ubiquitously or not. If specific GEiGS expression is required, one can choose from a few options (expression specific to a certain tissue, developmental stage, stress, heat/cold shock etc).

When all the required input is submitted, the computational process begins with searching among miRNA datasets (e.g. small RNA sequencing, microarray etc.) and filtering only relevant miRNAs that match the input criteria. Next, the selected mature miRNA sequences are aligned against the target sequence and miRNA with the highest complementary levels are filtered. These naturally target-complementary mature miRNA sequences are then modified to perfectly match the target's sequence. Then, the modified mature miRNA sequences are run through an algorithm that predicts siRNA potency and the top 20 with the highest silencing score are filtered. These final modified miRNA genes are then used to generate 200-500 nt ssDNA or 250-5000 nt dsDNA sequences as follows:

200-500 nt ssDNA oligos and 250-5000 nt dsDNA fragments are designed based on the genomic DNA sequence that flanks the modified miRNA. The pre-miRNA sequence is located in the center of the oligo. The modified miRNA's guide strand (silencing) sequence is 100% complementary to the target. However, the sequence of the modified passenger miRNA strand is further modified to preserve the original (unmodified) miRNA structure, keeping the same base pairing profile.

Next, differential sgRNAs are designed to specifically target the original unmodified miRNA gene, and not the modified swapping version. Finally, comparative restriction enzyme site analysis is performed between the modified and the original miRNA gene and differential restriction sites are summarized.

Therefore, the pipeline output includes:
a) 200-500 nt ssDNA oligo or 250-5000 nt dsDNA fragment sequence with minimally modified miRNA
b) 2-3 differential sgRNAs that target specifically the original miRNA gene and not the modified
c) List of differential restriction enzyme sites among the modified and original miRNA gene

### Target Genes

### Phytoene Desaturase Gene (PDS

### Rationale:

PDS is an essential gene in the chlorophyll biosynthesis pathway and loss of PDS function in plants results in albino phenotype [Fan et al., Sci Rep (2015) 5:12217]. When used as a target gene in genome editing (GE) strategy or RNAi, positively edited plants are easily identified by partial or complete loss of chlorophyll in leaves and other organs (bleaching).

### Methods:

miRNAs with ubiquitous expression profile are chosen (depends on the application, one might choose miRNAs with expression profile that is specific to a certain tissue, developmental stage, temperature, stress etc).

miRNAs are modified to siRNA targeting the PDS gene from Arabidopsis (see Table 1A, below). Following transfection and FACS sorting (RFP/GFP are used for identifying positive Cas9/sgRNA transfection events), protocolonies (or calli) are transferred into solid regeneration media (half strength MS+B5 vitamins, 20 g/l sucrose, 0.8% agar) until shoots are regenerated. Loss of pigmentation in these shoots indicates loss of function of the PDS gene and correct GE. No albino phenotype is observed in the control plantlets transfected with an oligo carrying random sequence.

### Green Fluorescent Protein (GFP) Gene

### Rationale:

GFP is a protein composed of 238 amino acid residues (26.9 kDa) that exhibits bright green fluorescence when exposed to light in the blue to ultraviolet range. Although many other marine organisms have similar green fluorescent proteins, GFP traditionally refers to the protein first isolated from the jellyfish Aequorea victoria. The GFP from A. victoria has a major excitation peak at a wavelength of 395 nm and a minor one at 475 nm. Its emission peak is at 509 nm, which is in the lower green portion of the visible spectrum. The fluorescence quantum yield (QY) of GFP is 0.79. The GFP from the sea pansy (Renilla reniformis) has a single major excitation peak at 498 nm. GFP makes for an excellent tool in many areas of biology due to its ability to form internal chromophores without requiring any accessory cofactors, gene products, or enzymes/substrates other than molecular oxygen.

### Methods:

miRNAs with ubiquitous expression profile are chosen (depends on the application, one might choose miRNAs with expression profile that is specific to a certain tissue, developmental stage, temperature, stress etc).

miRNAs are modified into siRNA targeting the GFP gene (see Table 1A, below). Following transfection FACS sorting is performed, isolating mCherry-expressing protoplasts (mCherry is used for identifying positive Cas9/sgRNA transfection events) with no or low GFP signal. In the control (oligo with non-target siRNA sequence), all protoplasts express mCherry and GFP. Next, candidate successful GE protoplast (mCherry positive and GFP negative) are regenerated into plants for further analyses. Protoplasts are also qualitatively documented under the microscope. For quantification analysis and ratios FACS analysis was used.

TABLE 1A
Target Genes IDs

| Gene name | Query sequence ID | Query sequence organism |
|---|---|---|
| PDS | NM_001340908.1 | Arabidopsis |
| | (SEQ ID NO: 25) | |
| | NM_117498 | |
| | (SEQ ID NO: 26) | |
| ADH1 | NC_003070.9 | Arabidopsis |
| eGFP | AFA52654 | Aequorea victoria |
| | (SEQ ID NO: 27) | |

siRNA Design

Target-specific siRNAs are designed by publically available siRNA-designers such as ThermoFisher Scientific's “BLOCK-iT™ RNAi Designer” and Invivogen's “Find siRNA sequences”.

sgRNAs Design

sgRNAs are designed to target the endogenous miRNA genes using the publically available sgRNA designer, as previously described in Park et al., Bioinformatics (2015) 31(24): 4014-4016. Two sgRNAs are designed for each cassette, but a single sgRNA is expressed per cell to initiate gene swapping. sgRNAs correspond to the pre-miRNA sequence that is modified post swapping.

In order to maximize the chance of efficient sgRNA choice, two different publicly available algorithms (CRISPER Design: www(dot)crispr(dot)mit(dot)edu:8079/ and CHOPCHOP: www(dot)chopchop(dot)cbu(dot)uib(dot)no/) are used and the top scoring sgRNA from each algorithm is selected.

Swapping ssDNA Oligo Design:

400 b ssDNA oligo is designed based on the genomic DNA sequence of the miRNA gene. The pre-miRNA sequence is located in the center of the oligo. Next, the double stranded siRNA sequences are swapped with the mature miRNA sequences in a way that the guide (silencing) siRNA strand is kept 100% complementary to the target. The sequence of the passenger siRNA strand is modified to preserve the original miRNA structure, keeping the same base pairing profile.

### Swapping Plasmid DNA Design

4000 bp dsDNA fragment is designed based on the genomic DNA sequence of the miRNA gene. The pre-miRNA sequence is located in the center of the dsDNA fragment. Next, the double stranded siRNA sequences are swapped with the mature miRNA sequences in a way that the guide (silencing) siRNA strand is kept 100% complementary to the target. The sequence of the passenger siRNA strand is modified to preserve the original miRNA structure, keeping the same base pairing profile. Finally, the fragment is cloned into a standard vector (e.g. pBluescript).

### Long Plasmids for Swapping:

Plasmid-1: GEiGS_mir173_si-GFP_1 (SEQ ID NO: 31)
Plasmid-2: GEiGS_mir173_si-GFP_2 (SEQ ID NO: 32)
Plasmid-3: GEiGS_mir173_si-PDS_1 (SEQ ID NO: 33)
Plasmid-4: GEiGS_mir173_si-PDS_2 (SEQ ID NO: 34)
Plasmid-5: GEiGS_mir390a_si-GFP_1 (SEQ ID NO: 35)
Plasmid-6: GEiGS_mir390a_si-GFP_2 (SEQ ID NO: 36)
Plasmid-7: GEiGS_mir390a_si-PDS_1 (SEQ ID NO: 37)
Plasmid-8: GEiGS_mir390a_si-PDS_2 (SEQ ID NO: 38)
   sgRNAs Sequences:
   Arabidopsis mir-390A:

1.
(SEQ ID NO: 1)
CTATCCATCCTGAGTTTCATTGG;
2.
(SEQ ID NO: 2)
AAGAATCTGTAAAGCTCAGGAGG;

Arabidopsis mir-173:

1.
(SEQ ID NO: 3)
CTTGCAGAGAGAAATCACAGTGG;
2.
(SEQ ID NO: 4)
GCTTACACAGAGAATCACAGAGG;

List of Endogenous miRNA that are Swapped:
1. Arabidopsis mir-390A
2. Arabidopsis mir-173
   ssDNA Oligos Used for Gene Swapping:
Oligo-1: GEiGS_mir173_si-GFP_1 (5′→3′) (SEQ ID NO: 5)
Oligo-2: GEiGS_mir173_si-GFP_2 (5′→3′) (SEQ ID NO: 6)
Oligo-3: GEiGS_mir173_si-PDS_1 (5′→3′) (SEQ ID NO: 7)
Oligo-4: GEiGS_mir173_si-PDS_2 (5′→3′) (SEQ ID NO: 8)
Oligo-5: GEiGS_mir390a_si-GFP_1 (5′→3′) (SEQ ID NO: 9)
Oligo-6: GEiGS_mir390a_si-GFP_2 (5′→3′) (SEQ ID NO: 10)
Oligo-7: GEiGS_mir390a_si-PDS_1 (5′→3′) (SEQ ID NO: 11)
Oligo-8: GEiGS_mir390a_si-PDS_2 (5′→3′) (SEQ ID NO: 12)
   sgRNA Cloning

The transfection plasmid utilized was composed of 4 modules comprising of
1) mCherry driven by the CsVMV promoter terminated by a G7 termination sequence;
2) 2×35S::hCas9-35S-ter i.e. hCas9 driven by the 35S promoter terminated by AtuNos termination sequence;
3) AtU6-26 and/or U6 synthetic promoter driving sgRNA for guide 1;

### Plasmid Design

For transient expression, a plasmid containing three transcriptional units is used. The first transcriptional unit contains CsVMV promoter driving expression of mCherry and the G7 terminator. The next transcriptional unit consists of 2x-35S promoter-driving expression of Cas9 and the 35S terminator. The third contains the Arabidopsis U6 promoter expressing sgRNA to target miRNA genes (each vector comprises a single sgRNAs).

Design and Cloning of CRISPR/CAS9 to Target miR-173 and miR-390 and Introducing SWAPs to Target GFP, AtPDS3 and AtADH1

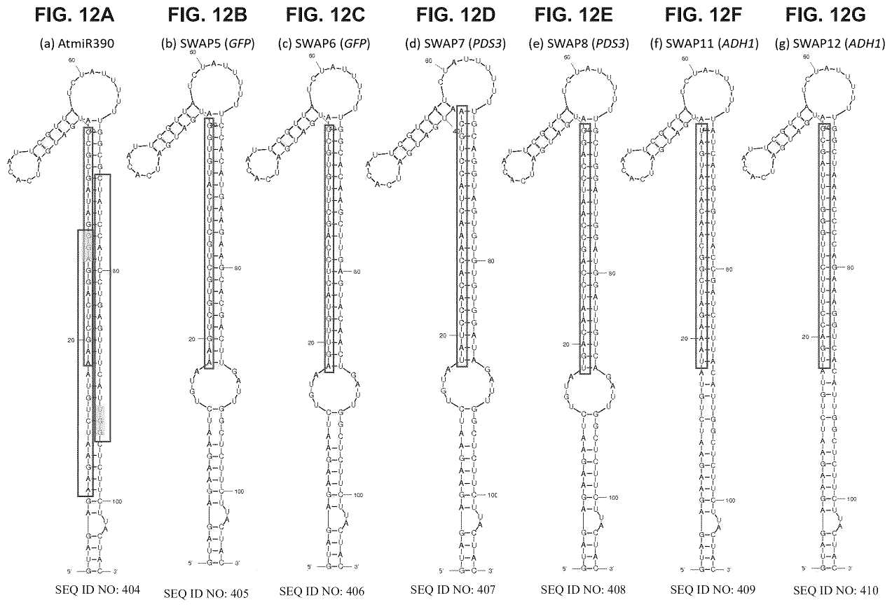

The present inventors have designed changes in the sequences of mature miR-173 and miR-390, in their genomic context, to target GFP, AtPDS3 or AtADH1, by producing small RNA that reverse complements the target genes, visualized in FIGS. 12A-G and 13A-G. In addition, to maintain the secondary structure of the miRNA precursor transcript, further changes in the pri-miRNA were carried out, as specified in FIGS. 12A-G, 13A-G, 14A-D and 15A-D and Table 2 (below). These fragments were cloned into PUC plasmids and named DONORs and the DNA fragments are referred as SWAPs. For sequences for modifying miR-173-SWAP1 and SWAP2 to target GFP, SWAP3 and SWAP4 to target AtPDS3 and SWAP9 and SWAP10 to target AtADH1 (see Table 2, below). For sequences for modifying miR-390-SWAP5 and SWAP6 to target GFP, SWAP7 and SWAP8 to target AtPDS3 and SWAP11 and SWAP12 to target AtADH1 (see Table 2, below).

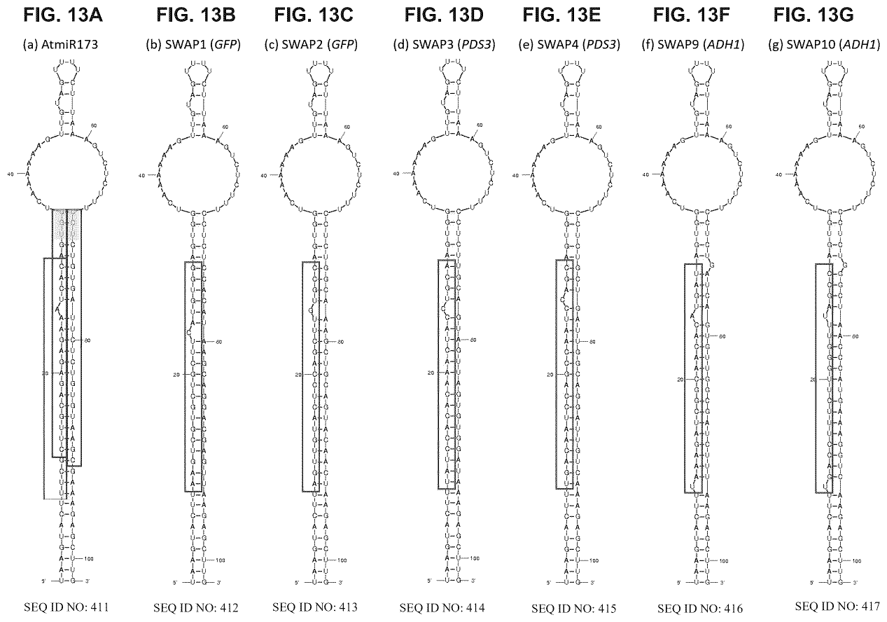

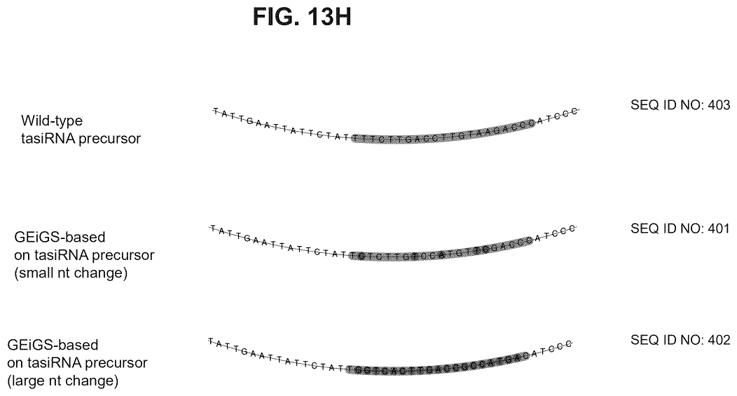

Guide RNAs targeting miR-173 and miR-390 were introduced into CRISPR/CAS9 vector system in order to generate a DNA cleavage in the desired miRNA loci. These were co-introduced to the plants with the DONOR vectors via gene bombardment protocol, to introduce desired modifications through Homologous DNA Repair (HDR). These guide RNAs are specified in Table 2, below, and illustrated in FIGS. 12A and 13A.

TABLE 2
Sequences and oligos used in the experiments

| SEQ ID NO: | Aim |
|---|---|
| 39 | miR173 |
| 40 | miR390 |
| 41 | sgRNA sequence used for miR173 targeting in CRISPR/CAS9 system-GEiGS#4 |
| 42 | sgRNA sequence used for miR173 targeting in CRISPR/CAS9 system-GEiGS#5 |
| 43 | sgRNA sequence used for miR390 targeting in CRISPR/CAS9 system-GEiGS#1 |
| 44 | sgRNA sequence used for miR390 targeting in CRISPR/CAS9 system-GEiGS#3 |
| 45 | mature GEiGS-siRNA targeting GFP-used in SWAP5 (based on miR390) |
| | and in SWAP1 (based on miR173) |
| 46 | Complementary strand of mature GEiGS-siRNA targeting GFP-used in |
| | SWAP5 (based on miR390) and in SWAP1 (based on miR173) |
| 47 | mature GEiGS-siRNA targeting GFP-used in SWAP6 (based on miR390) |
| | and in SWAP2 (based on miR173) |
| 48 | Complementary strand of mature GEiGS-siRNA targeting GFP-used in |
| | SWAP6 (based on miR390) and in SWAP2 (based on miR173) |
| 49 | mature GEiGS-siRNA targeting AtPDS3-used in SWAP7 (based on miR390) and |
| | in SWAP3 (based on miR173) |
| 50 | Complementary strand of mature GEiGS-siRNA targeting AtPDS3-used in |
| | SWAP7 (based on miR390) and in SWAP3 (based on miR173) |
| 51 | mature GEiGS-siRNA targeting AtPDS3-used in SWAP8 (based on miR390) and |
| | in SWAP4 (based on miR173) |
| 52 | Complementary strand of mature GEiGS-siRNA targeting AtPDS3-used in |
| | SWAP8 (based on miR390) and in SWAP4 (based on miR173) |
| 53 | mature GEiGS-siRNA targeting AtADH1-used in SWAP11 (based on |
| | miR390) and in SWAP9 (based on miR173) |
| 54 | Complementary strand of mature GEiGS-siRNA targeting AtADH1-used in |
| | SWAP11 (based on miR390) and in SWAP9 (based on miR173) |
| 55 | mature GEiGS-siRNA targeting AtADH1-used in SWAP12 (based on |
| | miR390) and in SWAP10 (based on miR173) |
| 56 | Complementary strand of mature GEiGS-siRNA targeting AtADH1-used in |
| | SWAP12 (based on miR390) and in SWAP10 (based on miR173) |
| 57 | Primary transcript of miR173 (pri-miR173) |
| 58 | Primary transcript of SWAP1 (used in Donor vector for targeting GFP) |
| 59 | Primary transcript of SWAP2 (used in Donor vector for targeting GFP) |
| 60 | Primary transcript of SWAP3 (used in Donor vector for targeting PDS3) |
| 61 | Primary transcript of SWAP4 (used in Donor vector for targeting PDS3) |
| 62 | Primary transcript of SWAP9 (used in Donor vector for targeting ADH1) |
| 63 | Primary transcript of SWAP10 (used in Donor vector for targeting ADH1) |
| 64 | Primary transcript of miR390 (pri-miR390) |
| 65 | Primary transcript of SWAP5 (used in Donor vector for targeting GFP) |
| 66 | Primary transcript of SWAP6 (used in Donor vector for targeting GFP) |
| 67 | Primary transcript of SWAP7 (used in Donor vector for targeting PDS3) |
| 68 | Primary transcript of SWAP8(used in Donor vector for targeting PDS3) |
| 69 | Primary transcript of SWAP11 (used in Donor vector for targeting ADH1) |
| 70 | Primary transcript of SWAP12 (used in Donor vector for targeting ADH1) |
| 71 | Sequence of miR173 loci |
| 72 | Oligo sequence of SWAP1 (used in Donor vector for modification of miR173 |
| | for targeting GFP) |
| 73 | Oligo sequence of SWAP2 (used in Donor vector for modification of miR173 |
| | for targeting GFP) |
| 74 | Oligo sequence of SWAP3 (used in Donor vector for modification of miR173 |
| | for targeting PDS3) |
| 75 | Oligo sequence of SWAP4 (used in Donor vector for modification of miR173 |
| | for targeting PDS3) |
| 76 | Oligo sequence of SWAP9 (used in Donor vector for modification of miR173 |
| | for targeting ADH1) |
| 77 | Oligo sequence of SWAP10 (used in Donor vector for modification of miR173 |
| | for targeting ADH1) |
| 78 | Oligo sequence of miR390 loci |
| 79 | Oligo sequence of SWAP5 (used in Donor vector for modification of miR390 |
| | for targeting GFP) |
| 80 | Oligo sequence of SWAP6 (used in Donor vector for modification of miR390 |
| | for targeting GFP) |
| 81 | Oligo sequence of SWAP7 (used in Donor vector for modification of miR390 |
| | for targeting PDS3) |
| 82 | Oligo sequence of SWAP8(used in Donor vector for modification of miR390 |
| | for targeting PDS3) |
| 83 | Oligo sequence of SWAP11 (used in Donor vector for modification of miR390 |
| | for targeting ADH1) |
| 84 | Oligo sequence of SWAP12 (used in Donor vector for modification of miR390 |
| | for targeting ADH1) |
| 85 | qRT for housekeeping gene-18S expression (NC_037304)-Forward primer |
| 86 | qRT for housekeeping gene-18S expression (NC_037304)-Reverse primer |
| 87 | qRT for analysis of PDS3 expression (AT4G14210)-Forward primer |
| 88 | qRT for analysis of PDS3 expression (AT4G14210)-Reverse primer |
| 89 | qRT for analysis of ADH1 expression (AT1G77120)-Forward primer |
| 90 | qRT for analysis of ADH1 expression (AT1G77120)-Reverse primer |
| 91 | Forward primer for internal amplification of miR390 and its modified versions |
| 92 | Reverse primer for internal amplification of miR390 and its modified versions |
| 93 | Forward primer for external amplification of miR390 and its modified |
| | versions-primary reaction |
| 94 | Reverse for external amplification of miR390 and its modified versions- |
| | primary reaction |
| 95 | Forward primer for external amplification of miR390 and its modified |
| | versions-nested reaction |
| 96 | Reverse for external amplification of miR390 and its modified versions- |
| | nested reaction |
| 97 | Forward primer for internal amplification of miR173 and its modified versions |
| 98 | Reverse primer for internal amplification of miR173 and its modified versions |
| 99 | Forward primer for external amplification of miR173 and its modified |
| | versions-primary reaction |
| 100 | Reverse for external amplification of miR173 and its modified versions- |
| | primary reaction |
| 101 | Forward primer for external amplification of miR173 and its modified |
| | versions-nested reaction |
| 102 | Reverse for external amplification of miR173 and its modified versions- |
| | nested reaction |

Table 2, cont.

### Protoplasts Isolation

Protoplasts were isolated by incubating plant material (e.g. leaves, calli, cell suspensions) in a digestion solution (1% cellulase, 0.5% macerozyme, 0.5% driselase, 0.4 M mannitol, 154 mM NaCl, 20 mM KCl, 20 mM MES pH 5.6, 10 mM CaCl₂)) for 4-24 hours at room temperature and gentle shaking. After digestion, remaining plant material was washed with W5 solution (154 mM NaCl, 125 mM CaCl₂), 5 mM KCl, 2 mM MES pH5.6) and protoplasts suspension was filtered through a 40 μm strainer. After centrifugation at 80 g for 3 minutes at room temperature, protoplasts were resuspended in 2 ml W5 buffer and precipitated by gravity in ice. The final protoplast pellet was resuspended in 2 ml of MMg (0.4 M mannitol, 15 mM MgCl2, 4 mM MES pH 5.6) and protoplast concentration was determined using a hemocytometer. Protoplasts viability was estimated using Trypan Blue staining.

### Polyethylene Glycol (PEG)-Mediated Plasmid Transfection

PEG-transfection of protoplasts was effected using a modified version of the strategy reported by Wang [Wang et al., Scientia Horticulturae (2015) 191: p. 82-89]. Protoplasts were resuspended to a density of 2-5×10⁶ protoplasts/ml in MMg solution. 100-200 μl of protoplast suspension was added to a tube containing the plasmid. The plasmid:protoplast ratio greatly affects transformation efficiency therefore a range of plasmid concentrations in protoplast suspension, 5-300 μg/μl, were assayed. PEG solution (100-200 μl) was added to the mixture and incubated at 23° C. for various lengths of time ranging from 10-60 minutes. PEG4000 concentration was optimized, a range of 20-80% PEG4000 in 200-400 mM mannitol, 100-500 mM CaCl₂ solution was assayed. The protoplasts were then washed in W5 and centrifuged at 80 g for 3 minutes, prior resuspension in 1 ml W5 and incubated in the dark at 23° C. After incubation for 24-72 hours fluorescence was detected by microscopy.

### FACS Sorting of Fluorescent Protein-Expressing Cells

24-72 hours after plasmid/RNA delivery, cells were collected and sorted for fluorescent protein expression using a flow cytometer in order to enrich for mCherry/Editing agent expressing cells as previously described [Chiang et al., Sci Rep (2016) 6: 24356]. This enrichment step allows bypassing antibiotic selection and collecting only cells transiently expressing the fluorescent protein, Cas9 and the sgRNA. These cells can be further tested for editing of the target gene by HR yielding successful swapping events and loss of the corresponding gene expression.

### Bombardment and Plant Regeneration

### Arabidopsis Root Preparation:

Chlorine gas sterilized Arabidopsis (cv. Col-0) seeds were sown on MS minus sucrose plates and vernalised for three days in the dark at 4° C., followed by germination vertically at 25° C. in constant light. After two weeks, roots were excised into 1 cm root segments and placed on Callus Induction Media (CIM: ½ MS with B5 vitamins, 2% glucose, pH 5.7, 0.8% agar, 2 mg/l IAA, 0.5 mg/l 2,4-D, 0.05 mg/l kinetin) plates. Following six days incubation in the dark, at 25° C., the root segments were transferred onto filter paper discs and placed onto CIMM plates, (½ MS without vitamins, 2% glucose, 0.4 M mannitol, pH 5.7 and 0.8% agar) for 4-6 hours, in preparation for bombardment.

### Bombardment

Plasmid constructs were introduced into the root tissue via the PDS-1000/He Particle Delivery (Bio-Rad; PDS-1000/He System #1652257), several preparative steps, outlined below, were required for this procedure to be carried out.

### Gold Stock Preparation

40 mg of 0.6 μm gold (Bio-Rad; Cat: 1652262) was mixed with 1 ml of 100% ethanol, pulse centrifuged to pellet and the ethanol removed. This wash procedure was repeated another two times.

Once washed the pellet was resuspended in 1 ml of sterile distilled water and dispensed into 1.5 ml tubes of 50 μl aliquot working volumes.

### Bead Preparation

In short, the following was performed:

A single tube was sufficient gold to bombard 2 plates of Arabidopsis roots, (2 shots per plate), therefore each tube was distributed between 4 (1,100 psi) Biolistic Rupture disks (Bio-Rad; Cat: 1652329).

Bombardments requiring multiple plates of the same sample, tubes were combined and volumes of DNA and CaCl₂)/spermidine mixture adjusted accordingly, in order to maintain sample consistency and minimize overall preparations.

The following protocol summarises the process of preparing one tube of gold, these should be adjusted according to number of tubes of gold used.

All subsequent processes were carried out at 4° C. in an Eppendorf thermomixer.

Plasmid DNA samples were prepared, each tube comprising 11 μg of DNA added at a concentration of 1000 ng/μl
1) 493 μl ddH2O was added to 1 aliquot (7 μl) of spermidine (Sigma-Aldrich; S0266), giving a final concentration of 0.1 M spermidine. 1250 μl 2.5M CaCl₂) was added to the spermidine mixture, vortexed and placed on ice.
2) A tube of pre-prepared gold was placed into the thermomixer, and rotated at a speed of 1400 rpm.
3) 11 μl of DNA was added to the tube, vortexed, and placed back into the rotating thermomixer.
4) To bind, DNA/gold particles, 70 μl of spermidine CaCl₂ mixture was added to each tube (in the thermomixer).
5) The tubes were vigorously vortexed for 15-30 seconds and placed on ice for about 70-80 seconds.
6) The mixture was centrifuged for 1 minute at 7000 rpm, the supernatant was removed and placed on ice.
7) 500 μl 100% ethanol was added to each tube and the pellet was resuspended by pipetting and vortexed.
8) The tubes were centrifuged at 7000 rpm for 1 minute.
9) The supernatant was removed and the pellet resuspended in 50 μl 100% ethanol, and stored on ice.

### Macro Carrier Preparation

The following was performed in a laminar flow cabinet:
1) Macro carriers (Bio-Rad; 1652335), stopping screens (Bio-Rad; 1652336), and macro carrier disk holders were sterilized and dried.
2) Macro carriers were placed flatly into the macro carrier disk holders.
3) DNA coated gold mixture was vortexed and spread (5 μl) onto the center of each Biolistic Rupture disk.

Ethanol was allowed to evaporate.

### PDS-1000 (Helium Particle Delivery System)

In short, the following was performed:

The regulator valve of the helium bottle was adjusted to at least 1300 psi incoming pressure. Vacuum was created by pressing vac/vent/hold switch and holding the fire switch for 3 seconds. This ensured helium was bled into the pipework.

1100 psi rupture disks were placed into isopropanol and mixed to remove static.
1) One rupture disk was placed into the disk retaining cap.
2) Microcarrier launch assembly was constructed (with a stopping screen and a gold containing microcarrier).
3) Petri dish Arabidopsis root callus was placed 6 cm below the launch assembly.
4) Vacuum pressure was set to 27 inches of Hg (mercury) and helium valve was opened (at approximately 1100 psi).
5) Vacuum was released; microcarrier launch assembly and the rupture disk retaining cap were removed.
6) Bombardment on the same tissue (i.e. each plate was bombarded 2 times).
7) Bombarded roots were subsequently placed on CIM plates, in the dark, at 25° C., for additional 24 hours.

### Co-Bombardments

When bombarding GEiGS plasmids combinations, 5 μg (1000 ng/μl) of the sgRNA plasmid was mixed with 8.5 μg (1000 ng/μl) swap plasmid and 11 μl of this mixture was added to the sample. If bombarding with more GEiGS plasmids at the same time, the concentration ratio of sgRNA plasmids to swap plasmids used was 1:1.7 and 11 μg (1000 ng/μl) of this mixture was added to the sample. If co-bombarding with plasmids not associated with GEiGS swapping, equal ratios were mixed and 11 μg (1000 ng/μl) of the mixture was added to each sample.

### Plant Regeneration

For shoot regeneration, modified protocol from Valvekens et al. [Valvekens, D. et al., Proc Natl Acad Sci USA (1988) 85(15): 5536-5540] was carried out. Bombarded roots were placed on Shoot Induction Media (SIM) plates, which included ½ MS with B5 vitamins, 2% glucose, pH 5.7, 0.8% agar, 5 mg/l 2 iP, 0.15 mg/l IAA. Plates were left in 16 hours light at 25° C.-8 hours dark at 23° C. cycles. After 10 days, plates were transferred to MS plates with 3% sucrose, 0.8% agar for a week, then transferred to fresh similar plates. Once plants regenerated, they were excised from the roots and placed on MS plates with 3% sucrose, 0.8% agar, until analysed.

### Colony Formation and Plant Regeneration

The fluorescent protein positive cells were partly sampled and used for DNA extraction and genome editing (GE) testing and partly plated at high dilution in liquid medium to allow colony formation for 28-35 days. Colonies were picked, grown and split into two aliquots. One aliquot was used for DNA extraction and genome editing (GE) testing and CRISPR DNA-free testing (see below), while the others were kept in culture until their status was verified. Only the ones clearly showing to be GE and CRISPR DNA-free were selected forward. Colonies were grown in culture medium in for about 6-10 weeks. Protocolonies (or calli) were subcultured into regeneration media (e.g. half strength MS+B5 vitamins, 20 g/l sucrose). Regenerated plantlets were placed on solidified media (0.8% agar) at a low light intensity at 28° C. After 2 months, plantlets were transferred to soil and placed in a glasshouse at 80-100% humidity.

### Virus Inoculation and DNA Delivery to Arabidopsis Seedlings

Sap from Arabidopsis leaves infected with TuMV infectious clone p35S::TuMV-GFP (0.1 mg/ml) are used for mechanical inoculations.

### Plant Propagation

Clones that were sequenced and predicted to have lost the expression of the target genes and found to be free of the CRISPR system DNA/RNA were propagated for generation in large quantities and in parallel were differentiated to generate seedlings from which functional assay is performed to test the desired trait.

### Phenotypic Analysis

As described above, such as by looking at the pigmentation, florescence or morphology dependent on the target gene.

### Allyl Alcohol Selection

For selection of plants with allyl alcohol, 10 days post bombardment, roots were placed on SIM media. Roots were immersed in 30 mM allyl alcohol (Sigma-Aldrich, US) for 2 hours. Then the roots were washed three times with MS media, and placed on MS plates with 3% sucrose, 0.8% agar. Regeneration process was carried on as previously described.

### Genotyping

Tissue samples were treated and amplicons amplified in accordance to the manufacturers recommendations. MyTaq Plant-PCR Kit (BioLine BIO 25056) for short internal amplification and Phire Plant Direct PCR Kit (Thermo Scientific; F-130WH) for longer external amplifications. Oligos used for these amplifications are specified in Table 2, above. Different modifications in the miRNA loci were identified through different digestion patterns of the amplicons, as follows:

For modifications of miR-390-internal amplicon was 978 base pairs long, and for external amplification it was 2629 base pairs. For the identification of swap 7, digestion with NlaIII resulted in a fragment size of 636 base pairs, while in the wt version it was cleaved to 420 and 216 long fragments. For the identification of swap 8, digestion with Hpy188I resulted in fragments size of 293 and 339 base pairs, while in the wt version this site was absent and resulted in a 632-long fragment. For the identification of swaps 11 and 12, digestion with BccI resulted in a fragment size of 662 base pairs, while in the wt version it was cleaved to 147 and 417 long fragments.

For modifications of miR-173-internal amplicon was 574 base pairs long, and for nested external amplification it was 466 base pairs. For the identification of swap 3, digestion with BslI resulted in fragments size of 217 and 249 base pairs in the external amplicon and 317 and 149 in the internal one. In the wt version this site was absent and resulted in a 466-long fragment in the external amplicon and 574 in the internal reaction. For the identification of swap 4, digestion with BtsαI resulted in fragments size of 212 and 254 base pairs in the external amplicon and 212 and 362 in the internal one. In the wt version, this site was absent and resulted in a 466-long fragment in the external amplicon and 574 in the internal reaction. For the identification of swap 9, digestion with NlaIII resulted in fragments size of 317 and 149 base pairs in the external amplicon and 317 and 244 in the internal one. In the wt version, this site was absent and resulted in a 466-long fragment in the external amplicon and 561 in the internal reaction. For the identification of swap 10, digestion with NlaIII resulted in fragments size of 375 and 91 base pairs in the external amplicon and 375 and 186 in the internal one. In the wt version, this site was absent and resulted in a 466-long fragment in the external amplicon and 561 in the internal reaction.

### DNA and RNA Isolation

Samples were harvested into liquid nitrogen and stored in −80° C. until processed. Grinding of tissue was carried out in tubes placed in dry ice, using plastic Tissue Grinder Pestles (Axygen, US). Isolation of DNA and total RNA from ground tissue was carried out using RNA/DNA Purification kit (cat. 48700; Norgen Biotek Corp., Canada), according to manufacturer's instructions. In the case of low 260/230 ratio (<1.6), of the RNA fraction, isolated RNA was precipitated overnight in −20° C., with 1 μl glycogen (cat. 10814010; Invitrogen, US) 10% V/V sodium acetate, 3 M pH 5.5 (cat. AM9740, Invitrogen, US) and 3 times the volume of ethanol. The solution was centrifuged for 30 minutes in maximum speed, at 4° C. This was followed by two washes with 70% ethanol, airdrying for 15 minutes and resuspending in Nuclease-free water (cat. 10977035; Invitrogen, US).

Reverse Transcription (RT) and Quantitative Real-Time PCR (qRT-PCR)

One microgram of isolated total RNA was treated with DNase I according to manufacturer's manual (AMPD1; Sigma-Aldrich, US). The sample was reverse transcribed, following the instructor's manual of High-Capacity cDNA Reverse Transcription Kit (cat 4368814; Applied Biosystems, US).

For gene expression, Quantitative Real Time PCR (qRT-PCR) analysis was carried out on CFX96 Touch™ Real-Time PCR Detection System (BioRad, US) and SYBR® Green JumpStart™ Taq ReadyMix™ (S4438, Sigma-Aldrich, US), according to manufacturer's′ protocols, and analysed with Bio-RadCFX manager program (version 3.1). For the analysis of AtADH1 (AT1G77120) the following primer set was used: Forward GTTGAGAGTGTTGGAGAAGGAG SEQ ID NO: 367 and reverse CTCGGTGTTGATCCTGAGAAG SEQ ID NO: 368; For the analysis of AtPDS3 (AT4G14210), the following primer set was used: Forward GTACTGCTGGTCCTTTGCAG SEQ ID NO: 369 and reverse AGGAGCACTACGGAAGGATG SEQ ID NO: 370; For endogenous calibration gene, the 18S ribosomal RNA gene (NC_037304) was used—Forward ACACCCTGGGAATTGGTTT SEQ ID NO: 371 and reverse GTATGCGCCAATAAGACCAC SEQ ID NO: 372.

### Example 1A

### Genome Editing Induced Gene Silencing (GEiGS)

In order to design GEiGS oligos, template non-coding RNA molecules (precursors) that are processed and give raise to derivate small silencing RNA molecules (matures) are required. Two sources of precursors and their corresponding mature sequences were used for generating GEiGS oligos. For miRNAs, sequences were obtained from the miRBase database [Kozomara, A. and Griffiths-Jones, S., Nucleic Acids Res (2014) 42: D68,ÄìD73]. tasiRNA precursors and matures were obtained from the tasiRNAdb database [Zhang, C. et al, Bioinformatics (2014) 30: 1045,Äì1046].

Silencing targets were chosen in a variety of host organisms (see Table 1B, above). siRNAs were designed against these targets using the siRNA rules software [Holen, T., RNA (2006) 12: 1620,Äì1625.]. Each of these siRNA molecules was used to replace the mature sequences present in each precursor, generating “naive” GEiGS oligos. The structure of these naive sequences was adjusted to approach the structure of the wild type precursor as much as possible using the ViennaRNA Package v2.6 [Lorenz, R. et al., ViennaRNA Package 2.0. Algorithms for Molecular Biology (2011) 6: 26]. After the structure adjustment, the number of sequences and secondary structure changes between the wild type and the modified oligo were calculated. These calculations are essential to identify potentially functional GEiGS oligos that require minimal sequence changes with respect to the wild type.

CRISPR/cas9 small guide RNAs (sgRNAs) against the wild type precursors were generated using the CasOT software [Xiao, A. et al., Bioinformatics (2014) 30: 1180,Äì1182](see Table 1B, above). sgRNAs were selected where the modifications applied to generate the GEiGS oligo affect the PAM region of the sgRNA, rendering it ineffective against the modified oligo.

### Example 1B

### Gene Silencing of Endogenous Plant Gene—PDS

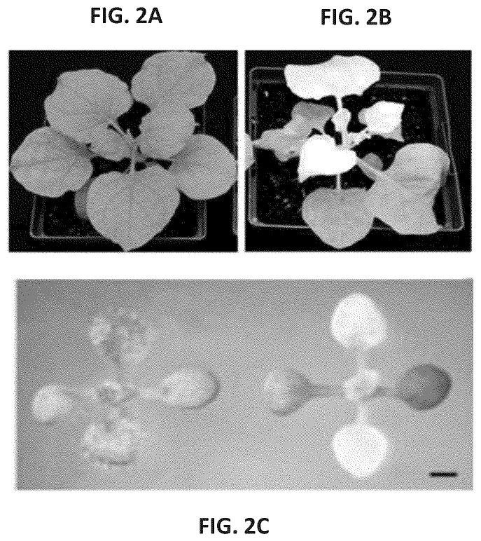

In order to establish a high-throughput screening for quantitative evaluation of endogenous gene silencing using Genome Editing Induced Gene Silencing (GEiGS), the present inventors considered several potential visual markers. The present inventors chose to focus on genes involved in pigment accumulation, such as those encoding for phytoene desaturase (PDS). Silencing of PDS causes photobleaching (FIG. 2B) which allows to use it as robust seedling screening after gene editing as proof-of-concept (POC). FIGS. 2A-C show a representative experiment with N. benthamiana and Arabidopsis plants silenced for PDS. Plants show the characteristic photobleaching phenotype observed in plants with diminished amounts of carotenoids.

In the POC experiment, choosing siRNAs was carried out as follows:

In order to initiate the RNAi machinery in Arabidopsis or Nicotiana benthamiana against the PDS gene using GEiGS application, there is a need to identify effective 21-24 bp siRNA targeting PDS. Two approaches are used in order to find active siRNA sequences: 1) screening the literature—since PDS silencing is a well-known assay in many plants, the present inventors are identifying well characterized short siRNA sequences in different plants that might be 100% match to the gene in Arabidopsis or Nicotiana benthamiana. 2) There are many public algorithms that are being used to predict which siRNA will be effective in initiating gene silencing to a given gene. Since the predictions of these algorithms are not 100%, the present inventors are using only sequences that are the outcome of at least two different algorithms.

In order to use siRNA sequences that silence the PDS gene, the present inventors are swapping them with a known endogenous non-coding RNA gene sequence using the CRISPR/Cas9 system (e.g. changing a miRNA sequence, changing a long dsRNA sequence, creating antisense RNA, changing tRNA etc.). There are many databases of characterized non-coding RNAs e.g. miRNAs; the present inventors are choosing several known Arabidopsis or Nicotiana benthamiana endogenous non-coding RNAs e.g. miRNAs with different expression profiles (e.g. low constitutive expression, highly expressed, induced in stress etc.). For example, in order to swap the endogenous miRNA sequence with siRNA targeting PDS gene, the present inventors are using the HR approach (Homologous Recombination). Using HR, two options are contemplated: using a donor ssDNA oligo sequence of around 250-500 nt which includes, for example, the modified miRNA sequence in the middle or using plasmids carrying 1 Kb-4 Kb insert which is almost 100% identical to the miRNA surrounding in the plant genome except the 2×21 bp of the miRNA and the *miRNA that is changed to the siRNA of the PDS (500-2000 bp up and downstream the siRNA, as illustrated in FIG. 1). The transfection includes the following constructs: CRISPR:Cas9/GFP sensor to track and enrich for positive transformed cells, gRNAs that guides the Cas9 to produce a double stranded break (DSB) which is repaired by HR depending on the insertion vector/oligo. The insertion vector/oligo contains two continuous regions of homology surrounding the targeted locus that are replaced (i.e. miRNA) and is modified to carry the mutation of interest (i.e. siRNA). If plasmid is used, the targeting construct comprises or is free from restriction enzymes-recognition sites and is used as a template for homologous recombination ending with the replacement of the miRNA with the siRNA of choice. After transfection to protoplasts, FACS is used to enrich for Cas9/sgRNA-transfected events, protoplasts are regenerated to plants and bleached seedlings are screened and scored (see FIG. 1). As control, protoplasts are transfected with an oligo carrying a random non-PDS targeting sequence. The positive edited plants are expected to produce siRNA sequences targeting PDS and therefore PDS gene is silenced and seedling are seen as white compared to the control with no gRNA. It is important to note that after the swap, the edited miRNA will still be processed as miRNA because the original base-pairing profile is kept. However, the newly edited processed miRNA has a high complementary to the target (e.g. 100%), and therefore, in practice, the newly edited small RNA will act as siRNA.

### Example 2

### Gene Silencing of “Endogenous” Transgene—GFP

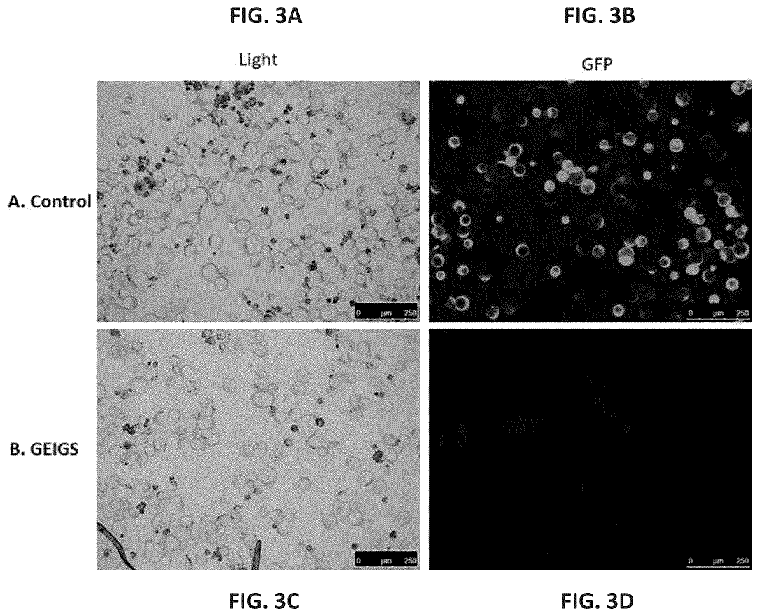

Another quick and robust approach to check the efficiency of GEiGS is by silencing a transgene which is also a marker gene like GFP (green fluorescent protein). There are few easy options to assess the effectiveness of the GFP silencing in the cell, e.g. FACS analysis, PCR and microscopy. In order to show POC of GFP silencing using GEiGS, the present inventors are using a transgenic Arabidopsis or tobacco lines stably expressing GFP. Protoplasts from GFP expressing plants are used with GEiGS methodology to modify endogenous non-coding RNA e.g. miRNA to act as siRNA potent to initiate the RNA silencing mechanism targeting the GFP gene. The positive edited plants are expected to be silenced for GFP expression as illustrated in FIGS. 3A-D. Furthermore, GFP silencing in plants is well characterized and there are many available short RNA sequences (siRNA) that can be utilized to initiate GFP silencing. Therefore, for gene swapping, the present inventors are using publically available tools to generate siRNA specific to GFP or are using known siRNA molecules available from the literature.

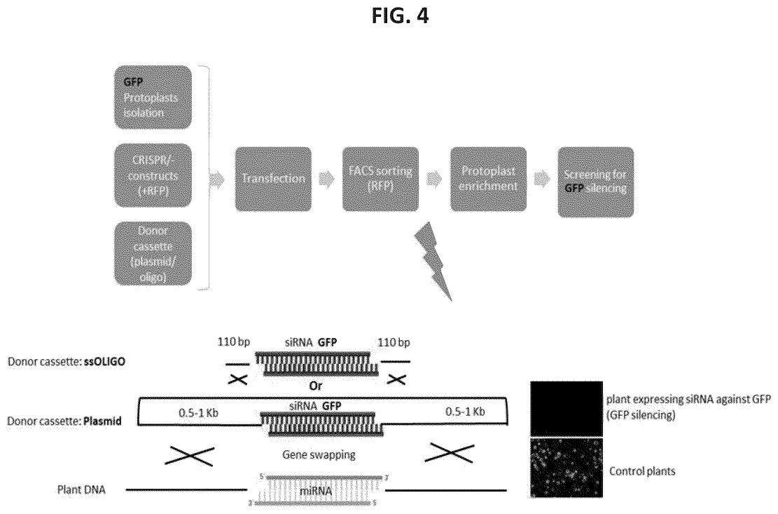

In order to use siRNA sequences that will silence the GFP gene, the present inventors are swapping them with a known endogenous non-coding RNA e.g. miRNA gene sequence using the CRISPR/Cas9 system (e.g. changing a miRNA sequence, changing a long dsRNA sequence, creating antisense RNA, changing tRNA etc.). There are many databases of characterized non-coding RNAs e.g. miRNAs, the present inventors are choosing several known Arabidopsis or Nicotiana benthamiana non-coding RNAs e.g. miRNAs with different expression profiles (e.g. low constitutive expression, highly expressed, induced in stress etc.). For example, in order to swap the endogenous miRNA sequence with siRNA, the present inventors are using the HR approach. In HR two options are contemplated: using a donor oligo sequence of around 250-500 bp which includes, for example, the siRNA sequence in the middle or using plasmids expressing 1 Kb-4 Kb insert which is almost 100% identical to the miRNA surrounding in the plant genome except the 2×21 bp of the miRNA and the *miRNA that are changed to the siRNA of the GFP (500-2000 bp up and downstream the siRNA, see FIG. 1). The transfection includes the following constructs: CRISPR:Cas9/RFP sensor to track and enrich for positive transformed cells using e.g. FACS analysis, gRNAs that guides the Cas9 to produce a DSB which is repaired by HR depending on the insertion vector/oligo. The insertion vector contains two continuous regions of homology surrounding the targeted locus that are replaced (i.e. miRNA) and is modified to carry the mutation of interest (i.e. siRNA). The targeting construct comprises or is free from restriction enzymes-recognition sites and is used as a template for homologous recombination ending with the replacement of the miRNA with the siRNA of choice. After transfection to protoplasts, FACS is used to enrich for positive transfected events (using the red fluorescent protein (RFP) marker), enriched protoplasts are scored for GFP silencing under a microscope (FIG. 4). The positive edited protoplasts are expected to produce siRNA sequences targeting GFP and therefore GFP expression of the transgene is expected to be silenced as compared to control protoplasts. GFP is a faster method than PDS since the two last steps of recovery and regeneration are not necessary, the scoring can be done on the protoplasts/cells level.

### Example 3

### Gene Silencing of Exogenous Transgene—GFP in Arabidopsis

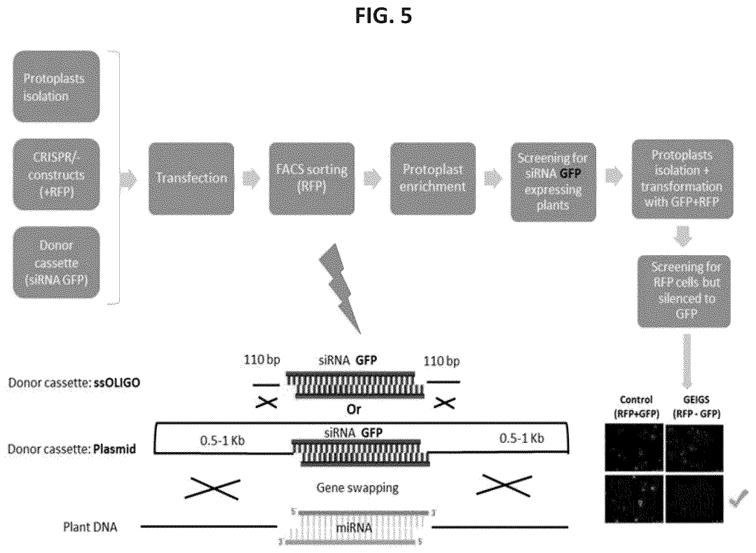

In addition to the former example of GFP silencing, another way to demonstrate the efficiency of GEiGS is by silencing a marker gene like GFP (green fluorescent protein) in a transient GFP transformation assay. In this example, first plant cells (e.g. Arabidopsis) are treated using GEiGS to express small siRNA molecules targeting GFP (method for utilizing siGFP are discussed in Example 2 above). Control protoplasts (e.g. GEiGS-PDS) and edited protoplasts using GEiGS (expressing siGFP) are then transfected with a plasmid expressing separately two markers (sensor) GFP+RFP. Protoplast which express only RFP but not GFP in the GEiGS treatment are the results of GFP silencing due to siGFP expression (as illustrated in FIG. 5).

### Example 4

### Immunized Plants to Virus Infection, Silencing of Exogenous Virus Gene (Using GFP as Marker)

In order to prove that GEiGS is a robust method for plant immunization with the ability to knock down exogenous genes, the present inventors are providing an example of silencing of a virus gene. There are various viruses that infect different plant species and that can be used in the present POC: TuMV, CMV, TMV etc.

Turnip mosaic virus (TuMV) is transmitted non-persistently by aphids and causes prevalent diseases of cruciferous crops in many parts of the world. TuMV genome, which is single-stranded, is a positive-sense RNA molecule of approximately 10,000 nt (accession number NC_002509). TuMV has the same typical potyvirus genetic organization previously discussed by Urcuqui-Inchima et al. [Urcuqui-Inchima et al., Virus Res. (2001) 74: 157-175]. The symptoms of TuMV are mottling in broad, yellow, circular, and irregular areas. The oldest leaves often become bright yellow all over. The lamina often becomes necrotic. Extensive use was made of TuMV-GFP and suppressor-deficient TuMV-AS9-GFP to expose antiviral silencing activities in Arabidopsis. Wild-type plants were immune to TuMV-AS9-GFP, but immunity was effectively suppressed by loss of DCL2 and DCL4, indicating that TuMV normally masks the effects of a siRNA dependent antiviral response [Hernan Garcia-Ruiz et al., The Plant Cell (2010) 22: 481-496].

Cucumber mosaic virus (CMV) is a plant pathogenic virus in the family Bromoviridae. It is the type member of the plant virus genus, Cucumovirus. This virus has a worldwide distribution and a very wide host range. In fact it has the reputation of having the widest host range of any known plant virus. It can be transmitted from plant to plant both mechanically by sap and by aphids in a stylet-borne fashion. This virus was first found in cucumbers (Cucumis sativus) showing mosaic symptoms in 1934, hence the name Cucumber mosaic. An expression CMV-based expression vector that utilizes the mutant 3a MP for CP-independent cell-to-cell movement was developed. This new vector [Fujiki et al., Virology (2008) 381(1): 136-142] was incorporated into an agrobacterium binary vector and delivered into plants via agroinfiltration. The results demonstrate that this novel CMV-based expression vector holds great promise for recombinant protein production.

Tobacco mosaic virus (TMV), a single-stranded RNA virus that commonly infects solanaceous plants, a plant family that includes many species such as petunias, tomatoes and tobacco. The virus causes a mosaic pattern of brown spots on the surface of leaves. The virus doesn't usually cause the plant to die, but can seriously stunt its growth. Lower leaves can suffer from ‘mosaic burn’ in hot and dry weather, where large areas of the leaf die. This virus cannot get into plants on its own. Plants are usually infected via plant wounds after human handling or via contaminated equipment. Once inside the plant, the virus releases its genetic code (RNA). The plant gets confused by this code, mistaking it for its own, and starts to produce virus proteins. Virus-based expression systems in plants are particularly attractive versus alternative transient expression systems due to the high level of gene multiplication and concomitant elevated levels of expression achievable within a short period of time while minimizing impairment of host activities. TMV is one of the most extensively studied plant viruses and has thus become a natural choice for vector development. TMV-based vectors have led to recombinant protein yield as high as 80% of total soluble protein. Agroinfection is inexpensive and reproducible, making it a preferred method of delivering viral expression vectors into plant tissues as part of the T-DNA of a binary vector carried by Agrobacterium tumefaciens.

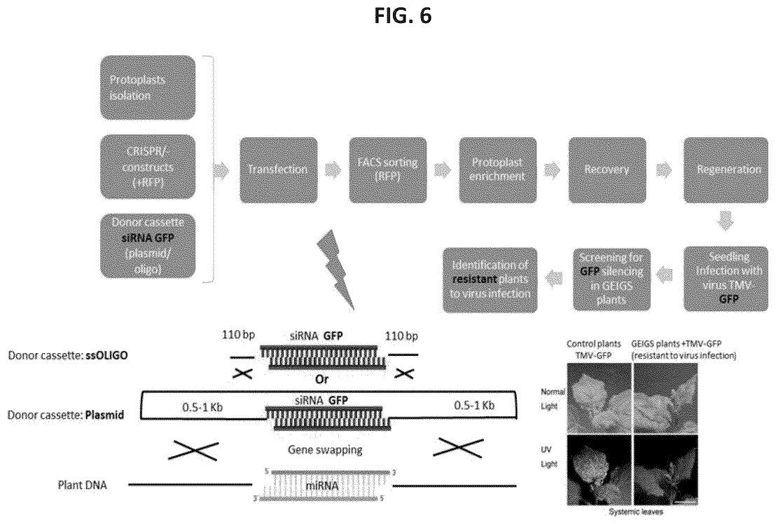

The present inventors are using TuMV-GFP for infection of Arabidopsis or TMV-GFP for tobacco plants. In order to create plants resistant to virus infection, the present inventors are using an engineered virus that expresses GFP upon plant infection. Using such a virus will enable to use the same constructs as described in Example 3, above. The difference being that now the GFP is expressed from the virus infection. Control plants that are infected with virus-GFP (CMV or TMV) show expression of GFP under the microscope (FIG. 6) however, GEiGS plants engineered to express siRNA GFP are expected to show reduced levels of GFP (FIG. 6). Accordingly, generating GEiGS plants with no GFP expression after infection with Virus-GFP will demonstrate that RNAi silencing of exogenous gene was achieved and that GEiGS is an effective method to immune plants against viruses and potentially other pathogens. There are few easy options to assess the effectiveness of the GFP silencing in the cell, such as the use FACS analysis, PCR and microscopy. GFP silencing in plants is well characterized and there are many available short RNA sequences (siRNA) that are active in initiating GFP silencing. Therefore, for gene swapping, the present inventors are using a few known siRNA molecules available from the literature.

In order to use siRNA sequences that will silence the GFP gene, the present inventors are swapping them with a known endogenous non-coding RNA e.g. miRNA gene sequence using the CRISPR/Cas9 system (as discussed above, there are many other options to introduce these siRNA sequences, like changing long dsRNA sequences, creating antisense RNA, changing tRNA etc.). There are many databases of characterized endogenous non-coding RNA e.g. miRNAs, the present inventors are choosing several known Arabidopsis or Nicotiana benthamiana non-coding RNA e.g. miRNAs with different expression profiles (e.g. low constitutive expression, highly expressed, induced in stress etc.). For example, in order to swap the endogenous miRNA sequence with siRNA, the present inventors are using the HR approach. In HR two options are contemplated: using a donor oligo sequence of around 250-500 bp which includes, for example, the siRNA sequence in the middle or using plasmids expressing 1 Kb-4 Kb insert which is almost 100% identical to the miRNA surrounding in the plant genome except the 2×21 bp of the miRNA and the *miRNA that are changed to the siRNA of the GFP (500-2000 bp up and downstream the siRNA, see FIG. 1). The transfection includes the following constructs: CRISPR:Cas9/RFP sensor to track and enrich for positive transformed cells using e.g. FACS analysis, gRNAs that guides the Cas9 to produce a DSB which is repaired by HR depending on the insertion vector/oligo. The insertion vector contains two continuous regions of homology surrounding the targeted locus that are replaced (i.e. miRNA) and is modified to carry the mutation of interest (i.e. siRNA). The targeting construct comprises or is free from restriction enzymes-recognition sites and is used as a template for homologous recombination ending with the replacement of the miRNA with the siRNA of choice. After transfection to protoplasts, FACS is used to enrich for positive transfected events, protoplasts are regenerated to plants and plants are infected with the virus by mechanical inoculations. Plants are scored for GFP silencing under microscope (as described in FIG. 6). The positive edited protoplasts with GEiGS are expected to produce siRNA sequences targeting GFP and therefore the virus GFP gene expression is expected to be silenced compared to control unedited plants.

### Example 5

### Banana Plant Resistant to Nematode

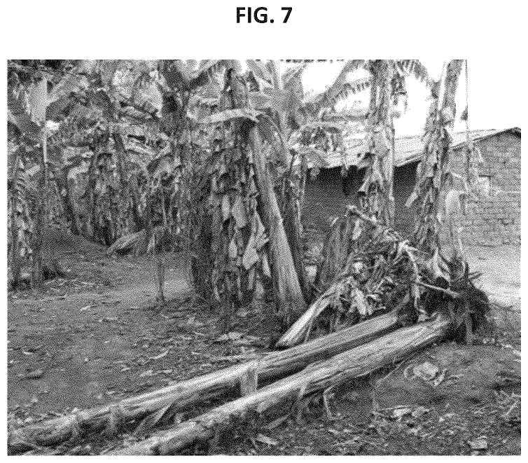

The damage to banana productivity due to nematodes is tremendous, reaching up to 50% of yield loss in untreated soils. The problem is accentuated in traditional banana plantations where mono cropping is a common practice. Banning of nematicides like methyl bromide in various parts of the world exacerbated the problem and leaves farmers with inappropriate and unreliable alternatives. Radopholus similis, the burrowing nematode, is the most economically important nematode parasite of banana in the world. Infection by burrowing nematode causes toppling disease of banana, yellows disease of pepper and spreading decline of citrus. These diseases are the result of burrowing nematode infection destroying root tissue, leaving plants with little to no support or ability to take up water and translocate nutrients. Because of the damage that it causes to citrus, ornamentals and other agricultural industries, worldwide, burrowing nematode is one of the most regulated nematode plant pests (FIG. 7).

RNA interference (RNAi) has emerged as an invaluable gene-silencing tool for functional analysis in a wide variety of organisms, particularly the free-living model nematode Caenorhabditis elegans. An increasing number of studies have described its application to plant parasitic nematodes. Genes expressed in a range of cell types are silenced when nematodes take up double stranded RNA (dsRNA) or short interfering RNAs (siRNAs) that elicit a systemic RNAi response. Extensive siRNA studies with C. elegans suggest that successfully preventing nematodes from completing their life cycle is attributed to silencing genes that are expressed early in embryonic development. In R. similis such candidate genes might be Calreticulin13 (CRT) or the gene collagen 5 (col-5). CRT is a Ca2⁺-binding multifunctional protein that plays key roles in the parasitism, immune evasion, reproduction and pathogenesis of many animal parasites and plant nematodes. Therefore, CRT is a promising target for controlling R. similis. Col-5 belongs to the collagen genes of nematodes encode proteins that have a diverse range of functions. Among their most abundant products are the cuticular collagens, which include about 80% of the proteins present in the nematode cuticle. The structures of these collagens have been found to be strikingly similar in the free-living and parasitic nematode species studied so far, and the genes that encode them appear to constitute a large multigene family whose expression is subject to developmental regulation.

By utilizing GEiGS, the present inventors are creating banana plants expressing siRNA molecules that are transmitted from their roots to nematodes upon feeding, and subsequently induce the silencing of nematode genes. The silencing of genes essential for succession in the life cycle inhibits nematode propagation and abolishes damages caused by nematodes. The present inventors are changing a few banana endogenous non-coding RNA e.g. miRNA sequences with short sequences from the CRT or the col-5 genes. GEiGS is used in Banana protoplasts that are regenerated to plantlets and are then screened with different nematodes for resistance.

### Example 6

### Banana Plant Resistant to Fusarium oxysporum

The genus Fusarium includes several species of fungi that are broadly spread in soil and organic substrates worldwide. Fusarium oxysporum is one of the most relevant species of this genus and is the causal agent of root rots, damping-off and wilt diseases in more than 100 plants species, including a wide range of economically important horticultural crops, flowers, trees, and a number of field crops such as cabbage, banana, and cotton. Fusarium oxysporum is a devastating pathogen causing extensive yield losses in a variety of crops and development of sustainable, environmentally friendly methods to improve crop resistance is crucial. F. oxysporum consists of over 120 forma specialis of pathogenic strains determined by their primary host plants. All strains of F. oxysporum are saprophytic, being able to grow and survive for long periods on organic matter in soil making it very difficult to control. Its pathogenic life cycle starts with spore germination upon recognition of a suitable host. Once the hyphae is formed, the pathogen enters its host by directly penetrating the roots and colonizes it within the xylem by producing microconidia which leads to mycelium formation. Colonization and toxin production by the pathogen results in blockage of the host vascular system, causing characteristic disease symptoms including vasculature yellowing, vein clearing, chlorosis, and necrosis in leaf veins and leaves, leaf detachment and wilting. After the plant dies, the fungus sporulates on the decayed leaf surfaces. F. oxysporum is most prevalent in tropical and subtropical regions and it is expected that its geographical range will extend due to climate change. Current control methods for Fusarium wilt are very limited with crop rotations being ineffective due to the large host range and its persistence in soil. Management of Fusarium wilt is mainly done through cultural practices and farm hygiene which only reduce the transmission of inoculum while soil sterilization can only be performed in glasshouses. Soil fumigation using broad-spectrum biocides such as methyl bromide is expensive and has many hazardous effects on the environment.

Hu z. have used Host-Delivered RNA interference technology to partially silence three different genes (FOW2, FRP1, and OPR) in the hemi-biotrophic fungus F. oxysporum f. sp. Conglutinans [Hu et al., Front Chem. (2015) 20 (3):1]. Expression of double stranded RNA (dsRNA) molecules targeting fungal pathogen genes was achieved in a number of transgenic Arabidopsis lines. F. oxysporum infecting the transgenic lines displayed substantially reduced mRNA levels on all three targeted genes, with an average of 75, 83, and 72% reduction for FOW2, FRP1, and OPR, respectively. The silencing of pathogen genes had a clear positive effect on the ability of the transgenic lines to fight infection. All transgenic lines displayed enhanced resistance to F. oxysporum with delayed disease symptom development, especially FRP1 and OPR lines. Survival rates after fungal infection were higher in the transgenic lines compared to control wild type plants which consistently showed survival rates of 10%, with FOW2 lines showing 25% survival; FRP1 lines 30-50% survival and OPR between 45 and 70% survival. The down-regulation effect was specific for the targeted genes without unintended effects in related genes (Hu Z. (2015) supra). It was shown that in fungi, both long and short dsRNAs are equally internalized and induce RNAi to silence target genes.

The present inventors are utilizing GEiGS in order to create Banana plants resistant to F. oxysporum by changing few endogenous non-coding RNA e.g. miRNAs sequences to specifically target the fungi genes like FOW2, FRP1 and OPR. Edited Protoplasts are regenerated to plantlets and are challenged with F. oxysporum in a controlled environment, resistant plants are verified to express the relevant siRNA.

### Example 7

### Coffee Tree Resistant to Nematode

Coffea is a genus of flowering plants whose seeds, called coffee beans, are used to make various coffee beverages and products. It is a member of the family Rubiaceae. They are shrubs or small trees native to tropical and southern Africa and tropical Asia. Coffee ranks as one of the world's most valuable and widely traded commodity crops and is an important export product of several countries, including those in Central and South America, the Caribbean and Africa. A steady decline in coffee production has been attributed to biotic and socio-economic constraints. Among the less studied biotic constraints are nematodes.

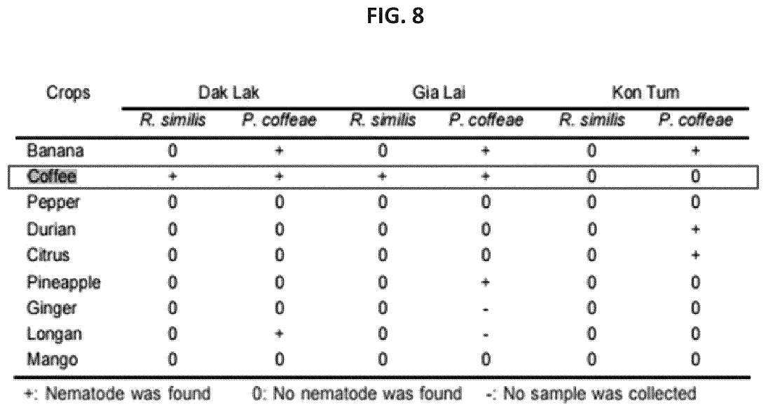

Plant-parasitic nematodes are regarded as a severe constraint to coffee production in the world and especially in Vietnam (FIG. 8). The dominant and most important species are Radopholus arabocoffeae and Pratylenchus coffeae. Both species are responsible for the death of plants younger than 5 years old. Traditionally, the main method to control P. coffeae is by chemical means there is no particular control strategy against R. arabocoffeae.

The present inventors are utilizing GEiGS strategy (as described in Example 5 above) to create Coffea canephora (Robusta) trees expressing siRNA molecules that are transmitted from their roots to nematodes upon feeding, and subsequently inducing the silencing of nematode genes. The silencing of genes essential for succession in the life cycle inhibits nematode propagation and abolishes damages caused by nematodes. The present inventors are thus changing a few endogenous non-coding RNA e.g. miRNA sequences with short sequences from the nematode genes. GEiGS is used in coffee protoplasts that are regenerated to plantlets and then screened with different nematodes for resistance.

### Example 8

### Generation of Plants with Modified Endogenous miRNA to Target Different Genes

Minimal modifications in the genomic loci of a miRNA, in its recognition sequence (which will mature to a miRNA) can lead to a new system to regulate new genes, in a non-transgenic manner. Therefore, an agrobacterium-free transient expression method was used, to introduce these modifications by bombardment of Arabidopsis roots, and their regeneration for further analysis. The present inventors had chosen to target two genes, PDS3 and ADH1 in Arabidopsis plants.

Carotenoids play an important role in many physiological processes in plants and the phytoene desaturase gene (PDS3) encodes one of the important enzymes in the carotenoid biosynthesis pathway, its silencing produces an albino/bleached phenotype. Accordingly, plants with reduced expression of PDS3 exhibit reduced chlorophyll levels, up to complete albino and dwarfism.

Alcohol dehydrogenase (ADH1) comprises a group of dehydrogenase enzymes which catalyse the interconversion between alcohols and aldehydes or ketones with the concomitant reduction of NAD+ or NADP+. The principal metabolic purpose for this enzyme is the breakdown of alcoholic toxic substances within tissues. Plants harbouring reduced ADH1 expression exhibit increase tolerance to allyl alcohol. Accordingly, plants with reduced ADH1 are resistant to the toxic effect of allyl alcohol, therefore their regeneration was carried out with allyl alcohol selection.

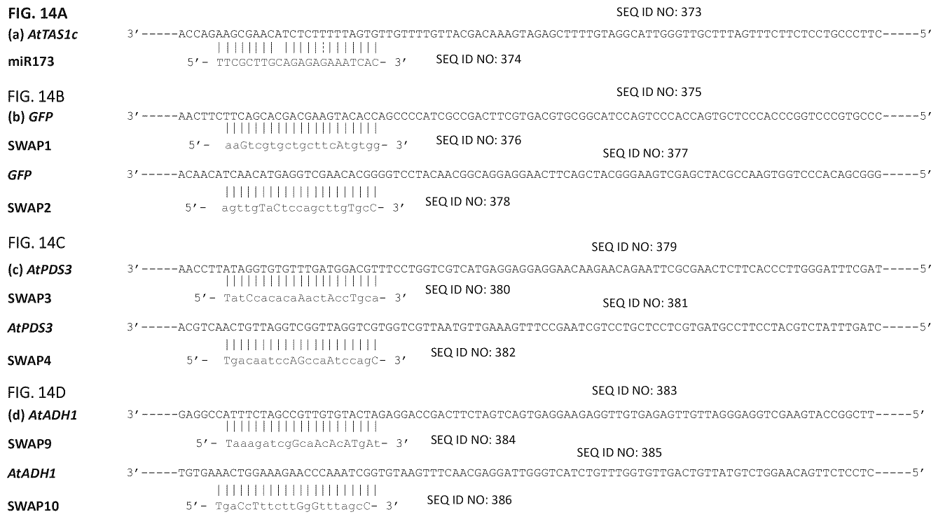

Two well-established miRNAs were chosen to be modified, miR-173 and miR-390, that were previously shown to be expressed throughout plant development [Zielezinski A et al., BMC Plant Biology (2015) 15: 144]. To introduce the modification, a 2-component system was used. First, the CRISPR/CAS9 system was used, to generate a cleavage in the miR-173 and miR-390 loci, through designed specific guide RNAs (FIGS. 12A and 13A; and Table 2, above), to promote homologous DNA repair (HDR) in the site. Second, A DONOR sequence, with the desired modification of the miRNA sequence, to target the newly assigned genes, was introduced as a template for the HDR (FIGS. 12A-G, 13A-G, 14A-D and 15A-D; Table 2, above). In addition, since the secondary structure of the primary transcript of the miRNA (pri-miRNA) is important for the correct biogenesis and activity of the mature miRNA, further modifications were introduced in the complementary strand in the pri-miRNA and analysed in mFOLD (www(dot)unafold(dot)rna(dot)Albany(dot)edu) for structure conservation (FIGS. 12A-G and 13A-G). In total, two guides were designed for each miRNA loci, and two different DONOR sequences (modified miRNA sequences) were designed for each gene (FIGS. 14A-D and 15A-D, and Table 2, above).

### Example 9

### Bombardment and Plant Regeneration

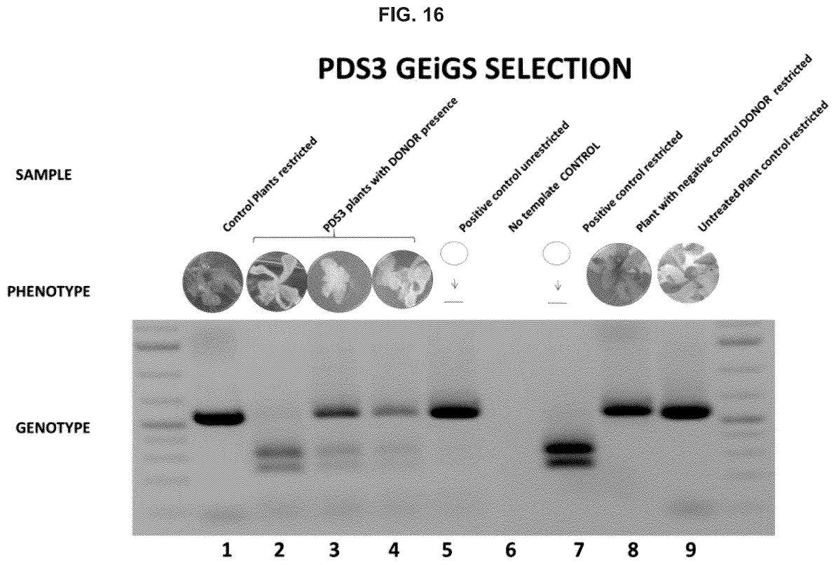

GEiGS constructs were bombarded into pre-prepared roots (as discussed in detail in the materials and experimental procedures section, above) and regenerated. Plantlets were selected via bleached phenotype for PDS3 transformants and survival on allyl alcohol treatment for ADH1 transformants. In order to validate Swap compared to no Swap, i.e. retained wild type, these plants were subsequently screened for insertion through specific primers spanning the modified region followed by restriction digest (FIG. 16).

### Example 10

### Genotype Validation of Phenotype Selection

As discussed above, the Proof of Concept (POC) for the gene editing system was established using well known phenotypic traits, Phytoene desaturase (PDS3) and Alcohol desaturase (ADH1) as targets.

As mentioned above, plants harbouring reduced ADH1 expression exhibit increase tolerance to allyl alcohol. Therefore, bombarded plants for modified miRNA to target ADH1 were regenerated in media containing 30 mM allyl alcohol and compared to the regeneration rate of control plants. 118 GEiGS #3+SWAP11 allyl alcohol selected plants survived, compared to 51 control plants on allyl alcohol media (data not shown). Of the selected GEiGS #3+SWAP11, 5 were shown to harbour the DONOR (data not shown). The large amount of plants regenerating in the DONOR-treated plants, might be due to transient expression, during the bombardment process, as well.

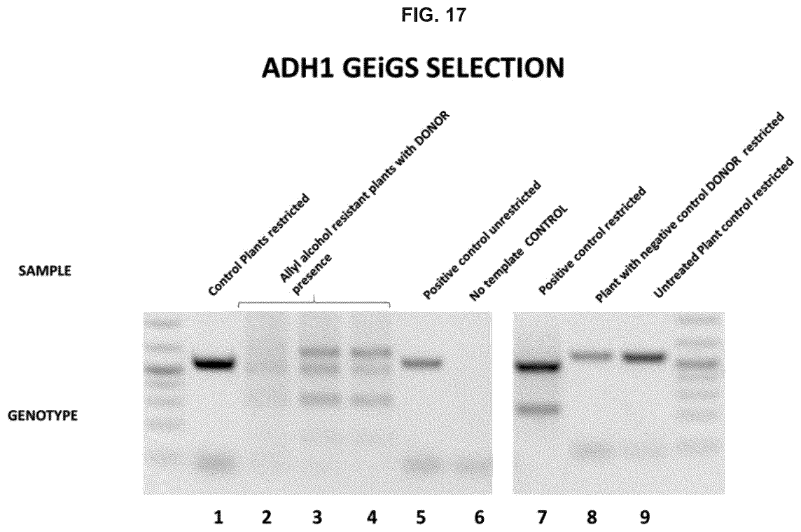

Thus, PDS3 and ADH1 selection through bleached phenotype (FIG. 16) and allyl alcohol selection (FIG. 17), respectively, give an ideal means for transformed plantlet selection for genotyping.

Swap region of 4 kb was assessed primarily through internal primers and specific amplicon differentiation of original wild type to insertion via restriction enzyme digestion variation.

ADH1 (FIG. 17) showed a comparative genotype of allyl alcohol selected plants with the expected DONOR presence restriction pattern when compared to restricted and non-restricted DONOR plasmid. PDS3 (FIG. 16) showed a comparison of bombarded samples phenotypes with and without DONOR and their respective differential restriction enzyme digestion patterns compared to that of restricted and non-restricted DONOR plasmid. These results provided a clear association of PDS3 albino/bleached phenotype to the expected restriction pattern. Subsequent external PCR combining specific internal, within the Swap region, in conjunction with external primer, outside and specific to the genomic region to swap into was carried out (data not shown). Further validation of the Swap was obtained through Sanger sequencing of the PCR amplicons, in order to assess heterozygous, homozygous, or presence of DONOR Swap (data not shown).

### Example 11

### Modified miRNA Reduce the Expression of their New Target Gene

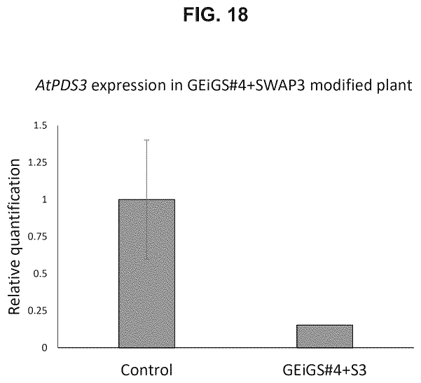

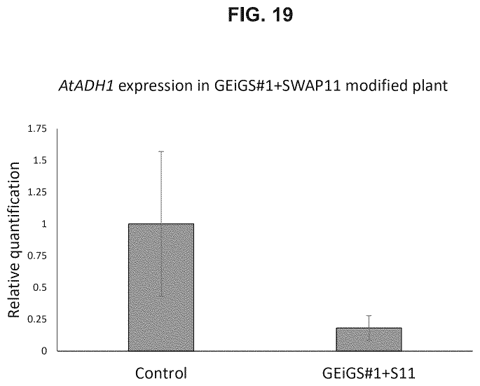

In order to verify the potential of the modified miRNAs in the GEiGS system to down regulate the expression of their newly designated targets, gene expression analysis was carried out using qRT-PCR (quantitative Real-Time PCR). RNA was extracted and reverse transcribed, from the positively identified regenerated plants and compared to regenerated plants, treated in parallel, but were not introduced with the relevant modifying constructs. In the case, where miR-173 was modified to target PDS3 (GEiGS #4+SWAP4), a reduction of 83% in the gene expression level, on average, was observed (FIG. 18). In plants with modified miR-390 to target ADH1 (GEiGS #3+SWAP11), a similar change in gene expression was observed, 82% of the levels in the control plants (FIG. 19). Taken together, these results substantiate the gene editing methods of modifying endogenous miRNAs to successfully target new genes and reduce their expression, by replacing the target recognition sequence in the miRNA transcript in the endogenous locus.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of modifying a gene encoding or processed into a non-coding RNA molecule having no RNA silencing activity in a plant cell, the method comprising introducing into the plant cell a DNA editing agent conferring a silencing specificity of said non-coding RNA molecule towards a target RNA of interest, thereby modifying the gene encoding or processed into the non-coding RNA molecule.

2. A method of modifying a gene encoding or processed into a RNA silencing molecule to a target RNA in a plant cell, the method comprising introducing into the plant cell a DNA editing agent which redirects a silencing specificity of said RNA silencing molecule towards a second target RNA, said target RNA and said second target RNA being distinct, thereby modifying the gene encoding the RNA silencing molecule.

3. (canceled)

4. The method of claim 2, wherein the gene encoding the RNA silencing molecule is endogenous to the plant cell.

5. The method of claim 2, wherein said modifying of said gene encoding or processed into said RNA silencing molecule comprises imparting said RNA silencing molecule with at least 45% complementarity towards said second target RNA.

6. (canceled)

7. The method of claim 2, wherein said silencing specificity of said RNA silencing molecule is determined:
i) by measuring a RNA or protein level of said second target RNA;
ii) phenotypically, optionally wherein said determined phenotypically is effected by determination of at least one plant phenotype selected from the group consisting of a plant leaf coloring, a flower coloring, a growth rate, a plant size, a crop yield, a fruit trait, a biotic stress resistance, and an abiotic stress resistance; and/or
iii) genotypically, optionally wherein a plant phenotype is determined prior to a plant genotype or a plant genotype is determined prior to a plant phenotype.

8-13. (canceled)

14. The method of claim 2, wherein said RNA silencing molecule is processed from a precursor, optionally wherein said RNA silencing molecule is a RNA interference (RNAi) molecule selected from the group consisting of a small interfering RNA (siRNA), a short hairpin RNA (shRNA), a microRNA (miRNA), a Piwi-interacting RNA (piRNA) and trans-acting siRNA (tasiRNA).

15. (canceled)

16. The method of claim 1, wherein said non-coding RNA molecule is selected from the group consisting of a small nuclear RNA (snRNA), a small nucleolar RNA (snoRNA), a long non-coding RNA (lncRNA), a ribosomal RNA (rRNA), transfer RNA (tRNA), a repeat-derived RNA, and a transposable element RNA, optionally wherein said RNAi molecule is designed such that a sequence of said RNAi molecule is modified to preserve originality of structure and to be recognized by cellular RNAi factors.

17. (canceled)

18. The method of claim 2, wherein said modifying of said gene is effected by a modification selected from the group consisting of a deletion, an insertion, a point mutation and a combination thereof, and/or wherein said modification:
i) is in a stem region of said RNA silencing molecule;
ii) is in a loop region of said RNA silencing molecule;
iii) is in a non-structured region of said RNA silencing molecule;
iv) is in a stem region and a loop region of said RNA silencing molecule;
v) is in a stem region and a loop region and in non-structured region of said RNA silencing molecule; and/or
vi) comprises a modification of at most 200 nucleotides.

19-24. (canceled)

25. The method of claim 2, wherein said method further comprises introducing into the plant cell donor oligonucleotides.

26. The method of claim 2, wherein said DNA editing agent comprises at least one gRNA operatively linked to a plant expressible promoter.

27. The method of claim 2, wherein said DNA editing agent does not comprise an endonuclease, or wherein said DNA editing agent comprises an endonuclease, and optionally wherein said endonuclease comprises Cas9.

28. (canceled)

29. The method of claim 2, wherein said DNA editing agent is of a DNA editing system selected from the group consisting of a meganuclease, a zinc finger nucleases (ZFN), a transcription-activator like effector nuclease (TALEN) and CRISPR, optionally wherein said DNA editing agent is applied to the cell as DNA, RNA or RNP.

30-31. (canceled)

32. The method of claim 2, wherein said DNA editing agent is linked to a reporter for monitoring expression in a plant cell, optionally wherein said reporter is a fluorescent protein.

33. (canceled)

34. The method of claim 2, wherein said second target RNA is endogenous to the plant cell, or wherein said second target RNA is exogenous to the plant cell.

35-36. (canceled)

37. A plant or plant cell generated according to the method of claim 2.

38. (canceled)

39. A method of producing a plant with reduced expression of a target gene, the method comprising:
(a) breeding a plant comprising the plant cell of claim 37, optionally wherein said breeding comprises crossing or selfing; and
(b) selecting for progeny plants that have reduced expression of said second target RNA, and which do not comprise said DNA editing agent,
thereby producing said plant with reduced expression of a target gene.

40. (canceled)

41. A method of generating a plant with a phenotype selected from the group consisting of increased stress tolerance, increased yield, increased growth rate, increased yield quality, pathogen tolerance, pathogen resistance, pest tolerance, pest resistance, and herbicide resistance, the method comprising modifying a gene encoding or processed into the RNA silencing molecule in a plant cell according to claim 2, wherein said second target RNA is of a gene selected from the group consisting of:
(i) a gene of the plant conferring sensitivity to stress, decreased yield, decreased growth rate decreased yield quality, sensitivity to said pathogen, sensitivity to said pest, or sensitivity to said herbicide;
(ii) a gene of the pathogen; and
(iii) a gene of the pest;
thereby generating the plant.

42-46. (canceled)

47. A plant or plant seed generated according to the method of claim 41, optionally wherein said plant is non-genetically modified (non-GMO) and/or wherein said plant is selected from the group consisting of a crop, a flower and a tree.

48-50. (canceled)

51. The method of claim 2, wherein:
i) following introducing the DNA editing agent into the plant cell, the plant cell is heterozygous for the modification; or
ii) the DNA editing agent generates two or more double stranded DNA breaks in the gene encoding the RNA silencing molecule.

52. A modified plant cell which is homozygous or heterozygous for a DNA editing event, wherein said DNA editing event is the modification of a gene encoding or processed into a RNA silencing molecule to a target RNA in said plant cell, using a method comprising introducing into the plant cell a DNA editing agent to generate the modification, wherein the modification redirects a silencing specificity of said RNA silencing molecule towards a second target RNA, said target RNA and said second target RNA being distinct.
